(19) United States
(12) Patent Application Publication
(10) Pub. No.: US 2026/0265246 A1
(43) Pub. Date: Sep. 10, 2026

(54) HETEROCYCLIC COMPOUNDS AS 5HT2A BIASED AGONISTS

(71) Applicants: Jain MANISH, Chapel Hill, NC (US); Samuel SLOCUM, Chapel Hill, NC (US); Georgios SKINIOTIS, Stanford, CA (US); Ximena BARROS, Stanford, CA (US); ICAHN SCHOOL OF MEDICINE AT MOUNT SINAI, New York, NY (US); THE UNIVERSITY OF NORTH CAROLINA AT CHAPEL HILL, Chapel Hill, NC (US); THE BOARD OF TRUSTEES OF THE LELAND STANFORD JUNIOR UNIVERSITY, Stanford, CA (US); THE REGENTS OF THE UNIVERSITY OF CALIFORNIA, Oakland, CA (US)

(72) Inventors: Manish Jain, Chapel Hill, NC (US); Samuel Slocum, Chapel Hill, NC (US); Georgios Skiniotis, Stanford, CA (US); Ximena Barros Alvarez, Stanford, CA (US); Jian Jin, New York, NY (US); H. Umit Kaniskan, New York, NY (US); Ning Sun, New York, NY (US); Renhong Sun, New York, NY (US); Yan Xiong, New York, NY (US); Yudao Shen, New York, NY (US); Zhongli Xu, New York, NY (US); Xing Qiu, New York, NY (US); Chao Qian, New York, NY (US); Xiangyang Song, New York, NY (US); Zhijie Deng, New York, NY (US); Bryan Roth, Chapel Hill, NC (US); Jeffrey Diberto, Chapel Hill, NC (US); Kuglae Kim, Chapel Hill, NC (US); Carl-Mikael Suomivuori, Stanford, CA (US); Marc A. Daemgen, Stanford, CA (US); Ron Dror, Stanford, CA (US); Brian Shoichet, Oakland, CA (US); Anat Levit, Oakland, CA (US)

(21) Appl. No.: 18/720,506

(22) PCT Filed: Dec. 16, 2022
(86) PCT No.: PCT/US2022/053168

§ 371 Date: Jun. 14, 2024

Related U.S. Application Data
(60) Provisional application No. 63/290,602, filed on Dec. 16, 2021.
(60) Provisional application No. 63/407,529, filed on Sep. 16, 2022.

(51) Int. Cl. C07D 471/04; A61K 31/416; A61K 31/4355; A61K 31/437; A61K 31/439; A61K 31/4439; A61K 31/454; A61K 31/46; A61K 31/496; A61K 31/506; A61K 31/5377; A61K 31/55; C07B 59/00; C07D 209/14; C07D 231/56; C07D 401/04; C07D 403/14; C07D 405/14; C07D 451/02; C07D 487/08

(52) U.S. Cl. CPC C07D 471/04; A61K 31/416; A61K 31/4355; A61K 31/437; A61K 31/439; A61K 31/4439; A61K 31/454; A61K 31/46; A61K 31/496; A61K 31/506; A61K 31/5377; A61K 31/55; C07B 59/002; C07D 209/14; C07D 231/56; C07D 401/04; C07D 403/14; C07D 405/14; C07D 451/02; C07D 487/08

(57) ABSTRACT

Disclosed are compounds that are 5HT2A Gq-biased agonists and methods for use of such compounds in 5HT2A mediated diseases.

### STATEMENT REGARDING GOVERNMENT FUNDING

This invention was made with government support under HR00112020029 awarded by the Defense Advanced Research Projects Agency. The government has certain rights in the invention.

### TECHNICAL FIELD

This disclosure relates to small-molecule heterocyclic compounds, which are Gq-biased agonists of the G protein-coupled receptor 5HT2A. This disclosure also pertains to methods of use thereof for the treatment of diseases in a subject in need thereof.

### BACKGROUND

Depression, anxiety and substance abuse represent major unsolved medical problems, as safe, effective and rapid treatments are currently unavailable. Commonly prescribed antidepressant medications frequently take weeks-months for full efficacy and many patients do not achieve symptom relief and remain disabled. Therefore, new treatments are needed.

The 5HT2A receptor, a G protein-coupled receptor, is a target of much interest, owing to its role in psychiatric disorders including psychosis, depression, dyskinesias, and hallucination (Slocum et al., 2021). Progress towards therapeutic leads against this target, has been slowed by the need for selectivity versus related off-targets, such as 5HT2B receptor (Hutcheson et al., 2011; McCorvy and Roth, 2015; Roth, 2007), versus other receptors such as the serotonin transporter SERT, and for functional selectivity in signaling (i.e., for G protein vs. β-arrestin recruitment)(Kim et al., 2020; Slocum et al., 2021); these features make 5HT2A a therapeutically worthy yet challenging target. Gq-biased 5HT2A agonists, which selectively activate 5HT2A-mediated Gq signaling over β-arrestin recruitment, are highly sought-after therapeutics. To date, Gq-biased 5HT2A agonists that are selective for 5HT2A over 5HT2B and SERT are unprecedented.

### SUMMARY

Disclosed are heterocyclic small-molecules heterocyclic compounds, which are Gq-biased agonists of 5HT2A and are selective for 5HT2A over 5HT2B and 5HT2C. These compounds are effective therapeutics for the treatment of psychiatric disorders such as depression, anxiety and substance abuse.

In some embodiments, provided herein are compounds having the structure of FORMULA 1, or a pharmaceutically acceptable salt or solvate thereof:

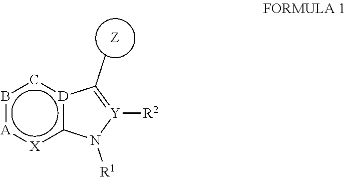

A is selected from N, CH or CR⁶;
B is selected from N, CH or CR⁵;
C is selected from N, CH or CR⁴;
D is selected from N or C;
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R²³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²³ and R²⁴, R²³ and R²⁵, R²⁴ and R²⁵ together with the atom to which they are connected form an optionally substituted 3-8 membered cycloalkyl or heterocyclyl ring; and
\textcircled{z} is at each occurrence independently selected from an optionally substituted 3-10 membered carbocyclyl, optionally substituted 3-10 membered heterocyclyl, optionally substituted 4-13 membered fused carbocyclyl, optionally substituted 4-13 membered fused heterocyclyl, optionally substituted 4-13 membered bridged carbocyclyl, optionally substituted 4-13 membered bridged heterocyclyl, optionally substituted 4-13 membered spiro carbocyclyl, optionally substituted 4-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl.

In an embodiment, \textcircled{z} at each occurrence can be selected from the following groups, or their optionally substituted analogs, wherein * denotes the attachment:

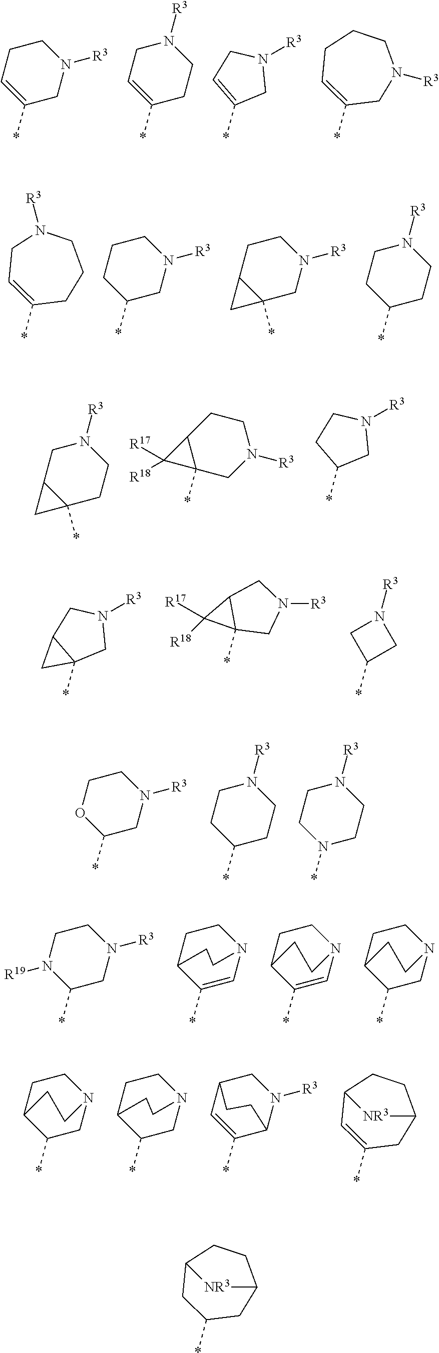

wherein,
R³ at each occurrence, are independently selected from hydrogen, methyl, ethyl, n-propyl, C₁-C₈alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R²⁶ and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²⁶ and R²⁷, together with the atom to which they are connected form an optionally substituted 3-8 membered cycloalkyl or heterocyclyl ring;
R¹⁷ and R¹⁸ at each occurrence, are independently selected from hydrogen, halogen, C₁-C₈ alkyl, oxo, Ph, CN, NO₂, OR²⁸, SR²⁸, NR²⁸R²⁹, C(O)R²⁸, C(O)OR²⁹, C(O)NR²⁸R²⁹, S(O)R²⁸, S(O)₂R²⁸, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³¹S(O)₂NR²⁸R²⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C3-C8 cycloalkoxy, optionally substituted 3-8 membered heterocyclyl, optionally substituted aryl, and optionally substituted heteroaryl;
wherein;
   R²⁸, R²⁹, and R³⁰ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²⁸ and R²⁹, R²⁸ and R³⁰, R²⁹ and R³⁰ together with the atom to which they are connected form an optionally substituted 3-20 membered cycloalkyl or heterocyclyl ring; and pharmaceutically acceptable salts thereof.

In another embodiment, the 5HT2A agonist is a compound of FORMULA 2:

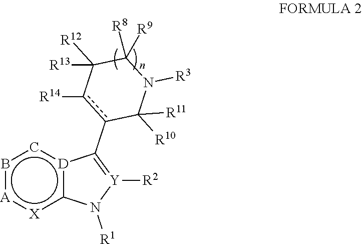

wherein;
definitions of A, B, C, D, X, Y, R¹, R² and R³ are the same as for FORMULA 1
n is selected from 0, 1 or 2;
R⁸, R⁹, R¹⁰, R¹¹, R¹², R¹³ and R¹⁴, at each occurrence, are independently selected from hydrogen, halogen, C₁-C₈ alkyl, oxo, Ph, CN, NO₂, OR³¹, SR³¹, NR³¹R³², C(O)R³, C(O)OR³, C(O)NR³¹R³², S(O)R³, S(O)₂R³, S(O)₂NR³¹R³², NR³³C(O)OR³, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R³¹ and R³², R³¹ and R³³, R³² and R³³ together with the atom to which they are connected form an optionally substituted 3-8 membered cycloalkyl or heterocyclyl ring;
and pharmaceutically acceptable salts thereof.

In another embodiment, the 5HT2A agonist is a compound of FORMULAE 2A or 2B:

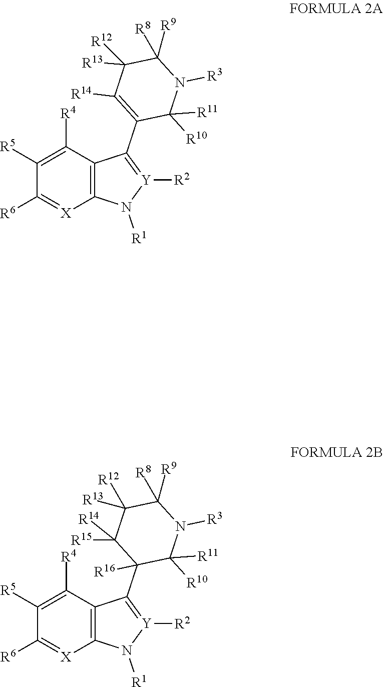

wherein;
X is selected from N, CH or CR⁷;
Y is selected from N or C;
the definitions of R¹, R², R³, R⁴, R⁵, R⁶, R⁷ are the same as for FORMULA 1;
and definitions of R⁸, R⁹, R¹⁰, R¹¹, R¹², R¹³ and R¹⁴ are the same as for FORMULA 2;
R¹⁵ and R¹⁶, at each occurrence, are independently selected from hydrogen, halogen, C₁-C₈ alkyl, oxo, Ph, CN, NO₂, OR³⁴, SR³⁴, NR³⁴R³⁵, C(O)R³⁴, C(O)OR³⁴, C(O)NR³⁴R³⁵, S(O)R³⁴, S(O)₂R³⁴, S(O)₂NR³⁴R³⁵, NR³⁶C(O)OR³⁴, NR³⁶C(O)R³⁴, NR³⁶C(O)NR³⁴R³⁵, NR³⁶S(O)R³⁴, NR³⁶S(O)₂R³⁴, NR³⁶S(O)₂NR³⁴R³⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R³⁴, R³⁵, and R³⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R³⁴ and R³⁵, R³⁴ and R³⁶, R³⁵ and R³⁶ together with the atom to which they are connected form an optionally substituted 3-8 membered cycloalkyl or heterocyclyl ring; and
optionally, R¹⁵ and R¹⁶ together with the atom to which they are connected form an optionally substituted 3-20 membered cycloalkyl or heterocyclyl ring; and
pharmaceutically acceptable salts thereof.

In another embodiment, the 5HT2A agonist is a compound of FORMULAE 2C or 2D:

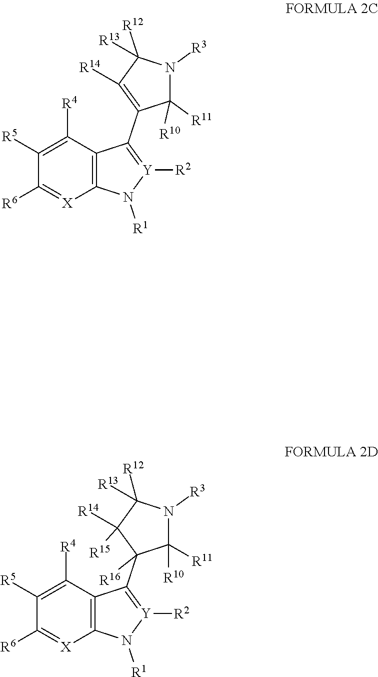

wherein,
X is selected from N, CH or CR⁷
Y is selected from N or C;
The definitions of R¹, R², R³, R⁴, R⁵, R⁶, R⁷, R⁸, R¹¹, R¹², R¹³, R¹⁴, R¹⁵ an R¹⁶ are the same as for FORMULAE 2A and 2B;
and pharmaceutically acceptable salts thereof.

In another embodiment, the 5HT2A agonist is a compound of FORMULAE 2E or 2F:

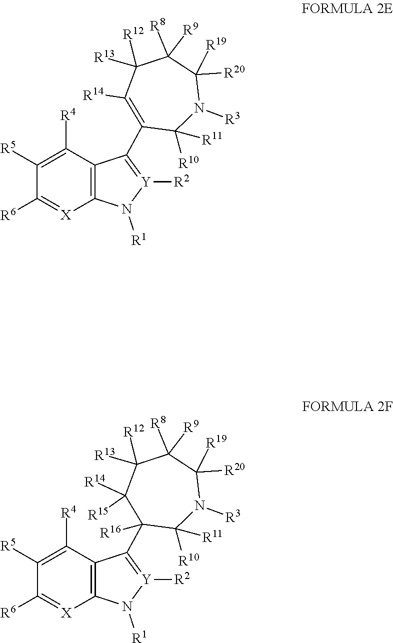

wherein,
X is selected from N, CH or CR⁷;
Y is selected from N or C;
The definitions of R, RZ, R³, R⁴, R¹, R⁶, R⁷, R⁸, R¹¹, R¹², R¹³, R¹⁴, R¹⁵ and R¹⁶ are the same as for FORMULAE 2A and 2B;
R¹⁹ and R²⁰, at each occurrence, are independently selected from hydrogen, halogen, C₁-C₈ alkyl, oxo, Ph, CN, NO₂, OR³⁷, SR³⁷, NR³⁷R³⁸, C(O)R³⁷, C(O)OR³⁸, C(O)NR³⁷R³⁸, S(O)R³⁷, S(O)₂R³⁷, S(O)₂NR³⁷R³⁸, NR³⁹C(O)OR³⁷, NR³⁹C(O)R³⁷, NR³⁹C(O)NR³⁷R³⁸, NR³⁹S(O)R³⁷, NR³⁹S(O)₂R³⁷, NR³⁶S(O)₂NR³⁷R³⁸, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R³⁷, R³⁸, and R³⁹ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R³⁷ and R³⁸, R³⁷ and R³⁹, R³⁸ and R³⁹ together with the atom to which they are connected form an optionally substituted 3-8 membered cycloalkyl or heterocyclyl ring; and
pharmaceutically acceptable salts thereof.

In another embodiment, the 5HT2A agonist is a compound of FORMULA 3:

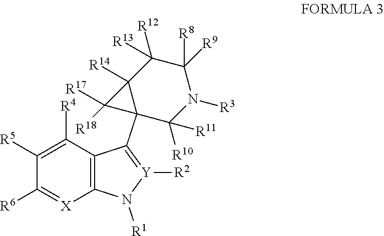

wherein;
X is selected from N, CH or CR⁷;
Y is selected from N or C;
The definitions of R¹, R², R³, R⁴, R⁵, R⁶, R⁷, R¹⁷ and R¹⁸ are the same as for FORMULA 1;
and definitions of R⁸, R⁹, R¹⁰, R¹¹, R¹², R¹³ and R¹⁴ are the same as for FORMULA 2A; and
pharmaceutically acceptable salts thereof.

In another embodiment, the 5HT2A agonist is a compound of FORMULA 4:

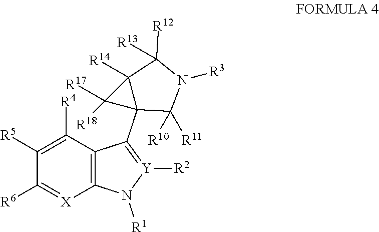

wherein;
X is selected from N, CH or CR⁷;
Y is selected from N or C;
The definitions of R¹, R², R³, R⁴, R⁵, R⁶, R⁷, R¹⁷ and R¹⁸ are the same as for FORMULA 1;
And definitions of R¹⁰, R¹¹, R¹², R¹³ and R¹⁴ are the same as for FORMULA 2A;
and pharmaceutically acceptable salts thereof.

In another embodiment, the 5HT2A agonist is a compound of FORMULA 5:

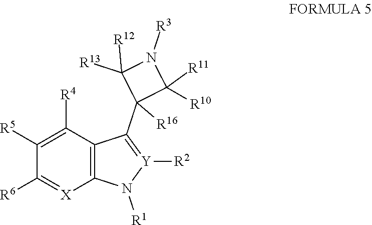

wherein;
X is selected from N, CH or CR⁷;
Y is selected from N or C;
the definitions of R¹, R², R³, R⁴, R⁵, R⁶, R⁷ are the same as for FORMULA 1;
and definitions of R¹⁰, R¹¹, R¹², R¹³ and R¹⁶ are the same as for FORMULA 2A; and
pharmaceutically acceptable salts thereof.

In some aspects, the compound is selected from the following: NS131-179, NS131-178, NS131-177, NS136-006, NS131-169, NS131-168, NS131-167, NS131-173, NS131-180, RS134-52, RS134-48, NS131-185, NS131-170, RS134-45, RS134-40, NS131-184, RS134-49, RS134-53, RS134-41, RS134-46, NS131-172, RS134-38, RS134-65, RS134-62, RS134-70, NS136-081, RS134-73, RS134-72, NS136-092, NS136-091, NS136-096, NS136-095, NS136-102, NS136-101, NS136-115, NS136-116, NS136-117, NS136-118, NS136-119, NS136-120, NS136-109, NS136-110, NS136-111, NS136-112, RS134-37, RS134-56, NS136-002, NS136-004, RS130-132, YX129-177C, YX129-180C, YX143-19, YX143-20, YX143-2, YX143-21, NS144-042, NS144-043, NS144-044, YS135-44, YS135-45, YS135-34, YS135-32, YS135-38, YS135-41, YS135-39, YX143-14A-2, NS144-019, NS144-021, YX143-15, YX143-16, YX143-17C, YX143-18C, NS144-047, NS144-048, NS144-049, NS136-128, NS136-129, NS136-130, NS136-131, NS136-150, NS136-151, NS136-152, NS136-166, NS144-011, NS136-158, NS136-167, NS136-159, NS136-135, NS136-136, NS136-137, NS144-046, NS144-045, NS136-140, NS136-141, NS136-142, NS136-143, NS136-153, NS136-154, NS136-155, NS136-175, NS144-016, NS136-160, NS136-176, NS136-161, NS136-144, NS136-145, NS136-146, NS144-051, NS144-050, YX143-41C, YX143-42C, YX143-43D, NS144-059-2, NS144-054-2, NS144-067, NS144-085, NS144-093, NS144-094, NS144-095, NS144-096, XQ148-012, XQ148-023, ZX147-015, ZX147-016, ZX147-017, ZX147-019, NS144-097, NS144-098, NS144-102, NS144-101, NS144-107, NS144-108, NS144-109, NS144-110, YS135-52, YS135-53, YS135-54, YS135-80, YS135-81, YS135-82, YS135-96, YS135-98, YS135-99, YS135-100, ZX147-026-01, ZX147-026-02, ZX147-027, ZX147-028, ZX147-029, ZX147-031, ZX147-054, ZX147-055, ZX147-056, ZX147-092, ZX147-093, ZX147-094, ZX147-095, ZX147-096, ZX147-097, ZX147-098, ZX147-099, ZX147-100, ZX147-128, ZX147-129, ZX147-130, ZX147-131, ZX147-137, ZX147-183, ZX156-011, ZX156-012, ZX156-014-1, ZX156-014-2, ZX156-019, ZX156-059, ZX156-069, ZX156-070, ZX156-071, ZX156-089, ZX156-090, ZX162-100, ZX162-031, ZX162-104, ZX162-105, ZX162-110, ZX162-111, ZX162-112, ZX162-113, ZX162-124, ZX162-126, ZX162-127, ZX162-128, ZX162-129, ZX162-138, ZX162-139, ZX162-140, ZX162-141, ZX162-147, ZX162-148, ZX162-151, ZX162-173, ZX162-174, ZX162-175, ZX162-176, YX143-103B, YX143-103C, YX143-105C, YX143-108, YX143-110B, YX143-112B, YX143-129, YX143-134C, YX143-138C, YX143-182C-1, YX143-183A, YX143-184B-1, YX143-184B-2, YX143-185B, YX143-186B, YX157-19A, YX157-20A, YX157-29B, YX157-42B, YX157-51B, YX157-51C, YX157-55A, XS159-153, XS159-155, XS159-160, XS159-163, XS159-180, XS159-186, XS165-3, XS165-5, XS165-8, XQ148-93, XQ158-012, XQ158-055, XQ158-056, XQ158-078, XQ158-093A, XQ158-082, XQ158-115, XQ158-164, XQ158-167, XQ158-168, ZD160-34, ZD160-140, ZD160-141, ZD160-149, ZD160-11, ZD160-133, ZD160-130, ZD160-131, QC166-005, QC166-008, QC-166-032, XQ148-86, QC166-096, QC166-097, QC179-001, QC179-002, QC179-025, QC-179-032, QC-179-033, QC179-038, QC179-039, QC179-040, ZX167-072, ZX167-077, ZX167-074, ZX167-090, ZX167-091, ZX162-100-1 (Enantiomer 1 of ZX162-100), ZX162-100-2 (Enantiomer 2 of ZX162-100), ZX162-031-1 (Enantiomer 1 of ZX162-031), ZX162-031-2 (Enantiomer 2 of ZX162-031), ZX167-074-1 (Enantiomer 1 of ZX167-074), ZX167-074-2 (Enantiomer 2 of ZX167-074), ZX177-057, ZX177-058, ZX177-058BY, ZX177-059, ZX177-060 and analogs thereof.

In some aspects, the compound is selected from the following: NS131-179, NS131-178, NS131-177, NS136-006, NS131-169, NS131-168, NS131-167, NS131-173, NS131-180, RS134-52, RS134-48, NS131-185, NS131-170, RS134-45, RS134-40, NS131-184, RS134-49, RS134-53, RS134-41, RS134-46, NS131-172, RS134-38, RS134-65, RS134-62, RS134-70, NS136-081, RS134-73, RS134-72, NS136-092, NS136-091, NS136-096, NS136-095, NS136-102, NS136-101, NS136-115, NS136-116, NS136-117, NS136-118, NS136-119, NS136-120, RS134-37, RS134-56, NS136-002, NS136-004, YS135-34, YS135-32, YS135-38, YS135-41, YS135-39, YX143-14A-2, NS144-019, NS144-021, YX143-15, YX143-16, YX143-17C, YX143-18C, NS144-047, NS144-048, NS144-049, NS136-128, NS136-129, NS136-130, NS136-131, NS136-150, NS136-151, NS136-152, NS136-166, NS144-011, NS136-158, NS136-167, NS136-159, NS136-140, NS136-141, NS136-142, NS136-143, NS136-153, NS136-154, NS136-155, NS136-175, NS144-016, NS136-160, NS136-176, NS136-161, NS144-093, NS144-094, NS144-095, NS144-096, YS135-80, YS135-81, YS135-82, YS135-96, YS135-98, ZX156-069, and analogs thereof.

In some aspects, the compound is selected from the following: RS130-132, YX129-177C, YX129-180C, YX143-19, YX143-20, YX143-2, YX143-21, NS144-042, NS144-043, NS144-044, YS135-44, YS135-45, NS136-135, NS136-136, NS136-137, NS144-046, NS144-045, NS136-144, NS136-145, NS136-146, NS144-051, NS144-050, YX143-41C, YX143-42C, YX143-43D, NS144-059-2, NS144-054-2, NS144-067, NS144-085, XQ148-012, XQ148-023, ZX147-015, ZX147-016, ZX147-017, ZX147-019, NS144-097, NS144-098, NS144-102, NS144-101, NS144-107, NS144-108, NS144-109, NS144-110, YS135-52, YS135-53, YS135-54, YS135-99, YS135-100, ZX147-026-01, ZX147-026-02, ZX147-027, ZX147-028, ZX147-029, ZX147-054, ZX147-055, ZX147-056, ZX147-092, ZX147-093, ZX147-094, ZX147-095, ZX147-096, ZX147-097, ZX147-098, ZX147-099, ZX147-100, ZX147-128, ZX147-129, ZX147-130, ZX147-137, ZX147-183, ZX156-019, ZX156-059, ZX156-070, ZX156-071, ZX156-089, ZX156-090, XQ148-93, XQ158-012, XQ158-055, XQ158-056, XQ148-86, and analogs thereof.

In some aspects, the compound is selected from the following: YX143-103B, YX143-103C, YX143-105C, YX143-108, YX143-110B, YX143-112B, YX143-129, YX143-134C, YX143-138C, YX143-182C-1, YX143-183A, YX143-184B-1, YX143-184B-2, YX143-185B, YX143-186B, YX157-19A, YX157-20A, YX157-29B, YX157-42B, YX157-51B, YX157-51C, YX157-55A, and analogs thereof.

In some aspects, the compound is selected from the following: XS159-180, XS159-186, XS165-3, XS165-5, XS165-8, XQ158-078, XQ158-093A, XQ158-082, XQ158-115, XQ158-164, XQ158-167, XQ158-168, ZD160-34, ZD160-140, ZD160-141, ZD160-149, ZD160-11, ZD160-133, ZD160-130, ZD160-131, and analogs thereof.

In some aspects, the compound is selected from the following: QC166-005, QC166-008, QC-166-032, QC166-096, QC166-097, QC179-001, QC179-002, QC179-025, QC-179-032, QC-179-033, QC179-038, QC179-039, QC179-040, and analogs thereof.

In some aspects, the compound is selected from the following: ZX162-100, ZX162-031, ZX162-104, ZX162-105, ZX162-110, ZX162-111, ZX162-112, ZX162-113, ZX162-124, ZX162-126, ZX162-127, ZX162-128, ZX162-129, ZX162-138, ZX162-139, ZX162-140, ZX162-141, ZX162-147, ZX162-148, ZX162-151, ZX162-173, ZX162-174, ZX162-175, ZX162-176, XS159-153, XS159-155, XS159-160, XS159-163, ZX167-072, ZX167-077, ZX167-074, ZX167-090, ZX167-091, ZX162-100-1 (Enantiomer 1 of ZX162-100), ZX162-100-2 (Enantiomer2 of ZX162-100), ZX162-031-1 (Enantiomer 1 of ZX162-031), ZX162-031-2 (Enantiomer 2 of ZX162-031), ZX167-074-1 (Enantiomer 1 of ZX167-074), ZX167-074-2 (Enantiomer 2 of ZX167-074), ZX177-057, ZX177-058, ZX177-058BY, ZX177-059, ZX177-060 and analogs thereof.

In some aspects, the compound is selected from the following: NS136-109, NS136-110, NS136-111, NS136-112, NS136-145, RS134-40, RS134-45, RS134-48, RS134-46, YX143-19 and analogs thereof.

In some aspects, the compound is selected from the following: YX143-108, YX143-129, YX143-134C, ZX147-031, ZX147-131, ZX162-031, ZX162-031-1, ZX162-100, ZX162-100-2, ZX162-105, ZX167-074, ZX167-091, QC166-008, QC166-096, QC166-097, and analogs thereof.

In some aspects, the compound is selected from the following: ZX162-031, ZX162-031-1, ZX162-100, ZX162-100-2, ZX162-105, ZX167-074, ZX167-091, QC166-008, QC166-096, QC166-097, and analogs thereof.

In some aspects, the compound is selected from the following: 3-(3-azabicyclo[4.1.0]heptan-1-yl)-7-chloro-1H-indazole (ZX162-031); 3-(3-azabicyclo[3.1.0]hexan-1-yl)-7-chloro-1H-indazole (ZX162-100); 3-(3-azabicyclo[3.1.0]hexan-1-yl)-7-chloro-1H-indole (ZX167-074); and 3-(3-azabicyclo[3.1.0]hexan-1-yl)-7-methyl-1H-indole (ZX167-091), including its pure enantiomers, mixtures of enantiomers, and pharmaceutically acceptable salts, solvent complexes, morphological forms, or deuterated and fluorinated derivatives thereof.

In some aspects, the compound is selected from the following:
3-(azetidin-3-yl)-7-chloro-1H-indole (QC166-008);
3-(azetidin-3-yl)-7-methyl-1H-indole (QC166-096); and
3-(azetidin-3-yl)-7-fluoro-1H-indole (QC166-097),
   and pharmaceutically acceptable salts, solvent complexes, morphological forms, or deuterated and fluorinated derivatives thereof.

In an embodiment, the disclosure includes a pharmaceutical composition, including a 5HT2A agonist as disclosed above, and pharmaceutically acceptable carrier.

In an embodiment, the pharmaceutical composition is formulated to be administered orally, parenterally, intradermally, subcutaneously, topically, and/or rectally.

In an embodiment, disclosed is a method of treating a psychiatric disorder, including administering to a subject in need thereof, a 5HT2A agonist as disclosed above.

In an embodiment, the psychiatric disorder is depression, anxiety, psychosis, dyskinesias, hallucination or substance abuse.

The materials, methods, and examples are illustrative only and not intended to be limiting.

All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1A-1C. Bioluminescence Resonance Energy Transfer Assays. A) The BRET2-based TRUPATH platform is used to measure the dissociation of the Gαq subunit from its cognate Gβγ dimer. The RLuc and GFP2 components provide a BRET signal (GFP2/RLuc) that is highest when the heterotrimer is intact in the absence of drug or receptor-mediated dissociation, and decreases upon dissociation in a concentration-dependent manner. B) The BRET1-based beta-arrestin2 recruitment is used to measure the recruitment of beta-arrestin2 to 5HT2A receptor C-terminally tagged with RLuc. Here, the RLuc and mVenys provide a BRET signal (mVenus/RLuc) that is lowest in the absence of drug or recruitment, and increases upon recruitment to the receptor in a concentration-dependent manner. C) Plotting of the data in a semi-logarithmic fashion allows determination of the efficacy (Emax) and potency (EC50).

FIG. 2A-2C. BRET Data from Select Compounds. 5HT2A receptor displaying biased signaling towards Gαq (blue) versus beta-arrestin2 (red) signaling in response to novel compounds as measured in the BRET assays. Biased signaling is represented by either preferential efficacy, potency, or both through the G protein over beta-arrestin2 pathway.

FIG. 3. Calcium Mobilization Assays. The calcium flux assays depend upon conical Gαq-mediated signaling, in which the Gαq subunit activates phospholipase C, which hydrolyzes phosphatidylinositol 4,5-bisphosphate into diacyclygerol (not shown) and inositol 1,4,5-triphosphate (IP3). IP3 activates IP3-gated channels on the endoplasmic reticulum, gating the release of intracellular calcium. This calcium then binds to the Fluo-4 dye to produce a signal, with the extent of calcium release—and thus signal produced—increasing in a concentration-dependent manner.

FIG. 4A-4C. Calcium Mobilization Data from Select Compounds. Comparison of compound-induced calcium flux at 5HT2A, 5HT2B, and 5HT2C, with a subset of compounds displaying selective activation 5HT2A alone.

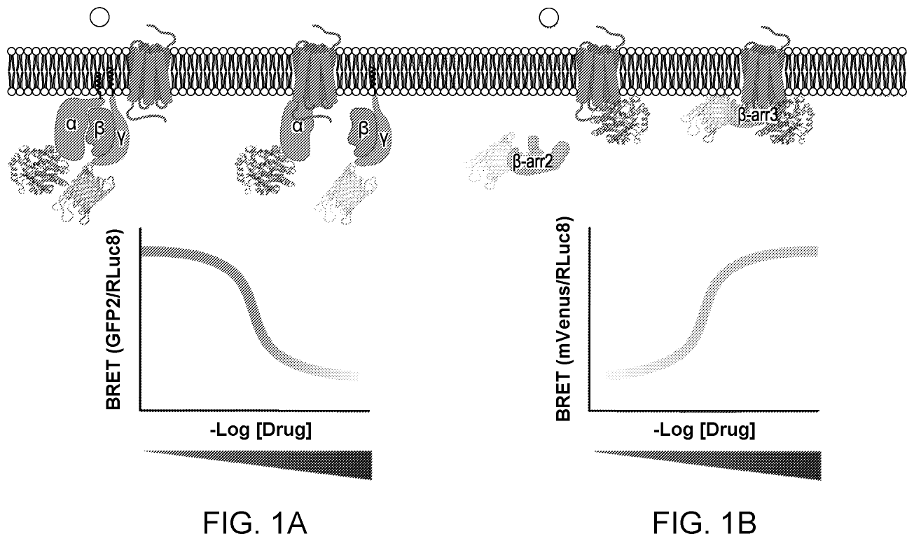

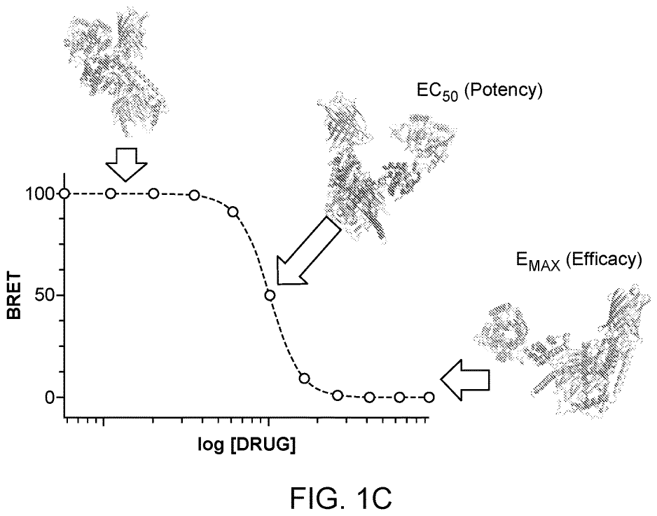

### DETAILED DESCRIPTION

In an embodiment, 5HT2A agonist is a compound of FORMULA 1:

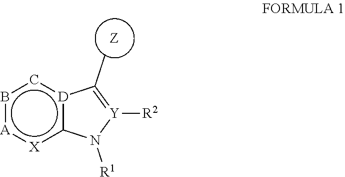

A is selected from N, CH or CR⁶;
B is selected from N, CH or CR⁵;
C is selected from N, CH or CR⁴;
D is selected from N or C;
X is selected from N, CH or CR⁷;
Y is selected from N or C;
R¹, R² at each occurrence, are independently selected from hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R²³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²³ and R²⁴, R²³ and R²⁵, R²⁴ and R²⁵ together with the atom to which they are connected form an optionally substituted 3-8 membered cycloalkyl or heterocyclyl ring; and
\textcircled{z} is at each occurrence independently selected from an optionally substituted 3-10 membered carbocyclyl, optionally substituted 3-10 membered heterocyclyl, optionally substituted 4-13 membered fused carbocyclyl, optionally substituted 4-13 membered fused heterocyclyl, optionally substituted 4-13 membered bridged carbocyclyl, optionally substituted 4-13 membered bridged heterocyclyl, optionally substituted 4-13 membered spiro carbocyclyl, optionally substituted 4-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl.

In an embodiment, \textcircled{z} at each occurrence can be selected from the following groups, or their optionally substituted analogs, wherein * denotes the attachment:

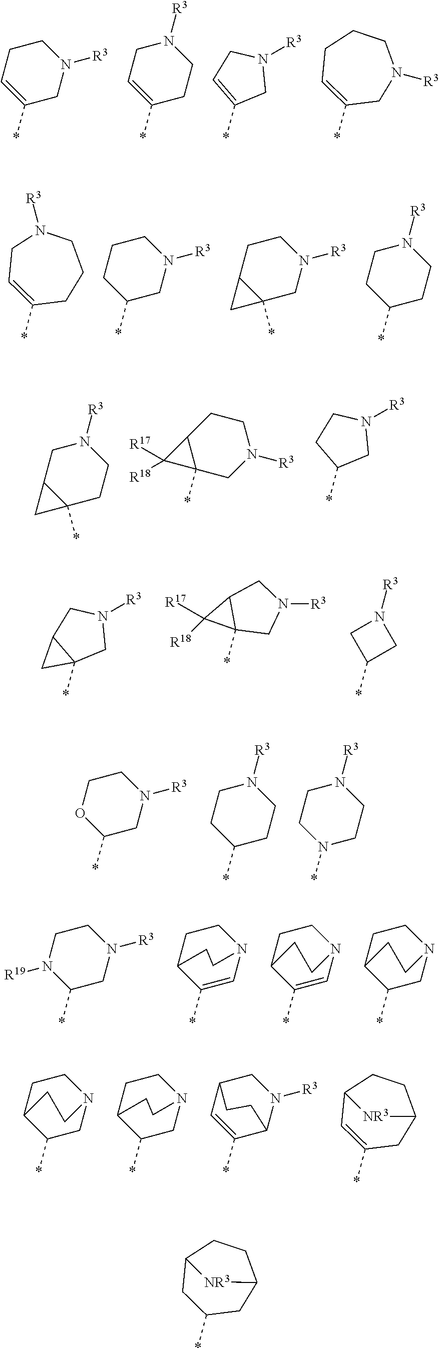

wherein,
R³ at each occurrence, are independently selected from hydrogen, methyl, ethyl, n-propyl, C₁-C₈ alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R²⁶ and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²⁶ and R²⁷, together with the atom to which they are connected form an optionally substituted 3-8 membered cycloalkyl or heterocyclyl ring;
R¹⁷ and R¹⁸ at each occurrence, are independently selected from hydrogen, halogen, C₁-C₈ alkyl, oxo, Ph, CN, NO₂, OR²⁸, SR²⁸, NR²⁸R²⁹, C(O)R²⁸, C(O)OR²⁹, C(O)NR²⁸R²⁹, S(O)R²⁸, S(O)₂R²⁸, S(O)₂NR²⁸R²⁹, NR³⁰C(O)OR²⁸, NR³⁰C(O)R²⁸, NR³⁰C(O)NR²⁸R²⁹, NR³¹S(O)R²⁸, NR³⁰S(O)₂R²⁸, NR³¹S(O)₂NR²⁸R²⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein;
   R²⁸, R²⁹, and R³⁰ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R²⁸ and R²⁹, R²⁸ and R³⁰, R²⁹ and R³⁰ together with the atom to which they are connected form an optionally substituted 3-20 membered cycloalkyl or heterocyclyl ring;
and pharmaceutically acceptable salts thereof.

In another embodiment, the 5HT2A agonist is a compound of FORMULA 2:

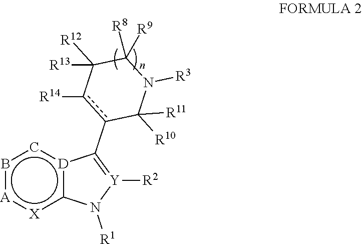

wherein;
definitions of A, B, C, D, X, Y, R¹, R² and R³ are the same as for FORMULA 1
n is selected from 0, 1 or 2;
R⁸, R⁹, R¹⁰, R¹¹, R¹², R¹³ and R¹⁴, at each occurrence, are independently selected from hydrogen, halogen, C₁-C₈ alkyl, oxo, Ph, CN, NO₂, OR³¹, SR³¹, NR³¹R³², C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R³¹ and R³², R³¹ and R³³, R³² and R³³ together with the atom to which they are connected form an optionally substituted 3-8 membered cycloalkyl or heterocyclyl ring;
and pharmaceutically acceptable salts thereof.

In another embodiment, the 5HT2A agonist is a compound of FORMULAE 2A or 2B:

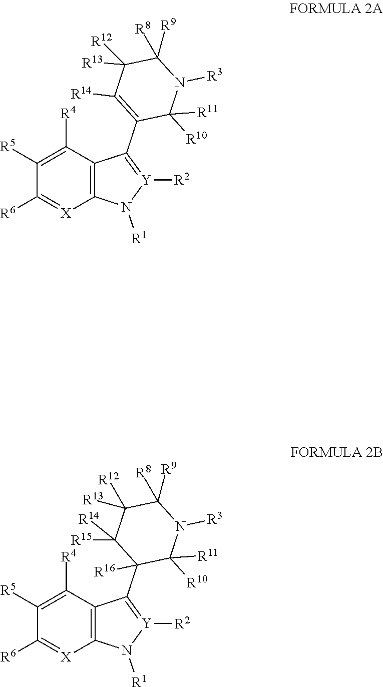

wherein;
X is selected from N, CH or CR⁷;
Y is selected from N or C;
the definitions of R¹, R², R³, R⁴, R⁵, R⁶, R⁷ are the same as for FORMULA 1;
and definitions of R⁸, R⁹, R¹⁰, R¹¹, R¹², R¹³ and R¹⁴ are the same as for FORMULA 2;
R¹⁵ and R¹⁶, at each occurrence, are independently selected from hydrogen, halogen, C₁-C₈alkyl, oxo, Ph, CN, NO₂, OR³⁴, SR³⁴, NR³⁴R³⁵, C(O)R³⁴, C(O)OR³⁴, C(O)NR³⁴R³⁵, S(O)R³⁴, S(O)₂R³⁴, S(O)₂NR³⁴R³⁵, NR³⁶C(O)OR³⁴, NR³⁶C(O)R³⁴, NR³⁶C(O)NR³⁴R³⁵, NR³⁶S(O)R³⁴, NR³⁶S(O)₂R³⁴, NR³⁶S(O)₂NR³⁴R³⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R³⁴, R³⁵, and R³⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R³⁴ and R³⁵, R³⁴ and R³⁶, R³⁵ and R³⁶ together with the atom to which they are connected form an optionally substituted 3-8 membered cycloalkyl or heterocyclyl ring; and
optionally, R¹⁵ and R¹⁶ together with the atom to which they are connected form an optionally substituted 3-20 membered cycloalkyl or heterocyclyl ring; and
pharmaceutically acceptable salts thereof.

In another embodiment, the 5HT2A agonist is a compound of FORMULAE 2C or 2D:

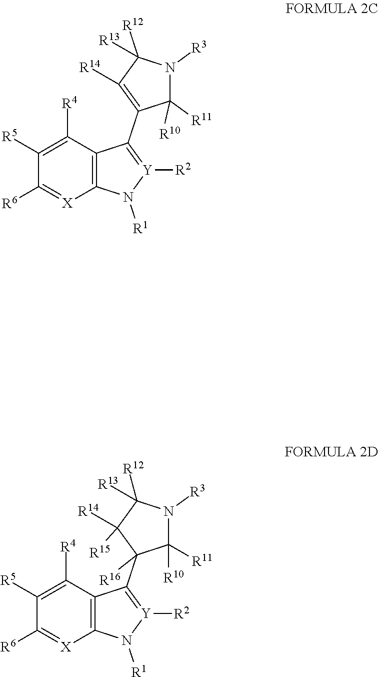

wherein,
X is selected from N, CH or CR⁷;
Y is selected from N or C;
The definitions of R¹, R², R³, R⁴, R⁵, R⁶, R⁷, R⁸, R¹¹, R¹², R¹³, R¹⁴, R¹⁵ an R¹⁶ are the same as for FORMULAE 2A and 2B;
and pharmaceutically acceptable salts thereof.

In another embodiment, the 5HT2A agonist is a compound of FORMULAE 2E or 2F:

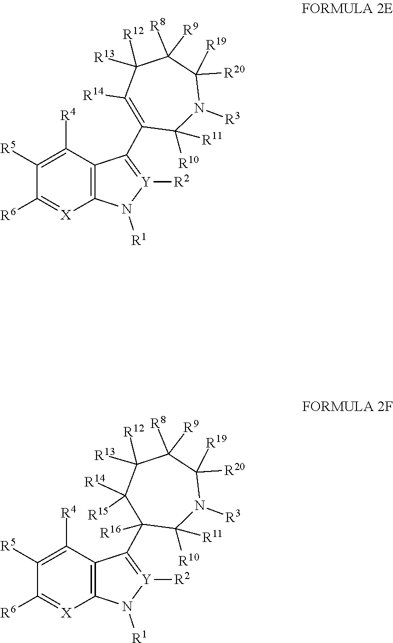

wherein,
X is selected from N, CH or CR⁷;
Y is selected from N or C;
The definitions of R¹, R², R³, R⁴, R⁵, R⁶, R⁷, R⁸, R¹¹, R¹², R¹³, R¹⁴, R¹⁵ and R¹⁶ are the same as for FORMULAE 2A and 2B;
R¹⁹ and R²⁰, at each occurrence, are independently selected from hydrogen, halogen, C₁-C₈ alkyl, oxo, Ph, CN, NO₂, OR³⁷, SR³⁷, NR³⁷R³⁸, C(O)R³⁷, C(O)OR³⁸, C(O)NR³⁷R³⁸, S(O)R³⁷, S(O)₂R³⁷, S(O)₂NR³⁷R³⁸, NR³⁹C(O)OR³⁷, NR³⁹C(O)R³⁷, NR³⁹C(O)NR³⁷R³⁸, NR³⁹S(O)R³⁷, NR³⁹S(O)₂R³⁷, NR⁶S(O)₂NR³⁷R³⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
   R³⁷, R³⁸, and R³⁹ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
   R³⁷ and R³⁸, R³⁷ and R³⁹, R³⁸ and R³⁹ together with the atom to which they are connected form an optionally substituted 3-8 membered cycloalkyl or heterocyclyl ring; and
pharmaceutically acceptable salts thereof.

In another embodiment, the 5HT2A agonist is a compound of FORMULA 3:

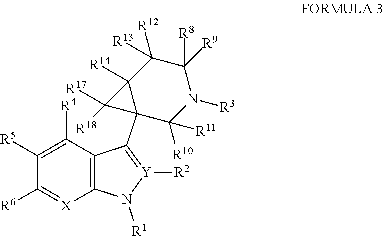

wherein;
X is selected from N, CH or CR⁷;
Y is selected from N or C;
The definitions of R¹, R², R³, R⁴, R⁵, R⁶, R⁷, R¹⁷ and R¹⁸ are the same as for FORMULA 1;
and definitions of R⁸, R⁹, R¹⁰, R¹¹, R¹², R¹³ and R¹⁴ are the same as for FORMULA 2A; and
pharmaceutically acceptable salts thereof.

In another embodiment, the 5HT2A agonist is a compound of FORMULA 4:

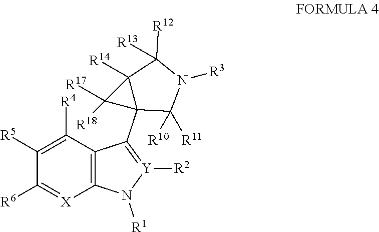

wherein;
X is selected from N, CH or CR⁷;
Y is selected from N or C;
The definitions of R¹, R², R³, R⁴, R⁵, R⁶, R⁷, R¹⁷ and R¹⁸ are the same as for FORMULA 1;
And definitions of R¹⁰, R¹¹, R¹², R¹³ and R¹⁴ are the same as for FORMULA 2A;
and pharmaceutically acceptable salts thereof.

In another embodiment, the 5HT2A agonist is a compound of FORMULA 5:

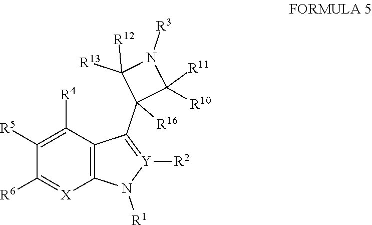

wherein;
X is selected from N, CH or CR⁷;
Y is selected from N or C;
the definitions of R¹, R², R³, R⁴, R⁵, R⁶, R⁷ are the same as for FORMULA 1;
and definitions of R¹⁰, R¹¹, R¹², R¹³ and R¹⁶ are the same as for FORMULA 2A; and
pharmaceutically acceptable salts thereof.

In some aspects, compound is a compound selected from those synthesized in the Examples below: NS131-179, NS131-178, NS131-177, NS136-006, NS131-169, NS131-168, NS131-167, NS131-173, NS131-180, RS134-52, RS134-48, NS131-185, NS131-170, RS134-45, RS134-40, NS131-184, RS134-49, RS134-53, RS134-41, RS134-46, NS131-172, RS134-38, RS134-65, RS134-62, RS134-70, NS136-081, RS134-73, RS134-72, NS136-092, NS136-091, NS136-096, NS136-095, NS136-102, NS136-101, NS136-115, NS136-116, NS136-117, NS136-118, NS136-119, NS136-120, NS136-109, NS136-110, NS136-111, NS136-112, RS134-37, RS134-56, NS136-002, NS136-004, RS130-132, YX129-177C, YX129-180C, YX143-19, YX143-20, YX143-2, YX143-21, NS144-042, NS144-043, NS144-044, YS135-44, YS135-45, YS135-34, YS135-32, YS135-38, YS135-41, YS135-39, YX143-14A-2, NS144-019, NS144-021, YX143-15, YX143-16, YX143-17C, YX143-18C, NS144-047, NS144-048, NS144-049, NS136-128, NS136-129, NS136-130, NS136-131, NS136-150, NS136-151, NS136-152, NS136-166, NS144-011, NS136-158, NS136-167, NS136-159, NS136-135, NS136-136, NS136-137, NS144-046, NS144-045, NS136-140, NS136-141, NS136-142, NS136-143, NS136-153, NS136-154, NS136-155, NS136-175, NS144-016, NS136-160, NS136-176, NS136-161, NS136-144, NS136-145, NS136-146, NS144-051, NS144-050, YX143-41C, YX143-42C, YX143-43D, NS144-059-2, NS144-054-2, NS144-067, NS144-085, NS144-093, NS144-094, NS144-095, NS144-096, XQ148-012, XQ148-023, ZX147-015, ZX147-016, ZX147-017, ZX147-019, NS144-097, NS144-098, NS144-102, NS144-101, NS144-107, NS144-108, NS144-109, NS144-110, YS135-52, YS135-53, YS135-54, YS135-80, YS135-81, YS135-82, YS135-96, YS135-98, YS135-99, YS135-100, ZX147-026-01, ZX147-026-02, ZX147-027, ZX147-028, ZX147-029, ZX147-031, ZX147-054, ZX147-055, ZX147-056, ZX147-092, ZX147-093, ZX147-094, ZX147-095, ZX147-096, ZX147-097, ZX147-098, ZX147-099, ZX147-100, ZX147-128, ZX147-129, ZX147-130, ZX147-131, ZX147-137, ZX147-183, ZX156-011, ZX156-012, ZX156-014-1, ZX156-014-2, ZX156-019, ZX156-059, ZX156-069, ZX156-070, ZX156-071, ZX156-089, ZX156-090, ZX162-100, ZX162-031, ZX162-104, ZX162-105, ZX162-110, ZX162-111, ZX162-112, ZX162-113, ZX162-124, ZX162-126, ZX162-127, ZX162-128, ZX162-129, ZX162-138, ZX162-139, ZX162-140, ZX162-141, ZX162-147, ZX162-148, ZX162-151, ZX162-173, ZX162-174, ZX162-175, ZX162-176, YX143-103B, YX143-103C, YX143-105C, YX143-108, YX143-110B, YX143-112B, YX143-129, YX143-134C, YX143-138C, YX143-182C-1, YX143-183A, YX143-184B-1, YX143-184B-2, YX143-185B, YX143-186B, YX157-19A, YX157-20A, YX157-29B, YX157-42B, YX157-51B, YX157-51C, YX157-55A, XS159-153, XS159-155, XS159-160, XS159-163, XS159-180, XS159-186, XS165-3, XS165-5, XS165-8, XQ148-93, XQ158-012, XQ158-055, XQ158-056, XQ158-078, XQ158-093A, XQ158-082, XQ158-115, XQ158-164, XQ158-167, XQ158-168, ZD160-34, ZD160-140, ZD160-141, ZD160-149, ZD160-11, ZD160-133, ZD160-130, ZD160-131, QC166-005, QC166-008, QC-166-032, XQ148-86, QC166-096, QC166-097, QC179-001, QC179-002, QC179-025, QC-179-032, QC-179-033, QC179-038, QC179-039, QC179-040, ZX167-072, ZX167-077, ZX167-074, ZX167-090, ZX167-091, ZX162-100-1 (Enantiomer 1 of ZX162-100), ZX162-100-2 (Enantiomer 2 of ZX162-100), ZX162-031-1 (Enantiomer 1 of ZX162-031), ZX162-031-2 (Enantiomer 2 of ZX162-031), ZX167-074-1 (Enantiomer 1 of ZX167-074), ZX167-074-2 (Enantiomer 2 of ZX167-074), ZX177-057, ZX177-058, ZX177-058BY, ZX177-059, ZX177-060 and analogs thereof.

In some aspects, compound is a compound selected from those synthesized in the Examples below, including, but not limited to: NS131-179, NS131-178, NS131-177, NS136-006, NS131-169, NS131-168, NS131-167, NS131-173, NS131-180, RS134-52, RS134-48, NS131-185, NS131-170, RS134-45, RS134-40, NS131-184, RS134-49, RS134-53, RS134-41, RS134-46, NS131-172, RS134-38, RS134-65, RS134-62, RS134-70, NS136-081, RS134-73, RS134-72, NS136-092, NS136-091, NS136-096, NS136-095, NS136-102, NS136-101, NS136-115, NS136-116, NS136-117, NS136-118, NS136-119, NS136-120, RS134-37, RS134-56, NS136-002, NS136-004, YS135-34, YS135-32, YS135-38, YS135-41, YS135-39, YX143-14A-2, NS144-019, NS144-021, YX143-15, YX143-16, YX143-17C, YX143-18C, NS144-047, NS144-048, NS144-049, NS136-128, NS136-129, NS136-130, NS136-131, NS136-150, NS136-151, NS136-152, NS136-166, NS144-011, NS136-158, NS136-167, NS136-159, NS136-140, NS136-141, NS136-142, NS136-143, NS136-153, NS136-154, NS136-155, NS136-175, NS144-016, NS136-160, NS136-176, NS136-161, NS144-093, NS144-094, NS144-095, NS144-096, YS135-80, YS135-81, YS135-82, YS135-96, YS135-98, ZX156-069, and analogs thereof.

In some aspects, compound is a compound selected from those synthesized in the Examples below, including, but not limited to: RS130-132, YX129-177C, YX129-180C, YX143-19, YX143-20, YX143-2, YX143-21, NS144-042, NS144-043, NS144-044, YS135-44, YS135-45, NS136-135, NS136-136, NS136-137, NS144-046, NS144-045, NS136-144, NS136-145, NS136-146, NS144-051, NS144-050, YX143-41C, YX143-42C, YX143-43D, NS144-059-2, NS144-054-2, NS144-067, NS144-085, XQ148-012, XQ148-023, ZX147-015, ZX147-016, ZX147-017, ZX147-019, NS144-097, NS144-098, NS144-102, NS144-101, NS144-107, NS144-108, NS144-109, NS144-110, YS135-52, YS135-53, YS135-54, YS135-99, YS135-100, ZX147-026-01, ZX147-026-02, ZX147-027, ZX147-028, ZX147-029, ZX147-054, ZX147-055, ZX147-056, ZX147-092, ZX147-093, ZX147-094, ZX147-095, ZX147-096, ZX147-097, ZX147-098, ZX147-099, ZX147-100, ZX147-128, ZX147-129, ZX147-130, ZX147-137, ZX147-183, ZX156-019, ZX156-059, ZX156-070, ZX156-071, ZX156-089, ZX156-090, XQ148-93, XQ158-012, XQ158-055, XQ158-056, XQ148-86, and analogs thereof.

In some aspects, compound is a compound selected from those synthesized in the Examples below, including, but not limited to: YX143-103B, YX143-103C, YX143-105C, YX143-108, YX143-110B, YX143-112B, YX143-129, YX143-134C, YX143-138C, YX143-182C-1, YX143-183A, YX143-184B-1, YX143-184B-2, YX143-185B, YX143-186B, YX157-19A, YX157-20A, YX157-29B, YX157-42B, YX157-51B, YX157-51C, YX157-55A, and analogs thereof.

In some aspects, compound is a compound selected from those synthesized in the Examples below, including, but not limited to: XS159-180, XS159-186, XS165-3, XS165-5, XS165-8, XQ158-078, XQ158-093A, XQ158-082, XQ158-115, XQ158-164, XQ158-167, XQ158-168, ZD160-34, ZD160-140, ZD160-141, ZD160-149, ZD160-11, ZD160-133, ZD160-130, ZD160-131, and analogs thereof.

In some aspects, compound is a compound selected from those synthesized in the Examples below, including, but not limited to: QC166-005, QC166-008, QC-166-032, QC166-096, QC166-097, QC179-001, QC179-002, QC179-025, QC-179-032, QC-179-033, QC179-038, QC179-039, QC179-040, and analogs thereof.

In some aspects, compound is a compound selected from those synthesized in the Examples below, including, but not limited to: ZX162-100, ZX162-031, ZX162-104, ZX162-105, ZX162-110, ZX162-111, ZX162-112, ZX162-113, ZX162-124, ZX162-126, ZX162-127, ZX162-128, ZX162-129, ZX162-138, ZX162-139, ZX162-140, ZX162-141, ZX162-147, ZX162-148, ZX162-151, ZX162-173, ZX162-174, ZX162-175, ZX162-176, XS159-153, XS159-155, XS159-160, XS159-163, ZX167-072, ZX167-077, ZX167-074, ZX167-090, ZX167-091, ZX162-100-1 (Enantiomer 1 of ZX162-100), ZX162-100-2 (Enantiomer 2 of ZX162-100), ZX162-031-1 (Enantiomer 1 of ZX162-031), ZX162-031-2 (Enantiomer 2 of ZX162-031), ZX167-074-1 (Enantiomer 1 of ZX167-074), ZX167-074-2 (Enantiomer 2 of ZX167-074), ZX177-057, ZX177-058, ZX177-058BY, ZX177-059, ZX177-060 and analogs thereof.

In some aspects, compound is a compound selected from those synthesized in the Examples below, including, but not limited to: NS136-109, NS136-110, NS136-111, NS136-112, NS136-145, RS134-40, RS134-45, RS134-48, RS134-46, YX143-19 and analogs thereof.

In some aspects, compound is a compound selected from those synthesized in the Examples below, including, but not limited to: YX143-108, YX143-129, YX143-134C, ZX147-031, ZX147-131, ZX162-031, ZX162-031-1, ZX162-100, ZX162-100-2, ZX162-105, ZX167-074, ZX167-091, QC166-008, QC166-096, QC166-097, and analogs thereof.

In some aspects, compound is a compound selected from those synthesized in the Examples below, including, but not limited to: ZX162-031, ZX162-031-1, ZX162-100, ZX162-100-2, ZX162-105, ZX167-074, ZX167-091, QC166-008, QC166-096, QC166-097, and analogs thereof.

In some aspects, compound is a compound selected from those synthesized in the Examples below, including, but not limited to:
a) 3-(3-azabicyclo[4.1.0]heptan-1-yl)-7-chloro-1H-indazole (ZX162-031) including its pure enantiomers, mixtures of enantiomers, and pharmaceutically acceptable salts, solvent complexes, morphological forms, or deuterated and fluorinated derivatives thereof.
b) 3-(3-azabicyclo[3.1.0]hexan-1-yl)-7-chloro-1H-indazole (ZX162-100) including its pure enantiomers, mixtures of enantiomers, and pharmaceutically acceptable salts, solvent complexes, morphological forms, or deuterated and fluorinated derivatives thereof
c) 3-(3-azabicyclo[3.1.0]hexan-1-yl)-7-chloro-1H-indole (ZX167-074) including its pure enantiomers, mixtures of enantiomers, and pharmaceutically acceptable salts, solvent complexes, morphological forms, or deuterated and fluorinated derivative thereof
d) 3-(3-azabicyclo[3.1.0]hexan-1-yl)-7-methyl-1H-indole (ZX167-091) including its pure enantiomers, mixtures of enantiomers, and pharmaceutically acceptable salts, solvent complexes, morphological forms, or deuterated and fluorinated derivatives thereof.

In some aspects, compound is a compound selected from those synthesized in the Examples below, including, but not limited to:
a. 3-(azetidin-3-yl)-7-chloro-1H-indole (QC166-008), and pharmaceutically acceptable salts, solvent complexes, morphological forms, or deuterated and fluorinated derivatives thereof
b. 3-(azetidin-3-yl)-7-methyl-1H-indole (QC166-096), and pharmaceutically acceptable salts, solvent complexes, morphological forms, or deuterated and fluorinated derivatives thereof
c. 3-(azetidin-3-yl)-7-fluoro-1H-indole (QC166-097), and pharmaceutically acceptable salts, solvent complexes, morphological forms, or deuterated and fluorinated derivatives thereof.

In some aspects of the disclosed methods, the compounds can be administered by any of several routes of administration including, e.g., orally, parenterally, intradermally, subcutaneously, topically, and/or rectally.

Any of the above-described methods can further include treating the subject with one or more additional therapeutic regimens for treatment.

As used herein, the terms “about” and “approximately” are defined as being within plus or minus 10% of a given value or state, preferably within plus or minus 5% of said value or state.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### Synthesis and Testing of the Compounds

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

### Definition of Terms

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein witch is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s).

Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofirazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent 0 atoms or two adjacent S atoms. A heterocyclyl group may include fused, bridged or spirocyclic ring systems. In certain embodiments, a hetercyclyl group comprises 3 to 10 ring atoms (3-10 membered heterocyclyl). In certain embodiments, a hetercyclyl group comprises 3 to 8 ring atoms (3-8 membered heterocyclyl). In certain embodiments, a hetercyclyl group comprises 4 to 8 ring atoms (4-8 membered heterocyclyl). In certain embodiments, a hetercyclyl group comprises 3 to 6 ring atoms (3-6 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O—alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Pharmaceutical Compositions

In some aspects, the compositions and methods described herein include the manufacture and use of pharmaceutical compositions and medicaments that include one or more compounds as disclosed herein. Also included are the pharmaceutical compositions themselves.

In some aspects, the compositions disclosed herein can include other compounds, drugs, or agents used for the treatment. For example, in some instances, pharmaceutical compositions disclosed herein can be combined with one or more (e.g., one, two, three, four, five, or less than ten) compounds.

In some aspects, the pH of the compositions disclosed herein can be adjusted with pharmaceutically acceptable acids, bases, or buffers to enhance the stability of the compounds or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the compounds disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., alkali metal (e.g., sodium), alkaline earth metal (e.g., magnesium), ammonium salts. The invention also envisions the quaternization of any basic nitrogen-containing groups of the compounds disclosed herein. Water or oil-soluble or dispersible products can be obtained by such quaternization.

In some aspects, the pharmaceutical compositions disclosed herein can include an effective amount of one or more compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome. In some aspects, pharmaceutical compositions can further include one or more additional compounds, drugs, or agents used for the treatment in amounts effective for causing an intended effect or physiological outcome.

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein and one or more additional compounds (e.g., one or more additional compounds, drugs, or agents used for the treatment of Alzeimers Disease (AD) or any other age related condition or disease, including conditions or diseases known to be associated with or caused by AD), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising a compound, including pharmaceutical compositions (indicated below as ‘X’) disclosed herein in the following methods:

Substance X for use as a medicament in the treatment of one or more diseases or conditions disclosed herein. Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of AD or age related diseases.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering. In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the compositions and methods of the present invention.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention.

As used herein, the term “preventing a disease” in a subject means for example, to stop the development of one or more symptoms of a disease in a subject before they occur or are detectable, e.g., by the patient or the patient's doctor. Preferably, the disease does not develop at all, i.e., no symptoms of the disease are detectable. However, it can also mean delaying or slowing of the development of one or more symptoms of the disease. Alternatively, or in addition, it can mean decreasing the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

### EXAMPLES

The following Examples describe the synthesis of exemplary 5HT2A agonist compounds according to the present invention.

### Synthetic Procedures and Characterization Data

Prep-HPLC was used in the final product purifications unless otherwise noted.

### Method A:

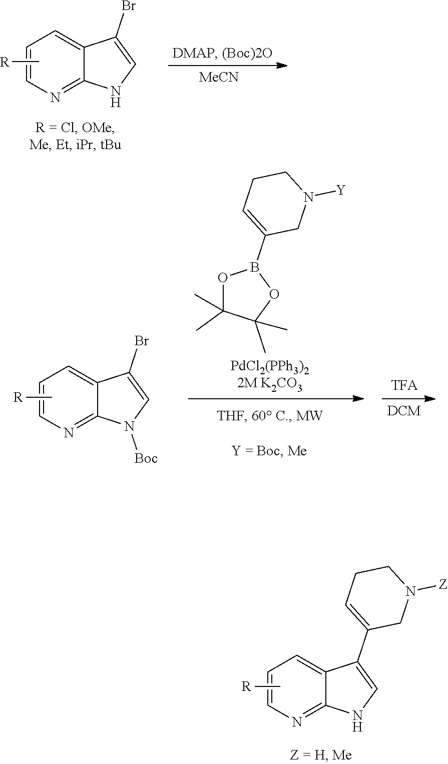

### Example 1

### Synthesis of NS131-179

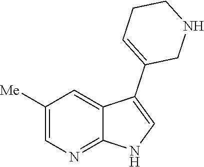 [SMILES: Cc1cnc2[nH]cc(C3=CCCNC3)c2c1]

5-methyl-3-(1,2,5,6-tetrahydropyridin-3-yl)-1H-pyrrolo[2,3-b]pyridine (NS131-179). NS131-179 was synthesized following the method A. To a solution of 3-bromo-5-methyl-1H-pyrrolo[2,3-b]pyridine (211 mg, 1 mmol, 1 equiv) in MeCN (4 mL) were added DMAP (147 mg, 1.2 mmol, 1.2 equiv), (Boc)₂O (240 mg, 1.1 mmol, 1.1 equiv). After being stirred for 2 h at room temperature, the resulting mixture was purified by silica gel (10% ethyl acetate in hexane) to afford tert-butyl 3-bromo-5-methyl-1H-pyrrolo[2,3-b]pyridine-1-carboxylate (295 mg, 95%, yellow solid) as an intermediate, then to a solution of the intermediate (31.1 mg, 0.1 mmol, 1 equiv) in THF (1 mL) were added tert-butyl 5-(4,4,5,5-tetramethyl-1,3,2-dioxaborolan-2-yl)-3,6-dihydropyridine-1(2H)-carboxylate (31 mg, 0.1 mmol, 1 equiv), 2M K₂CO₃ solution (0.15 mL, 0.3 mmol, 3 equiv), Pd(PPh₃)₂Cl₂ (7.0 mg, 0.01 mmol, 0.1 equiv), and the atmosphere evacuated and backfilled with nitrogen three times. After being stirred for 1 h at 60° C. by microwave, the resulting mixture was purified by preparative HPLC (10%-100% acetonitrile/0.1% TFA in H₂O) to get the crude compound, then added 0.5 mL DCM and 0.5 mL TFA, stirred for 2 h at rt, evaporated and the resulting mixture was purified by preparative HPLC (10%-100% acetonitrile/0.1% TFA in H₂O) to give NS131-179 as a white solid (24 mg, 55%). ¹H NMR (600 MHz, Methanol-d₄) δ 8.54-8.50 (m, 1H), 8.26-8.22 (m, 1H), 7.65 (s, 1H), 6.53-6.48 (m, 1H), 4.09 (q, J=2.1 Hz, 2H), 3.43 (t, J=6.2 Hz, 2H), 2.69-2.63 (m, 2H), 2.55 (s, 3H). LRMS (ESI) m/z: calcd for C₁₃H₁₆N₃⁺ [M+H]⁺, 214.13; found, 214.21.

### Example 2

### Synthesis of NS131-178

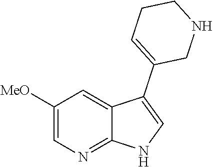 [SMILES: COc1cnc2[nH]cc(C3=CCCNC3)c2c1]

5-methoxy-3-(1,2,5,6-tetrahydropyridin-3-yl)-1H-pyrrolo[2,3-b]pyridine (NS131-178). NS131-178 was synthesized following the standard procedure for preparing NS131-179 from 3-bromo-5-methoxy-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 23.4 mg, 51%) ¹H NMR (600 MHz, Methanol-d₄) δ 8.07 (s, 1H), 7.94 (d, J=2.5 Hz, 1H), 7.54 (s, 1H), 6.40 (tt, J=4.1, 1.8 Hz, 1H), 4.08 (q, J=2.1 Hz, 2H), 3.93 (s, 3H), 3.42 (t, J=6.2 Hz, 2H), 2.69-2.63 (m, 2H). LRMS (ESI) m/z: calcd for C₁₃H₁₆N₃O⁻ [M+H]⁺, 230.13; found, 230.11.

### Example 3

### Synthesis of NS131-177

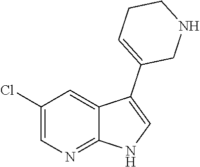 [SMILES: Clc1cnc2[nH]cc(C3=CCCNC3)c2c1]

5-chloro-3-(1,2,5,6-tetrahydropyridin-3-yl)-1H-pyrrolo[2,3-b]pyridine (NS131-177). NS131-177 was synthesized following the standard procedure for preparing NS131-179 from 3-bromo-5-chloro-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 9.0 mg, 49%) ¹H NMR (600 MHz, Methanol-d₄) δ 8.26 (d, J=2.2 Hz, 1H), 8.23 (d, J=2.2 Hz, 1H), 7.57 (s, 1H), 6.40 (tt, J=4.1, 1.8 Hz, 1H), 4.07 (q, J=2.1 Hz, 2H), 3.42 (t, J=6.2 Hz, 2H), 2.67-2.64 (m, 2H). LRMS (ESI) m/z: calcd for C₁₂H₁₃N₃Cl⁺ [M+H]⁺, 234.08; found, 234.19.

### Method B:

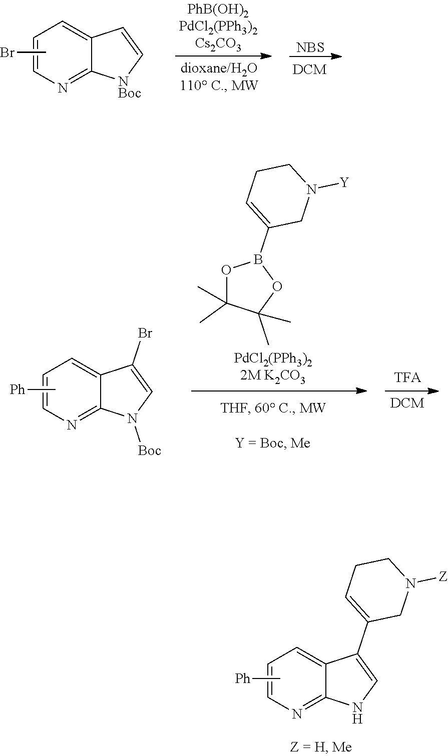

### Example 4

### Synthesis of NS136-006

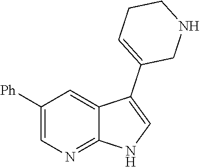 [SMILES: C1=C(c2c[nH]c3ncc(-c4ccccc4)cc23)CNCC1]

5-phenyl-3-(1,2,5,6-tetrahydropyridin-3-yl)-1H-pyrrolo[2,3-b]pyridine (NS136-006). NS136-006 was synthesized following the method B. To a solution of tert-butyl 5-bromo-1H-pyrrolo[2,3-b]pyridine-1-carboxylate (297 mg, 1 mmol, 1 equiv) in dioxane (5 mL) and water (0.5 mL) were added PhB(OH)₂ (146 mg, 1.2 mmol, 1.2 equiv), Pd(PPh₃)₂Cl₂ (7 mg, 0.01 mmol, 0.01 equiv), Cs₂CO₃ (651.6 mg, 2 mmol, 2 equiv). After being stirred for 2 h at 110° C. by microwave, the resulting mixture was purified by silica gel (10% to 20% ethyl acetate in hexane) to afford tert-butyl 5-phenyl-1H-pyrrolo[2,3-b]pyridine-1-carboxylate (230 mg, 78%, yellow solid). Then to a solution of tert-butyl 5-phenyl-1H-pyrrolo[2,3-b]pyridine-1-carboxylate (58.8 mg, 0.2 mmol, 1 equiv) in DCM (2 mL) was added NBS (28 mg, 0.22 mmol, 1.1 equiv), after being stirred for 2 h at rt, the resulting mixture was purified by silica gel (20% ethyl acetate in hexane) to afford the bromo-substituted compound (71.7 mg, 96%) as an intermediate, then follow the same procedure with NS131-179, to a solution of the last step intermediate (37.3 mg, 0.1 mmol, 1 equiv) in THF (1 mL) were added tert-butyl 5-(4,4,5,5-tetramethyl-1,3,2-dioxaborolan-2-yl)-3,6-dihydropyridine-1(2H)-carboxylate (31 mg, 0.1 mmol, 1 equiv), 2M K₂CO₃ solution (0.15 mL, 0.3 mmol, 3 equiv), Pd(PPh₃)₂Cl₂ (7.0 mg, 0.01 mmol, 0.1 equiv), and the atmosphere evacuated and backfilled with nitrogen three times. After being stirred for 1 h at 60° C. by microwave, the resulting mixture was purified by preparative HPLC (10%-100% acetonitrile/0.1% TFA in H₂O) to get the crude compound, then added 0.5 mL DCM and 0.5 mL TFA, stirred for 2 h at rt, evaporated and the resulting mixture was purified by preparative HPLC (10%-100% acetonitrile/0.1% TFA in H₂O) to give NS136-006 as a white solid (9.3 mg, 18%) ¹H NMR (600 MHz, Methanol-d₄) δ 8.52 (s, 1H), 8.46 (s, 1H), 7.68 (d, J=7.6 Hz, 2H), 7.57 (s, 1H), 7.50 (t, J=7.6 Hz, 2H), 7.41-7.36 (m, 1H), 6.51 (s, 1H), 4.12 (d, J=2.4 Hz, 2H), 3.43 (t, J=6.2 Hz, 2H), 2.73-2.64 (m, 2H). LRMS (ESI) m/z: calcd for C₁₈H₁₈N₃⁻ [M+H]⁺, 276.15; found, 276.27.

### Example 5

### Synthesis of NS131-169

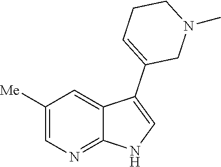 [SMILES: Cc1cnc2[nH]cc(C3=CCCN(C)C3)c2c1]

5-methyl-3-(1-methyl-1,2,5,6-tetrahydropyridin-3-yl)-1H-pyrrolo[2,3-b]pyridine (NS131-169). NS131-169 was synthesized following the standard procedure for preparing NS131-179 from 3-bromo-5-methyl-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 14 mg, 34%) ¹H NMR (600 MHz, Methanol-d₄) δ 8.38 (t, J=1.4 Hz, 1H), 8.20 (s, 1H), 7.59 (s, 1H), 6.47 (dt, J=4.2, 2.1 Hz, 1H), 4.35 (d, J=15.8 Hz, 1H), 4.02 (d, J=15.8 Hz, 1H), 3.67 (s, 1H), 3.07 (s, 3H), 2.78 (s, 1H), 2.72-2.67 (m, 1H), 2.52 (s, 3H). LRMS (ESI) m/z: calcd for C₁₄H₁₈N₃⁺ [M+H]⁺, 228.15; found, 228.04.

### Example 6

### Synthesis of NS131-168

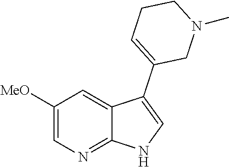 [SMILES: COc1cnc2[nH]cc(C3=CCCN(C)C3)c2c1]

5-methoxy-3-(1-methyl-1,2,5,6-tetrahydropyridin-3-yl)-1H-pyrrolo[2,3-b]pyridine (NS131-168). NS131-168 was synthesized following the standard procedure for preparing NS131-179 from 3-bromo-5-methoxy-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 17.4 mg, 41%) ¹H NMR (600 MHz, Methanol-d₄) δ 8.03 (d, J=2.6 Hz, 1H), 7.82 (d, J=2.6 Hz, 1H), 7.50 (s, 1H), 6.40-6.37 (m, 1H), 4.34 (d, J=15.7 Hz, 1H), 4.00 (d, J=15.9 Hz, 1H), 3.92 (s, 3H), 3.66 (dd, J=12.3, 6.1 Hz, 1H), 3.34 (s, 1H), 3.07 (s, 3H), 2.78 (d, J=8.7 Hz, 1H), 2.69 (d, J=19.2 Hz, 1H). LRMS (ESI) m/z: calcd for Cl₄H₁₈N₃O⁺ [M+H]⁺, 244.14; found, 244.15.

### Example 7

### Synthesis of NS131-167

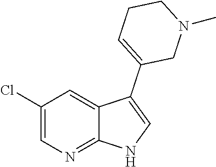 [SMILES: CN1CCC=C(c2c[nH]c3ncc(Cl)cc23)C1]

5-chloro-3-(1-methyl-1,2,5,6-tetrahydropyridin-3-yl)-1H-pyrrolo[2,3-b]pyridine (NS131-167). NS131-167 was synthesized following the standard procedure for preparing NS131-179 from 3-bromo-5-chloro-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 16.3 mg, 38%) ¹H NMR (600 MHz, Methanol-d₄) δ 8.27 (d, J=2.2 Hz, 1H), 8.23 (d, J=2.3 Hz, 1H), 7.58 (s, 1H), 6.38 (dq, J=4.2, 1.8 Hz, 1H), 4.34 (d, J=15.7 Hz, 1H), 4.03-3.97 (m, 1H), 3.66 (dd, J=12.1, 5.9 Hz, 1H), 3.30-3.27 (m, 1H), 3.07 (s, 3H), 2.82-2.74 (m, 1H), 2.68 (d, J=19.1 Hz, 1H). LRMS (ESI) m/z: calcd for C₁₃H₁₅N₃Cl⁺ [M+H]⁺, 248.09; found, 248.19.

### Example 8

### Synthesis of NS131-173

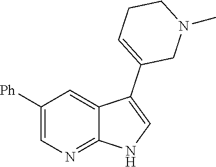 [SMILES: CN1CCC=C(c2c[nH]c3ncc(-c4ccccc4)cc23)C1]

3-(1-methyl-1,2,5,6-tetrahydropyridin-3-yl)-5-phenyl-1H-pyrrolo[2,3-b]pyridine (NS131-173). NS131-173 was synthesized following the standard procedure for preparing NS136-006 from tert-butyl 5-bromo-1H-pyrrolo[2,3-b]pyridine-1-carboxylate and 1-methyl-5-(4,4,5,5-tetramethyl-1,3,2-dioxaborolan-2-yl)-1,2,3,6-tetrahydropyridine. (white solid, 20.3 mg, 39%) ¹H NMR (600 MHz, Methanol-d₄) δ 8.55-8.52 (m, 1H), 8.50 (d, J=2.0 Hz, 1H), 7.71-7.67 (m, 2H), 7.61 (s, 1H), 7.50 (t, J=7.8 Hz, 2H), 7.42-7.38 (m, 1H), 6.51 (dt, J=4.2, 2.0 Hz, 1H), 4.39 (d, J=15.8 Hz, 1H), 4.05 (d, J=15.8 Hz, 1H), 3.70-3.65 (m, 1H), 3.36-3.33 (m, 1H), 3.08 (s, 3H), 2.79 (d, J=7.0 Hz, 1H), 2.71 (d, J=19.0 Hz, 1H). LRMS (ESI) m/z: calcd for Cl₉H₂₀N₃⁺ [M+H]⁺, 290.17; found, 290.24.

### Example 9

### Synthesis of NS131-180

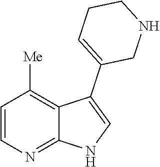 [SMILES: Cc1ccnc2[nH]cc(C3=CCCNC3)c12]

4-methyl-3-(1,2,5,6-tetrahydropyridin-3-yl)-1H-pyrrolo[2,3-b]pyridine (NS131-180). NS131-180 was synthesized following the standard procedure for preparing NS131-179 from 3-bromo-4-methyl-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 12.8 mg, 29%) ¹H NMR (600 MHz, Methanol-d₄) δ 8.30 (s, 1H), 7.62 (s, 1H), 7.37 (d, J=5.9 Hz, 1H), 6.12 (tt, J=3.9, 1.9 Hz, 1H), 3.97 (q, J=2.3 Hz, 2H), 3.44 (t, J=6.2 Hz, 2H), 2.83 (s, 3H), 2.66-2.62 (m, 2H). LRMS (ESI) m/z: calcd for Cl₃H₁₆N₃[M+H], 214.13; found, 214.25.

### Example 10

### Synthesis of RS134-52

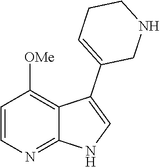 [SMILES: COc1ccnc2[nH]cc(C3=CCCNC3)c12]

4-methoxy-3-(1,2,5,6-tetrahydropyridin-3-yl)-1H-pyrrolo[2,3-b]pyridine (RS134-52). RS134-52 was synthesized following the standard procedure for preparing NS131-179 from 3-bromo-4-methoxy-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 16.5 mg, 36%) ¹H NMR (600 MHz, Methanol-d₄) δ 8.36 (d, J=6.8 Hz, 1H), 7.47 (s, 1H), 7.17 (d, J=6.8 Hz, 1H), 6.18 (tt, J=3.9, 1.8 Hz, 1H), 4.24 (s, 3H), 4.06 (q, J=2.2 Hz, 2H), 3.41 (t, J=6.3 Hz, 2H), 2.62-2.59 (m, 2H). LRMS (ESI) m/z: calcd for Cl₃H₁₆N₃O⁺ [M+H]⁺, 230.13; found, 230.32.

### Example 11

### Synthesis of RS134-48

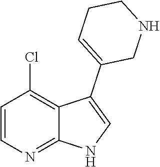 [SMILES: Clc1ccnc2[nH]cc(C3=CCCNC3)c12]

4-chloro-3-(1,2,5,6-tetrahydropyridin-3-yl)-1H-pyrrolo[2,3-b]pyridine (RS134-48). RS134-48 was synthesized following the standard procedure for preparing NS131-179 from 3-bromo-4-chloro-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 10.2 mg, 22%) ¹H NMR (600 MHz, Methanol-d₄) δ 8.17 (dd, J=5.4, 2.8 Hz, 1H), 7.46 (d, J=4.5 Hz, 1H), 7.21 (t, J=5.3 Hz, 1H), 6.07 (tt, J=3.9, 1.9 Hz, 1H), 4.02 (q, J=2.2 Hz, 2H), 3.41 (t, J=6.2 Hz, 2H), 2.62-2.58 (m, 2H). LRMS (ESI) m/z: calcd for C₁₂H₁₃N₃Cl⁺ [M+H]⁺, 234.08; found, 234.35.

### Example 12

### Synthesis of NS131-185

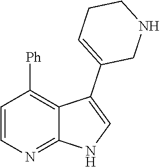 [SMILES: C1=C(c2c[nH]c3nccc(-c4ccccc4)c23)CNCC1]

4-phenyl-3-(1,2,5,6-tetrahydropyridin-3-yl)-1H-pyrrolo[2,3-b]pyridine (NS131-185). NS131-185 was synthesized following the standard procedure for preparing NS136-006 from tert-butyl 4-bromo-1H-pyrrolo[2,3-b]pyridine-1-carboxylate and tert-butyl 5-(4,4,5,5-tetramethyl-1,3,2-dioxaborolan-2-yl)-3,6-dihydropyridine-1(2H)-carboxylate. (white solid, 11.6 mg, 23%) ¹H NMR (600 MHz, Methanol-d₄) δ 8.47-8.42 (m, 1H), 7.68 (s, 1H), 7.58 (dt, J=5.1, 2.2 Hz, 3H), 7.56-7.52 (m, 2H), 7.41 (d, J=5.6 Hz, 1H), 5.48 (tt, J=4.0, 1.9 Hz, 1H), 3.38 (q, J=2.3 Hz, 2H), 3.04 (t, J=6.2 Hz, 2H), 2.19-2.15 (m, 2H). LRMS (ESI) m/z: calcd for C₁₈H₁₈N₃⁺ [M+H]⁺, 276.15; found, 276.29.

### Example 13

### Synthesis of NS131-170

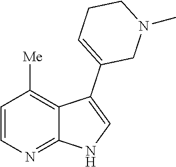 [SMILES: Cc1ccnc2[nH]cc(C3=CCCN(C)C3)c12]

4-methyl-3-(1-methyl-1,2,5,6-tetrahydropyridin-3-yl)-1H-pyrrolo[2,3-b]pyridine (NS131-170). NS131-170 was synthesized following the standard procedure for preparing NS131-179 from 3-bromo-4-methyl-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 10.3 mg, 25%) ¹H NMR (600 MHz, Methanol-d₄) δ 8.21 (d, J=5.5 Hz, 1H), 7.51 (s, 1H), 7.19 (d, J=5.5 Hz, 1H), 6.09 (dq, J=4.0, 2.0 Hz, 1H), 4.17 (d, J=16.3 Hz, 1H), 3.94 (d, J=16.4 Hz, 1H), 3.67 (s, 1H), 3.06 (s, 3H), 2.74 (s, 4H), 2.65 (d, J=22.0 Hz, 1H). LRMS (ESI) m/z: calcd for C₁₄H₁₈N₃⁺ [M+H]⁺, 228.15; found, 228.08.

### Example 14

### Synthesis of RS134-45

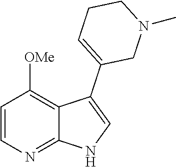 [SMILES: COc1ccnc2[nH]cc(C3=CCCN(C)C3)c12]

4-methoxy-3-(1-methyl-1,2,5,6-tetrahydropyridin-3-yl)-1H-pyrrolo[2,3-b]pyridine (RS134-45). RS134-45 was synthesized following the standard procedure for preparing NS131-179 from 3-bromo-4-methoxy-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 14.6 mg, 31%) ¹H NMR (600 MHz, Methanol-d₄) δ 8.37 (d, J=6.9 Hz, 1H), 7.49 (s, 1H), 7.19 (d, J=6.8 Hz, 1H), 6.20 (tt, J=3.8, 1.8 Hz, 1H), 4.31-4.26 (m, 1H), 4.25 (s, 3H), 4.00-3.93 (m, 1H), 3.65 (t, J=9.8 Hz, 1H), 3.33 (d, J=11.4 Hz, 1H), 3.06 (s, 3H), 2.79-2.70 (m, 1H), 2.63 (d, J=19.3 Hz, 1H). LRMS (ESI) m/z: calcd for C₁₄H₁₈N₃O⁺ [M+H]⁺, 244.14; found, 244.22.

### Example 15

### Synthesis of RS134-40

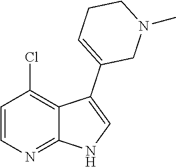 [SMILES: CN1CCC=C(c2c[nH]c3nccc(Cl)c23)C1]

4-chloro-3-(1-methyl-1,2,5,6-tetrahydropyridin-3-yl)-1H-pyrrolo[2,3-b]pyridine (RS134-40). RS134-40 was synthesized following the standard procedure for preparing NS131-179 from 3-bromo-4-chloro-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 12.4 mg, 26%) ¹H NMR (600 MHz, Methanol-d₄) δ 8.21 (d, J=5.4 Hz, 1H), 7.52 (d, J=2.0 Hz, 1H), 7.26 (dd, J=5.5, 2.3 Hz, 1H), 6.07 (tt, J=3.8, 1.9 Hz, 1H), 4.23 (d, J=16.2 Hz, 1H), 3.95 (dq, J=16.2, 2.6 Hz, 1H), 3.69-3.62 (m, 1H), 3.35-3.31 (m, 1H), 3.05 (s, 3H), 2.78-2.71 (m, 1H), 2.67-2.58 (m, 1H). LRMS (ESI) m/z: calcd for C₁₃H₁₅N₃Cl⁺ [M+H]⁺, 248.09; found, 248.22.

### Example 16

### Synthesis of NS131-184

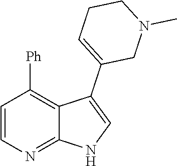 [SMILES: CN1CCC=C(c2c[nH]c3nccc(-c4ccccc4)c23)C1]

3-(1-methyl-1,2,5,6-tetrahydropyridin-3-yl)-4-phenyl-1H-pyrrolo[2,3-b]pyridine (NS131-184). NS131-184 was synthesized following the standard procedure for preparing NS136-006 from tert-butyl 4-bromo-1H-pyrrolo[2,3-b]pyridine-1-carboxylate and 1-methyl-5-(4,4,5,5-tetramethyl-1,3,2-dioxaborolan-2-yl)-1,2,3,6-tetrahydropyridine. (white solid, 12.5 mg, 24%) ¹H NMR (600 MHz, Methanol-d₄) δ 8.44 (d, J=5.6 Hz, 1H), 7.70 (s, 1H), 7.58 (dq, J=4.6, 2.8, 2.2 Hz, 3H), 7.53 (dt, J=6.8, 2.2 Hz, 2H), 7.40 (d, J=5.6 Hz, 1H), 5.37 (tt, J=3.9, 1.9 Hz, 1H), 3.86 (d, J=16.2 Hz, 1H), 3.39 (s, 1H), 3.21 (d, J=16.0 Hz, 1H), 2.86 (t, J=5.6 Hz, 1H), 2.81 (s, 3H), 2.36 (d, J=11.8 Hz, 1H), 2.09-1.99 (m, 1H). LRMS (ESI) m/z: calcd for Cl₉H₂₀N₃⁺ [M+H]⁺, 290.17; found, 290.29.

### Example 17

### Synthesis of RS134-49

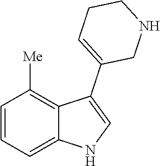 [SMILES: Cc1cccc2[nH]cc(C3=CCCNC3)c12]

4-methyl-3-(1,2,5,6-tetrahydropyridin-3-yl)-1H-indole (RS134-49). RS134-49 was synthesized following the standard procedure for preparing NS131-179 from tert-butyl 3-bromo-4-methyl-1H-indole-1-carboxylate and tert-butyl 5-(4,4,5,5-tetramethyl-1,3,2-dioxaborolan-2-yl)-3,6-dihydropyridine-1(2H)-carboxylate. (white solid, 6.9 mg, 21%) ¹H NMR (600 MHz, Methanol-d₄) δ 7.21 (d, J=8.2 Hz, 1H), 7.15 (s, 1H), 7.01 (dd, J=8.2, 7.1 Hz, 1H), 6.80 (dt, J=7.1, 0.9 Hz, 1H), 5.93 (tt, J=3.7, 1.8 Hz, 1H), 3.91 (q, J=2.2 Hz, 2H), 3.39 (t, J=6.2 Hz, 2H), 2.61-2.53 (m, 5H). LRMS (ESI) m/z: calcd for Cl₄H₁₇N₂⁺ [M+H]⁺, 213.14; found, 213.43.

### Method C:

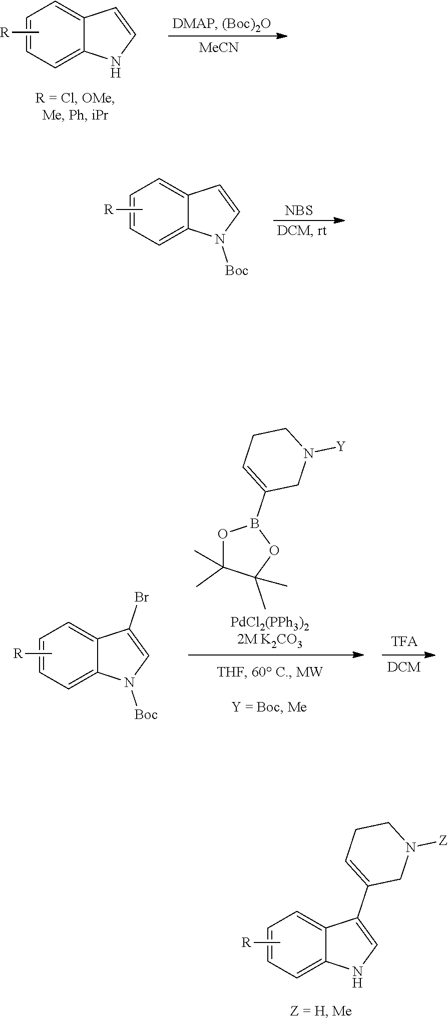

### Example 18

### Synthesis of RS134-53

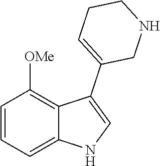 [SMILES: COc1cccc2[nH]cc(C3=CCCNC3)c12]

4-methoxy-3-(1,2,5,6-tetrahydropyridin-3-yl)-1H-indole (RS134-53). RS134-53 was synthesized following the method C. To a solution of 4-methoxy-1H-indole (147 mg, 1 mmol, 1 equiv) in MeCN (4 mL) were added DMAP (147 mg, 1.2 mmol, 1.2 equiv), (Boc)₂O (240 mg, 1.1 mmol, 1.1 equiv). After being stirred for 2 h at room temperature, the resulting mixture was purified by silica gel (10% ethyl acetate in hexane) to afford tert-butyl 4-methoxy-1H-indole-1-carboxylate (240 mg, 97%, yellow solid). Then to a solution of tert-butyl 5-phenyl-1H-pyrrolo[2,3-b]pyridine-1-carboxylate (240 mg, 1 mmol, 1 equiv) in DCM (3 mL) was added NBS (195.8 mg, 1.1 mmol, 1.1 equiv), after being stirred for 1 h at rt, the resulting mixture was purified by silica gel (10% ethyl acetate in hexane) to afford the bromo-substituted compound (163 mg, 50%) as an intermediate, then follow the same procedure with NS131-179, to a solution of the last step intermediate (32.6 mg, 0.1 mmol, 1 equiv) in THF (1 mL) were added tert-butyl 5-(4,4,5,5-tetramethyl-1,3,2-dioxaborolan-2-yl)-3,6-dihydropyridine-1(2H)-carboxylate (31 mg, 0.1 mmol, 1 equiv), 2M K₂CO₃ solution (0.15 mL, 0.3 mmol, 3 equiv), Pd(PPh₃)₂Cl₂ (7.0 mg, 0.01 mmol, 0.1 equiv), and the atmosphere evacuated and backfilled with nitrogen three times. After being stirred for 1 h at 60° C. by microwave, the resulting mixture was purified by preparative HPLC (10%-100% acetonitrile/0.1% TFA in H₂O) to get the crude compound, then added 0.5 mL DCM and 0.5 mL TFA, stirred for 2 h at rt, evaporated and the resulting mixture was purified by preparative HPLC (10%-100% acetonitrile/0.1% TFA in H₂O) to give RS134-53 as a white solid (12 mg, 35%). ¹H NMR (600 MHz, Methanol-d₄) δ 7.11 (s, 1H), 7.06 (t, J=7.9 Hz, 1H), 7.00 (dd, J=8.1, 0.7 Hz, 1H), 6.55 (d, J=7.7 Hz, 1H), 5.98 (tt, J=3.9, 1.8 Hz, 1H), 4.12 (q, J=2.1 Hz, 2H), 3.93 (s, 3H), 3.37 (t, J=6.3 Hz, 2H), 2.58-2.55 (m, 2H). LRMS (ESI) m/z: calcd for C₁₄H₁₇N₂₀⁺ [M+H], 229.13; found, 229.32.

### Example 19

### Synthesis of RS134-41

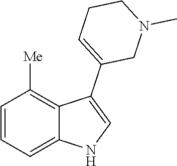 [SMILES: Cc1cccc2[nH]cc(C3=CCCN(C)C3)c12]

4-methyl-3-(1-methyl-1,2,5,6-tetrahydropyridin-3-yl)-1H-indole (RS134-41). RS134-41 was synthesized following the standard procedure for preparing NS131-179 from tert-butyl 3-bromo-4-methyl-1H-indole-1-carboxylate and 1-methyl-5-(4,4,5,5-tetramethyl-1,3,2-dioxaborolan-2-yl)-1,2,3,6-tetrahydropyridine. (white solid, 13.3 mg, 39%)¹H NMR (600 MHz, Methanol-d₄) δ 7.21 (d, J=8.2 Hz, 1H), 7.17 (s, 1H), 7.02 (t, J=7.7 Hz, 1H), 6.80 (d, J=7.1 Hz, 1H), 5.93 (tt, J=4.1, 1.9 Hz, 1H), 4.12 (d, J=16.4 Hz, 1H), 3.92-3.84 (m, 1H), 3.63 (dd, J=12.4, 6.1 Hz, 1H), 3.28 (dd, J=11.7, 5.2 Hz, 1H), 3.03 (s, 3H), 2.76-2.69 (m, 1H), 2.60 (d, J=19.5 Hz, 1H), 2.56 (s, 3H). LRMS (ESI) m/z: calcd for C₁₅H₁₉N₂⁺ [M+H]⁺, 227.15; found, 227.38.

### Example 20

### Synthesis of RS134-46

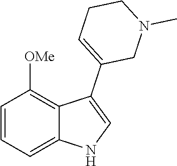 [SMILES: COc1cccc2[nH]cc(C3=CCCN(C)C3)c12]

4-methoxy-3-(1-methyl-1,2,5,6-tetrahydropyridin-3-yl)-1H-indole (RS134-46). RS134-46 was synthesized following the standard procedure for preparing RS134-53 from 4-methoxy-1H-indole and 1-methyl-5-(4,4,5,5-tetramethyl-1,3,2-dioxaborolan-2-yl)-1,2,3,6-tetrahydropyridine. (white solid, 11.1 mg, 31%) ¹H NMR (600 MHz, Methanol-d₄) δ 7.13 (s, 1H), 7.07 (t, J=8.0 Hz, 1H), 7.00 (d, J=8.2 Hz, 1H), 6.56 (d, J=7.8 Hz, 1H), 5.98 (q, J=2.9, 1.8 Hz, 1H), 4.40 (d, J=15.8 Hz, 1H), 4.00-3.95 (m, 1H), 3.93 (s, 3H), 3.63-3.57 (m, 1H), 3.30-3.26 (m, 1H), 3.04 (s, 3H), 2.72-2.66 (m, 1H), 2.63-2.55 (m, 1H). LRMS (ESI) m/z: calcd for C₁₅H₁₉N₂O⁺ [M+H]⁺, 243.15; found, 243.37.

### Example 21

### Synthesis of NS131-172

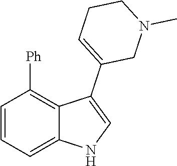 [SMILES: CN1CCC=C(c2c[nH]c3cccc(-c4ccccc4)c23)C1]

3-(1-methyl-1,2,5,6-tetrahydropyridin-3-yl)-4-phenyl-1H-indole (NS131-172). NS131-172 was synthesized following the standard procedure for preparing RS134-53 from 4-phenyl-1H-indole and 1-methyl-5-(4,4,5,5-tetramethyl-1,3,2-dioxaborolan-2-yl)-1,2,3,6-tetrahydropyridine. (white solid, 10.1 mg, 25%) ¹H NMR (600 MHz, Methanol-d₄) δ 7.49-7.43 (m, 3H), 7.43-7.39 (m, 3H), 7.31 (d, J=2.2 Hz, 1H), 7.23 (dd, J=8.2, 7.2 Hz, 1H), 7.01 (dd, J=7.2, 1.0 Hz, 1H), 5.44 (dq, J=3.9, 1.9 Hz, 1H), 3.62 (d, J=16.1 Hz, 1H), 3.34 (s, 1H), 2.93 (d, J=16.1 Hz, 1H), 2.71 (s, 4H), 2.37 (s, 1H), 2.09 (d, J=18.9 Hz, 1H). LRMS (ESI) m/z: calcd for C₂₀H₂₁N₂⁺ [M+H]⁺, 289.17; found, 289.29.

### Example 22

### Synthesis of RS134-38

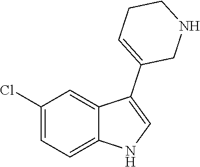 [SMILES: Clc1ccc2[nH]cc(C3=CCCNC3)c2c1]

5-chloro-3-(1,2,5,6-tetrahydropyridin-3-yl)-1H-indole (RS134-38). RS134-38 was synthesized following the standard procedure for preparing NS131-179 from tert-butyl 3-bromo-5-chloro-1H-indole-1-carboxylate and tert-butyl 5-(4,4,5,5-tetramethyl-1,3,2-dioxaborolan-2-yl)-3,6-dihydropyridine-1(2H)-carboxylate. (white solid, 8.3 mg, 24%) ¹H NMR (600 MHz, Methanol-d₄) δ 7.78 (d, J=2.0 Hz, 1H), 7.40 (s, 1H), 7.37 (d, J=8.6 Hz, 1H), 7.14 (dd, J=8.6, 2.0 Hz, 1H), 6.37-6.31 (m, 1H), 4.05 (q, J=2.1 Hz, 2H), 3.41 (t, J=6.2 Hz, 2H), 2.68-2.61 (m, 2H). LRMS (ESI) m/z: calcd for C₁₃H₁₄ClN₂⁺ [M+H]⁺, 233.08; found, 233.22.

### Example 23

### Synthesis of RS134-65

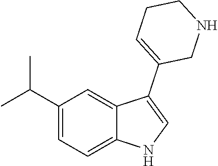 [SMILES: CC(C)c1ccc2[nH]cc(C3=CCCNC3)c2c1]

5-isopropyl-3-(1,2,5,6-tetrahydropyridin-3-yl)-1H-indole (RS134-65). RS134-65 was synthesized following the standard procedure for preparing RS134-53 from 5-isopropyl-1H-indole and tert-butyl 5-(4,4,5,5-tetramethyl-1,3,2-dioxaborolan-2-yl)-3,6-dihydropyridine-1(2H)-carboxylate. (white solid, 11.4 mg, 32%) ¹H NMR (600 MHz, Methanol-d₄) δ 7.64-7.60 (m, 1H), 7.32 (d, J=8.4 Hz, 1H), 7.29 (s, 1H), 7.08 (dd, J=8.4, 1.6 Hz, 1H), 6.36 (tt, J=4.1, 1.8 Hz, 1H), 4.06 (q, J=2.1 Hz, 2H), 3.40 (t, J=6.2 Hz, 2H), 3.03-2.96 (m, 1H), 2.67-2.63 (m, 2H), 1.30 (d, J=6.9 Hz, 6H). LRMS (ESI) m/z: calcd for C₁₆H₂₁N₂⁺ [M+H]⁺, 241.17; found, 241.40.

### Example 24

### Synthesis of RS134-62

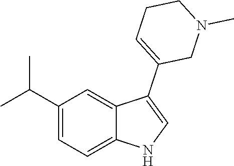 [SMILES: CC(C)c1ccc2[nH]cc(C3=CCCN(C)C3)c2c1]

5-isopropyl-3-(1-methyl-1,2,5,6-tetrahydropyridin-3-yl)-1H-indole (RS134-62). RS134-62 was synthesized following the standard procedure for preparing RS134-53 from 5-isopropyl-1H-indole and 1-methyl-5-(4,4,5,5-tetramethyl-1,3,2-dioxaborolan-2-yl)-1,2,3,6-tetrahydropyridine. (white solid, 12.1 mg, 33%) ¹H NMR (600 MHz, Methanol-d₄) δ 7.62 (s, 1H), 7.32 (dd, J=8.2, 3.6 Hz, 2H), 7.09 (d, J=7.6 Hz, 1H), 6.37 (s, 1H), 4.32 (d, J=15.9 Hz, 1H), 4.00 (d, J=14.6 Hz, 1H), 3.65 (s, 1H), 3.36-3.33 (m, 1H), 3.06 (q, J=5.2, 4.3 Hz, 3H), 3.03-2.96 (m, 1H), 2.78 (s, 1H), 2.68 (d, J=29.9 Hz, 1H), 1.30 (d, J=7.4 Hz, 6H). LRMS (ESI) m/z: calcd for C₁₇H₂N₂⁺ [M+H]⁺, 255.19; found, 255.33.

### Example 25

### Synthesis of RS134-70

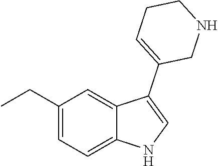 [SMILES: CCc1ccc2[nH]cc(C3=CCCNC3)c2c1]

5-ethyl-3-(1,2,5,6-tetrahydropyridin-3-yl)-1H-indole (RS134-70). RS134-70 was synthesized following the standard procedure for preparing RS134-53 from 5-ethyl-1H-indole and tert-butyl 5-(4,4,5,5-tetramethyl-1,3,2-dioxaborolan-2-yl)-3,6-dihydropyridine-1(2H)-carboxylate. (white solid, 11.9 mg, 35%) ¹H NMR (600 MHz, Methanol-d₄) δ 7.60 (s, 1H), 7.31 (d, J=8.3 Hz, 1H), 7.29 (s, 1H), 7.04 (dd, J=8.3, 1.6 Hz, 1H), 6.38 (tt, J=4.0, 1.7 Hz, 1H), 4.06 (q, J=2.1 Hz, 2H), 3.41 (t, J=6.2 Hz, 2H), 2.74 (q, J=7.5 Hz, 2H), 2.67-2.64 (m, 2H), 1.27 (t, J=7.6 Hz, 3H). LRMS (ESI) m/z: calcd for C₁₅H₁₉N₂⁺ [M+H]f, 227.15; found, 227.38.

### Example 26

### Synthesis of NS136-081

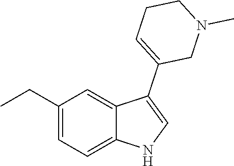 [SMILES: CCc1ccc2[nH]cc(C3=CCCN(C)C3)c2c1]

5-ethyl-3-(1-methyl-1,2,5,6-tetrahydropyridin-3-yl)-1H-indole (NS136-081). NS136-081 was synthesized following the standard procedure for preparing RS134-53 from 5-ethyl-1H-indole and 1-methyl-5-(4,4,5,5-tetramethyl-1,3,2-dioxaborolan-2-yl)-1,2,3,6-tetrahydropyridine. (white solid, 5.3 mg, 15%) ¹H NMR (600 MHz, Methanol-d₄) δ 7.62-7.59 (m, 1H), 7.31 (t, J=4.2 Hz, 2H), 7.04 (dd, J=8.3, 1.6 Hz, 1H), 6.37 (dt, J=4.1, 1.9 Hz, 1H), 4.32 (d, J=15.6 Hz, 1H), 4.00 (d, J=15.7 Hz, 1H), 3.65 (dd, J=12.2, 5.9 Hz, 1H), 3.33 (s, 1H), 3.06 (s, 3H), 2.74 (q, J=7.6 Hz, 4H), 1.27 (t, J=7.6 Hz, 3H). LRMS (ESI) m/z: calcd for C₁₆H₂₁N₂⁺ [M+H]⁺, 241.17; found, 241.28.

### Example 27

### Synthesis of RS134-73

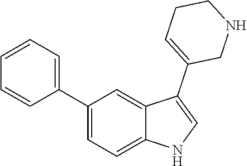 [SMILES: C1=C(c2c[nH]c3ccc(-c4ccccc4)cc23)CNCC1]

5-phenyl-3-(1,2,5,6-tetrahydropyridin-3-yl)-1H-indole (RS134-73). RS134-73 was synthesized following the standard procedure for preparing RS134-53 from 5-phenyl-1H-indole and tert-butyl 5-(4,4,5,5-tetramethyl-1,3,2-dioxaborolan-2-yl)-3,6-dihydropyridine-1(2H)-carboxylate. (white solid, 7.4 mg, 19%) ¹H NMR (600 MHz, Methanol-d₄) δ 7.99 (d, J=2.0 Hz, 1H), 7.65-7.60 (m, 2H), 7.48 (d, J=8.4 Hz, 1H), 7.45-7.40 (m, 3H), 7.38 (d, J=2.0 Hz, 1H), 7.28 (dd, J=8.3, 6.5 Hz, 1H), 6.45-6.43 (m, 1H), 4.09 (dd, J=3.9, 2.0 Hz, 2H), 3.41 (t, J=6.1 Hz, 2H), 2.68-2.64 (m, 2H). LRMS (ESI) m/z: calcd for C₁₉H₁₉N₂⁺ [M+H]⁺, 275.15; found, 275.31.

### Example 28

### Synthesis of RS134-72

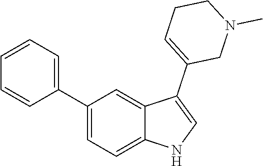 [SMILES: CN1CCC=C(c2c[nH]c3ccc(-c4ccccc4)cc23)C1]

3-(1-methyl-1,2,5,6-tetrahydropyridin-3-yl)-5-phenyl-1H-indole (RS134-72). RS134-72 was synthesized following the standard procedure for preparing RS134-53 from 5-phenyl-1H-indole and 1-methyl-5-(4,4,5,5-tetramethyl-1,3,2-dioxaborolan-2-yl)-1,2,3,6-tetrahydropyridine. (white solid, 8.5 mg, 21%) ¹H NMR (600 MHz, Methanol-d₄) δ 8.01-7.98 (m, 1H), 7.63 (dd, J=7.9, 1.4 Hz, 2H), 7.50-7.45 (m, 2H), 7.45-7.39 (m, 3H), 7.29 (t, J=7.3 Hz, 1H), 6.44 (q, J=4.3, 3.0 Hz, 1H), 4.35 (d, J=15.7 Hz, 1H), 4.03 (d, J=15.6 Hz, 1H), 3.66 (dd, J=12.2, 6.2 Hz, 1H), 3.34 (d, J=5.0 Hz, 1H), 3.07 (s, 3H), 2.79 (t, J=8.2 Hz, 1H), 2.70 (d, J=19.3 Hz, 1H). LRMS (ESI) m/z: calcd for C₂₀H₂₁N₂ [M+H]⁺, 289.17; found, 289.25.

### Example 29

### Synthesis of NS136-092

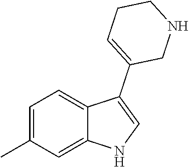 [SMILES: Cc1ccc2c(C3=CCCNC3)c[nH]c2c1]

6-methyl-3-(1,2,5,6-tetrahydropyridin-3-yl)-1H-indole (NS136-092). NS136-092 was synthesized following the standard procedure for preparing RS134-53 from 6-methyl-1H-indole and tert-butyl 5-(4,4,5,5-tetramethyl-1,3,2-dioxaborolan-2-yl)-3,6-dihydropyridine-1(2H)-carboxylate. (white solid, 6.2 mg, 21%) ¹H NMR (400 MHz, Methanol-d₄) δ 7.39-7.33 (m, 1H), 7.13 (s, 1H), 6.84 (d, J=8.1 Hz, 1H), 6.40 (dd, J=7.8, 4.8 Hz, 2H), 4.11 (s, 2H), 3.40 (p, J=4.7 Hz, 2H), 2.64 (dd, J=8.3, 4.0 Hz, 2H), 2.41 (t, J=2.4 Hz, 3H). LRMS (ESI) m/z: calcd for C₁₉H₁₉N₂⁺ [M+H]⁺, 213.14; found, 213.28.

### Example 30

### Synthesis of NS136-091

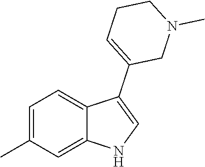

6-methyl-3-(1-methyl-1,2,5,6-tetrahydropyridin-3-yl)-1H-indole (NS136-091). NS136-091 was synthesized following the standard procedure for preparing RS134-53 from 6-methyl-1H-indole and 1-methyl-5-(4,4,5,5-tetramethyl-1,3,2-dioxaborolan-2-yl)-1,2,3,6-tetrahydropyridine. (white solid, 7.1 mg, 23%) ¹H NMR (400 MHz, Methanol-d₄) δ 7.37 (d, J=8.1 Hz, 1H), 7.13 (s, 1H), 6.87-6.82 (m, 1H), 6.41 (d, J=6.5 Hz, 2H), 4.40 (d, J=15.8 Hz, 1H), 4.02 (d, J=15.8 Hz, 1H), 3.65 (d, J=9.8 Hz, 1H), 2.70 (t, J=19.6 Hz, 2H), 2.41 (d, J=3.4 Hz, 3H). LRMS (ESI) m/z: calcd for C₁₅H₁₉N₂⁺ [M+H]⁺, 227.15; found, 227.12.

### Example 31

### Synthesis of NS136-096

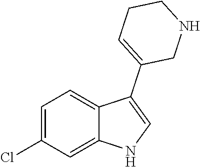 [SMILES: Clc1ccc2c(C3=CCCNC3)c[nH]c2c1]

6-chloro-3-(1,2,5,6-tetrahydropyridin-3-yl)-1H-indole (NS136-096). NS136-096 was synthesized following the standard procedure for preparing RS134-53 from 6-chloro-1H-indole and tert-butyl 5-(4,4,5,5-tetramethyl-1,3,2-dioxaborolan-2-yl)-3,6-dihydropyridine-1(2H)-carboxylate. (white solid, 6.0 mg, 19%) ¹H NMR (400 MHz, Methanol-d₄) δ 7.77 (d, J=8.4 Hz, 1H), 7.43-7.34 (m, 2H), 7.08 (d, J=8.6 Hz, 1H), 6.38 (s, 1H), 4.06 (s, 2H), 3.41 (p, J=4.9 Hz, 2H), 2.64 (s, 2H). LRMS (ESI) m/z: calcd for C₁₃H₁₄ClN₂⁺ [M+H]⁺, 233.08; found, 233.23.

### Example 32

### Synthesis of NS136-095

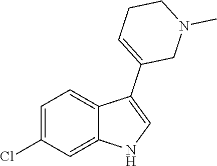 [SMILES: CN1CCC=C(c2c[nH]c3cc(Cl)ccc23)C1]

6-chloro-3-(1-methyl-1,2,5,6-tetrahydropyridin-3-yl)-1H-indole (NS136-095). NS136-095 was synthesized following the standard procedure for preparing RS134-53 from 6-chloro-1H-indole and 1-methyl-5-(4,4,5,5-tetramethyl-1,3,2-dioxaborolan-2-yl)-1,2,3,6-tetrahydropyridine. (white solid, 6.9 mg, 21%) ¹H NMR (400 MHz, Methanol-d₄) δ 7.77 (d, J=8.4 Hz, 111), 7.43-7.34 (m, 2H), 7.08 (d, J=8.6 Hz, 1H), 6.38 (s, 1H), 4.06 (s, 2H), 3.41 (p, J=4.9 Hz, 2H), 2.64 (s, 2H). LRMS (ESI) m/z: calcd for C₁₄H₁₆ClN₂⁺ [M+H]⁺, 247.10; found, 247.25.

### Example 33

### Synthesis of NS136-102

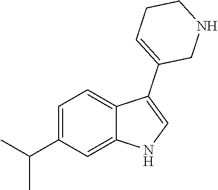 [SMILES: CC(C)c1ccc2c(C3=CCCNC3)c[nH]c2c1]

6-isopropyl-3-(1,2,5,6-tetrahydropyridin-3-yl)-1H-indole (NS136-102). NS136-102 was synthesized following the standard procedure for preparing RS134-53 from 6-isopropyl-1H-indole and tert-butyl 5-(4,4,5,5-tetramethyl-1,3,2-dioxaborolan-2-yl)-3,6-dihydropyridine-1(2H)-carboxylate. (white solid, 7.8 mg, 22%) ¹H NMR (400 MHz, Methanol-d₄) δ 7.43-7.37 (m, 1H), 7.19 (s, 1H), 6.95-6.88 (m, 1H), 6.41 (t, J=6.6 Hz, 2H), 4.12 (s, 2H), 3.41 (q, J=5.3 Hz, 2H), 3.01-2.92 (m, 1H), 2.63 (d, J=8.2 Hz, 2H), 1.31-1.27 (m, 6H). LRMS (ESI) m/z: calcd for C₁₆H₂₁N₂⁺ [M+H]⁺, 241.17; found, 241.32.

### Example 34

### Synthesis of NS136-101

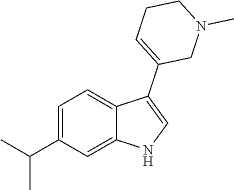 [SMILES: CC(C)c1ccc2c(C3=CCCN(C)C3)c[nH]c2c1]

6-isopropyl-3-(1-methyl-1,2,5,6-tetrahydropyridin-3-yl)-1H-indole (NS136-101). NS136-101 was synthesized following the standard procedure for preparing RS134-53 from 6-isopropyl-1H-indole and 1-methyl-5-(4,4,5,5-tetramethyl-1,3,2-dioxaborolan-2-yl)-1,2,3,6-tetrahydropyridine. (white solid, 4.1 mg, 11%) ¹H NMR (400 MHz, Methanol-d₄) δ 7.45-7.37 (m, 1H), 7.19 (s, 1H), 6.96-6.89 (m, 1H), 6.43 (s, 2H), 4.41 (d, J=15.7 Hz, 1H), 4.03 (d, J=15.7 Hz, 1H), 3.65 (s, 1H), 3.08 (d, J=2.7 Hz, 3H), 2.96 (d, J=9.3 Hz, 1H), 2.85-2.64 (m, 2H), 1.29 (dt, J=6.2, 2.8 Hz, 6H). LRMS (ESI) m/z: calcd for C₁₇H₂₃N₂⁺ [M+H]⁺, 255.19; found, 255.28.

### Example 35

### Synthesis of NS136-115

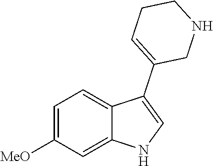 [SMILES: COc1ccc2c(C3=CCCNC3)c[nH]c2c1]

6-methoxy-3-(1,2,5,6-tetrahydropyridin-3-yl)-1H-indole (NS136-115). NS136-115 was synthesized following the standard procedure for preparing RS134-53 from 6-methoxy-1H-indole and tert-butyl 5-(4,4,5,5-tetramethyl-1,3,2-dioxaborolan-2-yl)-3,6-dihydropyridine-1(2H)-carboxylate. (white solid, 4.1 mg, 12%) ¹H NMR (400 MHz, Methanol-d₄) δ 7.67 (t, J=5.6 Hz, 1H), 7.21 (t, J=3.4 Hz, 1H), 6.92 (s, 1H), 6.76 (dd, J=8.7, 4.5 Hz, 1H), 6.37 (s, 1H), 4.05 (s, 2H), 3.83 (d, J=3.9 Hz, 3H), 3.40 (d, J=5.9 Hz, 2H), 2.63 (d, J=8.1 Hz, 2H). LRMS (ESI) m/z: calcd for Cl₄H₁₇N₂O⁺ [M+H]⁺, 229.13; found, 229.25.

### Example 36

### Synthesis of NS136-116

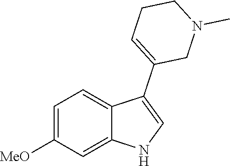 [SMILES: COc1ccc2c(C3=CCCN(C)C3)c[nH]c2c1]

6-methoxy-3-(1-methyl-1,2,5,6-tetrahydropyridin-3-yl)-1H-indole (NS136-116). NS136-116 was synthesized following the standard procedure for preparing RS134-53 from 6-methoxy-1H-indole and 1-methyl-5-(4,4,5,5-tetramethyl-1,3,2-dioxaborolan-2-yl)-1,2,3,6-tetrahydropyridine. (white solid, 5.0 mg, 14%) ¹H NMR (600 MHz, Methanol-d₄) δ 7.67 (d, J=8.8 Hz, 1H), 7.23 (s, 1H), 6.92 (d, J=2.3 Hz, 1H), 6.76 (dd, J=8.9, 2.4 Hz, 1H), 6.36 (s, 1H), 4.31 (d, J=15.7 Hz, 1H), 3.98 (d, J=15.8 Hz, 1H), 3.82 (s, 3H), 3.69-3.62 (m, 1H), 3.06 (s, 3H), 2.80-2.73 (m, 1H), 2.66 (d, J=19.1 Hz, 2H). LRMS (ESI) m/z: calcd for C₁₅H₁₉N₂O⁺ [M+H]⁺, 243.15; found, 243.19.

### Example 37

### Synthesis of NS136-117

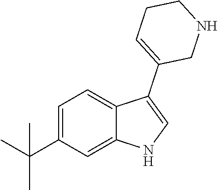 [SMILES: CC(C)(C)c1ccc2c(C3=CCCNC3)c[nH]c2c1]

6-(tert-butyl)-3-(1,2,5,6-tetrahydropyridin-3-yl)-1H-indole (NS136-117). NS136-117 was synthesized following the standard procedure for preparing RS134-53 from 6-(tert-butyl)-1H-indole and tert-butyl 5-(4,4,5,5-tetramethyl-1,3,2-dioxaborolan-2-yl)-3,6-dihydropyridine-1(2H)-carboxylate. (white solid, 4.8 mg, 13%) ¹H NMR (600 MHz, Methanol-d₄) δ 7.72 (d, J=8.5 Hz, 1H), 7.40 (s, 1H), 7.28 (s, 1H), 7.21 (d, J=8.6 Hz, 1H), 6.40 (s, 1H), 4.07 (s, 2H), 3.41 (t, J=6.3 Hz, 2H), 2.69-2.59 (m, 2H), 1.52-1.34 (m, 9H). LRMS (ESI) m/z: calcd for C₁₇H₂₃N₂⁺ [M+H]⁺, 255.19; found, 255.26.

### Example 38

### Synthesis of NS136-118

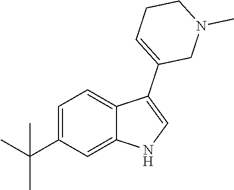 [SMILES: CN1CCC=C(c2c[nH]c3cc(C(C)(C)C)ccc23)C1]

6-(tert-butyl)-3-(1-methyl-1,2,5,6-tetrahydropyridin-3-yl)-1H-indole (NS136-118). NS136-118 was synthesized following the standard procedure for preparing RS134-53 from 6-(tert-butyl)-1H-indole and 1-methyl-5-(4,4,5,5-tetramethyl-1,3,2-dioxaborolan-2-yl)-1,2,3,6-tetrahydropyridine. (white solid, 4.2 mg, 11%) ¹H NMR (600 MHz, Methanol-d₄) δ 7.73 (d, J=8.5 Hz, 1H), 7.45-7.39 (m, 1H), 7.30 (s, 1H), 7.25-7.19 (m, 1H), 6.39 (s, 1H), 4.33 (d, J=15.7 Hz, 1H), 4.00 (d, J=16.1 Hz, 1H), 3.65 (s, 1H), 3.06 (s, 3H), 2.78 (s, 1H), 2.69 (s, 1H), 1.37 (d, J=2.6 Hz, 9H). LRMS (ESI) m/z: calcd for C₁₈H₂₅N₂⁺ [M+H]⁺, 269.20; found, 269.34.

### Example 39

### Synthesis of NS136-119

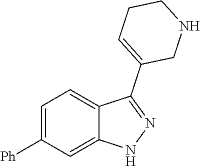 [SMILES: C1=C(c2n[nH]c3cc(-c4ccccc4)ccc23)CNCC1]

6-phenyl-3-(1,2,5,6-tetrahydropyridin-3-yl)-1H-indole (NS136-119). NS136-119 was synthesized following the standard procedure for preparing RS134-53 from 6-phenyl-1H-indole and tert-butyl 5-(4,4,5,5-tetramethyl-1,3,2-dioxaborolan-2-yl)-3,6-dihydropyridine-1(2H)-carboxylate. (white solid, 4.3 mg, 11%) ¹H NMR (600 MHz, Methanol-d₄) δ 7.88 (dd, J=8.4, 2.4 Hz, 1H), 7.69-7.62 (m, 3H), 7.47-7.37 (m, 4H), 7.33-7.29 (m, 1H), 6.45 (tt, J=3.9, 1.8 Hz, 1H), 4.16-4.09 (m, 2H), 3.43 (t, J=6.1 Hz, 2H), 2.69-2.65 (m, 2H). LRMS (ESI) m/z: calcd for C₁₉H₁₉N₂⁺ [M+H]⁺, 275.15; found, 275.32.

### Example 40

### Synthesis of NS136-120

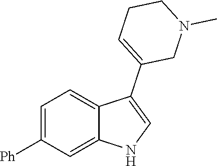 [SMILES: CN1CCC=C(c2c[nH]c3cc(-c4ccccc4)ccc23)C1]

3-(1-methyl-1,2,5,6-tetrahydropyridin-3-yl)-6-phenyl-1H-indole (NS136-120). NS136-120 was synthesized following the standard procedure for preparing RS134-53 from 6-phenyl-1H-indole and 1-methyl-5-(4,4,5,5-tetramethyl-1,3,2-dioxaborolan-2-yl)-1,2,3,6-tetrahydropyridine. (white solid, 4.0 mg, 10%) ¹H NMR (600 MHz, Methanol-d₄) δ 7.92-7.85 (m, 1H), 7.65 (d, J=9.2 Hz, 3H), 7.50-7.37 (m, 4H), 7.30 (t, J=7.4 Hz, 1H), 6.44 (s, 1H), 4.36 (d, J=15.7 Hz, 1H), 4.03 (d, J=15.7 Hz, 1H), 3.66 (d, J=9.8 Hz, 1H), 3.42 (s, 1H), 3.12 (s, 3H), 2.81 (d, J=18.4 Hz, 1H), 2.70 (d, J=19.7 Hz, 1H). LRMS (ESI) m/z: calcd for C₂₀H₂₁N₂⁺ [M+H]⁺, 289.17; found, 289.25.

### Example 41

### Synthesis of NS136-109

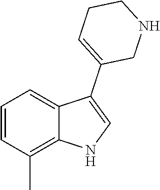 [SMILES: Cc1cccc2c(C3=CCCNC3)c[nH]c12]

7-methyl-3-(1,2,5,6-tetrahydropyridin-3-yl)-1H-indole (NS136-109). NS136-109 was synthesized following the standard procedure for preparing RS134-53 from 7-methyl-1H-indole and tert-butyl 5-(4,4,5,5-tetramethyl-1,3,2-dioxaborolan-2-yl)-3,6-dihydropyridine-1(2H)-carboxylate. (white solid, 3.9 mg, 12%) ¹H NMR (600 MHz, Methanol-d₄) δ 7.33 (dd, J=6.9, 2.0 Hz, 1H), 6.93-6.89 (m, 2H), 6.64-6.61 (m, 1H), 6.47 (s, 1H), 4.13 (q, J=2.0 Hz, 2H), 3.41 (d, J=6.2 Hz, 2H), 2.68-2.64 (m, 2H), 2.51 (s, 3H). LRMS (ESI) m/z: calcd for C₁₉H₁₉N₂⁺ [M+H]⁺, 213.14; found, 213.23.

### Example 42

### Synthesis of NS136-111

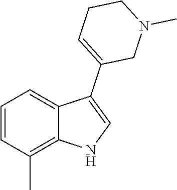 [SMILES: Cc1cccc2c(C3=CCCN(C)C3)c[nH]c12]

7-methyl-3-(1-methyl-1,2,5,6-tetrahydropyridin-3-yl)-1H-indole (NS136-111). NS136-111 was synthesized following the standard procedure for preparing RS134-53 from 7-methyl-1H-indole and 1-methyl-5-(4,4,5,5-tetramethyl-1,3,2-dioxaborolan-2-yl)-1,2,3,6-tetrahydropyridine. (white solid, 5.1 mg, 15%) ¹H NMR (600 MHz, Methanol-d₄) δ 7.64 (d, J=8.0 Hz, 1H), 7.35 (d, J=1.9 Hz, 1H), 7.01 (t, J=7.5 Hz, 1H), 6.97 (d, J=7.0 Hz, 1H), 6.38 (td, J=4.3, 3.9, 1.8 Hz, 1H), 4.34 (d, J=15.7 Hz, 1H), 4.01 (d, J=15.7 Hz, 1H), 3.65 (dd, J=12.3, 6.1 Hz, 1H), 3.06 (s, 3H), 2.67 (d, J=18.8 Hz, 1H), 2.49 (s, 3H). LRMS (ESI) m/z: calcd for C₁₅H₁₉N₂⁺ [M+H]⁺, 227.15; found, 227.19.

### Example 43

### Synthesis of NS136-110

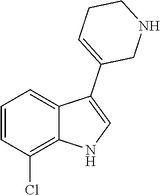 [SMILES: Clc1cccc2c(C3=CCCNC3)c[nH]c12]

7-chloro-3-(1,2,5,6-tetrahydropyridin-3-yl)-1H-indole (NS136-110). NS136-110 was synthesized following the standard procedure for preparing RS134-53 from 7-chloro-1H-indole and tert-butyl 5-(4,4,5,5-tetramethyl-1,3,2-dioxaborolan-2-yl)-3,6-dihydropyridine-1(2H)-carboxylate. (white solid, 4.0 mg, 11%) ¹H NMR (600 MHz, Methanol-d₄) δ 7.76 (d, J=8.2 Hz, 1H), 7.42 (s, 1H), 7.20 (d, J=8.0 Hz, 1H), 7.08 (d, J=8.1 Hz, 1H), 4.08 (s, 2H), 2.65 (s, 2H), 2.04 (d, J=2.4 Hz, 2H). LRMS (ESI) m/z: calcd for Cl₃H₁₄ClN₂⁺ [M+H]f, 233.08; found, 233.27.

### Example 44

### Synthesis of NS136-112

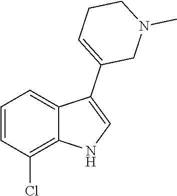 [SMILES: CN1CCC=C(c2c[nH]c3c(Cl)cccc23)C1]

7-chloro-3-(1-methyl-1,2,5,6-tetrahydropyridin-3-yl)-1H-indole (NS136-112). NS136-112 was synthesized following the standard procedure for preparing RS134-53 from 7-chloro-1H-indole and 1-methyl-5-(4,4,5,5-tetramethyl-1,3,2-dioxaborolan-2-yl)-1,2,3,6-tetrahydropyridine. (white solid, 4.0 mg, 11%) ¹H NMR (600 MHz, Methanol-d₄) δ 7.76 (d, J=8.0 Hz, 1H), 7.44 (s, 1H), 7.20 (d, J=7.9 Hz, 1H), 7.12-7.06 (m, 1H), 6.40 (s, 1H), 4.34 (d, J=15.8 Hz, 1H), 4.01 (d, J=16.0 Hz, 1H), 3.67 (d, J=10.6 Hz, 1H), 3.40-3.34 (m, 1H), 3.13-3.04 (m, 3H), 2.78 (d, J=19.8 Hz, 1H), 2.68 (d, J=19.2 Hz, 1H). LRMS (ESI) m/z: calcd for C₁₄H₁₆ClN₂⁺ [M+H], 247.10; found, 247.23.

### Example 45

### Synthesis of RS134-37

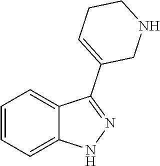 [SMILES: C1=C(c2n[nH]c3ccccc23)CNCC1]

3-(1,2,5,6-tetrahydropyridin-3-yl)-1H-indazole (RS134-37). RS134-37 was synthesized following the standard procedure for preparing NS131-179 from 3-bromo-1H-indazole and tert-butyl 5-(4,4,5,5-tetramethyl-1,3,2-dioxaborolan-2-yl)-3,6-dihydropyridine-1(2H)-carboxylate. (white solid, 19.2 mg, 61%) ¹H NMR (600 MHz, Methanol-d₄) δ 7.98 (d, J=8.3 Hz, 1H), 7.53 (d, J=8.4 Hz, 1H), 7.40 (ddd, J=8.2, 6.8, 1.0 Hz, 1H), 7.21 (ddd, J=8.1, 6.8, 0.9 Hz, 1H), 6.83 (tt, J=4.1, 1.8 Hz, 1H), 4.27 (q, J=2.2 Hz, 2H), 3.45 (t, J=6.2 Hz, 2H), 2.73-2.69 (m, 2H). LRMS (ESI) m/z: calcd for C₁₂H₁₄N₃⁻ [M+H]⁺, 200.12; found, 200.34.

### Example 46

### Synthesis of RS134-56

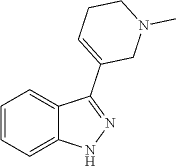 [SMILES: CN1CCC=C(c2n[nH]c3ccccc23)C1]

3-(1-methyl-1,2,5,6-tetrahydropyridin-3-yl)-1H-indazole (RS134-56). RS134-56 was synthesized following the standard procedure for preparing NS131-179 from 3-bromo-1H-indazole and 1-methyl-5-(4,4,5,5-tetramethyl-1,3,2-dioxaborolan-2-yl)-1,2,3,6-tetrahydropyridine. (white solid, 22.3 mg, 68%) ¹H NM/R (600 MHz, Methanol-d₄) δ 7.98 (dd, J=8.2, 1.1 Hz, 1H), 7.54 (dd, J=8.4, 1.0 Hz, 1H), 7.41 (ddd, J=8.2, 6.9, 1.0 Hz, 1H), 7.21 (ddd, J=8.0, 6.9, 0.9 Hz, 1H), 6.83 (tt, J=3.6, 1.7 Hz, 1H), 4.67-4.60 (m, 1H), 4.12-4.08 (m, 1H), 3.73-3.66 (m, 1H), 3.39-3.32 (m, 1H), 3.09 (s, 3H), 2.87-2.81 (m, 1H), 2.79-2.70 (m, 1H). LRMS (ESI) m/z: calcd for Cl₃H₁₆N₃[M+H]⁺, 214.13; found, 214.33.

### Example 47

### Synthesis of NS136-002

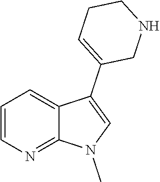 [SMILES: Cn1cc(C2=CCCNC2)c2cccnc21]

1-methyl-3-(1,2,5,6-tetrahydropyridin-3-yl)-1H-pyrrolo[2,3-b]pyridine (NS136-002). NS136-002 was synthesized following the standard procedure for preparing NS131-179 from 3-bromo-1-methyl-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 9.7 mg, 22%) ¹H NMR (600 MHz, Methanol-d₄) δ 8.41 (dd, J=8.0, 1.4 Hz, 1H), 8.35 (dd, J=5.0, 1.4 Hz, 1H), 7.56 (s, 1H), 7.29 (dd, J=8.0, 5.0 Hz, 1H), 6.46 (ft, J=4.1, 1.8 Hz, 1H), 4.08 (q, J=2.1 Hz, 2H), 3.90 (s, 3H), 3.42 (t, J=6.2 Hz, 2H), 2.69-2.65 (m, 2H). LRMS (ESI) m/z: calcd for C₁₃H₁₆N₃⁺ [M+H]⁺, 214.13; found, 214.26.

### Method D:

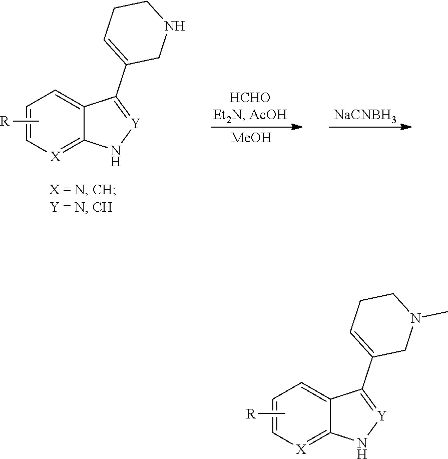

### Example 48

### Synthesis of NS136-004

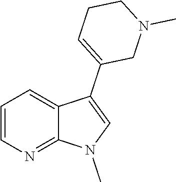 [SMILES: CN1CCC=C(c2cn(C)c3ncccc23)C1]

1-methyl-3-(1-methyl-1,2,5,6-tetrahydropyridin-3-yl)-1H-pyrrolo[2,3-b]pyridine (NS136-004). NS136-004 was synthesized following the method D. To a solution of 1-methyl-3-(1,2,5,6-tetrahydropyridin-3-yl)-1H-pyrrolo[2,3-b]pyridine (8.8 mg, 0.02 mmol, 1 equiv) in MeOH (1 mL) were added Et₃N (3 drops), AcOH (5 drops), HCHO (10 mg). After being stirred for 1 h at room temperature, the resulting mixture was added NaCNBH₃ (3.8 mg, 0.06 mmol, 3 equiv), stirred for 1 h at rt, the resulting mixture was purified by preparative HPLC (10%-100% acetonitrile/0.1% TFA in H₂O) to give NS136-004 as a white solid (7.6 mg, 83%). ¹H NMR (600 MHz, Methanol-d₄) δ 8.33 (d, J=6.5 Hz, 2H), 7.53 (s, 1H), 7.26-7.21 (m, 1H), 6.46-6.42 (m, 1H), 4.33 (d, J=15.7 Hz, 1H), 4.01 (d, J=16.2 Hz, 1H), 3.88 (d, J=1.1 Hz, 3H), 3.67 (dd, J=12.0, 6.4 Hz, 1H), 3.34 (s, 1H), 3.08 (s, 3H), 2.84-2.74 (m, 1H), 2.70 (s, 1H). LRMS (ESI) m/z: calcd for Cl₄H₁₈N₃⁺ [M+H]⁺, 228.15; found, 228.32.

### Method E:

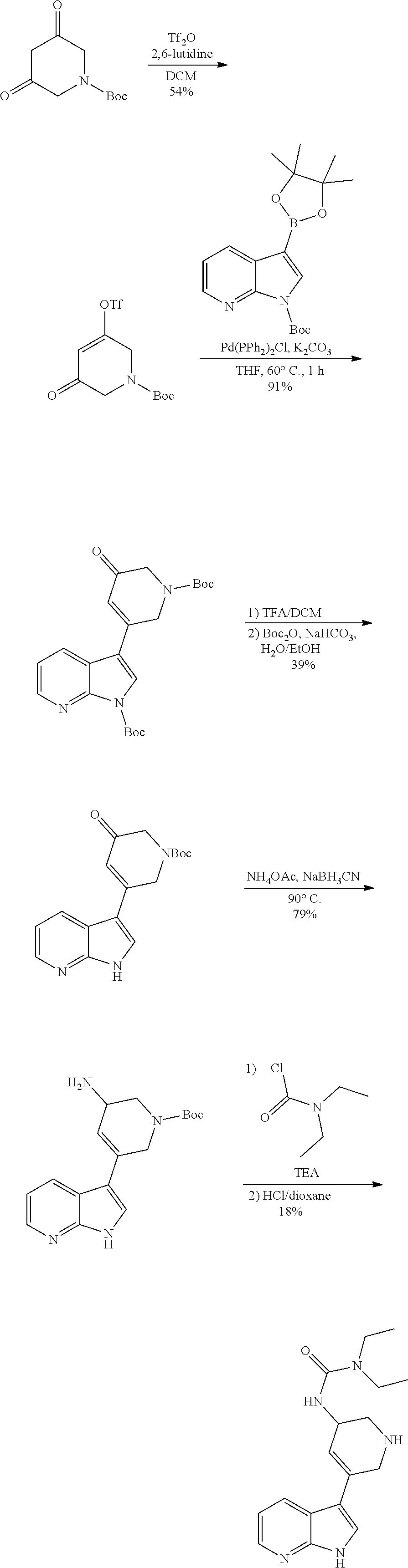

### Example 49

### Synthesis of RS130-132

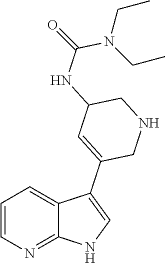 [SMILES: CCN(CC)C(=O)NC1C=C(c2c[nH]c3ncccc23)CNC1]

3-(5-(1H-pyrrolo[2,3-b]pyridin-3-yl)-1,2,3,6-tetrahydropyridin-3-yl)-1,1-diethylurea (RS130-132). RS130-132 was synthesized following method E. To a solution of tert-butyl 3,5-dioxopiperidine-1-carboxylate (2 g, 9.4 mmol, 1 equiv) and 2,6-lutidine (2 g, 18.8 mmol, 2 equiv) in DCM (40 mL) was added Tf₂O (1.2 mL, 7 mmol, 0.75 equiv), then stirred for 30 min at 0° C., the resulting mixture was stirred at rt for 1 h. The mixture was washed with 1N HCl, extracted by DCM, dried by Na₂SO₄, purified by silica gel (0% to 50% ethyl acetate in hexane) to afford tert-butyl 3-oxo-5-(((trifluoromethyl)sulfonyl)oxy)-3,6-dihydropyridine-1(2H)-carboxylate (1.76 g, 73%, yellow oil). Then to a solution of tert-butyl 3-oxo-5-(((trifluoromethyl)sulfonyl)oxy)-3,6-dihydropyridine-1(2H)-carboxylate (3.4 g, 9.85 mmol, 1 equiv) in THF (2 mL) were added tert-butyl 3-(4,4,5,5-tetramethyl-1,3,2-dioxaborolan-2-yl)-1H-pyrrolo[2,3-b]pyridine-1-carboxylate (4.07 g, 1.2 equiv), 2M K₂CO₃ solution (14.8 mL, 3 equiv), Pd(PPh₃)₂Cl₂ (691 mg, 0.1 equiv), and the atmosphere evacuated and backfilled with nitrogen three times. After being stirred for 1 h at 60° C., the resulting mixture was purified by silica gel (0 to 30% ethyl acetate in hexane) to afford tert-butyl 3-(1-(tert-butoxycarbonyl)-5-oxo-1,2,5,6-tetrahydropyridin-3-yl)-1H-pyrrolo[2,3-b]pyridine-1-carboxylate (red oil, 3.7 g, 91%) as an intermediate. To a solution of the last step intermediate (769 mg, 1.86 mmol, 1 equiv) in 3 mL DCM, was added 3 mL TFA, stirred for 1 h at rt, evaporated, then to a solution of the crude compound (1.86 mmol, 1 equiv) in EtOH (2 mL) and water (2 mL) were added (Boc)₂O (400 mg, 1.86 mmol, 1 equiv), NaHCO₃ (156 mg, 1.86 mmol, 1 equiv), after being stirred for 1 h at rt, the resulting mixture was filtered and washed with water and methanol, then the filter cake was dried by vacuum to afford tert-butyl 3-oxo-5-(1H-pyrrolo[2,3-b]pyridin-3-yl)-3,6-dihydropyridine-1(2H)-carboxylate as a light yellow solid (350 mg, 60%). A mixture of tert-butyl 3-oxo-5-(1H-pyrrolo[2,3-b]pyridin-3-yl)-3,6-dihydropyridine-1(2H)-carboxylate (300 mg, 0.96 mmol, 1 equiv), NH₄OAc (738 mg, 9.6 mmol, 10 equiv) and NaBH₃CN (72.3 mg, 1.2 equiv) in methanol was stirred at 90° C. for 3 h, then the mixture was purified by preparative HPLC (10%-100% acetonitrile/0.1% TFA in H₂O) to get the compound tert-butyl 3-amino-5-(1H-pyrrolo[2,3-b]pyridin-3-yl)-3,6-dihydropyridine-1(2H)-carboxylate as a yellow solid (240 mg, 79%). To a solution of tert-butyl 3-amino-5-(1H-pyrrolo[2,3-b]pyridin-3-yl)-3,6-dihydropyridine-1(2H)-carboxylate (265 mg, 0.84 mmol, 1 equiv) in DCM (2 mL) were added TEA (0.234 mL, 2 equiv) and diethylcarbamic chloride (0.106 mL, 0.84 mmol, 1 equiv) at 0° C., then the mixture was stirred for 3 h at rt, evaporated and the resulting mixture was purified by C18 column (10%-100% acetonitrile/0.1% TFA in H₂O) to give the intermediate as a yellow oil, then add 2 mL 4N HCl in dioxane, stirred at rt for 1 h, purified by preparative HPLC (10%-100% acetonitrile/0.1% TFA in H₂O) to give the final compound RS130-132 as a yellow solid (47.4 mg, 18%). ¹H NMR (600 MHz, Methanol-d₄) δ 8.65 (dd, J=16.1, 8.0, 1.3 Hz, 1H), 8.41 (dd, J=4.9, 3.4 Hz, 1H), 7.64 (s, 1H), 7.60 (d, J=6.0 Hz, 1H), 7.53-7.48 (m, 1H), 4.24-4.17 (m, 1H), 3.58-3.55 (m, 1H), 3.39-3.34 (m, 1H), 3.31-3.28 (m, 4H), 3.21 (t, J=12.0 Hz, 1H), 3.11 (t, J=12.1 Hz, 1H), 1.19-1.10 (m, 6H). LRMS (ESI) m/z: calcd for C₁₄H₁₈N₃⁺ [M+H]⁺, 314.20; found, 314.60.

### Example 50

### Synthesis of YX129-177C

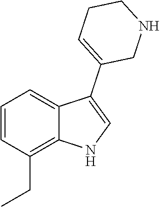 [SMILES: CCc1cccc2c(C3=CCCNC3)c[nH]c12]

7-ethyl-3-(1,2,5,6-tetrahydropyridin-3-yl)-1H-indole (YX129-177C). YX129-177C was synthesized following the standard procedure for preparing RS134-53 from 7-ethyl-1H-indole and tert-butyl 5-(4,4,5,5-tetramethyl-1,3,2-dioxaborolan-2-yl)-3,6-dihydropyridine-1(2H)-carboxylate. (2 mg, 10%) ¹H NMR (600 MHz, Methanol-d₄) δ 7.66 (t, J=7.3 Hz, 1H), 7.34 (d, J=6.9 Hz, 1H), 7.07 (q, J=7.2 Hz, 1H), 7.02 (t, J=6.8 Hz, 1H), 6.41 (s, 1H), 4.10 (s, 2H), 3.43 (q, J=6.3 Hz, 2H), 2.95-2.86 (m, 2H), 2.72-2.62 (m, 2H), 1.34 (q, J=7.4 Hz, 3H). HRMS (ESI-TOF) m/z: [M+H]⁺ calcd for C₁₅H₁₉N₂, 227.1543; found: 227.1560.

### Example 51

### Synthesis of YX129-180C

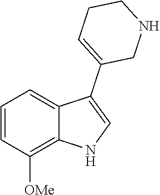 [SMILES: COc1cccc2c(C3=CCCNC3)c[nH]c12]

7-methoxy-3-(1,2,5,6-tetrahydropyridin-3-yl)-1H-indole (YX129-180C). YX129-180C was synthesized following the standard procedure for preparing RS134-53 from 7-methoxy-1H-indole and tert-butyl 5-(4,4,5,5-tetramethyl-1,3,2-dioxaborolan-2-yl)-3,6-dihydropyridine-1(2H)-carboxylate. (3 mg, 11%). ¹H NMR (600 MHz, Methanol-d₄) δ 7.26 (d, J=3.1 Hz, 1H), 6.89 (d, J=7.9 Hz, 1H), 6.66 (d, J=8.0 Hz, 1H), 6.58 (d, J=3.1 Hz, 1H), 6.28 (s, 1H), 4.13-4.06 (m, 2H), 3.98 (s, 3H), 3.45 (t, J=6.3 Hz, 2H), 2.69-2.61 (m, 2H). HRMS (ESI-TOF) m/z: [M+H]f calcd for Cl₄H₁₇N₂O, 229.1335; found: 229.1321.

### Method F:

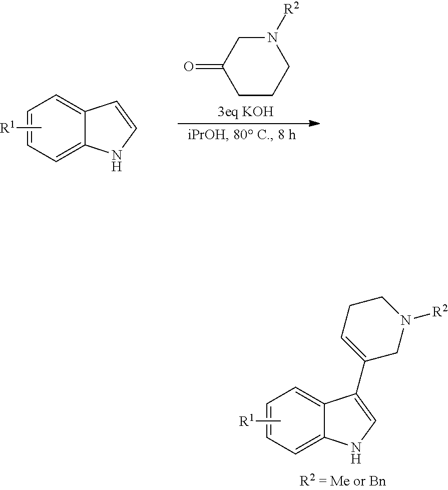

### Example 52

### Synthesis of YX143-19

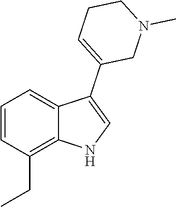 [SMILES: CCc1cccc2c(C3=CCCN(C)C3)c[nH]c12]

7-ethyl-3-(1-methyl-1,2,5,6-tetrahydropyridin-3-yl)-1H-indole (YX143-19). YX143-19 was synthesized following the method F. To a solution of 7-ethyl-1H-indole (29 mg, 0.2 mmol, 1 equiv) in ⁱPrOH (2 mL) were added KOH (56 mg, 5 equiv) and 1-methylpiperidin-3-one HCl salt (89.4 mg, 0.6 mmol, 3 equiv) at rt, then the mixture was stirred for 8 h at 80° C., evaporated and the resulting mixture was purified by C18 column (10%-100% acetonitrile/0.1% TFA in H₂O) to give the product as a yellow oil (18 mg, 60%). ¹H NMR (600 MHz, Methanol-d₄) δ 7.66 (d, J=8.0 Hz, 1H), 7.36 (s, 1H), 7.10-7.04 (m, 1H), 7.02 (d, J=7.0 Hz, 1H), 6.41-6.34 (m, 1H), 4.40-4.29 (m, 1H), 4.04-3.95 (m, 1H), 3.69-3.60 (m, 1H), 3.31-3.24 (m, 1H), 3.06 (s, 3H), 2.91 (q, J=7.6 Hz, 2H), 2.84-2.74 (m, 1H), 2.71-2.61 (m, 1H), 1.34 (t, J=7.6 Hz, 3H). HRMS (ESI-TOF) m/z: [M+H]⁺ calcd for C₁₆H₂₁N₂, 241.1699; found: 241.1693.

### Example 53

### Synthesis of YX143-20

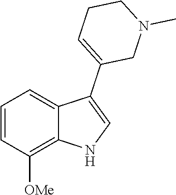 [SMILES: COc1cccc2c(C3=CCCN(C)C3)c[nH]c12]

7-methoxy-3-(1-methyl-1,2,5,6-tetrahydropyridin-3-yl)-1H-indole (YX143-20). YX143-20 was synthesized following the standard procedure for preparing YX143-19 from 7-methoxy-1H-indole and 1-methylpiperidin-3-one. (20 mg, 58%). ¹H NMR (600 MHz, Methanol-d₄) δ 7.40 (d, J=8.1 Hz, 1H), 7.31 (s, 1H), 7.05 (t, J=7.9 Hz, 1H), 6.72 (d, J=7.7 Hz, 1H), 6.40-6.33 (m, 1H), 4.32 (d, J=15.7 Hz, 1H), 4.01-3.92 (m, 4H), 3.68-3.60 (m, 1H), 3.32-3.23 (m, 1H), 3.05 (s, 3H), 2.84-2.73 (m, 1H), 2.70-2.59 (m, 1H). HRMS (ESI-TOF) m/z: [M+H]⁺ calcd for C₁₅H₁₉N₂O, 243.1492; found: 243.1488.

### Example 54

### Synthesis of YX143-2

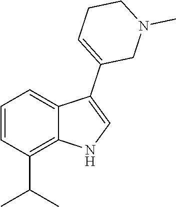 [SMILES: CC(C)c1cccc2c(C3=CCCN(C)C3)c[nH]c12]

7-isopropyl-3-(1-methyl-1,2,5,6-tetrahydropyridin-3-yl)-1H-indole (YX143-2). YX143-2 was synthesized following the standard procedure for preparing YX143-19 from 7-methoxy-1H-indole and 1-methylpiperidin-3-one. (3 mg, 20%). ¹H NMR (600 MHz, Methanol-d₄) δ 7.66 (dd, J=6.6, 2.5 Hz, 1H), 7.36 (d, J=1.9 Hz, 1H), 7.12-7.06 (m, 2H), 6.41-6.37 (m, 1H), 4.40-4.28 (m, 1H), 4.08-3.96 (m, 1H), 3.72-3.62 (m, 1H), 3.40-3.35 (m, 2H), 3.08 (s, 3H), 2.85-2.65 (m, 2H), 1.37 (d, J=6.9 Hz, 6H). HRMS (ESI-TOF) m/z: [M+H]⁺ calcd for C₁₇H₂₃N₂, 255.1856; found: 255.1833.

### Example 55

### Synthesis of YX143-21

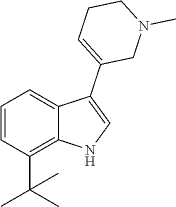 [SMILES: CN1CCC=C(c2c[nH]c3c(C(C)(C)C)cccc23)C1]

7-(tert-butyl)-3-(1-methyl-1,2,5,6-tetrahydropyridin-3-yl)-1H-indole (YX143-21). YX143-21 was synthesized following the standard procedure for preparing YX143-19 from 7-methoxy-1H-indole and 1-methylpiperidin-3-one. (21 mg, 62%). ¹H NMR (600 MHz, Methanol-d₄) δ 7.69 (dd, J=8.0, 1.0 Hz, 1H), 7.35 (s, 1H), 7.15-7.10 (m, 1H), 7.06 (t, J=7.7 Hz, 1H), 6.37-6.29 (m, 1H), 4.33 (d, J=15.7 Hz, 1H), 4.04-3.94 (m, 1H), 3.69-3.63 (m, 1H), 3.31-3.26 (m, 1H), 3.06 (s, 3H), 2.83-2.73 (m, 1H), 2.71-2.59 (m, 1H), 1.50 (s, 9H). HRMS (ESI-TOF) m/z: [M+H]f calcd for C₁₈H₂₅N₂, 269.1012; found: 269.1001.

### Example 56

### Synthesis of NS144-042

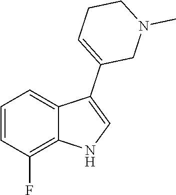 [SMILES: CN1CCC=C(c2c[nH]c3c(F)cccc23)C1]

7-fluoro-3-(1-methyl-1,2,5,6-tetrahydropyridin-3-yl)-1H-indole (NS144-042). NS144-042 was synthesized following the standard procedure for preparing YX143-19 from 7-fluoro-1H-indole and 1-methylpiperidin-3-one. (yellow oil, 7.2 mg, 10%) ¹H NMR (600 MHz, Methanol-d₄) δ 7.60 (d, J=8.1 Hz, 1H), 7.41 (s, 1H), 7.05 (m, 1H), 6.91 (dd, J=11.3, 7.8 Hz, 1H), 6.40 (d, J=4.4 Hz, 1H), 4.33 (d, J=15.7 Hz, 1H), 4.01 (d, J=15.8 Hz, 1H), 3.66 (dd, J=12.5, 6.1 Hz, 1H), 3.33 (d, J=5.1 Hz, 1H), 3.06 (s, 3H), 2.78 (d, J=9.7 Hz, 1H), 2.67 (d, J=19.1 Hz, 1H). LRMS (ESI) m/z: calcd for C₁₄H₁₆FN₂⁺ [M+H]⁺, 231.13; found, 231.27.

### Example 57

### Synthesis of NS144-043

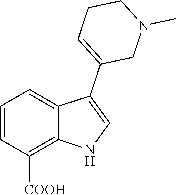 [SMILES: CN1CCC=C(c2c[nH]c3c(C(=O)O)cccc23)C1]

3-(1-methyl-1,2,5,6-tetrahydropyridin-3-yl)-1H-indole-7-carboxylic acid (NS144-043). NS144-043 was synthesized following the standard procedure for preparing YX143-19 from 1H-indole-7-carbonitrile and 1-methylpiperidin-3-one. (yellow oil, 6.8 mg, 9%) ¹H NMR (600 MHz, Methanol-d₄) δ 8.04 (dd, J=7.9, 2.1 Hz, 1H), 7.75-7.68 (m, 1H), 7.49-7.44 (m, 1H), 7.19 (td, J=7.7, 2.2 Hz, 1H), 6.43-6.36 (m, 1H), 4.34 (d, J=15.6 Hz, 1H), 4.01 (d, J=15.9 Hz, 1H), 3.69-3.62 (m, 1H), 3.39 (s, 1H), 3.07 (s, 3H), 2.79 (s, 1H), 2.70 (d, J=7.5 Hz, 1H). LRMS (ESI) m/z: calcd for C₁₅H₁₇N₂O₂⁺ [M+H]⁺, 257.13; found, 257.41.

### Example 58

### Synthesis of NS144-044

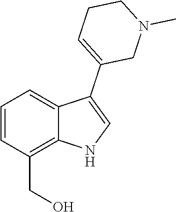 [SMILES: CN1CCC=C(c2c[nH]c3c(CO)cccc23)C1]

(3-(1-methyl-1,2,5,6-tetrahydropyridin-3-yl)-1H-indol-7-yl)methanol (NS144-044). NS144-044 was synthesized following the standard procedure for preparing YX143-19 from (1H-indol-7-yl)methanol and 1-methylpiperidin-3-one. (yellow oil, 7.9 mg, 11%) ¹H NMR (600 MHz, Methanol-d₄) δ 7.76 (d, J=8.1 Hz, 1H), 7.38 (d, J=2.7 Hz, 1H), 7.16 (d, J=7.0 Hz, 1H), 7.12-7.07 (m, 1H), 6.39 (d, J=4.3 Hz, 1H), 4.91 (s, 2H), 4.34 (d, J=15.5 Hz, 1H), 4.01 (d, J=15.4 Hz, 1H), 3.69-3.62 (m, 1H), 3.07 (d, J=3.0 Hz, 3H), 2.79 (d, J=20.7 Hz, 1H), 2.68 (d, J=19.4 Hz, 1H). LRMS (ESI) m/z: calcd for C₁₅H₁₉N₂O⁺ [M+H]⁺, 243.15; found, 243.42.

### Example 59

### Synthesis of YS135-44

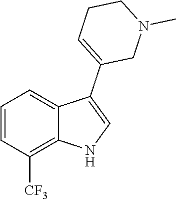 [SMILES: CN1CCC=C(c2c[nH]c3c(C(F)(F)F)cccc23)C1]

3-(1-methyl-1,2,5,6-tetrahydropyridin-3-yl)-7-(trifluoromethyl)-1H-indole (YS135-44). YS135-44 was synthesized following the standard procedure for preparing YX143-19 from 7-(trifluoromethyl)-1H-indole and 1-methylpiperidin-3-one. (15 mg, 30%) ¹H NMR (600 MHz, Methanol-d₄) δ 7.98 (d, J=8.1 Hz, 1H), 7.45-7.30 (m, 2H), 7.15 (t, J=7.8 Hz, 1H), 6.39-6.25 (m, 1H), 4.32-4.16 (m, 1H), 4.03-3.87 (m, 1H), 3.64-3.50 (m, 1H), 3.28-3.21 (m, 1H), 2.97 (s, 3H), 2.77-2.55 (m, 2H). LR-MS (ESI) m/z 281.3[M+H]⁺.

### Example 60

### Synthesis of YS135-45

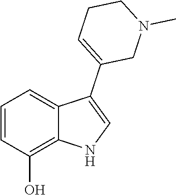 [SMILES: CN1CCC=C(c2c[nH]c3c(O)cccc23)C1]

3-(1-methyl-1,2,5,6-tetrahydropyridin-3-yl)-1H-indol-7-ol (YS135-45). YS135-45 was synthesized following the standard procedure for preparing YX143-19 from 1H-indol-7-ol and 1-methylpiperidin-3-one. (11 mg, 23%) ¹H NMR (600 MHz, Methanol-d₄) δ 7.34-7.13 (m, 2H), 6.90 (t, J=7.9 Hz, 1H), 6.57 (d, J=7.6 Hz, 1H), 6.39-6.29 (m, 1H), 4.31 (d, J=15.8 Hz, 1H), 4.03-3.91 (m, 1H), 3.69-3.60 (m, 1H), 3.34-3.29 (m, 1H), 3.05 (d, J=1.7 Hz, 3H), 2.81-2.58 (m, 2H). MS (ESI) m/z 229.1[M+H]⁺.

### Example 61

### Synthesis of YS135-34

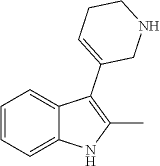 [SMILES: Cc1[nH]c2ccccc2c1C1=CCCNC1]

2-methyl-3-(1,2,5,6-tetrahydropyridin-3-yl)-1H-indole (YS135-34). YS135-34 was synthesized following the standard procedure for preparing RS134-53 from 2-methyl-1H-indole and tert-butyl 5-(4,4,5,5-tetramethyl-1,3,2-dioxaborolan-2-yl)-3,6-dihydropyridine-1(2H)-carboxylate. (5 mg, yield 9%). ¹H NMR (600 MHz, Methanol-d₄) δ 7.63 (t, J=8.2 Hz, 1H), 7.44 (d, J=7.9 Hz, 1H), 7.32-7.20 (m, 1H), 7.08-6.89 (m, 1H), 6.01-5.84 (m, 1H), 4.03-3.77 (m, 2H), 3.47-3.38 (m, 2H), 2.61 (d, J=8.8, 3.0 Hz, 1H), 2.49-2.31 (m, 4H). MS (ESI) m/z 213.2[M+H]⁺.

### Example 62

### Synthesis of YS135-32

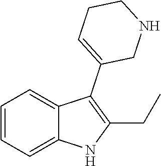 [SMILES: CCc1[nH]c2ccccc2c1C1=CCCNC1]

2-ethyl-3-(1,2,5,6-tetrahydropyridin-3-yl)-1H-indole (YS135-32). YS135-32 was synthesized following the standard procedure for preparing RS134-53 from 2-ethyl-1H-indole and tert-butyl 5-(4,4,5,5-tetramethyl-1,3,2-dioxaborolan-2-yl)-3,6-dihydropyridine-1(2H)-carboxylate. (10 mg, yield 15%). 11H NMR (600 MHz, Methanol-d₄) δ 7.77-7.56 (m, 1H), 7.28 (s, 1H), 7.07-6.86 (m, 2H), 6.01-5.72 (m, 1H), 3.98-3.87 (m, 1H), 3.50-3.45 (m, 1H), 3.42 (d, J=6.3 Hz, 1H), 3.36-3.31 (m, 1H), 2.84-2.68 (m, 3H), 2.62-2.60 (m, 1H), 1.30 (dt, J=12.0, 7.6 Hz, 3H). MS (ESI) m/z 227.1[M+H]⁺.

### Example 63

### Synthesis of YS135-38

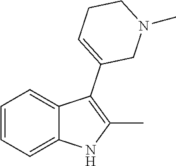 [SMILES: Cc1[nH]c2ccccc2c1C1=CCCN(C)C1]

2-methyl-3-(1-methyl-1,2,5,6-tetrahydropyridin-3-yl)-1H-indole (YS135-38). YS135-38 was synthesized following the standard procedure for preparing RS134-53 from 2-methyl-1H-indole and 1-methyl-5-(4,4,5,5-tetramethyl-1,3,2-dioxaborolan-2-yl)-1,2,3,6-tetrahydropyridine. (7 mg, yield 20%). ¹H NMR (600 MHz, Methanol-d₄) δ 7.55-7.41 (m, 1H), 7.34-7.19 (m, 1H), 7.16-6.93 (m, 2H), 5.96 (dd, J=4.9, 2.6 Hz, 1H), 4.17 (d, J=16.2 Hz, 1H), 4.05-3.93 (m, 1H), 3.69 (dd, J=12.3, 6.3 Hz, 1H), 3.42-3.32 (m, 1H), 3.06 (s, 3H), 2.82-2.76 (m, 1H), 2.68 (d, J=5.2 Hz, 1H), 2.45 (s, 3H). MS (ESI) m/z 227.2 [M+H]⁺.

### Example 64

### Synthesis of YS135-41

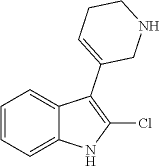 [SMILES: Clc1[nH]c2ccccc2c1C1=CCCNC1]

2-chloro-3-(1,2,5,6-tetrahydropyridin-3-yl)-1H-indole (YS135-41). YS135-41 was synthesized following the standard procedure for preparing RS134-53 from 2-chloro-1H-indole and tert-butyl 5-(4,4,5,5-tetramethyl-1,3,2-dioxaborolan-2-yl)-3,6-dihydropyridine-1(2H)-carboxylate. (3 mg, 5%) ¹H NMR (600 MHz, Methanol-d₄) δ 7.45 (d, J=8.0 Hz, 1H), 7.21 (d, J=8.1 Hz, 1H), 7.12-7.02 (m, 1H), 6.99 (dd, J=8.0, 6.9 Hz, 1H), 6.10 (dq, J=3.9, 2.1 Hz, 1H), 3.97 (q, J=2.3 Hz, 2H), 3.35 (t, J=6.3 Hz, 2H), 2.68-2.48 (m, 2H). MS (ESI) m/z 233.1 [M+H]⁺.

### Example 65

### Synthesis of YS135-39

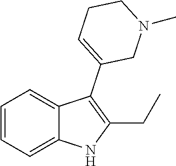 [SMILES: CCc1[nH]c2ccccc2c1C1=CCCN(C)C1]

2-ethyl-3-(1-methyl-1,2,5,6-tetrahydropyridin-3-yl)-1H-indole (YS135-39). YS135-39 was synthesized following the standard procedure for preparing RS134-53 from 2-ethyl-1H-indole and 1-methyl-5-(4,4,5,5-tetramethyl-1,3,2-dioxaborolan-2-yl)-1,2,3,6-tetrahydropyridine. (8 mg, yield 17%) ¹H NMR (600 MHz, Methanol-d₄) δ 7.36 (d, J=7.9 Hz, 1H), 7.19 (d, J=8.0 Hz, 1H), 7.04-6.92 (m, 1H), 6.89 (t, J=7.5 Hz, 1H), 5.82 (dp, J=3.9, 1.9 Hz, 1H), 4.02 (d, J=16.1 Hz, 1H), 3.90-3.75 (m, 1H), 3.56 (dd, J=12.5, 6.3 Hz, 1H), 3.31-3.23 (m, 1H), 2.93 (s, 3H), 2.76-2.60 (m, 3H), 2.52 (d, J=19.1 Hz, 1H), 1.22 (t, J=7.6 Hz, 3H). MS (ESI) m/z 241.2 [M+H]⁺.

### Example 66

### Synthesis of YX143-14A-2

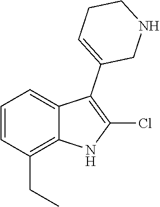 [SMILES: CCc1cccc2c(C3=CCCNC3)c(Cl)[nH]c12]

2-chloro-7-ethyl-3-(1,2,5,6-tetrahydropyridin-3-yl)-1H-indole (YX143-14A-2). YX143-14A-2 was synthesized following the standard procedure for preparing RS134-53 from 2-chloro-7-ethyl-1H-indole and tert-butyl 5-(4,4,5,5-tetramethyl-1,3,2-dioxaborolan-2-yl)-3,6-dihydropyridine-1(2H)-carboxylate. (2 mg, 13%). ¹H NMR (600 MHz, Methanol-d₄) δ 7.35 (dd, J=7.1, 2.0 Hz, 1H), 7.11-7.04 (m, 2H), 6.65-6.60 (m, 1H), 4.35-4.29 (m, 2H), 3.45 (t, J=6.2 Hz, 2H), 2.93 (q, J=7.6 Hz, 3H), 2.73-2.64 (m, 2H), 1.34 (t, J=7.6 Hz, 3H). HRMS (ESI-TOF) m/z: [M+H]⁺ calcd for C₁₅H₁₈ClN₂, 261.1153; found: 261.1158.

### Example 67

### Synthesis of NS144-019

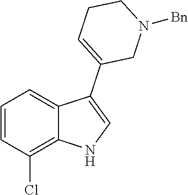 [SMILES: Clc1cccc2c(C3=CCCN(Cc4ccccc4)C3)c[nH]c12]

3-(1-benzyl-1,2,5,6-tetrahydropyridin-3-yl)-7-chloro-1H-indole (NS144-019). NS144-019 was synthesized following the standard procedure for preparing YX143-19 from 7-chloro-1H-indole and 1-benzylpiperidin-3-one. (yellow oil, 48 mg, 11%) ¹H NMR (600 MHz, Methanol-d₄) δ 7.72 (d, J=8.1 Hz, 1H), 7.58 (dd, J=6.7, 3.0 Hz, 2H), 7.53 (q, J=3.6 Hz, 3H), 7.35 (d, J=2.2 Hz, 1H), 7.19 (d, J=7.6 Hz, 1H), 7.07 (t, J=7.8 Hz, 1H), 6.40-6.36 (m, 1H), 4.51 (d, J=48.7 Hz, 2H), 4.15 (d, J=62.9 Hz, 2H), 3.65 (s, 1H), 2.69 (d, J=6.6 Hz, 2H). LRMS (ESI) m/z: calcd for C₂₀H₂₀N₂C₁⁺ [M+H]⁺, 323.13; found, 323.34.

### Example 68

### Synthesis of NS144-021

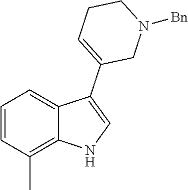 [SMILES: Cc1cccc2c(C3=CCCN(Cc4ccccc4)C3)c[nH]c12]

3-(1-benzyl-1,2,5,6-tetrahydropyridin-3-yl)-7-methyl-1H-indole (NS144-021). NS144-021 was synthesized following the standard procedure for preparing YX143-19 from 7-methyl-1H-indole and 1-benzylpiperidin-3-one. (yellow oil, 50 mg, 12%) ¹H NMR (600 MHz, Methanol-d₄) δ 7.71-7.48 (m, 7H), 7.27 (s, 1H), 7.01-6.95 (m, 2H), 6.42-6.36 (m, 1H), 4.61-4.44 (m, 2H), 4.25-4.08 (m, 2H), 3.66 (s, 1H), 2.70 (s, 2H), 2.49 (d, J=4.9 Hz, 3H). LRMS (ESI) m/z: calcd for C₂₁H₂₃N₂⁺ [M+H]⁺, 303.19; found, 303.35.

### Example 69

### Synthesis of YX143-15

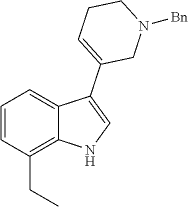 [SMILES: CCc1cccc2c(C3=CCCN(Cc4ccccc4)C3)c[nH]c12]

3-(1-benzyl-1,2,5,6-tetrahydropyridin-3-yl)-7-ethyl-1H-indole (YX143-15). YX143-15 was synthesized following the standard procedure for preparing YX143-19 from 7-ethyl-1H-indole and 1-benzylpiperidin-3-one. (12 mg, 50%). ¹H NMR (600 MHz, Chloroform-d) δ 8.08 (s, 1H), 7.74 (d, J=8.0 Hz, 1H), 7.45 (d, J=7.1 Hz, 2H), 7.37 (q, J=7.5 Hz, 2H), 7.34-7.24 (m, 1H), 7.13 (t, J=7.6 Hz, 1H), 7.10-7.04 (m, 2H), 6.30-6.24 (m, 1H), 3.74 (s, 2H), 3.45-3.39 (m, 2H), 2.86 (q, J=7.6 Hz, 3H), 2.71 (t, J=5.8 Hz, 2H), 2.47-2.40 (m, 2H), 1.38 (t, J=7.6 Hz, 3H).

HRMS (ESI-TOF) m/z: [M+H]⁺ calcd for C₂₂H₂₅N₂, 317.2012; found: 317.2001.

### Example 70

### Synthesis of YX143-16

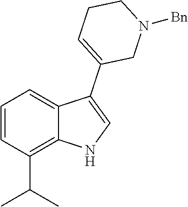 [SMILES: CC(C)c1cccc2c(C3=CCCN(Cc4ccccc4)C3)c[nH]c12]

3-(1-benzyl-1,2,5,6-tetrahydropyridin-3-yl)-7-(propan-2-yl)-1H-indole (YX143-16). YX143-16 was synthesized following the standard procedure for preparing YX143-19 from 7-isopropyl-1H-indole and 1-benzylpiperidin-3-one. (15 mg, 61%). ¹H NMR (600 MHz, Chloroform-d) δ 8.13 (s, 1H), 7.73 (d, J=7.8 Hz, 1H), 7.47-7.42 (m, 2H), 7.36 (t, J=7.6 Hz, 2H), 7.32-7.29 (m, 1H), 7.15 (t, J=7.5 Hz, 1H), 7.12-7.08 (m, 2H), 6.32-6.13 (m, 1H), 3.75 (s, 2H), 3.45-3.37 (m, 2H), 3.27-3.14 (m, 1H), 2.72 (t, J=5.8 Hz, 2H), 2.47-2.41 (m, 2H), 1.40 (d, J=6.9 Hz, 6H). HRMS (ESI-TOF) m/z: [M+H]⁺ calcd for C₂₃H₂₇N₂, 331.2169; found: 331.2155.

### Example 71

### Synthesis of YX143-17C

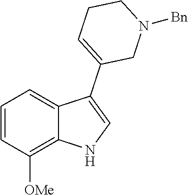 [SMILES: COc1cccc2c(C3=CCCN(Cc4ccccc4)C3)c[nH]c12]

3-(1-benzyl-1,2,5,6-tetrahydropyridin-3-yl)-7-methoxy-1H-indole (YX143-17C). YX143-17C was synthesized following the standard procedure for preparing YX143-19 from 7-methoxy-1H-indole and 1-benzylpiperidin-3-one. (15 mg, 55%). ¹H NMR (600 MHz, Methanol-d₄) δ 7.63-7.57 (m, 2H), 7.57-7.53 (m, 3H), 7.36 (d, J=8.1 Hz, 1H), 7.22 (s, 1H), 7.04 (t, J=7.9 Hz, 1H), 6.72 (d, J=7.7 Hz, 1H), 6.40-6.34 (m, 1H), 4.59-4.50 (m, 1H), 4.50-4.38 (m, 1H), 4.29-4.15 (m, 1H), 4.12-4.00 (m, 1H), 3.97 (s, 3H), 3.71-3.58 (m, 1H), 3.31-3.20 (m, 1H), 2.75-2.62 (m, 2H). HRMS (ESI-TOF) m/z: [M+H]⁺ calcd for C₂₁H₂₃N₂O, 319.1805; found: 319.1830.

### Example 72

### Synthesis of YX143-18C

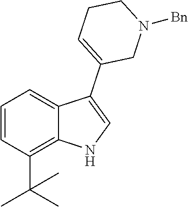 [SMILES: CC(C)(C)c1cccc2c(C3=CCCN(Cc4ccccc4)C3)c[nH]c12]

3-(1-benzyl-1,2,5,6-tetrahydropyridin-3-yl)-7-tert-butyl-1H-indole (YX143-18C). YX143-18C was synthesized following the standard procedure for preparing YX143-19 from 7-(tert-butyl)-1H-indole and 1-benzylpiperidin-3-one. (18 mg, 60%). ¹H NMR (600 MHz, Methanol-d₄) δ 7.65 (d, J=8.0 Hz, 1H), 7.62-7.57 (m, 2H), 7.58-7.53 (m, 3H), 7.26 (s, 1H), 7.12 (d, J=7.4 Hz, 1H), 7.05 (t, J=7.7 Hz, 1H), 6.39-6.34 (m, 1H), 4.61-4.53 (m, 1H), 4.53-4.43 (m, 1H), 4.28-4.16 (m, 1H), 4.16-4.03 (m, 1H), 3.72-3.63 (m, 1H), 3.33 (s, 1H), 3.31 (s, OH), 2.76-2.63 (m, 2H), 1.50 (s, 9H). HRMS (ESI-TOF) m/z: [M+H]⁺ calcd for C₂₄H₂₉N₂, 345.2325; found: 345.2338.

### Example 73

### Synthesis of NS144-047

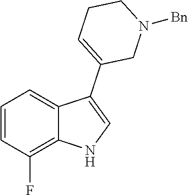 [SMILES: Fc1cccc2c(C3=CCCN(Cc4ccccc4)C3)c[nH]c12]

3-(1-benzyl-1,2,5,6-tetrahydropyridin-3-yl)-7-fluoro-1H-indole (NS144-047). NS144-047 was synthesized following the standard procedure for preparing YX143-19 from 7-fluoro-1H-indole and 1-benzylpiperidin-3-one. (yellow oil, 17.7 mg, 21%) ¹H NMR (600 MHz, Methanol-d₄) δ 7.62-7.52 (m, 6H), 7.32 (s, 1H), 7.04 (dt, J=8.1, 4.2 Hz, 1H), 6.90 (dd, J=11.2, 7.8 Hz, 1H), 6.43-6.36 (m, 1H), 4.62-4.42 (m, 2H), 4.27-4.08 (m, 2H), 3.66 (s, 1H), 3.40 (s, 1H), 2.70 (s, 2H). LRMS (ESI) m/z: calcd for C₂₀H₂₀FN₂⁺ [M+H]⁺, 307.16; found, 307.20.

### Example 74

### Synthesis of NS144-048

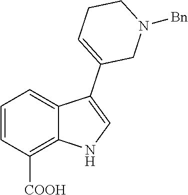 [SMILES: O=C(O)c1cccc2c(C3=CCCN(Cc4ccccc4)C3)c[nH]c12]

3-(1-benzyl-1,2,5,6-tetrahydropyridin-3-yl)-1H-indole-7-carboxylic acid (NS144-048). NS144-048 was synthesized following the standard procedure for preparing YX143-19 from 1H-indole-7-carbonitrile and 1-benzylpiperidin-3-one. (yellow oil, 13.5 mg, 15%) ¹H NMR (600 MHz, Methanol-d₄) δ 8.01 (d, J=7.9 Hz, 1H), 7.70 (d, J=7.6 Hz, 1H), 7.63-7.46 (m, 5H), 7.38 (s, 1H), 7.18 (t, J=7.7 Hz, 1H), 6.41 (s, 1H), 4.59-4.48 (m, 2H), 4.17 (d, J=46.4 Hz, 2H), 3.68 (s, 1H), 3.42 (s, 1H), 2.72 (s, 2H). LRMS (ESI) m/z: calcd for C₂₁H₂₁N₂O₂⁺ [M+H]⁺, 333.16; found, 333.29.

### Example 75

### Synthesis of NS144-049

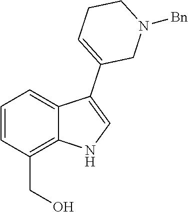 [SMILES: OCc1cccc2c(C3=CCCN(Cc4ccccc4)C3)c[nH]c12]

(3-(1-benzyl-1,2,5,6-tetrahydropyridin-3-yl)-1H-indol-7-yl)methanol (NS144-049). NS144-049 was synthesized following the standard procedure for preparing YX143-19 from (1H-indol-7-yl)methanol and 1-benzylpiperidin-3-one. (yellow oil, 11.3 mg, 13%) ¹H NMR (600 MHz, Methanol-d₄) δ 7.72 (d, J=8.0 Hz, 1H), 7.59 (dd, J=6.7, 3.0 Hz, 2H), 7.56-7.52 (m, 3H), 7.29 (s, 1H), 7.15 (d, J=7.1 Hz, 1H), 7.08 (t, J=7.6 Hz, 1H), 6.39 (t, J=2.0 Hz, 1H), 4.90 (s, 2H), 4.59-4.54 (m, 1H), 4.46 (d, J=12.9 Hz, 1H), 4.22 (d, J=15.6 Hz, 1H), 4.10 (d, J=16.0 Hz, 1H), 3.65 (s, 1H), 2.70 (d, J=6.6 Hz, 2H). LRMS (ESI) m/z: calcd for C₂₁H₂₃N₂O⁺ [M+H]⁺, 319.18; found, 319.25.

### Example 76

### Synthesis of NS136-128

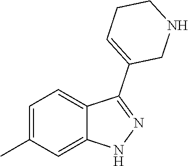 [SMILES: Cc1ccc2c(C3=CCCNC3)n[nH]c2c1]

6-methyl-3-(1,2,5,6-tetrahydropyridin-3-yl)-1H-indazole (NS136-128). NS136-128 was synthesized following the standard procedure for preparing NS131-179 from 3-bromo-6-methyl-1H-indazole and tert-butyl 5-(4,4,5,5-tetramethyl-1,3,2-dioxaborolan-2-yl)-3,6-dihydropyridine-1(2H)-carboxylate. (white solid, 22 mg, 67%) ¹H NMR (400 MHz, Methanol-d₄) δ 7.87-7.80 (m, 1H), 7.30 (d, J=4.5 Hz, 1H), 7.05 (t, J=6.5 Hz, 1H), 6.80 (s, 1H), 4.25 (s, 2H), 3.44 (q, J=6.1 Hz, 2H), 2.69 (d, J=7.7 Hz, 2H), 2.47 (t, J=3.7 Hz, 3H). LRMS (ESI) m/z: calcd for C₁₃H₁₆N₃⁺ [M+H]⁺, 214.13 found, 214.38.

### Example 77

### Synthesis of NS136-129

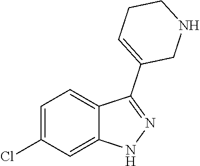 [SMILES: Clc1ccc2c(C3=CCCNC3)n[nH]c2c1]

6-chloro-3-(1,2,5,6-tetrahydropyridin-3-yl)-1H-indazole (NS136-129). NS136-129 was synthesized following the standard procedure for preparing NS131-179 from 3-bromo-6-chloro-1H-indazole and tert-butyl 5-(4,4,5,5-tetramethyl-1,3,2-dioxaborolan-2-yl)-3,6-dihydropyridine-1(2H)-carboxylate. (white solid, 22.2 mg, 64%) ¹H NMR (400 MHz, Methanol-d₄) δ 7.98-7.91 (m, 1H), 7.55 (d, J=4.4 Hz, 1H), 7.18 (t, J=6.6 Hz, 1H), 6.81 (s, 1H), 4.25 (s, 2H), 3.44 (q, J=6.1 Hz, 2H), 2.70 (d, J=7.9 Hz, 2H). LRMS (ESI) m/z: calcd for Cl₂H₁₃ClN₃⁺ [M+H]⁺, 234.08 found, 234.22.

### Example 78

### Synthesis of NS136-130

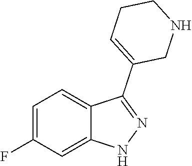 [SMILES: Fc1ccc2c(C3=CCCNC3)n[nH]c2c1]

6-fluoro-3-(1,2,5,6-tetrahydropyridin-3-yl)-1H-indazole (NS136-130). NS136-130 was synthesized following the standard procedure for preparing NS131-179 from 3-bromo-6-fluoro-1H-indazole and tert-butyl 5-(4,4,5,5-tetramethyl-1,3,2-dioxaborolan-2-yl)-3,6-dihydropyridine-1(2H)-carboxylate. (white solid, 13.6 mg, 41%) ¹H NMR (400 MHz, Methanol-d₄) δ 7.98 (dt, J=7.8, 3.9 Hz, 1H), 7.24-7.16 (m, 1H), 7.04-6.98 (m, 4.8 Hz, 1H), 6.82 (s, 1H), 4.25 (s, 2H), 3.45 (q, J=6.0 Hz, 2H), 2.71 (t, J=5.8 Hz, 2H). LRMS (ESI) m/z: calcd for Cl₂H₁₃FN₃⁺ [M+H]⁺, 218.11 found, 218.23.

### Example 79

### Synthesis of NS136-131

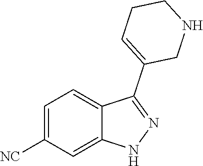 [SMILES: N#Cc1ccc2c(C3=CCCNC3)n[nH]c2c1]

3-(1,2,5,6-tetrahydropyridin-3-yl)-1H-indazole-6-carbonitrile (NS136-131). NS136-131 was synthesized following the standard procedure for preparing NS131-179 from 3-bromo-1H-indazole-6-carbonitrile and tert-butyl 5-(4,4,5,5-tetramethyl-1,3,2-dioxaborolan-2-yl)-3,6-dihydropyridine-1(2H)-carboxylate. (white solid, 19.6 mg, 58%) ¹H NMR (600 MHz, Methanol-d₄) δ 8.24-8.11 (m, 1H), 8.01 (s, 1H), 7.46 (d, J=8.6 Hz, 1H), 6.93-6.81 (m, 1H), 4.36-4.19 (m, 2H), 3.55-3.41 (m, 2H), 2.79-2.67 (m, 2H). LRMS (ESI) m/z: calcd for C₁₃H₁₃N₄⁺ [M+H]⁺, 225.11 found, 225.38.

### Example 80

### Synthesis of NS136-150

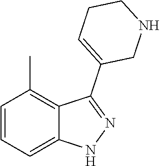 [SMILES: Cc1cccc2[nH]nc(C3=CCCNC3)c12]

4-methyl-3-(1,2,5,6-tetrahydropyridin-3-yl)-1H-indazole (NS136-150). NS136-150 was synthesized following the standard procedure for preparing NS131-179 from 3-bromo-4-methyl-1H-indazole and tert-butyl 5-(4,4,5,5-tetramethyl-1,3,2-dioxaborolan-2-yl)-3,6-dihydropyridine-1(2H)-carboxylate. (white solid, 20.6 mg, 63%) ¹H NMR (600 MHz, Methanol-d₄) δ 7.35 (d, J=8.4 Hz, 1H), 7.27 (dd, J=8.5, 6.9 Hz, 1H), 6.93 (d, J=6.9 Hz, 1H), 6.25 (dq, J=4.1, 2.0 Hz, 1H), 4.10 (q, J=2.3 Hz, 2H), 3.45 (t, J=6.2 Hz, 2H), 2.69-2.61 (m, 2H), 2.61 (s, 3H). LRMS (ESI) m/z: calcd for C₁₃H₁₆N₃⁺ [M+H]⁺, 214.13 found, 214.43.

### Example 81

### Synthesis of NS136-151

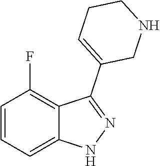 [SMILES: Fc1cccc2[nH]nc(C3=CCCNC3)c12]

4-fluoro-3-(1,2,5,6-tetrahydropyridin-3-yl)-1H-indazole (NS136-151). NS136-151 was synthesized following the standard procedure for preparing NS131-179 from 3-bromo-4-fluoro-1H-indazole and tert-butyl 5-(4,4,5,5-tetramethyl-1,3,2-dioxaborolan-2-yl)-3,6-dihydropyridine-1(2H)-carboxylate. (white solid, 17.9 mg, 54%) ¹H NMR (600 MHz, Methanol-d₄) δ 7.46-7.26 (m, 2H), 6.99-6.81 (m, 2H), 4.38-4.25 (m, 2H), 3.44 (q, J=6.4 Hz, 2H), 2.78-2.63 (m, 2H). LRMS (ESI) m/z: calcd for Cl₂H₁₃FN₃⁺ [M+H]⁺, 218.11 found, 218.28.

### Example 82

### Synthesis of NS136-152

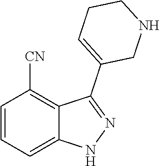 [SMILES: N#Cc1cccc2[nH]nc(C3=CCCNC3)c12]

3-(1,2,5,6-tetrahydropyridin-3-yl)-1H-indazole-4-carbonitrile (NS136-152). NS136-152 was synthesized following the standard procedure for preparing NS131-179 from 3-bromo-1H-indazole-4-carbonitrile and tert-butyl 5-(4,4,5,5-tetramethyl-1,3,2-dioxaborolan-2-yl)-3,6-dihydropyridine-1(2H)-carboxylate. (white solid, 20 mg, 59%) ¹H NMR (600 MHz, Methanol-d₄) δ 7.90 (d, J=8.5 Hz, 1H), 7.69 (d, J=7.1 Hz, 1H), 7.54 (t, J=8.1 Hz, 1H), 6.69-6.57 (m, 1H), 4.34-4.15 (m, 2H), 3.47 (t, J=6.4 Hz, 2H), 2.83-2.64 (m, 2H). LRMS (ESI) m/z: calcd for C₁₃H₁₃N₄⁺ [M+H]⁺, 225.11 found, 225.28.

### Example 83

### Synthesis of NS136-166

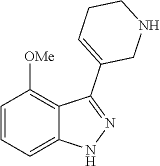 [SMILES: COc1cccc2[nH]nc(C3=CCCNC3)c12]

4-methoxy-3-(1,2,5,6-tetrahydropyridin-3-yl)-1H-indazole (NS136-166). NS136-166 was synthesized following the standard procedure for preparing NS131-179 from 3-bromo-4-methoxy-1H-indazole and tert-butyl 5-(4,4,5,5-tetramethyl-1,3,2-dioxaborolan-2-yl)-3,6-dihydropyridine-1(2H)-carboxylate. (white solid, 21 mg, 61%) ¹H NMR (600 MHz, Methanol-d₄) δ 7.32 (t, J=8.0 Hz, 1H), 7.08 (d, J=8.3 Hz, 1H), 6.84 (td, J=4.1, 2.0 Hz, 1H), 6.60 (d, J=7.7 Hz, 1H), 4.23 (q, J=2.2 Hz, 2H), 3.96 (s, 3H), 3.42 (t, J=6.3 Hz, 2H), 2.69-2.64 (m, 2H). LRMS (ESI) m/z: calcd for C₁₃H₁₆N₃O⁺ [M+H]⁺, 230.13 found, 230.32.

### Example 84

### Synthesis of NS144-011

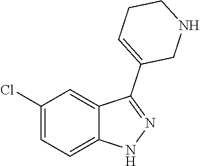 [SMILES: Clc1ccc2[nH]nc(C3=CCCNC3)c2c1]

5-chloro-3-(1,2,5,6-tetrahydropyridin-3-yl)-1H-indazole (NS144-011). NS144-011 was synthesized following the standard procedure for preparing NS131-179 from 3-bromo-5-chloro-1H-indazole and tert-butyl 5-(4,4,5,5-tetramethyl-1,3,2-dioxaborolan-2-yl)-3,6-dihydropyridine-1(2H)-carboxylate. (white solid, 23.6 mg, 68%) ¹H NMR (400 MHz, Methanol-d₄) δ 7.98 (t, J=4.7 Hz, 1H), 7.52 (t, J=6.9 Hz, 1H), 7.38 (d, J=7.9 Hz, 1H), 6.76 (s, 1H), 4.25 (d, J=6.2 Hz, 2H), 3.44 (p, J=5.9 Hz, 2H), 2.70 (d, J=7.8 Hz, 2H). LRMS (ESI) m/z: calcd for C₁₂H₁₃ClN₃⁺ [M+H]⁺, 234.08 found, 234.22.

### Example 85

### Synthesis of NS136-158

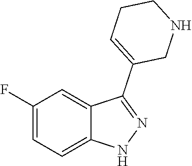 [SMILES: Fc1ccc2[nH]nc(C3=CCCNC3)c2c1]

5-fluoro-3-(1,2,5,6-tetrahydropyridin-3-yl)-1H-indazole (NS136-158). NS136-158 was synthesized following the standard procedure for preparing NS131-179 from 3-bromo-5-fluoro-1H-indazole and tert-butyl 5-(4,4,5,5-tetramethyl-1,3,2-dioxaborolan-2-yl)-3,6-dihydropyridine-1(2H)-carboxylate. (white solid, 29.2 mg, 88%) ¹H NMR (600 MHz, Methanol-d₄) δ 7.67 (dd, J=9.5, 2.4 Hz, 1H), 7.54 (dd, J=9.1, 4.3 Hz, 1H), 7.23 (td, J=9.0, 2.4 Hz, 1H), 6.76-6.69 (m, 1H), 4.26 (q, J=2.2 Hz, 2H), 3.45 (t, J=6.2 Hz, 2H), 2.75-2.66 (m, 2H). LRMS (ESI) m/z: calcd for C₁₂H₁₃FN₃⁺ [M+H]⁺, 218.11 found, 218.33.

### Example 86

### Synthesis of NS136-167

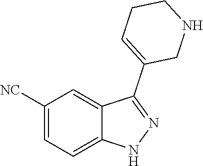 [SMILES: N#Cc1ccc2[nH]nc(C3=CCCNC3)c2c1]

3-(1,2,5,6-tetrahydropyridin-3-yl)-1H-indazole-5-carbonitrile (NS136-167). NS136-167 was synthesized following the standard procedure for preparing NS131-179 from 3-bromo-1H-indazole-5-carbonitrile and tert-butyl 5-(4,4,5,5-tetramethyl-1,3,2-dioxaborolan-2-yl)-3,6-dihydropyridine-1(2H)-carboxylate. (white solid, 20 mg, 59%)¹H NMR (600 MHz, Methanol-d₄) δ 8.52 (d, J=10.3 Hz, 1H), 7.78-7.60 (m, 2H), 7.00-6.84 (m, 1H), 4.43-4.20 (m, 2H), 3.55-3.38 (m, 2H), 2.85-2.65 (m, 2H). LRMS (ESI) m/z: calcd for C₁₃H₁₃N₄⁺ [M+H]⁺, 225.11 found, 225.28.

### Example 87

### Synthesis of NS136-159

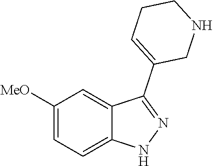 [SMILES: COc1ccc2[nH]nc(C3=CCCNC3)c2c1]

5-methoxy-3-(1,2,5,6-tetrahydropyridin-3-yl)-1H-indazole (NS136-159). NS136-159 was synthesized following the standard procedure for preparing NS131-179 from 3-bromo-5-methoxy-1H-indazole and tert-butyl 5-(4,4,5,5-tetramethyl-1,3,2-dioxaborolan-2-yl)-3,6-dihydropyridine-1(2H)-carboxylate. (white solid, 11 mg, 32%)¹H NMR (600 MHz, Methanol-d₄) δ 7.44 (dd, J=9.0, 2.5 Hz, 1H), 7.30 (s, 1H), 7.12-7.07 (m, 1H), 6.76 (s, 1H), 4.25 (s, 2H), 3.87 (d, J=2.3 Hz, 3H), 3.49-3.42 (m, 2H), 2.76-2.67 (m, 2H). LRMS (ESI) m/z: calcd for C₁₃H₁₆N₃O⁺ [M+H]⁺, 230.13 found, 230.37.

### Example 88

### Synthesis of NS136-135

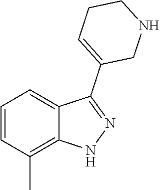 [SMILES: Cc1cccc2c(C3=CCCNC3)n[nH]c12]

7-methyl-3-(1,2,5,6-tetrahydropyridin-3-yl)-1H-indazole (NS136-135). NS136-135 was synthesized following the standard procedure for preparing NS131-179 from 3-bromo-7-methyl-1H-indazole and tert-butyl 5-(4,4,5,5-tetramethyl-1,3,2-dioxaborolan-2-yl)-3,6-dihydropyridine-1(2H)-carboxylate. (white solid, 17.4 mg, 53%) ¹H NMR (400 MHz, Methanol-d₄) δ 7.79 (t, J=6.7 Hz, 1H), 7.15 (dt, J=22.7, 6.8 Hz, 2H), 6.81 (s, 1H), 4.28 (s, 2H), 3.45 (q, J=6.2 Hz, 2H), 2.70 (d, J=6.3 Hz, 2H), 2.55 (t, J=4.0 Hz, 3H). LRMS (ESI) m/z: calcd for C₁₃H₁₆N₃⁺ [M+H]⁺, 214.13 found, 214.18.

### Example 89

### Synthesis of NS136-136

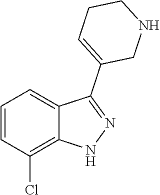 [SMILES: Clc1cccc2c(C3=CCCNC3)n[nH]c12]

7-chloro-3-(1,2,5,6-tetrahydropyridin-3-yl)-1H-indazole (NS136-136). NS136-136 was synthesized following the standard procedure for preparing NS131-179 from 3-bromo-7-chloro-1H-indazole and tert-butyl 5-(4,4,5,5-tetramethyl-1,3,2-dioxaborolan-2-yl)-3,6-dihydropyridine-1(2H)-carboxylate. (white solid, 13.9 mg, 40%) ¹H NMR (400 MHz, Methanol-d₄) δ 7.94 (t, J=6.9 Hz, 1H), 7.44 (d, J=7.2 Hz, 1H), 7.20 (dd, J=8.9, 4.9 Hz, 1H), 6.85 (s, 1H), 4.28 (s, 2H), 3.46 (q, J=6.1 Hz, 2H), 2.72 (d, J=5.9 Hz, 2H). LRMS (ESI) m/z: calcd for C₁₂H₁₃ClN₃⁺ [M+H]⁺, 234.08 found, 234.27.

### Example 90

### Synthesis of NS136-137

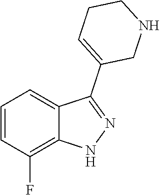 [SMILES: Fc1cccc2c(C3=CCCNC3)n[nH]c12]

7-fluoro-3-(1,2,5,6-tetrahydropyridin-3-yl)-1H-indazole (NS136-137). NS136-137 was synthesized following the standard procedure for preparing NS131-179 from 3-bromo-7-fluoro-1H-indazole and tert-butyl 5-(4,4,5,5-tetramethyl-1,3,2-dioxaborolan-2-yl)-3,6-dihydropyridine-1(2H)-carboxylate. (white solid, 17.6 mg, 53%) ¹H NMR (400 MHz, Methanol-d₄) δ 7.79 (t, J=6.5 Hz, 1H), 7.15 (p, J=6.7, 5.4 Hz, 2H), 6.85 (s, 1H), 4.28 (d, J=4.9 Hz, 2H), 3.45 (d, J=6.5 Hz, 2H), 2.77-2.64 (m, 2H). LRMS (ESI) m/z: calcd for Cl₂H₁₃FN₃⁺ [M+H]⁺, 218.11 found, 218.23.

### Example 91

### Synthesis of NS144-046

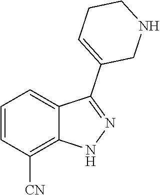 [SMILES: N#Cc1cccc2c(C3=CCCNC3)n[nH]c12]

3-(1,2,5,6-tetrahydropyridin-3-yl)-1H-indazole-7-carbonitrile (NS144-046). NS144-046 was synthesized following the standard procedure for preparing NS131-179 from 3-bromo-1H-indazole-7-carbonitrile and tert-butyl 5-(4,4,5,5-tetramethyl-1,3,2-dioxaborolan-2-yl)-3,6-dihydropyridine-1(2H)-carboxylate. (white solid, 16.6 mg, 49%) ¹H NMR (600 MHz, Methanol-d₄) δ 8.35 (dd, J=8.5, 2.8 Hz, 1H), 7.86 (dd, J=7.5, 2.8 Hz, 1H), 7.36 (dd, J=9.1, 6.4 Hz, 1H), 6.90 (td, J=4.1, 2.0 Hz, 1H), 4.34-4.26 (m, 2H), 3.46 (t, J=6.1 Hz, 2H), 2.73 (tt, J=4.3, 2.2 Hz, 2H). LRMS (ESI) m/z: calcd for C₁₃H₁₃N₄⁺ [M+H]⁺, 225.11 found, 225.33.

### Example 92

### Synthesis of NS144-045

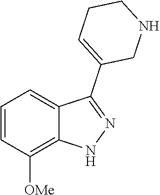 [SMILES: COc1cccc2c(C3=CCCNC3)n[nH]c12]

7-methoxy-3-(1,2,5,6-tetrahydropyridin-3-yl)-1H-indazole (NS144-045). NS144-045 was synthesized following the standard procedure for preparing NS131-179 from 3-bromo-7-methoxy-1H-indazole and tert-butyl 5-(4,4,5,5-tetramethyl-1,3,2-dioxaborolan-2-yl)-3,6-dihydropyridine-1(2H)-carboxylate. (white solid, 15.5 mg, 45%) ¹H NMR (600 MHz, Methanol-d₄) δ 8.16 (d, J=5.1 Hz, 1H), 7.01 (d, J=7.8 Hz, 1H), 6.82 (d, J=7.8 Hz, 1H), 6.40 (d, J=4.6 Hz, 1H), 4.18-4.12 (m, 2H), 4.02 (d, J=14.0 Hz, 3H), 3.45 (d, J=6.2 Hz, 2H), 2.75-2.61 (m, 2H). LRMS (ESI) m/z: calcd for C₁₃H₁₆N₃O⁻ [M+H]⁺, 230.13 found, 230.32.

### Example 93

### Synthesis of NS136-140

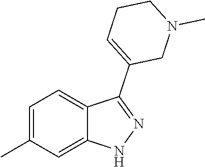

6-methyl-3-(1-methyl-1,2,5,6-tetrahydropyridin-3-yl)-1H-indazole (NS136-140). NS136-140 was synthesized following the method D which the standard procedure for preparing NS136-004. (white solid, 8.3 mg, 81%) ¹H NMR (600 MHz, Methanol-d₄) δ 7.95-7.75 (m, 1H), 7.31 (s, 1H), 7.13-6.99 (m, 1H), 6.80 (s, 1H), 4.60 (d, J=15.9 Hz, 1H), 4.09 (d, J=16.1 Hz, 1H), 3.68 (s, 1H), 3.38 (s, 1H), 3.09 (q, J=10.2 Hz, 3H), 2.90-2.69 (m, 2H), 2.47 (d, J=6.5 Hz, 3H). LRMS (ESI) m/z: calcd for C₁₄H₁₈N₃⁻ [M+H]⁺, 228.15 found, 228.27.

### Example 94

### Synthesis of NS136-141

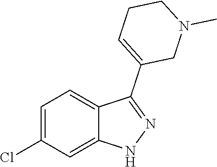 [SMILES: CN1CCC=C(c2n[nH]c3cc(Cl)ccc23)C1]

6-chloro-3-(1-methyl-1,2,5,6-tetrahydropyridin-3-yl)-1H-indazole (NS136-141). NS136-141 was synthesized following the method D which the standard procedure for preparing NS136-004. (white solid, 6.6 mg, 61%) ¹H NMR (600 MHz, Methanol-d₄) δ 7.95-7.75 (m, 1H), 7.31 (s, 1H), 7.13-6.99 (m, 1H), 6.80 (s, 1H), 4.60 (d, J=15.9 Hz, 1H), 4.09 (d, J=16.1 Hz, 1H), 3.68 (s, 1H), 3.38 (s, 1H), 3.09 (q, J=10.2 Hz, 3H), 2.90-2.69 (m, 2H), 2.47 (d, J=6.5 Hz, 3H). LRMS (ESI) m/z: calcd for C₃H₁₅ClN₃⁺ [M+H]⁺, 248.09 found, 248.27.

### Example 95

### Synthesis of NS136-142

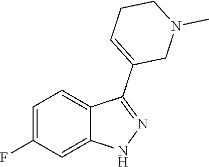 [SMILES: CN1CCC=C(c2n[nH]c3cc(F)ccc23)C1]

6-fluoro-3-(1-methyl-1,2,5,6-tetrahydropyridin-3-yl)-1H-indazole (NS136-142). NS136-142 was synthesized following the method D which the standard procedure for preparing NS136-004. (white solid, 7.5 mg, 72%) ¹H NMR (600 MHz, Methanol-d₄) δ 8.05-7.94 (m, 1H), 7.22 (dt, J=9.2, 2.6 Hz, 1H), 7.05-7.01 (m, 1H), 6.91-6.78 (m, 1H), 4.61 (d, J=16.3 Hz, 1H), 4.09 (d, J=16.4 Hz, 1H), 3.69 (s, 1H), 3.42-3.34 (m, 1H), 3.09 (d, J=2.2 Hz, 3H), 2.79 (d, J=33.6 Hz, 2H). LRMS (ESI) m/z: calcd for C₁₃H₁₅FN₃⁺ [M+H]⁺, 232.12 found, 232.37.

### Example 96

### Synthesis of NS136-143

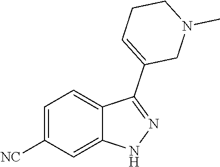 [SMILES: CN1CCC=C(c2n[nH]c3cc(C#N)ccc23)C1]

3-(1-methyl-1,2,5,6-tetrahydropyridin-3-yl)-1H-indazole-6-carbonitrile (NS136-143). NS136-143 was synthesized following the method D which the standard procedure for preparing NS136-004. (white solid, 7.2 mg, 68%) ¹H NMR (600 MHz, Methanol-d₄) δ 8.24-8.12 (m, 1H), 8.01 (s, 1H), 7.52-7.40 (m, 1H), 6.95-6.81 (m, 1H), 4.65 (d, J=16.2 Hz, 1H), 4.13 (d, J=16.2 Hz, 1H), 3.80-3.66 (m, 1H), 3.45-3.35 (m, 1H), 3.16-3.07 (m, 3H), 2.89-2.72 (m, 2H). LRMS (ESI) m/z: calcd for C₁₄H₁₅N₄⁺ [M+H]⁺, 239.13 found, 239.32.

### Example 97

### Synthesis of NS136-153

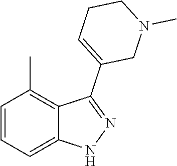 [SMILES: Cc1cccc2[nH]nc(C3=CCCN(C)C3)c12]

4-methyl-3-(1-methyl-1,2,5,6-tetrahydropyridin-3-yl)-1H-indazole (NS136-153). NS136-153 was synthesized following the method D which the standard procedure for preparing NS136-004. (white solid, 8.3 mg, 81%) ¹H NMR (600 MHz, Methanol-d₄) δ7.35 (d, J=8.4 Hz, 1H), 7.28 (dd, J=8.4, 6.9 Hz, 1H), 6.94 (d, J=6.9 Hz, 1H), 6.26 (dd, J=4.1, 2.1 Hz, 1H), 4.37 (d, J=16.4 Hz, 1H), 4.03 (d, J=16.4 Hz, 1H), 3.69 (s, 1H), 3.37 (s, 1H), 3.08 (s, 3H), 2.75 (d, J=26.5 Hz, 2H), 2.61 (s, 3H). LRMS (ESI) m/z: calcd for C₁₄H₁₈N₃⁺ [M+H]⁺, 228.15 found, 228.32.

### Example 98

### Synthesis of NS136-154

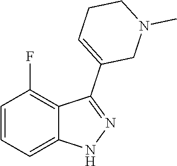 [SMILES: CN1CCC=C(c2n[nH]c3cccc(F)c23)C1]

4-fluoro-3-(1-methyl-1,2,5,6-tetrahydropyridin-3-yl)-1H-indazole (NS136-154). NS136-154 was synthesized following the method D which the standard procedure for preparing NS136-004. (white solid, 7.1 mg, 68%) ¹H NMR (600 MHz, Methanol-d₄) δ 7.43-7.27 (m, 2H), 6.87 (td, J=7.8, 2.5 Hz, 2H), 4.62 (s, 1H), 4.13 (s, 1H), 3.68 (s, 1H), 3.09 (s, 3H), 2.77 (d, J=30.9 Hz, 2H). LRMS (ESI) m/z: calcd for C₁₃H₁₅FN₃⁺ [M+H]⁺, 232.12 found, 232.32.

### Example 99

### Synthesis of NS136-155

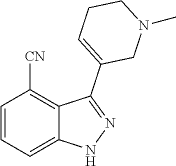 [SMILES: CN1CCC=C(c2n[nH]c3cccc(C#N)c23)C1]

3-(1-methyl-1,2,5,6-tetrahydropyridin-3-yl)-1H-indazole-4-carbonitrile (NS136-155). NS136-155 was synthesized following the method D which the standard procedure for preparing NS136-004. (white solid, 7.5 mg, 71%) ¹H NMR (600 MHz, Methanol-d₄) δ 7.91 (d, J=8.5 Hz, 1H), 7.70 (d, J=7.1 Hz, 1H), 7.55 (dd, J=8.5, 7.1 Hz, 1H), 6.65 (dq, J=4.0, 2.0 Hz, 1H), 4.53 (d, J=16.2 Hz, 1H), 4.09 (d, J=16.0 Hz, 1H), 3.71 (s, 1H), 3.39 (d, J=13.7 Hz, 1H), 3.09 (s, 3H), 2.80 (q, J=19.3, 14.5 Hz, 2H). LRMS (ESI) m/z: calcd for C₁₄H₁₅N₄⁺ [M+H]⁺, 239.13 found, 239.22.

### Example 100

### Synthesis of NS136-175

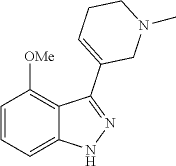 [SMILES: COc1cccc2[nH]nc(C3=CCCN(C)C3)c12]

4-methoxy-3-(1-methyl-1,2,5,6-tetrahydropyridin-3-yl)-1H-indazole (NS136-175). NS136-175 was synthesized following the method D which the standard procedure for preparing NS136-004. (white solid, 7.2 mg, 81%) ¹H NMR (600 MHz, Methanol-d₄) δ 7.32 (dd, J=9.6, 7.0 Hz, 1H), 7.09 (dd, J=8.0, 2.6 Hz, 1H), 6.87 (s, 1H), 6.61 (dd, J=7.9, 2.6 Hz, 1H), 4.55 (d, J=16.0 Hz, 1H), 4.08 (d, J=16.1 Hz, 1H), 3.97 (d, J=2.5 Hz, 3H), 3.70-3.61 (m, 1H), 3.35 (s, 1H), 3.13-2.99 (m, 3H), 2.85-2.67 (m, 2H). LRMS (ESI) m/z: calcd for C₁₄H₁₈N₃O⁺ [M+H]⁺, 244.14 found, 244.27.

### Example 101

### Synthesis of NS144-016

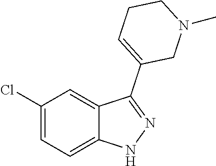 [SMILES: CN1CCC=C(c2n[nH]c3ccc(Cl)cc23)C1]

5-chloro-3-(1-methyl-1,2,5,6-tetrahydropyridin-3-yl)-1H-indazole (NS144-016). NS144-016 was synthesized following the method D which the standard procedure for preparing NS136-004. (white solid, 9.4 mg, 87%) ¹H NMR (600 MHz, Methanol-d₄) δ 8.00 (s, 1H), 7.60-7.46 (m, 1H), 7.39 (d, J=8.8 Hz, 1H), 6.78 (s, 1H), 4.62 (d, J=16.2 Hz, 1H), 4.09 (d, J=16.2 Hz, 1H), 3.79-3.64 (m, 1H), 3.35 (s, 1H), 3.09 (d, J=3.4 Hz, 3H), 2.92-2.71 (m, 2H). LRMS (ESI) m/z: calcd for Cl₃H₁₅ClN₃⁺ [M+H]⁺, 248.09 found, 248.27.

### Example 102

### Synthesis of NS136-160

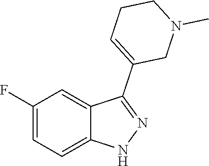 [SMILES: CN1CCC=C(c2n[nH]c3ccc(F)cc23)C1]

5-fluoro-3-(1-methyl-1,2,5,6-tetrahydropyridin-3-yl)-1H-indazole (NS136-160). NS136-160 was synthesized following the method D which the standard procedure for preparing NS136-004. (white solid, 8.7 mg, 84%) ¹H NMR (600 MHz, Methanol-d₄) δ 7.68 (dd, J=9.4, 2.3 Hz, 1H), 7.54 (dd, J=9.1, 4.3 Hz, 1H), 7.24 (td, J=9.0, 2.3 Hz, 1H), 6.75 (tt, J=3.9, 1.8 Hz, 1H), 4.62 (d, J=16.1 Hz, 1H), 4.10 (d, J=16.1 Hz, 1H), 3.71-3.68 (m, 1H), 3.36-3.33 (m, 1H), 3.09 (s, 3H), 2.85-2.69 (m, 2H). LRMS (ESI) m/z: calcd for Cl₃H₁₅FN₃⁺ [M+H]⁺, 232.12 found, 232.27.

### Example 103

### Synthesis of NS136-176

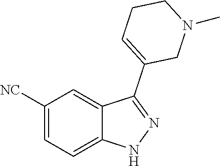 [SMILES: CN1CCC=C(c2n[nH]c3ccc(C#N)cc23)C1]

3-(1-methyl-1,2,5,6-tetrahydropyridin-3-yl)-1H-indazole-5-carbonitrile (NS136-176). NS136-176 was synthesized following the method D which the standard procedure for preparing NS136-004. (white solid, 6.8 mg, 64%) ¹H NMR (600 MHz, Methanol-d₄) δ 8.53 (s, 1H), 7.79-7.61 (m, 2H), 6.96-6.83 (m, 1H), 4.64 (d, J=16.2 Hz, 1H), 4.12 (d, J=16.3 Hz, 1H), 3.71 (s, 1H), 3.37 (s, 1H), 3.18-3.05 (m, 3H), 2.81 (d, J=28.5 Hz, 2H). LRMS (ESI) m/z: calcd for C₁₄H₁₅N₄⁺ [M+H]⁺, 239.13 found, 239.37.

### Example 104

### Synthesis of NS136-161

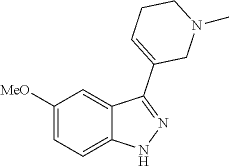 [SMILES: COc1ccc2[nH]nc(C3=CCCN(C)C3)c2c1]

5-methoxy-3-(1-methyl-1,2,5,6-tetrahydropyridin-3-yl)-1H-indazole (NS136-161). NS136-161 was synthesized following the method D which the standard procedure for preparing NS136-004. (white solid, 4.7 mg, 52%) ¹H NMR (600 MHz, Methanol-d₄) δ 7.44 (d, J=8.9 Hz, 1H), 7.30 (d, J=2.3 Hz, 1H), 7.09 (dd, J=9.2, 2.4 Hz, 1H), 6.81-6.73 (m, 1H), 4.62 (d, J=15.9 Hz, 1H), 4.09 (d, J=16.6 Hz, 1H), 3.96-3.85 (m, 3H), 3.69 (d, J=13.1 Hz, 1H), 3.09 (s, 3H), 2.81 (d, J=32.1 Hz, 2H). LRMS (ESI) m/z: calcd for Cl₄H₁₈N₃O⁺ [M+H]⁺, 244.14 found, 244.27.

### Example 105

### Synthesis of NS136-144

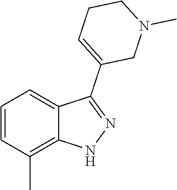 [SMILES: Cc1cccc2c(C3=CCCN(C)C3)n[nH]c12]

7-methyl-3-(1-methyl-1,2,5,6-tetrahydropyridin-3-yl)-1H-indazole (NS136-144). NS136-144 was synthesized following the method D which the standard procedure for preparing NS136-004. (white solid, 7.4 mg, 72%) ¹H NMR (600 MHz, Methanol-d₄) δ 7.79 (d, J=8.2 Hz, 1H), 7.18 (dt, J=6.9, 1.1 Hz, 1H), 7.12 (dd, J=8.2, 6.9 Hz, 1H), 6.81 (dt, J=4.2, 2.1 Hz, 1H), 4.64 (d, J=16.1 Hz, 1H), 4.11 (d, J=16.2 Hz, 1H), 3.69 (s, 1H), 3.35 (s, 1H), 3.09 (s, 3H), 2.79 (d, J=40.7 Hz, 2H), 2.55 (s, 3H). LRMS (ESI) m/z: calcd for C₁₄H₁₈N₃⁺ [M+H]⁺, 228.15 found, 228.37.

### Example 106

### Synthesis of NS136-145

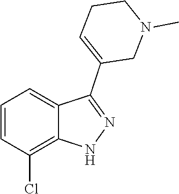 [SMILES: CN1CCC=C(c2n[nH]c3c(Cl)cccc23)C1]

7-chloro-3-(1-methyl-1,2,5,6-tetrahydropyridin-3-yl)-1H-indazole (NS136-145). NS136-145 was synthesized following the method D which the standard procedure for preparing NS136-004. (white solid, 7.0 mg, 64%) ¹H NMR (600 MHz, Methanol-d₄) δ 7.97-7.92 (m, 1H), 7.44 (dd, J=7.5, 0.7 Hz, 1H), 7.20 (dd, J=8.2, 7.5 Hz, 1H), 6.89-6.83 (m, 1H), 4.65 (d, J=16.1 Hz, 1H), 4.13 (d, J=16.3 Hz, 1H), 3.70 (d, J=6.2 Hz, 1H), 3.38 (d, J=11.2 Hz, 1H), 3.10 (s, 3H), 2.80 (d, J=30.4 Hz, 2H). LRMS (ESI) m/z: calcd for C₁₃H₁₅ClN₃⁺ [M+H]⁺, 248.09 found, 248.32.

### Example 107

### Synthesis of NS136-146

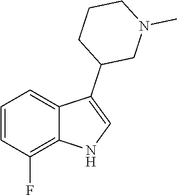 [SMILES: CN1CCCC(c2c[nH]c3c(F)cccc23)C1]

7-fluoro-3-(1-methyl-1,2,5,6-tetrahydropyridin-3-yl)-1H-indazole (NS136-146). NS136-146 was synthesized following the method D which the standard procedure for preparing NS136-004. (white solid, 7.5 mg, 72%) ¹H NMR (600 MHz, Methanol-d₄) δ 7.81-7.77 (m, 1H), 7.20-7.12 (m, 2H), 6.85 (dt, J=4.2, 1.9 Hz, 1H), 4.64 (d, J=16.1 Hz, 1H), 4.16-4.09 (m, 1H), 3.70 (dd, J=12.1, 6.1 Hz, 1H), 3.36 (td, J=11.4, 5.3 Hz, 1H), 3.09 (s, 3H), 2.85-2.81 (m, 1H), 2.79-2.72 (m, 1H). LRMS (ESI) m/z: calcd for C₁₃H₁₅FN₃⁺ [M+H]⁺, 232.12 found, 232.57.

### Example 108

### Synthesis of NS144-051

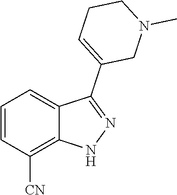 [SMILES: CN1CCC=C(c2n[nH]c3c(C#N)cccc23)C1]

3-(1-methyl-1,2,5,6-tetrahydropyridin-3-yl)-1H-indazole-7-carbonitrile (NS144-051). NS144-051 was synthesized following the method D which the standard procedure for preparing NS136-004. (white solid, 6.7 mg, 63%) ¹H NMR (600 MHz, Methanol-d₄) δ 8.36 (t, J=6.5 Hz, 1H), 7.94-7.84 (m, 1H), 7.45-7.30 (m, 1H), 6.90 (s, 1H), 4.66 (d, J=16.2 Hz, 1H), 4.15 (s, 1H), 3.71 (d, J=11.1 Hz, 1H), 3.48 (s, 1H), 3.18-3.05 (m, 3H), 2.81 (d, J=30.7 Hz, 2H). LRMS (ESI) m/z: calcd for C₁₄H₁₅N₄[M+H]⁺, 239.13 found, 239.33.

### Example 109

### Synthesis of NS144-050

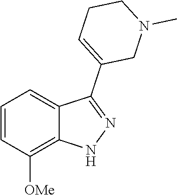 [SMILES: COc1cccc2c(C3=CCCN(C)C3)n[nH]c12]

7-methoxy-3-(1-methyl-1,2,5,6-tetrahydropyridin-3-yl)-1H-indazole (NS144-050). NS144-050 was synthesized following the method D which the standard procedure for preparing NS136-004. (white solid, 7.6 mg, 71%) ¹H NMR (600 MHz, Methanol-d₄) δ 8.17 (s, 1H), 7.02 (d, J=7.8 Hz, 1H), 6.82 (d, J=7.8 Hz, 1H), 6.44-6.35 (m, 1H), 4.34 (d, J=15.7 Hz, 1H), 4.15-4.07 (m, 111), 4.01 (s, 3H), 3.68 (dd, J=12.3, 6.2 Hz, 1H), 3.35 (dd, J=11.3, 5.4 Hz, 1H), 3.07 (s, 3H), 2.85-2.65 (m, 2H). LRMS (ESI) m/z: calcd for C₁₄H₁₈N₃O⁺ [M+H]⁺, 244.14 found, 244.25.

### Method G:

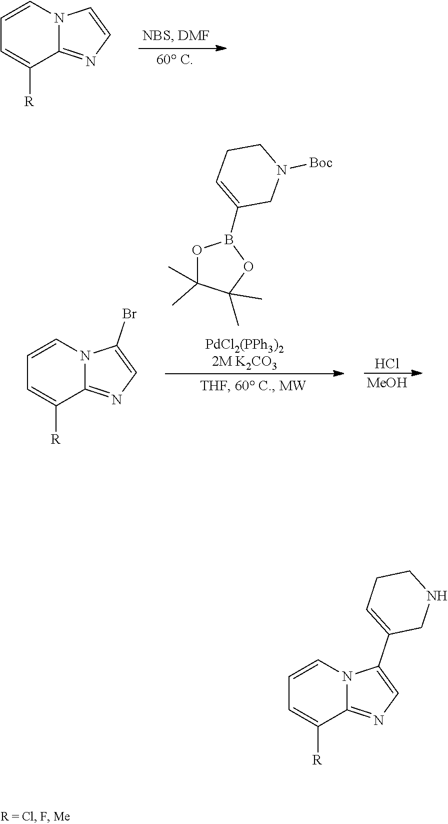

### Example 110

### Synthesis of YX143-41C

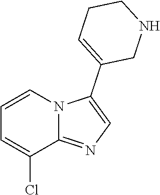 [SMILES: Clc1cccn2c(C3=CCCNC3)cnc12]

8-chloro-3-(1,2,5,6-tetrahydropyridin-3-yl)imidazo[1,2-a]pyridine (YX143-41C). YX143-41C was synthesized following the method G. Intermediate 8-chloroimidazo[1,2-a]pyridine (60 mg, 0.4 mmol), NBS (80 mg, 0.45 mmol) were dissolved in DMF (2 mL). The solution was heated at 60° C. After 2 h, the solution was cooled to room temperature, poured into water, and extracted with ethyl ether (3×5 mL). The organic layer was collected. After dried with Na₂SO₄, the solvent was removed, the residue was purified by ISCO to yield intermediate 3-bromo-8-chloroimidazo[1,2-a]pyridine (46 mg, 50%) as white solid. Intermediate 3-bromo-8-chloroimidazo[1,2-a]pyridine (23 mg, 0.1 mmol), boronic acid ester (44 mg, 0.15 mmol), PdCl₂(PPh₃)₂ (7 mg, 0.01 mmol) and K₂CO₃ (0.15 mL, 2M in water, 0.3 mmol) were mixed together with TUE (1 mL). The mixture was heated under microwave irritation at 60° C. for 1 h. after cooling down to room temperature, the mixture was filtered and the filtrate was collected and purified by prep-HPLC to get oil. The oil was treated with HCl in dioxane (1 mL, 4M, 4 mmol) for 0.5 h. Then the solvent was removed to yield the titled the compound as yellow solid. (24 mg, 80%). ¹H NMR (600 MHz, Methanol-d₄) δ 9.04 (s, 1H), 8.34 (s, 1H), 8.19 (s, 1H), 7.59 (s, 1H), 6.71 (s, 1H), 4.13 (s, 2H), 3.81-3.59 (m, 2H), 3.56 (s, 2H), 2.83 (s, 2H). HRMS (ESI-TOF) m/z: [M+H]⁺ calcd for Cl₂H₁₃N₃Cl, 234.0793; found: 234.0790.

### Example 111

### Synthesis of YX143-42C

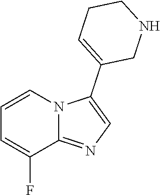 [SMILES: Fc1cccn2c(C3=CCCNC3)cnc12]

8-fluoro-3-(1,2,5,6-tetrahydropyridin-3-yl)imidazo[1,2-a]pyridine (YX143-42C). YX143-42C was synthesized following the standard procedure for preparing YX143-41C from 8-fluoroimidazo[1,2-a]pyridine and tert-butyl 5-(4,4,5,5-tetramethyl-1,3,2-dioxaborolan-2-yl)-3,6-dihydropyridine-1(2H)-carboxylate. (18 mg, 77%). ¹H NMR (600 MHz, Methanol-d₄) δ 8.91 (s, 1H), 8.32 (s, 1H), 7.92 (s, 1H), 7.57 (s, 1H), 6.71 (s, 1H), 4.12 (s, 2H), 3.78-3.61 (m, 2H), 3.56 (s, 2H), 2.82 (s, 2H). HRMS (ESI-TOF) m/z: [M+H]⁺ calcd for C₁₂H₁₃N₃F, 218.1088; found: 218.1201.

### Example 112

### Synthesis of YX143-43D

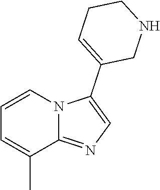 [SMILES: Cc1cccn2c(C3=CCCNC3)cnc12]

8-methyl-3-(1,2,5,6-tetrahydropyridin-3-yl)imidazo[1,2-a]pyridine (YX143-43D). YX143-43D was synthesized following the standard procedure for preparing YX143-41C from 8-methylimidazo[1,2-a]pyridine and tert-butyl 5-(4,4,5,5-tetramethyl-1,3,2-dioxaborolan-2-yl)-3,6-dihydropyridine-1(2H)-carboxylate. (15 mg, 81%). ¹H NMR (600 MHz, Methanol-d₄) δ 8.88 (s, 1H), 8.23 (s, 1H), 7.86 (s, 1H), 7.50 (s, 1H), 6.67 (s, 1H), 4.12 (s, 2H), 3.80-3.49 (m, 4H), 2.82 (s, 2H), 2.72 (s, 3H). HRMS (ESI-TOF) m/z: [M+H]⁺ calcd for C₁₃H₁₅N₃, 214.1399; found: 214.1387.

### Example 113

### Synthesis of NS144-059-2

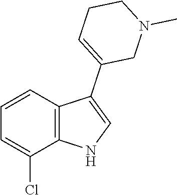 [SMILES: CN1CCC=C(c2c[nH]c3c(Cl)cccc23)C1]

7-chloro-3-(1-methylpiperidin-3-yl)-1H-indole (NS144-059-2). NS144-059-2 was synthesized following the method H. To a solution of 7-chloro-3-(1-methyl-1,2,5,6-tetrahydropyridin-3-yl)-1H-indole (8.5 mg, 0.03 mmol, 1 equiv) in MeOH (1 mL) were added Pd/C (20 mg) and filled with H₂ at rt, then the mixture was stirred for 20 min at rt, evaporated and the resulting mixture was purified by C18 column (10%-100% acetonitrile/0.1% TFA in H₂O) to give the product as the second peak (white solid, 4.0 mg, 40%) ¹H NMR (600 MHz, Methanol-d₄) δ 7.58 (dd, J=8.0, 0.9 Hz, 1H), 7.25 (s, 1H), 7.16 (d, J=7.6 Hz, 1H), 7.04 (t, J=7.8 Hz, 1H), 3.73-3.68 (m, 1H), 3.63-3.57 (m, 1H), 3.36-3.32 (m, 1H), 3.12-3.02 (m, 2H), 2.92 (s, 3H), 2.23-2.17 (m, 1H), 2.16-2.12 (m, 1H), 2.02-1.93 (m, 1H), 1.87-1.81 (m, 1H). LRMS (ESI) m/z: calcd for C₁₄H₁₈ClN₂⁺ [M+H]⁺, 249.12 found, 249.22.

### Example 114

### Synthesis of NS144-054-2

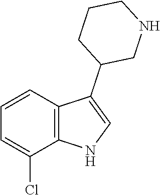 [SMILES: Clc1cccc2c(C3CCCNC3)c[nH]c12]

7-chloro-3-(piperidin-3-yl)-1H-indazole (NS144-054-2). NS144-054-2 was synthesized following the method H which the standard procedure for preparing NS144-059-2. (white solid, 3.8 mg, 47%) ¹H NMR (400 MHz, Methanol-d₄) δ 7.75 (d, J=8.1 Hz, 1H), 7.42 (d, J=7.4 Hz, 1H), 7.15 (t, J=7.8 Hz, 1H), 3.64-3.58 (m, 2H), 3.51 (dd, J=12.9, 10.3 Hz, 1H), 3.42-3.33 (m, 1H), 3.22-3.15 (m, 1H), 2.28 (d, J=9.9 Hz, 1H), 2.05-1.89 (m, 3H). LRMS (ESI) m/z: calcd for Cl₂H₁₅ClN₃⁺ [M+H]⁺, 236.09 found, 236.05.

### Example 115

### Synthesis of NS144-067

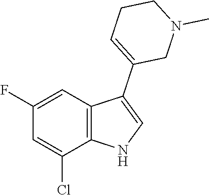 [SMILES: CN1CCC=C(c2c[nH]c3c(Cl)cc(F)cc23)C1]

7-chloro-5-fluoro-3-(1-methyl-1,2,5,6-tetrahydropyridin-3-yl)-1H-indole (NS144-067). NS144-067 was synthesized following the standard procedure for preparing YX143-19 from 7-chloro-5-fluoro-1H-indole and 1-methylpiperidin-3-one. (white solid, 13.3 mg, 35%) ¹H NMR (400 MHz, Methanol-d₄) δ 7.53-7.47 (m, 2H), 7.09 (dd, J=9.0, 2.2 Hz, 1H), 6.35-6.31 (m, 1H), 4.32 (d, J=15.8 Hz, 1H), 4.00 (d, J=15.9 Hz, 1H), 3.65 (s, 1H), 3.24 (s, 1H), 3.06 (s, 3H), 2.72 (d, J=19.4 Hz, 2H). LRMS (ESI) m/z: calcd for Cl₄H₁₅ClFN₂⁺ [M+H]⁺, 265.09 found, 265.13.

### Example 116

### Synthesis of NS144-085

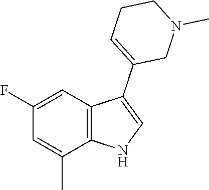 [SMILES: Cc1cc(F)cc2c(C3=CCCN(C)C3)c[nH]c12]

5-fluoro-7-methyl-3-(1-methyl-1,2,5,6-tetrahydropyridin-3-yl)-1H-indole (NS144-085). NS144-085 was synthesized following the standard procedure for preparing YX143-19 from 7-methyl-5-fluoro-1H-indole and 1-methylpiperidin-3-one. (white solid, 21 mg, 12%) ¹H NMR (600 MHz, Methanol-d₄) δ 7.41 (s, 1H), 7.31 (dd, J=10.2, 2.4 Hz, 1H), 6.81-6.76 (m, 1H), 6.30 (t, J=3.9 Hz, 1H), 4.32 (d, J=15.7 Hz, 1H), 4.01-3.96 (m, 1H), 3.65 (dd, J=12.5, 6.1 Hz, 1H), 3.29 (d, J=12.2 Hz, 1H), 3.06 (s, 3H), 2.75 (d, J=9.3 Hz, 1H), 2.67 (d, J=19.0 Hz, 1H), 2.49 (s, 3H). LRMS (ESI) m/z: calcd for C₁₅H₁₈FN₂⁺ [M+H]⁺, 245.14 found, 245.18.

### Example 117

### Synthesis of NS144-093

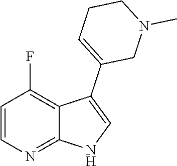 [SMILES: CN1CCC=C(c2c[nH]c3nccc(F)c23)C1]

4-fluoro-3-(1-methyl-1,2,5,6-tetrahydropyridin-3-yl)-1H-pyrrolo[2,3-b]pyridine (NS144-093). NS144-093 was synthesized following the standard procedure for preparing NS131-179 from 3-bromo-4-fluoro-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 14.3 mg, 62%) ¹H NMR (400 MHz, Methanol-d₄) δ 8.27 (dd, J=7.4, 5.6 Hz, 1H), 7.54 (s, 1H), 6.99 (dd, J=11.2, 5.6 Hz, 1H), 6.37-6.32 (m, 1H), 4.31 (d, J=15.9 Hz, 1H), 4.05-3.96 (m, 1H), 3.69-3.60 (m, 1H), 3.31-3.25 (m, 1H), 3.06 (s, 3H), 2.78-2.70 (m, 1H), 2.64 (d, J=19.2 Hz, 1H). LRMS (ESI) m/z: calcd for C₁₃H₁₅FN₃⁺ [M+H]⁺, 232.12 found, 232.18.

### Example 118

### Synthesis of NS144-094

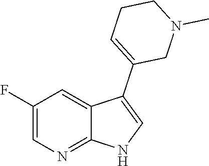 [SMILES: CN1CCC=C(c2c[nH]c3ncc(F)cc23)C1]

5-fluoro-3-(1-methyl-1,2,5,6-tetrahydropyridin-3-yl)-1H-pyrrolo[2,3-b]pyridine (NS144-094). NS144-094 was synthesized following the standard procedure for preparing NS131-179 from 3-bromo-5-fluoro-1H-pyrrolo[2,3-b]pyridine and 1-methyl-5-(4,4,5,5-tetramethyl-1,3,2-dioxaborolan-2-yl)-1,2,3,6-tetrahydropyridine. (white solid, 11 mg, 48%) ¹H NMR (400 MHz, Methanol-d₄) δ 8.18 (t, J=2.2 Hz, 1H), 8.04 (dd, J=9.5, 2.7 Hz, 1H), 7.60 (s, 1H), 6.39-6.33 (m, 1H), 4.34 (d, J=15.8 Hz, 1H), 4.04-4.95 (m, 1H), 3.68-3.60 (m, 1H), 3.28 (d, J=5.4 Hz, 1H), 3.07 (s, 3H), 2.84-2.72 (m, 1H), 2.67 (d, J=19.5 Hz, 1H). LRMS (ESI) m/z: calcd for C₁₃H₁₅FN₃⁺ [M+H]⁺, 232.12 found, 232.16.

### Example 119

### Synthesis of NS144-095

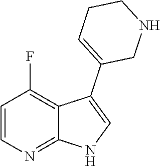 [SMILES: Fc1ccnc2[nH]cc(C3=CCCNC3)c12]

4-fluoro-3-(1,2,5,6-tetrahydropyridin-3-yl)-1H-pyrrolo[2,3-b]pyridine (NS144-095). NS144-095 was synthesized following the standard procedure for preparing NS131-179 from 3-bromo-4-fluoro-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 11.6 mg, 52%) ¹H NMR (400 MHz, Methanol-d₄) δ 8.27 (dd, J=7.3, 5.7 Hz, 1H), 7.53 (s, 1H), 7.00 (dd, J=11.1, 5.7 Hz, 1H), 6.36-6.32 (m, 1H), 4.07 (q, J=2.2 Hz, 2H), 3.40 (t, J=6.2 Hz, 2H), 2.63-2.58 (m, 2H). LRMS (ESI) m/z: calcd for C₁₂H₁₃FN₃⁺ [M+H]⁺, 218.11 found, 218.15.

### Example 120

### Synthesis of NS144-096

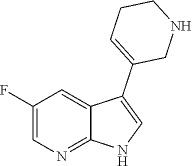 [SMILES: Fc1cnc2[nH]cc(C3=CCCNC3)c2c1]

5-fluoro-3-(1,2,5,6-tetrahydropyridin-3-yl)-1H-pyrrolo[2,3-b]pyridine (NS144-096). NS144-096 was synthesized following the standard procedure for preparing NS131-179 from 3-bromo-5-fluoro-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 11.1 mg, 49%) ¹H NMR (400 MHz, Methanol-d₄) δ 8.17 (t, J=2.2 Hz, 1H), 8.03 (dd, J=9.6, 2.6 Hz, 1H), 7.58 (s, 1H), 6.37 (tt, J=4.0, 1.8 Hz, 1H), 4.07 (q, J=2.2 Hz, 2H), 3.41 (t, J=6.2 Hz, 2H), 2.67-2.63 (m, 2H). LRMS (ESI) m/z: calcd for Cl₂H₁₃FN₃⁺ [M+H]⁺, 218.11 found, 218.15.

### Example 121

### Synthesis of XQ148-012

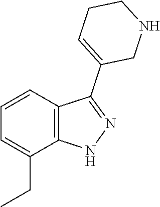 [SMILES: CCc1cccc2c(C3=CCCNC3)n[nH]c12]

7-ethyl-3-(1,2,5,6-tetrahydropyridin-3-yl)-1H-indazole (XQ148-012). XQ148-012 was synthesized following the standard procedure for preparing NS131-179 from 3-bromo-7-ethyl-1H-indazole and tert-butyl 5-(4,4,5,5-tetramethyl-1,3,2-dioxaborolan-2-yl)-3,6-dihydropyridine-1(2H)-carboxylate. (white solid, 10 mg, 6%) ¹H NMR (600 MHz, Methanol-d₄) δ 7.82 (d, J=8.2 Hz, 1H), 7.23 (d, J=7.0 Hz, 1H), 7.17 (t, J=7.6 Hz, 1H), 6.84 (s, 1H), 4.30 (s, 2H), 3.47 (t, J=6.2 Hz, 2H), 2.96 (q, J=7.6 Hz, 2H), 2.75-2.71 (m, 2H), 1.36 (t, J=7.6 Hz, 3H). LRMS (ESI) m/z: calcd for C₁₄H₁₈N₃⁺ [M+H], 228.15 found, 228.22.

### Example 122

### Synthesis of XQ148-023

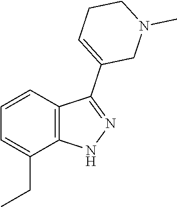 [SMILES: CCc1cccc2c(C3=CCCN(C)C3)n[nH]c12]

7-ethyl-3-(1-methyl-1,2,5,6-tetrahydropyridin-3-yl)-1H-indazole (XQ148-023). XQ148-023 was synthesized following the method D which the standard procedure for preparing NS136-004. (white solid, 5.4 mg, 64%) ¹H NMR (600 MHz, Methanol-d₄) δ 7.82 (d, J=8.2 Hz, 1H), 7.23 (d, J=7.0 Hz, 1H), 7.17 (t, J=7.6 Hz, 1H), 6.84 (s, 1H), 4.30 (s, 2H), 3.47 (t, J=6.2 Hz, 2H), 2.96 (q, J=7.6 Hz, 2H), 2.75-2.71 (m, 2H), 1.36 (t, J=7.6 Hz, 3H). LRMS (ESI) m/z: calcd for C₁₅H₂₀N₃⁺ [M+H]⁺, 242.17 found, 242.27.

### Method I

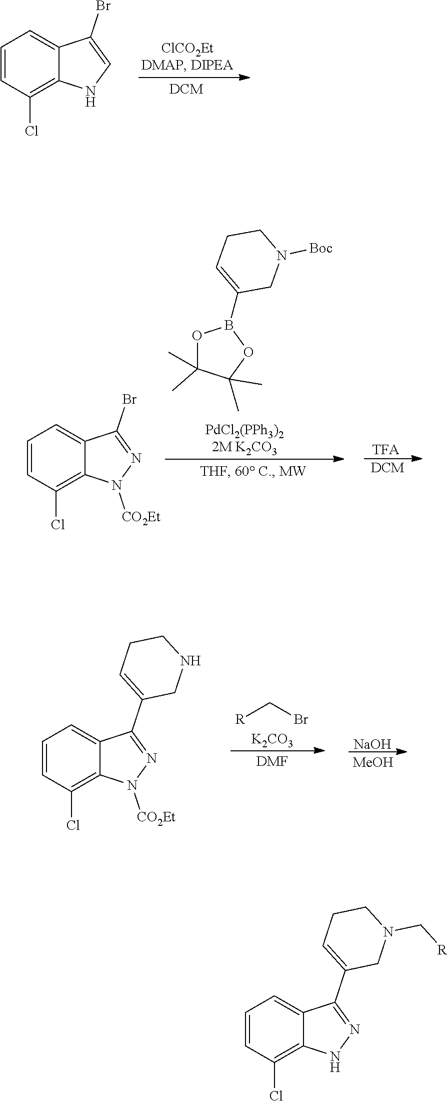

### Example 123

### Synthesis of ZX147-015

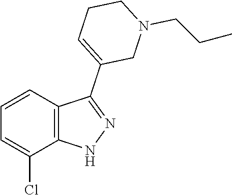 [SMILES: CCCN1CCC=C(c2n[nH]c3c(Cl)cccc23)C1]

7-chloro-3-(1-propyl-1,2,5,6-tetrahydropyridin-3-yl)-1H-indazole (ZX147-015). ZX147-015 was synthesized following the method I. 3-bromo-7-chloro-1H-indazole (100 mg, 0.43 mmol) was dissolved into 5 mL DCM, and DMAP (79 mg, 0.65 mmol) and DIEA (0.37 mL, 2.15 mmol) were added. Ethyl chloroformate (0.21 mL, 2.15 mmol) was added by drop and the mixture was reacted overnight. The mixture was quenched with NaHCO₃ (aq.), separated and the aqueous phase was extracted with DCM for 2 times. The organic phases were combined and concentrated. The residue was purified by flash chromatography (silica gel, PE/EA=5/1) to afford compound ethyl 3-bromo-7-chloro-1H-indazole-1-carboxylate (70 mg, 53%). [M+H]⁺, 305.05. Then the similar coupling reaction was used according to the preparing NS131-179 from tert-butyl 5-(4,4,5,5-tetramethyl-1,3,2-dioxaborolan-2-yl)-3,6-dihydropyridine-1(2H)-carboxylate (69% yield). The compound was dissolved into 3 mL DCM and 1 mL trifluoroacetate was added. The reaction mixture was stirred for 1 h, and all the organic solution and acid were removed under reduced pressure to yield the compound ethyl 7-chloro-3-(1,2,5,6-tetrahydropyridin-3-yl)-1H-indazole-1-carboxylate as crude product and used directly into the next step. Ethyl 7-chloro-3-(1,2,5,6-tetrahydropyridin-3-yl)-1H-indazole-1-carboxylate (22 mg, 0.055 mmol), 1-bromopropane (1.5 equiv) and K₂CO₃ (2.0 equiv) were dissolved into 1 mL DMF and stirred for 5 h. The reaction mixture was quenched with water and extracted with DCM for 3 times. The organic phases were combined and concentrated under reduced pressure. The residue was dissolved into 2 mL methanol and further hydrolyzed by 1 M NaOH (aq.) (2.0 equiv). After the reaction was completed, and the mixture was purified by prepared HPLC to give the product (white solid, 8.3 mg, 39%) ¹H NMR (400 MHz, Methanol-d₄) δ 7.85 (d, J=8.3 Hz, 1H), 7.35 (d, J=7.4 Hz, 1H), 7.11 (t, J=7.9 Hz, 1H), 6.76 (p, J=2.1 Hz, 1H), 4.59-4.49 (m, 1H), 4.03 (d, J=15.8 Hz, 1H), 3.64 (s, 1H), 3.29-3.15 (m, 3H), 2.82-2.59 (m, 2H), 1.81 (dt, J=9.4, 7.3 Hz, 2H), 0.99 (t, J=7.4 Hz, 3H). LRMS (ESI) m/z: calcd for C₁₅H₁₉ClN₃⁺ [M+H]⁺, 276.1262, found, 276.1282.

### Example 124

### Synthesis of ZX147-016

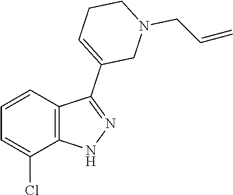 [SMILES: C=CCN1CCC=C(c2n[nH]c3c(Cl)cccc23)C1]

3-(1-allyl-1,2,5,6-tetrahydropyridin-3-yl)-7-chloro-1H-indazole (ZX147-016). ZX147-016 was synthesized following the method I which the standard procedure for preparing ZX147-015. (white solid, 7.8 mg, 37%) ¹H NMR (400 MHz, Methanol-d₄) δ 7.85 (d, J=8.1 Hz, 1H), 7.35 (d, J=7.4 Hz, 1H), 7.11 (t, J=7.9 Hz, 1H), 6.76 (p, J=2.3 Hz, 1H), 5.98 (ddt, J=17.3, 10.1, 7.2 Hz, 1H), 5.66-5.53 (m, 2H), 4.49 (s, 1H), 4.05 (s, 1H), 3.89 (d, J=7.3 Hz, 2H), 3.63 (s, 1H), 3.25 (s, 1H), 2.70 (s, 2H). LRMS (ESI) m/z: calcd for C₁₅H₁₇ClN₃⁺ [M+H]⁺, 274.1106, found, 274.1120.

### Example 125

### Synthesis of ZX147-017

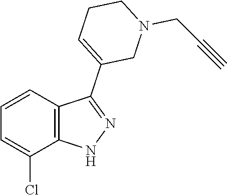 [SMILES: C#CCN1CCC=C(c2n[nH]c3c(Cl)cccc23)C1]

7-chloro-3-(1-(prop-2-yn-1-yl)-1,2,5,6-tetrahydropyridin-3-yl)-1H-indazole (ZX147-017). ZX147-017 was synthesized following the method I which the standard procedure for preparing ZX147-015. (white solid, 5.9 mg, 28%) ¹H NMR (400 MHz, Methanol-d₄) δ 7.85 (d, J=8.3 Hz, 1H), 7.35 (d, J=7.4 Hz, 1H), 7.11 (t, J=7.9 Hz, 1H), 6.77 (dt, J=4.1, 2.2 Hz, 1H), 4.39 (s, 2H), 4.22 (d, J=2.5 Hz, 2H), 3.54 (t, J=6.3 Hz, 2H), 3.32 (t, J=2.6 Hz, 1H), 2.73 (dtt, J=6.3, 4.0, 2.2 Hz, 2H). LRMS (ESI) m/z: calcd for C₁₅H₁₅ClN₃⁺ [M+H]⁻, 272.0949, found, 272.0955.

### Example 126

### Synthesis of ZX147-019

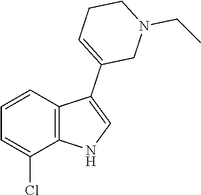 [SMILES: CCN1CCC=C(c2c[nH]c3c(Cl)cccc23)C1]

7-chloro-3-(1-ethyl-1,2,5,6-tetrahydropyridin-3-yl)-1H-indole (ZX147-019). ZX147-019 was synthesized following the standard procedure for preparing YX143-19 from 7-chloro-1H-indole and 1-ethylpiperidin-3-one. (white solid, 40 mg, 32%) ¹H NMR (400 MHz, Methanol-d₄) δ 7.67 (d, J=8.0 Hz, 1H), 7.38 (s, 1H), 7.10 (d, J=7.6 Hz, 1H), 6.99 (t, J=7.9 Hz, 1H), 6.31 (t, J=4.3 Hz, 1H), 4.21 (d, J=15.8 Hz, 1H), 3.91 (d, J=15.6 Hz, 1H), 3.61 (t, J=6.6 Hz, 1H), 3.28 (qd, J=7.4, 4.5 Hz, 2H), 3.19-3.07 (m, 1H), 2.61 (t, J=20.1 Hz, 2H), 1.37 (t, J=7.4 Hz, 3H). LRMS (ESI) m/z: calcd for C₁₅H₁ₛClN₂⁺ [M+H]f, 261.1153, found, 261.1094.

### Method K:

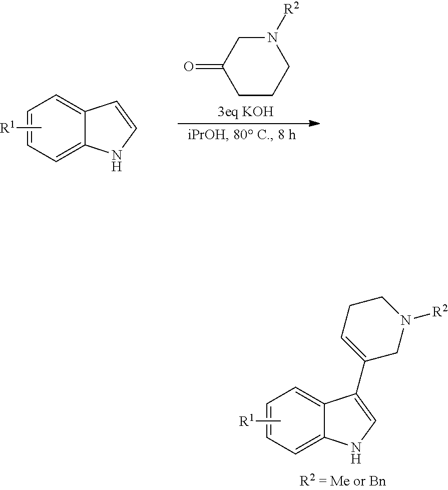

### Example 127

### Synthesis of NS144-097

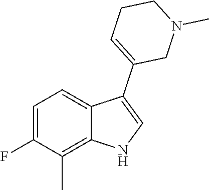 [SMILES: Cc1c(F)ccc2c(C3=CCCN(C)C3)c[nH]c12]

6-fluoro-7-methyl-3-(1-methyl-1,2,5,6-tetrahydropyridin-3-yl)-1H-indole (NS144-097). NS144-097 was synthesized following the Method K. To a solution of 6-fluoro-7-methyl-1H-indole (30 mg, 0.2 mmol, 1 equiv) in ⁱPrOH (2 mL) were added KOH (56 mg, 1.0 mmol, 5 equiv) and 1-methylpiperidin-3-one HCl salt (89.4 mg, 0.6 mmol, 3 equiv) at rt, then the mixture was stirred for 8 h at 80° C., evaporated and the resulting mixture was purified by prep-HPLC to give the product (white solid, 36 mg, 20%) ¹H NMR (400 MHz, Methanol-d₄) δ 7.59 (dd, J=8.8, 4.9 Hz, 1H), 7.36 (s, 1H), 6.86 (dd, J=10.2, 8.8 Hz, 1H), 6.35 (dt, J=4.4, 1.9 Hz, 1H), 4.32 (d, J=15.8 Hz, 1H), 3.97 (dd, J=15.9, 2.8 Hz, 1H), 3.64 (dd, J=12.4, 6.0 Hz, 1H), 3.30-3.24 (m, 1H), 3.05 (s, 3H), 2.81-2.71 (m, 1H), 2.65 (d, J=18.9 Hz, 1H), 2.39 (d, J=1.6 Hz, 3H). MS (ESI) m/z: calcd for C₁₅H₁₈FN₂⁺ [M+H]⁺, 245.1; found, 245.2.

### Example 128

### Synthesis of NS144-098

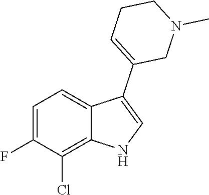 [SMILES: CN1CCC=C(c2c[nH]c3c(Cl)c(F)ccc23)C1]

7-chloro-6-fluoro-3-(1-methyl-1,2,5,6-tetrahydropyridin-3-yl)-1H-indole (NS144-098). NS144-098 was synthesized following the standard procedure for preparing NS144-097 from 7-chloro-6-fluoro-1H-indole and 1-methylpiperidin-3-one. (white solid, 20 mg, 13%) ¹H NMR (400 MHz, Methanol-d₄) δ 7.73 (dd, J=8.8, 4.5 Hz, 1H), 7.44 (s, 1H), 7.02 (dd, J=9.9, 8.8 Hz, 1H), 6.38 (dt, J=4.2, 2.0 Hz, 1H), 4.32 (d, J=15.8 Hz, 1H), 4.02-3.94 (m, 1H), 3.69-3.60 (m, 1H), 3.29 (d, J=8.2 Hz, 1H), 3.06 (s, 3H), 2.83-2.63 (m, 2H). MS (ESI) m/z: calcd for C₁₄H₁₅ClFN₂⁺ [M+H]⁺, 265.1; found, 265.1.

### Method L:

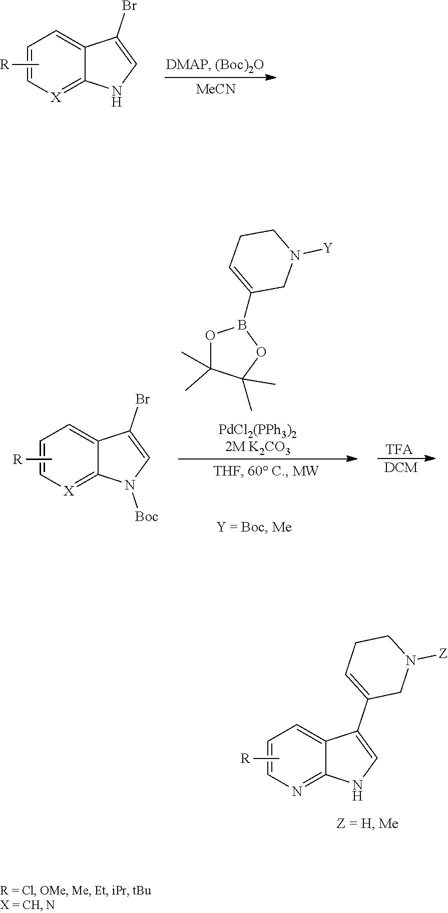

### Example 129

### Synthesis of NS144-102

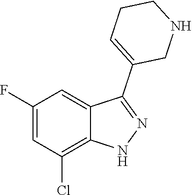 [SMILES: Fc1cc(Cl)c2[nH]nc(C3=CCCNC3)c2c1]

7-chloro-5-fluoro-3-(1,2,5,6-tetrahydropyridin-3-yl)-1H-indazole (NS144-102). NS144-102 was synthesized following the Method L. To a solution of 3-bromo-7-chloro-5-fluoro-1H-indazole (1 mmol, 1 equiv) in MeCN (4 mL) were added DMAP (147 mg, 1.2 mmol, 1.2 equiv), (Boc)₂O (240 mg, 1.1 mmol, 1.1 equiv). After being stirred for 2 h at room temperature, the resulting mixture was purified by silica gel (10% ethyl acetate in hexane) to afford tert-butyl 3-bromo-7-chloro-5-fluoro-1H-pyrrolo[2,3-b]pyridine-1-carboxylate as an intermediate. To a solution of the intermediate (0.1 mmol, 1 equiv) in THF (1 mL) were added tert-butyl 5-(4,4,5,5-tetramethyl-1,3,2-dioxaborolan-2-yl)-3,6-dihydropyridine-1(2H)-carboxylate (31 mg, 0.1 mmol, 1 equiv), 2M K₂CO₃ solution (0.15 mL, 0.3 mmol, 3 equiv), Pd(PPh₃)₂Cl₂ (7.0 mg, 0.01 mmol, 0.1 equiv), under nitrogen atmosphere. After being stirred for 1 h at 60° C. under microwave irradiation, the crude product was added DCM/TFA (1 mL, 1:1), and stirred for 2 h at rt, followed by purified by prep-HPLC to yield NS144-102 (white solid, 22.3 mg, 61%) ¹H NMR (400 MHz, Methanol-d₄) δ 8.09 (d, J=6.9 Hz, 1H), 7.38 (d, J=9.1 Hz, 1H), 6.77 (dq, J=4.0, 2.0 Hz, 1H), 4.24 (q, J=2.2 Hz, 2H), 3.44 (t, J=6.2 Hz, 2H), 2.73-2.68 (m, 2H). MS (ESI) m/z: calcd for Cl₂H₁₂ClFN₃⁺ [M+H]⁺, 252.1; found, 252.1.

### Example 130

### Synthesis of NS144-101

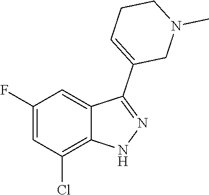 [SMILES: CN1CCC=C(c2n[nH]c3c(Cl)cc(F)cc23)C1]

7-chloro-5-fluoro-3-(1-methyl-1,2,5,6-tetrahydropyridin-3-yl)-1H-indazole (NS144-101). NS144-101 was synthesized following the standard procedure for preparing NS144-102 from 3-bromo-7-chloro-5-fluoro-1H-indazole and 1-methyl-5-(4,4,5,5-tetramethyl-1,3,2-dioxaborolan-2-yl)-1,2,3,6-tetrahydropyridine. (white solid, 20.5 mg, 54%) ¹H NMR (400 MHz, Methanol-d₄) δ 8.09 (d, J=6.9 Hz, 1H), 7.38 (d, J=9.1 Hz, 1H), 6.77 (dt, J=4.2, 2.0 Hz, 1H), 4.60 (d, J=16.2 Hz, 1H), 4.12-4.02 (m, 1H), 3.75-3.63 (m, 1H), 3.35 (dd, J=11.1, 5.7 Hz, 1H), 3.08 (s, 3H), 2.89-2.67 (m, 2H). MS (ESI) m/z: calcd for Cl₃H₁₄ClFN₃⁺ [M+H]⁺, 266.1; found, 266.2.

### Example 131

### Synthesis of NS144-107

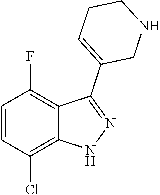 [SMILES: Fc1ccc(Cl)c2[nH]nc(C3=CCCNC3)c12]

7-chloro-4-fluoro-3-(1,2,5,6-tetrahydropyridin-3-yl)-1H-indazole (NS144-107). NS144-107 was synthesized following the standard procedure for preparing NS144-102 from 3-bromo-7-chloro-4-fluoro-1H-indazole and tert-butyl 5-(4,4,5,5-tetramethyl-1,3,2-dioxaborolan-2-yl)-3,6-dihydropyridine-1(2H)-carboxylate. (white solid, 25.3 mg, 69%) ¹H NMR (400 MHz, Methanol-d₄) δ 7.43-7.38 (m, 1H), 6.92-6.85 (m, 2H), 4.28 (t, J=2.2 Hz, 2H), 3.44 (t, J=6.2 Hz, 2H), 2.71-2.65 (m, 2H). MS (ESI) m/z: calcd for C₁₂H₁₂ClFN₃⁺ [M+H]⁺, 252.1; found, 252.1.

### Method M:

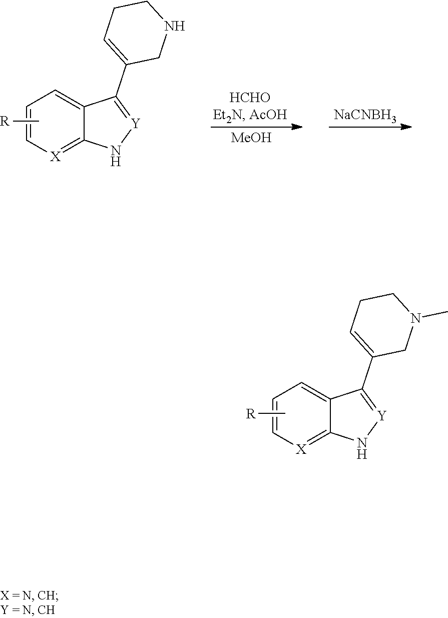

### Example 132

### Synthesis of NS144-108

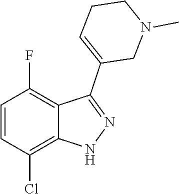 [SMILES: CN1CCC=C(c2n[nH]c3c(Cl)ccc(F)c23)C1]

7-chloro-4-fluoro-3-(1-methyl-1,2,5,6-tetrahydropyridin-3-yl)-1H-indazole (NS144-108). NS144-108 was synthesized following the Method M. To a solution of NS144-107 (0.02 mmol, 1 equiv) in MeOH (1 mL) were added Et₃N (3 drops), AcOH (5 drops), paraformaldehyde (10 mg). After being stirred for 1 h at room temperature, the resulting mixture was added NaCNBH₃ (3.8 mg, 0.06 mmol, 3 equiv), stirred for 1 h at rt, the resulting mixture was purified by prep-HPLC to give NS144-108 (white solid, 9.7 mg, 85%) ¹H NMR (400 MHz, Methanol-d₄) δ 7.41 (ddd, J=8.3, 3.9, 0.9 Hz, 1H), 6.94-6.85 (m, 2H), 4.63 (d, J=16.2 Hz, 1H), 4.13 (d, J=16.1 Hz, 1H), 3.68 (t, J=7.6 Hz, 1H), 3.36 (dd, J=11.1, 5.7 Hz, 1H), 3.09 (d, J=0.9 Hz, 3H), 2.86-2.68 (m, 2H). MS (ESI) m/z: calcd for C₁₃H₁₄ClFN₃⁺ [M+H]f, 266.1; found, 266.2.

### Example 133

### Synthesis of NS144-109

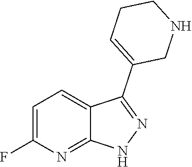 [SMILES: Fc1ccc2c(C3=CCCNC3)n[nH]c2n1]

6-fluoro-3-(1,2,5,6-tetrahydropyridin-3-yl)-1H-pyrrolo[2,3-b]pyridine (NS144-109). NS144-109 was synthesized following the standard procedure for preparing NS144-102 from 3-bromo-6-fluoro-1H-pyrrolo[2,3-b]pyridine and tert-butyl 5-(4,4,5,5-tetramethyl-1,3,2-dioxaborolan-2-yl)-3,6-dihydropyridine-1(2H)-carboxylate. (white solid, 24.5 mg, 55%) ¹H NMR (400 MHz, Methanol-d₄) δ 8.27 (t, J=6.5 Hz, 1H), 7.53 (s, 1H), 7.00 (dd, J=11.2, 5.6 Hz, 1H), 6.35 (td, J=4.3, 2.3 Hz, 1H), 4.07 (d, J=2.9 Hz, 2H), 3.40 (t, J=6.2 Hz, 2H), 2.66-2.61 (m, 2H). MS (ESI) m/z: calcd for C₂H₁₃FN₃⁺ [M+H]⁺, 218.1; found, 218.2.

### Example 134

### Synthesis of NS144-110

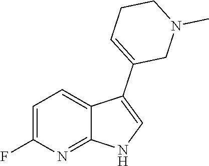 [SMILES: CN1CCC=C(c2c[nH]c3nc(F)ccc23)C1]

6-fluoro-3-(1-methyl-1,2,5,6-tetrahydropyridin-3-yl)-1H-pyrrolo[2,3-b]pyridine (NS144-110). NS144-110 was synthesized following the Method M from NS144-109. (white solid, 5.5 mg, 60%) ¹H NMR (400 MHz, Methanol-d₄) δ 8.25 (t, J=6.5 Hz, 1H), 7.52 (s, 1H), 6.96 (dd, J=11.3, 5.5 Hz, 1H), 6.35 (d, J=4.4 Hz, 1H), 4.31 (d, J=15.8 Hz, 1H), 4.01 (d, J=16.0 Hz, 1H), 3.65 (t, J=9.0 Hz, 1H), 3.28 (d, J=5.3 Hz, 1H), 3.06 (s, 3H), 2.78-2.59 (m, 2H). MS (ESI) m/z: calcd for C₁₃H₁₅FN₃⁺ [M+H]⁺, 232.1; found, 232.1.

### Example 135

### Synthesis of YS135-52

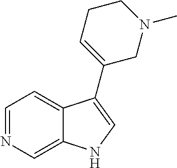 [SMILES: CN1CCC=C(c2c[nH]c3cnccc23)C1]

3-(1-methyl-1,2,5,6-tetrahydropyridin-3-yl)-1H-pyrrolo[2,3-c]pyridine (YS135-52). YS135-52 was synthesized following the Method L which the standard procedure for preparing NS144-102. ¹H NMR (400 MHz, MeOD) δ 9.13 (d, J=2.5 Hz, 1H), 8.45-8.17 (m, 3H), 6.80-6.43 (m, 1H), 4.54-4.00 (m, 2H), 3.83-3.64 (m, 1H), 3.52-3.34 (m, 1H), 3.11 (s, 3H), 2.89-2.59 (m, 2H). MS (ESI) m/z: 214.2 [M+H]⁺.

### Example 136

### Synthesis of YS135-53

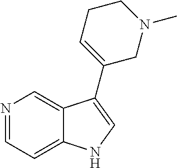 [SMILES: CN1CCC=C(c2c[nH]c3ccncc23)C1]

3-(1-methyl-1,2,5,6-tetrahydropyridin-3-yl)-1H-pyrrolo[3,2-c]pyridine (YS135-53). YS135-53 was synthesized following the Method L. ¹H NMR (400 MHz, MeOD) δ 9.40 (s, 1H), 8.42 (d, J=6.1 Hz, 1H), 8.11-7.90 (m, 2H), 6.81-6.39 (m, 1H), 4.50-3.93 (m, 2H), 3.79-3.44 (m, 2H), 3.11 (s, 3H), 2.88-2.64 (m, 2H). MS (ESI) m/z: [M+H]⁺ 214.3.

### Example 137

### Synthesis of YS135-54

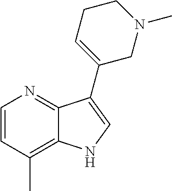 [SMILES: Cc1ccnc2c(C3=CCCN(C)C3)c[nH]c12]

7-methyl-3-(1-methyl-1,2,5,6-tetrahydropyridin-3-yl)-1H-pyrrolo[3,2-b]pyridine (YS135-54). YS135-54 was synthesized following the Method L. ¹H NMR (400 MHz, CDCl₃) δ 7.55-7.22 (m, 1H), 7.22-6.73 (m, 2H), 6.22 (t, J=9.0 Hz, 1H), 5.07-4.82 (m, 4H), 3.62 (s, 3H), 2.26-1.76 (m, 5H). MS (ESI) m/z: [M+H]⁺ 228.1.

### Example 138

### Synthesis of YS135-80

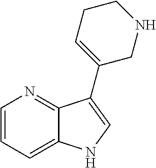 [SMILES: C1=C(c2c[nH]c3cccnc23)CNCC1]

3-(1,2,5,6-tetrahydropyridin-3-yl)-1H-pyrrolo[3,2-b]pyridine (YS135-80). YS135-80 was synthesized following the Method L. ¹H NMR (400 MHz, MeOD) δ 8.51 (dd, J=9.8, 7.0 Hz, 2H), 8.12 (s, 1H), 7.65 (dd, J=8.2, 5.9 Hz, 1H), 6.45 (s, 1H), 3.79-3.57 (m, 2H), 3.36 (t, J=6.2 Hz, 2H), 2.76-2.50 (m, 2H). MS (ESI) m/z: [M+H]⁺ 200.1.

### Example 139

### Synthesis of YS135-81

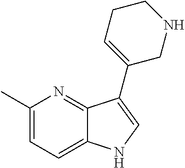 [SMILES: Cc1ccc2[nH]cc(C3=CCCNC3)c2n1]

5-methyl-3-(1,2,5,6-tetrahydropyridin-3-yl)-1H-pyrrolo[3,2-b]pyridine (YS135-81). YS135-81 was synthesized following the Method L. ¹H NMR (400 MHz, MeOD) δ 8.48 (dd, J=8.5, 2.1 Hz, 1H), 8.10 (s, 1H), 7.57 (d, J=8.2 Hz, 1H), 6.56-6.34 (m, 1H), 4.11-4.05 (m, 1H), 3.71 (d, J=2.6 Hz, 1H), 3.47 (t, J=6.2 Hz, 1H), 3.37-3.25 (m, 1H), 2.91 (s, 3H), 2.78-2.57 (m, 1H), 2.57-2.39 (m, 1H). MS (ESI) m/z: [M+H]⁺ 214.2.

### Example 140

### Synthesis of YS135-82

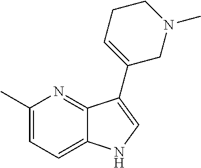

5-methyl-3-(1-methyl-1,2,5,6-tetrahydropyridin-3-yl)-1H-pyrrolo[3,2-b]pyridine (YS135-82). YS135-82 was synthesized following the Method L. ¹H NMR (400 MHz, MeOD) δ 8.36 (d, J=8.4 Hz, 1H), 7.98 (s, 1H), 7.46 (d, J=8.5 Hz, 1H), 6.58-6.12 (m, 1H), 4.24 (d, J=16.1 Hz, 1H), 3.88 (d, J=16.0 Hz, 1H), 3.69-3.46 (m, 1H), 3.31-3.23 (m, 1H), 2.97 (s, 3H), 2.80 (s, 3H), 2.76-2.40 (m, 2H). MS (ESI) m/z: [M+H]⁺ 228.1.

### Example 141

### Synthesis of YS135-96

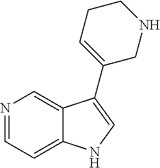 [SMILES: C1=C(c2c[nH]c3ccncc23)CNCC1]

3-(1,2,5,6-tetrahydropyridin-3-yl)-1H-pyrrolo[3,2-c]pyridine (YS135-96). YS135-96 was synthesized following the Method L. ¹H NMR (400 MHz, MeOD) δ 9.38 (d, J=1.1 Hz, 1H), 8.41 (d, J=6.8 Hz, 1H), 8.14-7.58 (m, 2H), 6.82-6.33 (m, 1H), 4.16 (q, J=2.0 Hz, 2H), 3.47 (t, J=6.2 Hz, 2H), 2.95-2.48 (m, 5H). MS (ESI) m/z: [M+H]⁺ 200.1.

### Example 142

### Synthesis of YS135-98

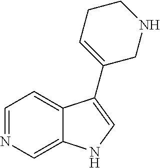 [SMILES: C1=C(c2c[nH]c3cnccc23)CNCC1]

3-(1,2,5,6-tetrahydropyridin-3-yl)-1H-pyrrolo[2,3-c]pyridine (YS135-98). YS135-98 was synthesized following the Method L. ¹H NMR (400 MHz, MeOD) δ 9.12 (d, J=1.0 Hz, 1H), 8.62-8.17 (m, 3H), 6.94-6.35 (m, 1H), 4.17 (d, J=2.3 Hz, 2H), 3.47 (t, J=6.2 Hz, 2H), 2.91-2.62 (m, 2H). MS (ESI) m/z: [M+H]⁺ 200.2.

### Example 143

### Synthesis of YS135-99

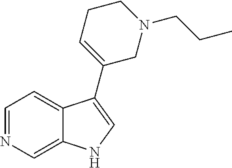 [SMILES: CCCN1CCC=C(c2c[nH]c3cnccc23)C1]

3-(1-propyl-1,2,5,6-tetrahydropyridin-3-yl)-1H-pyrrolo[2,3-c]pyridine (YS135-99). YS135-99 was synthesized following the Method M using propionaldehyde instead of formaldehyde. ¹H NMR (400 MHz, MeOD) δ 9.13 (d, J=2.0 Hz, 1H), 8.52-8.14 (m, 3H), 6.55 (tt, J=3.8, 2.0 Hz, 1H), 4.41 (d, J=16.0 Hz, 1H), 4.12 (d, J=16.0 Hz, 1H), 3.76 (d, J=8.1 Hz, 1H), 3.39-3.24 (m, 3H), 2.97-2.60 (m, 2H), 1.95-1.82 (m, 2H), 1.37-0.86 (m, 3H). MS (ESI) m/z: [M+H]⁺ 242.3.

### Example 144

### Synthesis of YS135-100

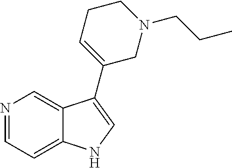 [SMILES: CCCN1CCC=C(c2c[nH]c3ccncc23)C1]

3-(1-propyl-1,2,5,6-tetrahydropyridin-3-yl)-1H-pyrrolo[3,2-c]pyridine (YS135-100). YS135-100 was synthesized following the Method M using propionaldehyde instead of formaldehyde. ¹H NMR (400 MHz, MeOD) δ 9.39 (s, 1H), 8.42 (d, J=6.6 Hz, 1H), 8.01 (d, J=7.1 Hz, 2H), 6.87-6.35 (m, 1H), 4.66-4.20 (m, 1H), 4.20-3.96 (m, 1H), 3.90-3.72 (m, 1H), 3.51-3.15 (m, 3H), 3.05-2.47 (m, 2H), 1.99-1.69 (m, 2H), 1.11 (t, J=1.6 Hz, 3H). MS (ESI) m/z: [M+H]⁺ 242.2.

### Examples 145 and 146

### Synthesis of Compounds ZX147-026-1 and ZX147-026-2:

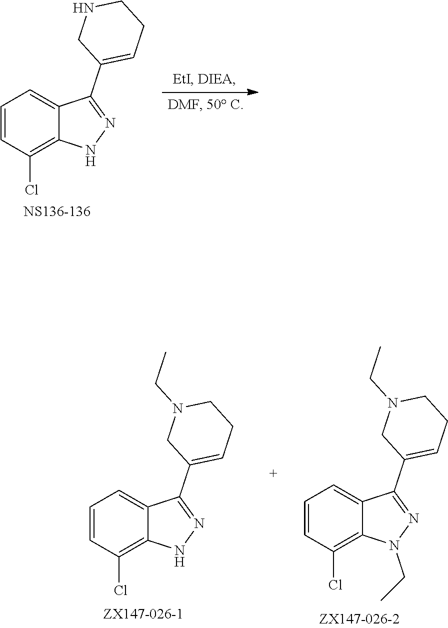 [SMILES: Clc1cccc2c(C3=CCCNC3)n[nH]c12 ; CCN1CCC=C(c2n[nH]c3c(Cl)cccc23)C1 ; CCN1CCC=C(c2nn(CC)c3c(Cl)cccc23)C1]

7-chloro-3-(1,2,5,6-tetrahydropyridin-3-yl)-1H-indazole (NS136-136). NS136-136 was synthesized following Method L from 3-bromo-7-chloro-1H-indazole and tert-butyl 5-(4,4,5,5-tetramethyl-1,3,2-dioxaborolan-2-yl)-3,6-dihydropyridine-1(2H)-carboxylate. (white solid, 13.9 mg, 40%) ¹H NMR (400 MHz, Methanol-d₄) δ 7.94 (t, J=6.9 Hz, 1H), 7.44 (d, J=7.2 Hz, 1H), 7.20 (dd, J=8.9, 4.9 Hz, 1H), 6.85 (s, 1H), 4.28 (s, 2H), 3.46 (q, J=6.1 Hz, 2H), 2.72 (d, J=5.9 Hz, 2H). MS (ESI) m/z: calcd for C₁₂H₁₃ClN₃⁺ [M+H]⁺, 234.1 found, 234.3.

The mixture of NS136-136 (25 mg, 0.075 mmol, 1 equiv.), EtI (23 mg, 2.0 equiv.) and DIEA (49 mg, 5.0 equiv.) in DMF (1 mL) was stirred at 50° C. in a sealed tube overnight. The reaction mixture was purified by preparative HPLC to yield ZX147-026-1 (white solid, 10 mg, 37% yield) and ZX147-026-2 (white solid, 8 mg, 26% yield).

7-chloro-3-(1-ethyl-1,2,5,6-tetrahydropyridin-3-yl)-1H-indazole (ZX147-026-1). ¹H NMR (400 MHz, Methanol-d₄) δ 7.95 (d, J=8.3 Hz, 1H), 7.45 (d, J=7.4 Hz, 1H), 7.21 (t, J=7.9 Hz, 1H), 6.86 (dt, J=4.1, 2.1 Hz, 1H), 4.64 (d, J=15.2 Hz, 1H), 4.11 (d, J=12.6 Hz, 1H), 3.81-3.69 (m, 1H), 3.41 (q, J=7.3 Hz, 2H), 3.35-3.25 (m, 1H), 2.85-2.75 (m, 2H), 1.47 (t, J=7.3 Hz, 3H). MS (ESI) m/z: [M+H]⁺ 262.2.

7-chloro-1-ethyl-3-(1-ethyl-1,2,5,6-tetrahydropyridin-3-yl)-1H-indazole (ZX147-026-2). ¹H NMR (400 MHz, Methanol-d₄) δ 7.96 (d, J=8.1 Hz, 1H), 7.45 (d, J=7.4 Hz, 1H), 7.22 (t, J=7.9 Hz, 1H), 6.87 (dp, J=4.0, 1.8 Hz, 1H), 4.52 (q, J=2.2 Hz, 2H), 3.66 (t, J=6.3 Hz, 2H), 3.55 (dh, J=21.0, 7.2 Hz, 4H), 2.82 (tq, J=6.4, 4.0, 3.2 Hz, 2H), 1.44 (t, J=7.3 Hz, 6H). MS (ESI) m/z: [M+H]⁺ 290.1.

### Examples 147 and 148

Synthesis of compounds ZX147-027 and ZX147-029:

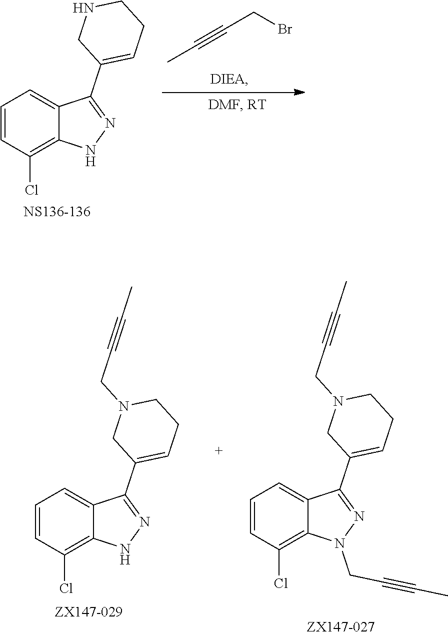

ZX147-027 and ZX147-029 were synthesized following similar procedure for preparing ZX147-026-1 and ZX147-026-2.

1-(but-2-yn-1-yl)-3-(1-(but-2-yn-1-yl)-1,2,5,6-tetrahydropyridin-3-yl)-7-chloro-1H-indazole (ZX147-029). Yield: 37%. ¹H NMR (400 MHz, Methanol-d₄) δ 7.94 (d, J=8.1 Hz, 1H), 7.44 (d, J=7.5 Hz, 1H), 7.20 (t, J=7.9 Hz, 1H), 6.85 (dp, J=4.1, 1.9 Hz, 1H), 4.71 (br, 2H), 4.21 (q, J=2.6 Hz, 2H), 3.60 (br, 2H), 2.82 (s, 2H), 1.96 (t, J=2.5 Hz, 3H). MS(ESI) [M+H]⁺: 286.4.

7-chloro-3-(1-isopropyl-1,2,5,6-tetrahydropyridin-3-yl)-1H-indazole (ZX147-027). ¹H NMR (400 MHz, Methanol-d₄) δ 7.95 (d, J=8.3 Hz, 1H), 7.44 (d, J=7.4 Hz, 1H), 7.21 (t, J=7.9 Hz, 1H), 6.87 (dt, J=4.1, 2.1 Hz, 1H), 4.66 (d, J=2.3 Hz, 2H), 4.46 (q, J=2.5 Hz, 4H), 3.82 (t, J=6.3 Hz, 2H), 2.86 (tq, J=6.4, 4.0, 3.2 Hz, 2H), 1.99 (t, J=2.5 Hz, 6H). MS(ESI) m/z: [M+H]f 337.9.

### Example 149

### Synthesis of Compound ZX147-028:

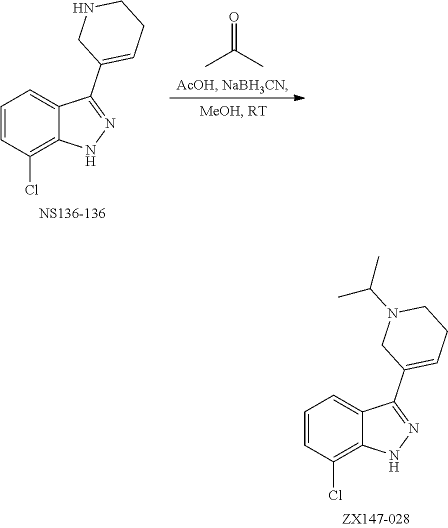

3-(1-(but-2-yn-1-yl)-1,2,5,6-tetrahydropyridin-3-yl)-7-chloro-1H-indazole (ZX147-028).

ZX147-028 was synthesized following Method M using acetone instead of paraformaldehyde (white solid, 65% yield)¹H NMR (400 MHz, Methanol-d₄) δ 7.85 (d, J=8.3 Hz, 1H), 7.34 (d, J=7.5 Hz, 1H), 7.10 (t, J=7.9 Hz, 1H), 6.75 (tt, J=3.9, 1.9 Hz, 1H), 4.41 (d, J=15.5 Hz, 1H), 4.10 (d, J=15.3 Hz, 1H), 3.70-3.55 (m, 2H), 3.29-3.16 (m, 1H), 2.86-2.60 (m, 2H), 1.39 (d, J=6.6 Hz, 6H). MS(ESI) m/z: [M+H]⁺ 276.3.

### Example 150

### Synthesis of ZX147-031:

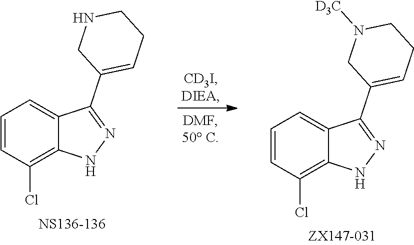 [SMILES: Clc1cccc2c(C3=CCCNC3)n[nH]c12 ; [2H]C([2H])([2H])N1CCC=C(c2n[nH]c3c(Cl)cccc23)C1]

7-chloro-3-(1-(methyl-d₃)-1,2,5,6-tetrahydropyridin-3-yl)-1H-indazole (ZX147-031).

The mixture of NS136-136 (25 mg, 0.075 mmol, 1.0 equiv.), CD₃I (16.5 mg, 0.11 mmol, 1.5 equiv.) and DIEA (65 uL, 0.38 mmol, 5.0 equiv.) in DMF (2 mL) was stirred at 50° C. in a sealed tube overnight. The reaction mixture was diluted by DCM (10 mL), washed with brine, dried over Na₂SO₄ and concentrated, followed by purified by silica gel chromatography (DCM-DCM/MeOH=5/1), further purified by prep-HPLC. (white solid, 20%). ¹H NMR (400 MHz, Methanol-d₄) δ 7.97 (d, J=8.3 Hz, 1H), 7.46 (d, J=7.5 Hz, 1H), 7.22 (t, J=7.9 Hz, 1H), 6.87 (tt, J=3.9, 1.9 Hz, 1H), 4.67 (d, J=15.9 Hz, 1H), 4.14 (d, J=16.3 Hz, 1H), 3.77-3.67 (m, 1H), 3.43-3.35 (m, 1H), 2.92-2.73 (m, 2H). HRMS (ESI-TOF) m/z: calcd for C₁₃H₁₂D₃ClN₃⁺ [M+H]⁺ 251.1137; found 251.1144.

### Example 151

### Synthesis of ZX147-054:

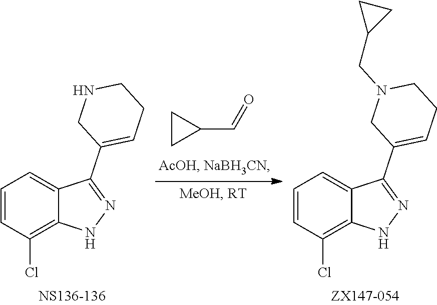

7-chloro-3-(1-(cyclopropylmethyl)-1,2,5,6-tetrahydropyridin-3-yl)-1H-indazole (ZX147-054). ZX147-054 was prepared according to the same procedure for ZX147-028 but using cyclopropanecarbaldehyde instead of acetone. Yield: 45%. ¹H NMR (400 MHz, Methanol-d₄) δ 7.96 (d, J=8.3 Hz, 1H), 7.46 (d, J=7.5 Hz, 1H), 7.29-7.16 (m, 1H), 6.87 (td, J=4.3, 1.9 Hz, 1H), 4.73 (s, 1H), 4.21 (s, 1H), 3.82 (s, 1H), 3.44 (s, 1H), 3.27 (d, J=7.4 Hz, 2H), 2.82 (s, 2H), 1.35-1.23 (m, 1H), 0.85 (d, J=8.1 Hz, 2H), 0.54 (d, J=4.9 Hz, 2H). MS (ESI) m/z: [M+H]⁺288.3.

### Example 152

### Synthesis of ZX147-055:

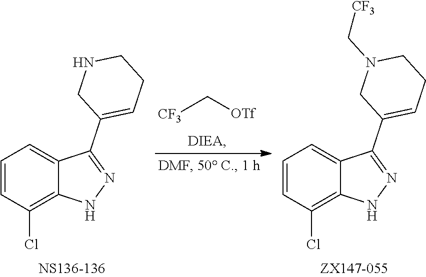

7-chloro-3-(1-(2,2,2-trifluoroethyl)-1,2,5,6-tetrahydropyridin-3-yl)-1H-indazole (ZX147-055). ZX147-055 was synthesized following similar procedure for preparing ZX147-031 (27% yield). ¹H NMR (400 MHz, Methanol-d₄) δ 7.81 (d, J=8.3 Hz, 1H), 7.31 (d, J=7.4 Hz, 1H), 7.06 (t, J=7.9 Hz, 1H), 6.61 (dq, J=4.3, 2.1 Hz, 1H), 3.95-3.88 (m, 2H), 3.52 (q, J=9.6 Hz, 2H), 3.06 (t, J=5.9 Hz, 2H), 2.50 (dt, J=6.4, 2.9 Hz, 2H). MS (ESI) m/z: [M+H]⁺ 316.4.

### Example 153

### Synthesis of ZX147-056:

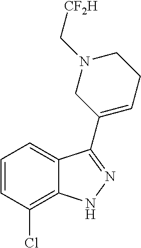 [SMILES: FC(F)CN1CCC=C(c2n[nH]c3c(Cl)cccc23)C1]

7-chloro-3-(1-(2,2-difluoroethyl)-1,2,5,6-tetrahydropyridin-3-yl)-1H-indazole (ZX147-056) ZX147-056 was prepared according to the similar procedure for ZX147-055 but using 2,2-difluoroethyl trifluoromethanesulfonate instead of 2,2,2-trifluoroethyl trifluoromethanesulfonate (36% yield). ¹H NMR (400 MHz, Methanol-d₄) δ 7.85 (d, J=8.4 Hz, 1H), 7.34 (d, J=7.6 Hz, 1H), 7.11 (t, J=8.0 Hz, 1H), 6.76 (s, 1H), 6.42 (tq, J=53.5, 3.3 Hz, 1H), 4.39 (s, 2H), 3.78 (tt, J=15.1, 3.2 Hz, 2H), 3.54 (t, J=6.3 Hz, 2H), 2.77-2.68 (m, 2H). MS (ESI) m/z: [M+H]⁺ 298.1.

### Example 154

### Synthesis of ZX147-092:

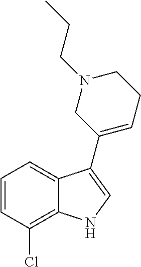 [SMILES: CCCN1CCC=C(c2c[nH]c3c(Cl)cccc23)C1]

7-chloro-3-(1-propyl-1,2,5,6-tetrahydropyridin-3-yl)-1H-indole (ZX147-092). ZX147-092 was synthesized following the standard procedure for preparing NS144-097 from 7-chloro-1H-indole and 1-propylpiperidin-3-one. ¹H NMR (400 MHz, Methanol-d₄) δ 7.76 (d, J=8.1 Hz, 1H), 7.47 (s, 1H), 7.20 (d, J=7.7 Hz, 1H), 7.08 (t, J=8.1 Hz, 1H), 6.40 (s, 1H), 4.31 (d, J=14.9 Hz, 1H), 4.02 (d, J=15.5 Hz, 1H), 3.76-3.67 (m, 1H), 3.29-3.23 (m, 3H), 2.72 (d, J=21.5 Hz, 2H), 1.90 (q, J=8.9, 8.0 Hz, 2H), 1.08 (t, J=8.3 Hz, 3H). MS (ESI) m/z: [M+H]⁺ 275.2.

### Example 155

### Synthesis of ZX147-093:

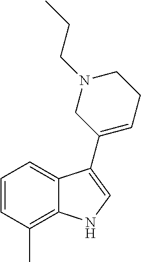 [SMILES: CCCN1CCC=C(c2c[nH]c3c(C)cccc23)C1]

7-methyl-3-(1-propyl-1,2,5,6-tetrahydropyridin-3-yl)-1H-indole (ZX147-093). ZX147-093 was synthesized following the standard procedure for preparing NS144-097 from 7-methyl-1H-indole and 1-propylpiperidin-3-one. ¹H NMR (400 MHz, Methanol-d₄) δ 7.64 (d, J=7.9 Hz, 1H), 7.38 (s, 1H), 7.04-6.94 (m, 2H), 6.38 (s, 1H), 4.31 (d, J=16.4 Hz, 1H), 4.01 (d, J=14.9 Hz, 1H), 3.73-3.68 (m, 1H), 3.30-3.20 (m, 3H), 2.84-2.60 (m, 2H), 2.49 (s, 3H), 1.89 (q, J=7.0 Hz, 2H), 1.08 (t, J=6.9 Hz, 3H). MS (ESI) m/z: [M+H]⁺ 255.4.

### Example 156

### Synthesis of ZX147-094:

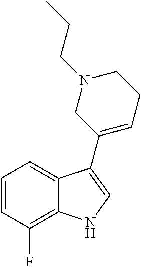 [SMILES: CCCN1CCC=C(c2c[nH]c3c(F)cccc23)C1]

7-fluoro-3-(1-propyl-1,2,5,6-tetrahydropyridin-3-yl)-1H-indole (ZX147-094). ZX147-094 was synthesized following the standard procedure for preparing NS144-097 from 7-fluoro-1H-indole and 1-propylpiperidin-3-one. ¹H NMR (400 MHz, Methanol-d₄) δ 7.60 (d, J=10.1 Hz, 1H), 7.44 (s, 1H), 7.09-7.01 (m, 1H), 6.93-6.89 (m, 1H), 6.40 (s, 1H), 4.29 (s, 1H), 4.02 (d, J=15.8 Hz, 1H), 3.78-3.64 (m, 1H), 3.30-3.22 (m, 3H), 2.86-2.61 (m, 2H), 1.89 (q, J=7.2 Hz, 2H), 1.08 (t, J=7.5 Hz, 3H). MS(ESI) m/z: [M+H]⁺ 259.4.

### Example 157

### Synthesis of ZX147-095:

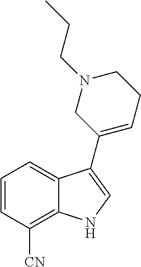 [SMILES: CCCN1CCC=C(c2c[nH]c3c(C#N)cccc23)C1]

3-(1-propyl-1,2,5,6-tetrahydropyridin-3-yl)-1H-indole-7-carbonitrile (ZX147-095). ZX147-095 was synthesized following the standard procedure for preparing NS144-097 from 1H-indole-7-carbonitrile and 1-propylpiperidin-3-one. ¹H NMR (400 MHz, Methanol-d₄) δ 8.14 (d, J=8.2 Hz, 1H), 7.60-7.55 (m, 2H), 7.23 (t, J=7.8 Hz, 1H), 6.42 (s, 1H), 4.32 (d, J=15.5 Hz, 1H), 4.03 (d, J=16.0 Hz, 1H), 3.79-3.68 (m, 1H), 3.30-3.24 (m, 3H), 2.85-2.63 (m, 2H), 1.90 (h, J=9.1 Hz, 2H), 1.08 (t, J=7.4 Hz, 3H). MS (ESI) m/z: [M+H]⁺ 265.4.

### Example 158

### Synthesis of ZX147-096:

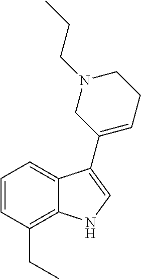 [SMILES: CCCN1CCC=C(c2c[nH]c3c(CC)cccc23)C1]

7-ethyl-3-(1-propyl-1,2,5,6-tetrahydropyridin-3-yl)-1H-indole (ZX147-096). ZX147-096 was synthesized following the standard procedure for preparing NS144-097 from 7-ethyl-1H-indole and 1-propylpiperidin-3-one. ¹H NMR (400 MHz, Methanol-d₄) δ 7.64 (d, J=8.0 Hz, 1H), 7.37 (s, 1H), 7.04 (t, J=7.6 Hz, 1H), 7.00 (d, J=7.2 Hz, 1H), 6.37 (s, 1H), 4.31 (d, J=16.3 Hz, 1H), 3.99 (d, J=13.7 Hz, 1H), 3.74-3.64 (m, 1H), 3.28-3.20 (m, 3H), 2.89 (q, J=7.6 Hz, 2H), 2.83-2.57 (m, 2H), 1.88 (q, J=8.9, 7.9 Hz, 2H), 1.32 (t, J=7.1 Hz, 3H), 1.07 (t, J=7.4 Hz, 3H). MS (ESI) m/z: [M+H]⁺ 269.5.

### Example 159

### Synthesis of ZX147-097:

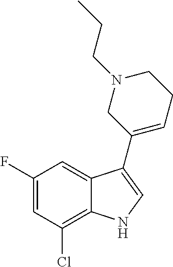 [SMILES: CCCN1CCC=C(c2c[nH]c3c(Cl)cc(F)cc23)C1]

7-ethyl-3-(1-propyl-1,2,5,6-tetrahydropyridin-3-yl)-1H-indole (ZX147-097). ZX147-097 was synthesized following the standard procedure for preparing NS144-097 from 7-chloro-5-fluoro-1H-indole and 1-propylpiperidin-3-one. ¹H NMR (400 MHz, Methanol-d₄) δ 7.53 (s, 1H), 7.47 (d, J=9.9 Hz, 1H), 7.06 (d, J=9.0 Hz, 1H), 6.31 (s, 1H), 4.36-4.22 (m, 1H), 4.01 (s, 1H), 3.70 (s, 1H), 3.30-3.20 (m, 3H), 2.81-2.59 (m, 2H), 1.89 (h, J=7.3 Hz, 2H), 1.07 (t, J=7.5 Hz, 3H). MS (ESI) m/z: [M+H]⁺ 293.2.

### Example 160

### Synthesis of ZX147-098:

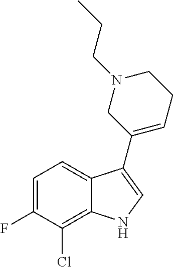 [SMILES: CCCN1CCC=C(c2c[nH]c3c(Cl)c(F)ccc23)C1]

7-ethyl-3-(1-propyl-1,2,5,6-tetrahydropyridin-3-yl)-1H-indole (ZX147-098). ZX147-098 was synthesized following the standard procedure for preparing NS144-097 from 7-chloro-6-fluoro-1H-indole and 1-propylpiperidin-3-one. ¹H NMR (400 MHz, Methanol-d₄) δ 7.72 (dd, J=7.6, 4.6 Hz, 1H), 7.47 (s, 1H), 7.00 (t, J=9.0 Hz, 1H), 6.37 (s, 1H), 4.29 (d, J=15.9 Hz, 1H), 3.99 (d, J=15.9 Hz, 1H), 3.73-3.67 (m, 1H), 3.30-3.20 (m, 3H), 2.78-2.65 (m, 2H), 1.89 (h, J=7.6 Hz, 2H), 1.07 (t, J=6.9 Hz, 3H). MS (ESI) m/z: [M+H]⁺ 293.2.

### Example 161

### Synthesis of ZX147-099:

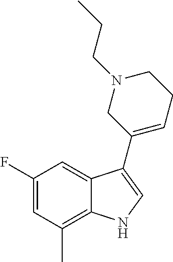 [SMILES: CCCN1CCC=C(c2c[nH]c3c(C)cc(F)cc23)C1]

5-fluoro-7-methyl-3-(1-propyl-1,2,5,6-tetrahydropyridin-3-yl)-1H-indole (ZX147-099). ZX147-099 was synthesized following the standard procedure for preparing NS144-097 from 5-fluoro-7-methyl-1H-indole and 1-propylpiperidin-3-one. ¹H NMR (400 MHz, Methanol-d₄) δ 7.44 (s, 1H), 7.31 (d, J=10.2 Hz, 1H), 6.78 (d, J=9.8 Hz, 1H), 6.30 (s, 1H), 4.29 (d, J=15.4 Hz, 1H), 3.99 (d, J=17.0 Hz, 1H), 3.76-3.66 (m, 1H), 3.29-3.21 (m, 3H), 2.80-2.62 (m, 2H), 2.49 (s, 3H), 1.89 (h, J=7.5 Hz, 2H), 1.07 (t, J=7.4 Hz, 3H). MS (ESI) m/z: [M+H]⁺ 273.2.

### Example 162

### Synthesis of ZX147-100:

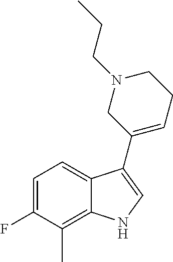 [SMILES: CCCN1CCC=C(c2c[nH]c3c(C)c(F)ccc23)C1]

6-fluoro-7-methyl-3-(1-propyl-1,2,5,6-tetrahydropyridin-3-yl)-1H-indole (ZX147-100). ZX147-100 was synthesized following the standard procedure for preparing NS144-097 from 6-fluoro-7-methyl-1H-indole and 1-propylpiperidin-3-one. ¹HNMR (400 MHz, Methanol-d₄) δ 7.59 (dd, J=8.4, 5.2 Hz, 1H), 7.39 (s, 1H), 6.90-6.83 (m, 1H), 6.37 (s, 1H), 4.29 (d, J=15.4 Hz, 1H), 4.00 (d, J=15.8 Hz, 1H), 3.76-3.66 (m, 1H), 3.29-3.21 (m, 3H), 2.83-2.61 (m, 2H), 2.39 (s, 3H), 1.90 (p, J=7.2 Hz, 2H), 1.08 (t, J=6.6 Hz, 3H). MS (ESI) m/z: [M+H]273.4.

### Example 163

### Synthesis of ZX147-128:

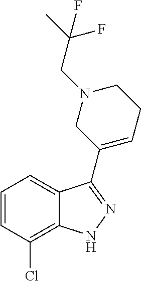 [SMILES: CC(F)(F)CN1CCC=C(c2n[nH]c3c(Cl)cccc23)C1]

7-chloro-3-(1-(2,2-difluoropropyl)-1,2,5,6-tetrahydropyridin-3-yl)-1H-indazole (ZX147-128). ZX147-128 was synthesized following the standard procedure for preparing ZX147-055 from NS136-136 and 2,2-difluoropropyl trifluoromethanesulfonate. ¹H NMR (400 MHz, Methanol-d₄) δ 7.96 (d, J=8.3 Hz, 1H), 7.44 (d, J=7.5 Hz, 1H), 7.20 (t, J=7.8 Hz, 1H), 6.87 (dt, J=4.1, 2.3 Hz, 1H), 4.01 (t, J=15.2 Hz, 2H), 3.68 (t, J=6.1 Hz, 2H), 2.88-2.77 (m, 2H), 1.84 (t, J=19.3 Hz, 3H). MS (ESI) m/z: [M+H]⁺ 312.2.

### Example 164

### Synthesis of ZX147-129:

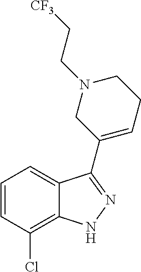 [SMILES: FC(F)(F)CCN1CCC=C(c2n[nH]c3c(Cl)cccc23)C1]

7-chloro-3-(1-(3,3,3-trifluoropropyl)-1,2,5,6-tetrahydropyridin-3-yl)-1H-indazole (ZX147-129). ZX147-129 was synthesized following the standard procedure for preparing ZX147-055 from NS136-136 and 3,3,3-trifluoropropyl trifluoromethanesulfonate. ¹H NMR (400 MHz, Methanol-d₄) δ 7.96 (d, J=8.3 Hz, 1H), 7.45 (d, J=7.5 Hz, 1H), 7.21 (t, J=7.8 Hz, 1H), 6.88 (hept, J=1.8 Hz, 1H), 4.47 (s, 2H), 3.71-3.51 (m, 4H), 3.05-2.88 (m, 2H), 2.86-2.76 (m, 2H). MS (ESI) m/z: [M+H]⁺ 330.3.

### Example 165

### Synthesis of ZX147-130:

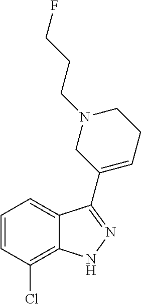 [SMILES: FCCCN1CCC=C(c2n[nH]c3c(Cl)cccc23)C1]

7-chloro-3-(1-(3-fluoropropyl)-1,2,5,6-tetrahydropyridin-3-yl)-1H-indazole (ZX147-129). ZX147-129 was synthesized following the standard procedure for preparing ZX147-055 from NS136-136 and 3-fluoropropyl trifluoromethanesulfonate. ¹H NMR (400 MHz, Methanol-d₄) δ 7.96 (d, J=8.3 Hz, 1H), 7.47-7.41 (m, 1H), 7.21 (t, J=7.9 Hz, 1H), 6.87 (p, J=2.1 Hz, 1H), 4.69 (t, J=5.5 Hz, 2H), 4.58 (t, J=5.5 Hz, 1H), 4.20 (s, 1H), 3.79 (s, 1H), 3.52 (dd, J=9.9, 6.2 Hz, 2H), 3.39 (s, 1H), 2.80 (s, 2H), 2.40-2.21 (m, 2H). MS (ESI) m/z: [M+H]⁺ 294.4.

### Example 166

### Synthesis of ZX147-131

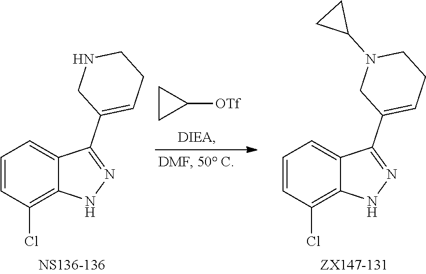

7-chloro-3-(1-cyclopropyl-1,2,5,6-tetrahydropyridin-3-yl)-1H-indazole (ZX147-131). ZX147-131 was prepared according to the same procedure for ZX147-031 using cyclopropyl trifluoromethanesulfonate instead of CD₃I and the reaction mixture was purified by preparative HPLC. (white solid, 24%). ¹H NMR (400 MHz, Methanol-d₄) δ 7.95 (dd, J=8.3, 0.8 Hz, 1H), 7.44 (dd, J=7.5, 0.8 Hz, 1H), 7.20 (t, J=7.9 Hz, 1H), 6.86 (p, J=2.1 Hz, 1H), 6.08 (ddt, J=17.3, 10.1, 7.3 Hz, 1H), 5.76-5.63 (m, 2H), 4.61 (s, 1H), 4.15 (s, 1H), 3.99 (d, J=7.3 Hz, 2H), 3.74 (s, 1H), 2.79 (s, 2H). MS (ESI) m/z: calcd for C₁₅H₁₇ClN₃⁺ [M+H]⁺ 274.1; found 274.3.

### Example 167

### Synthesis of ZX147-137

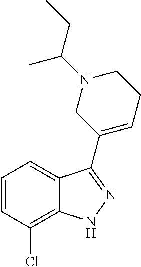 [SMILES: CCC(C)N1CCC=C(c2n[nH]c3c(Cl)cccc23)C1]

3-(1-(sec-butyl)-1,2,5,6-tetrahydropyridin-3-yl)-7-chloro-1H-indazole (ZX147-137). ZX147-137 was prepared according to the same procedure for ZX147-028 but using butan-2-one instead of acetone. ¹H NMR (400 MHz, Methanol-d₄) δ 7.84 (d, J=8.1 Hz, 1H), 7.33 (d, J=7.5 Hz, 1H), 7.09 (t, J=7.9 Hz, 1H), 6.74 (s, 1H), 4.40 (t, J=14.6 Hz, 1H), 4.23-4.08 (m, 1H), 3.56 (dd, J=12.4, 6.1 Hz, 1H), 3.49-3.35 (m, 1H), 3.34-3.19 (m, 1H), 2.85-2.58 (m, 2H), 1.99-1.82 (m, 1H), 1.64 (td, J=15.8, 6.5 Hz, 1H), 1.36 (d, J=6.6 Hz, 3H), 0.99 (t, J=7.4 Hz, 3H). MS (ESI) m/z: [M+H]⁺ 290.3.

### Example 168

### Synthesis of ZX147-183:

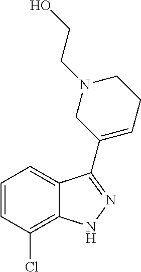 [SMILES: OCCN1CCC=C(c2n[nH]c3c(Cl)cccc23)C1]

2-(5-(7-chloro-1H-indazol-3-yl)-3,6-dihydropyridin-1(2H)-yl)ethan-1-ol (ZX147-183). ZX147-183 was prepared according to the same procedure for ZX147-031 but using 2-bromoethan-1-ol instead of CD₃I. ¹H NMR (400 MHz, Methanol-d₄) δ 7.95 (d, J=8.3 Hz, 1H), 7.45 (d, J=7.5 Hz, 1H), 7.20 (t, J=7.4 Hz, 1H), 6.86 (s, 1H), 4.67 (d, J=16.5 Hz, 1H), 4.23 (d, J=16.3 Hz, 1H), 3.99 (t, J=6.4 Hz, 2H), 3.80 (s, 1H), 3.49-3.44 (m, 2H), 3.44-3.33 (m, 1H), 2.92-2.69 (m, 2H). MS (ESI) m/z: [M+H]⁺ 278.4.

### Example 169

### Synthesis of ZX156-011:

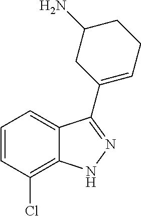 [SMILES: NC1CCC=C(c2n[nH]c3c(Cl)cccc23)C1]

3-(7-chloro-1H-indazol-3-yl)cyclohex-3-en-1-amine (ZX156-011). ZX156-011 was synthesized following the standard procedure for preparing NS144-102 from 3-bromo-7-chloro-1H-indazole and tert-butyl (3-(4,4,5,5-tetramethyl-1,3,2-dioxaborolan-2-yl)cyclohex-3-en-1-yl)carbamate. ¹H NMR (400 MHz, Methanol-d₄) δ 7.87 (d, J=8.2 Hz, 1H), 7.38 (d, J=7.4 Hz, 1H), 7.13 (t, J=7.9 Hz, 1H), 6.63 (s, 1H), 3.58 (t, J=13.2 Hz, 1H), 3.21 (d, J=17.2 Hz, 1H), 2.70-2.59 (m, 1H), 2.58-2.44 (m, 2H), 2.20-2.12 (m, 1H), 1.84 (q, J=10.4, 9.0 Hz, 1H). MS (ESI) m/z: [M+H]f 248.2.

### Example 170

### Synthesis of ZX156-012:

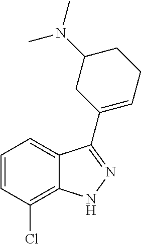 [SMILES: CN(C)C1CCC=C(c2n[nH]c3c(Cl)cccc23)C1]

3-(7-chloro-1H-indazol-3-yl)-N,N-dimethylcyclohex-3-en-1-amine (ZX156-012). ZX156-012 was prepared according to the same procedure for ZX147-028 from ZX156-011 and paraformaldehyde. ¹H NMR (400 MHz, Methanol-d₄) δ 7.90 (d, J=8.4 Hz, 1H), 7.41 (d, J=7.5 Hz, 1H), 7.15 (t, J=7.9 Hz, 1H), 6.65 (s, 1H), 3.72-3.62 (m, 1H), 3.27 (d, J=12.1 Hz, 1H), 2.98 (s, 6H), 2.85-2.74 (m, 1H), 2.71-2.60 (m, 1H), 2.60-2.47 (m, 1H), 2.30-2.24 (m, 1H), 1.94-1.81 (m, 1H). MS (ESI) m/z: [M+H]⁺ 276.3.

### Example 171 and 172

### Synthesis of ZX156-014-1 and 156-014-2:

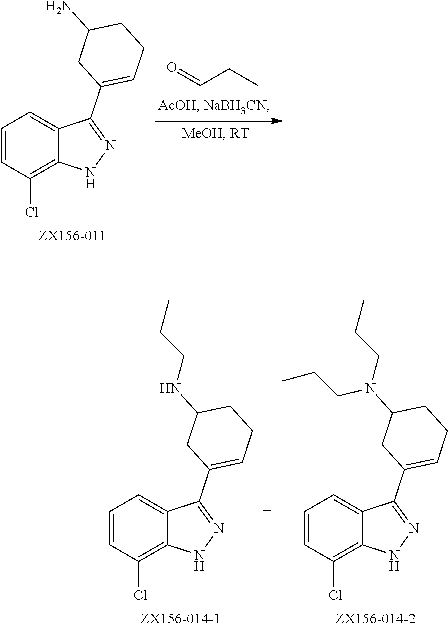

3-(7-chloro-1H-indazol-3-yl)-N-propylcyclohex-3-en-1-amine (ZX156-014-1). ZX156-014-1 was prepared according to the same procedure as ZX147-028 from ZX156-011 and propionaldehyde. ¹H NMR (400 MHz, Methanol-d₄) δ 7.90 (d, J=8.5 Hz, 1H), 7.41 (d, J=5.6 Hz, 1H), 7.15 (t, J=7.9 Hz, 1H), 6.67 (s, 1H), 3.54 (d, J=12.2 Hz, 1H), 3.38-3.26 (m, 1H), 3.13 (t, J=7.6 Hz, 2H), 2.71-2.45 (m, 3H), 2.31-2.23 (m, 1H), 1.87-1.72 (m, 3H), 1.08 (t, J=7.5 Hz, 3H). MS (ESI) m/z: [M+H]⁺ 290.3.

3-(7-chloro-1H-indazol-3-yl)-N,N-dipropylcyclohex-3-en-1-amine (ZX156-014-2). MS (ESI) m/z: [M+H]⁺ 332.2.

### Example 173

### Synthesis of ZX156-019.

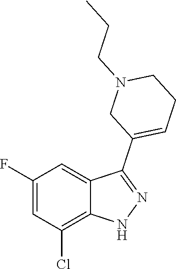 [SMILES: CCCN1CCC=C(c2n[nH]c3c(Cl)cc(F)cc23)C1]

7-chloro-5-fluoro-3-(1-propyl-1,2,5,6-tetrahydropyridin-3-yl)-1H-indazole (ZX156-019). ZX156-019 was prepared according to the same procedure for ZX147-028 from NS144-102 and propionaldehyde. ¹H NMR (400 MHz, Methanol-d₄) δ 8.13 (d, J=6.9 Hz, 1H), 7.40 (d, J=9.2 Hz, 1H), 6.81 (s, 1H), 4.60 (d, J=15.5 Hz, 1H), 4.09 (d, J=14.8 Hz, 1H), 3.73 (s, 1H), 3.38-3.24 (m, 2H), 2.78 (s, 2H), 1.90 (h, J=7.8 Hz, 2H), 1.08 (t, J=7.5 Hz, 3H). MS (ESI) m/z: [M+H]⁺ 294.3.

### Example 174

### Synthesis of ZX156-059.

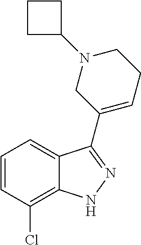 [SMILES: Clc1cccc2c(C3=CCCN(C4CCC4)C3)n[nH]c12]

7-chloro-3-(1-cyclobutyl-1,2,5,6-tetrahydropyridin-3-yl)-1H-indazole (ZX156-059). ZX156-059 was prepared according to the same procedure for ZX147-028 from NS136-136 and cyclobutanone. ¹H NMR (400 MHz, Methanol-d₄) δ 7.93 (d, J=8.4 Hz, 1H), 7.43 (d, J=7.5 Hz, 1H), 7.22-7.16 (m, 1H), 6.85 (s, 1H), 4.52 (s, 1H), 3.91 (p, J=9.3, 8.4 Hz, 2H), 3.63 (s, 1H), 3.17 (s, 1H), 2.77 (s, 2H), 2.51-2.42 (m, 2H), 2.42-2.28 (m, 2H), 1.94 (h, J=10.3, 9.9 Hz, 2H). MS (ESI) m/z: [M+H]⁺ 288.2.

### Example 175

### Step 1: Synthesis of NS136-152

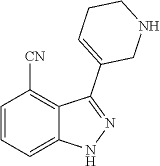 [SMILES: N#Cc1cccc2[nH]nc(C3=CCCNC3)c12]

3-(1,2,5,6-tetrahydropyridin-3-yl)-1H-indazole-4-carbonitrile (NS136-152). NS136-152 was synthesized following the standard procedure for preparing NS144-102 from 3-bromo-1H-indazole-4-carbonitrile and tert-butyl 5-(4,4,5,5-tetramethyl-1,3,2-dioxaborolan-2-yl)-3,6-dihydropyridine-1(2H)-carboxylate. (white solid, 20 mg, 59%) ¹H NMR (600 MHz, Methanol-d₄) δ 7.90 (d, J=8.5 Hz, 1H), 7.69 (d, J=7.1 Hz, 1H), 7.54 (t, J=8.1 Hz, 1H), 6.69-6.57 (m, 1H), 4.34-4.15 (m, 2H), 3.47 (t, J=6.4 Hz, 2H), 2.83-2.64 (m, 2H). MS (ESI) m/z: calcd for C₁₃H₁₃N₄⁺ [M+H]⁺, 225.1 found, 225.3.

### Step 2: Synthesis of ZX156-069.

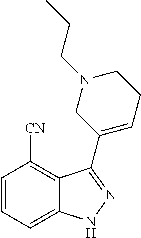 [SMILES: CCCN1CCC=C(c2n[nH]c3cccc(C#N)c23)C1]

3-(1-propyl-1,2,5,6-tetrahydropyridin-3-yl)-1H-indazole-4-carbonitrile (ZX156-069). ZX156-069 was prepared according to the same procedure for ZX147-028 from NS136-152 and propionaldehyde. ¹H NMR (400 MHz, Methanol-d₄) δ 7.91 (d, J=8.8 Hz, 1H), 7.70 (d, J=7.3 Hz, 1H), 7.56 (t, J=7.1 Hz, 1H), 6.65 (s, 1H), 4.54 (d, J=16.0 Hz, 1H), 4.10 (d, J=13.7 Hz, 1H), 3.76 (s, 1H), 3.36 (s, 1H), 2.79 (s, 2H), 1.89 (q, J=7.7 Hz, 2H), 1.08 (t, J=6.8 Hz, 3H). MS (ESI) m/z: [M+H]⁺ 266.7.

### Example 176

### Step 1: Synthesis of NS144-046

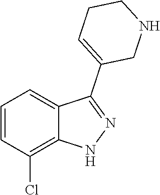 [SMILES: Clc1cccc2c(C3=CCCNC3)n[nH]c12]

3-(1,2,5,6-tetrahydropyridin-3-yl)-1H-indazole-7-carbonitrile (NS144-046). NS144-046 was synthesized following the standard procedure for preparing NS144-102 from 3-bromo-1H-indazole-7-carbonitrile and tert-butyl 5-(4,4,5,5-tetramethyl-1,3,2-dioxaborolan-2-yl)-3,6-dihydropyridine-1(2H)-carboxylate. (white solid, 16.6 mg, 49%) ¹H NMR (600 MHz, Methanol-d₄) δ 8.35 (dd, J=8.5, 2.8 Hz, 1H), 7.86 (dd, J=7.5, 2.8 Hz, 1H), 7.36 (dd, J=9.1, 6.4 Hz, 1H), 6.90 (td, J=4.1, 2.0 Hz, 1H), 4.34-4.26 (m, 2H), 3.46 (t, J=6.1 Hz, 2H), 2.73 (tt, J=4.3, 2.2 Hz, 2H). MS (ESI) m/z: calcd for C₁₃H₁₃N₄⁺ [M+H]⁺, 225.1 found, 225.3.

### Step 2: Synthesis of ZX156-070

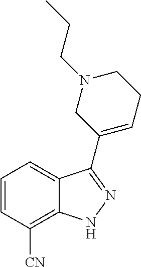 [SMILES: CCCN1CCC=C(c2n[nH]c3c(C#N)cccc23)C1]

3-(1-propyl-1,2,5,6-tetrahydropyridin-3-yl)-1H-indazole-7-carbonitrile (ZX156-070). ZX156-070 was prepared according to the same procedure as ZX147-028 from NS144-046 and propionaldehyde. ¹H NMR (400 MHz, Methanol-d₄) δ 8.35 (d, J=8.4 Hz, 1H), 7.86 (d, J=7.3 Hz, 1H), 7.36 (t, J=7.8 Hz, 1H), 6.90 (s, 1H), 4.65 (d, J=16.1 Hz, 1H), 4.14 (d, J=15.9 Hz, 1H), 3.75 (s, 1H), 3.38 (s, 1H), 2.80 (s, 2H), 1.90 (h, J=6.8, 6.3 Hz, 2H), 1.09 (t, J=6.9 Hz, 3H). MS (ESI) m/z: [M+H]⁺ 267.0.

### Example 177

### Synthesis of ZX156-071

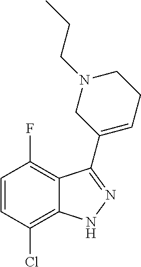 [SMILES: CCCN1CCC=C(c2n[nH]c3c(Cl)ccc(F)c23)C1]

7-chloro-4-fluoro-3-(1-propyl-1,2,5,6-tetrahydropyridin-3-yl)-1H-indazole (ZX156-071). ZX156-071 was prepared according to the same procedure as ZX147-028 from NS144-107 and propionaldehyde. ¹H NMR (400 MHz, Methanol-d₄) δ 7.43-7.40 (m, 1H), 6.93-6.89 (m, 1H), 6.87 (s, 1H), 4.63 (d, J=15.4 Hz, 1H), 4.14 (d, J=14.3 Hz, 1H), 3.73 (s, 1H), 3.29 (s, 1H), 2.75 (s, 2H), 1.90 (h, J=7.2 Hz, 2H), 1.09 (t, J=7.4 Hz, 3H). MS (ESI) m/z: [M+H]⁺ 294.8.

### Example 178

### Synthesis of ZX156-089

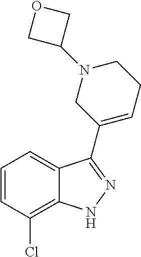 [SMILES: Clc1cccc2c(C3=CCCN(C4COC4)C3)n[nH]c12]

7-chloro-3-(1-(oxetan-3-yl)-1,2,5,6-tetrahydropyridin-3-yl)-1H-indazole (ZX156-089). ZX156-089 was prepared according to the same procedure as ZX147-028 from NS136-136 and oxetan-3-one. ¹H NMR (400 MHz, Methanol-d₄) δ 7.94 (d, J=8.2 Hz, 1H), 7.44 (d, J=7.5 Hz, 1H), 7.20 (t, J=7.9 Hz, 1H), 6.88 (s, 1H), 4.98 (d, J=8.1 Hz, 2H), 4.96-4.91 (m, 2H), 4.64 (p, J=6.8, 6.2 Hz, 1H), 4.32 (s, 2H), 3.46 (s, 2H), 2.82 (s, 2H). MS (ESI) m/z: [M+H]⁺ 290.0.

### Example 179

### Synthesis of ZX156-090

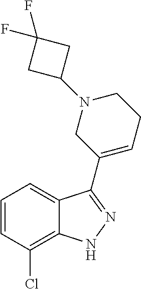 [SMILES: FC1(F)CC(N2CCC=C(c3n[nH]c4c(Cl)cccc34)C2)C1]

7-chloro-3-(1-(3,3-difluorocyclobutyl)-1,2,5,6-tetrahydropyridin-3-yl)-1H-indazole (ZX156-090). ZX156-090 was prepared according to the same procedure as ZX147-028 from NS136-136 and 3,3-difluorocyclobutan-1-one. ¹H NMR (400 MHz, Methanol-d₄) δ 7.95 (d, J=8.3 Hz, 1H), 7.45 (d, J=7.5 Hz, 1H), 7.20 (t, J=7.9 Hz, 1H), 6.87 (s, 1H), 4.34 (s, 2H), 3.94 (h, J=7.4 Hz, 1H), 3.48 (s, 2H), 3.25-3.12 (m, 2H), 3.11-2.98 (m, 2H), 2.81 (s, 2H). MS (ESI) m/z: [M+H]f 324.3.

### Example 180

### Synthesis of Compound ZX162-100

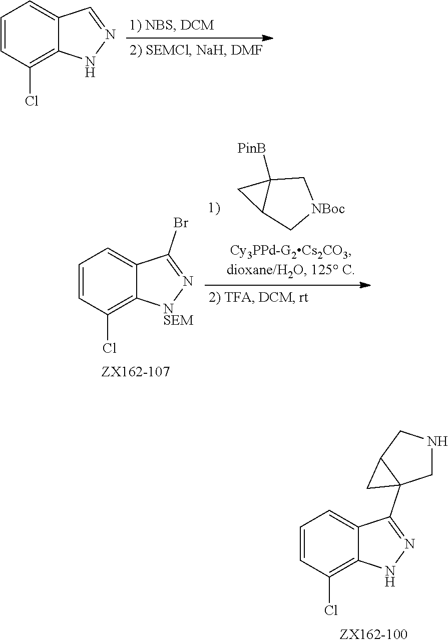

3-(3-azabicyclo[3.1.0]hexan-1-yl)-7-chloro-1H-indazole (ZX162-100). Indazole (2.0 g, 13.1 mmol, 1.0 equiv.), NBS (2.8 g, 15.7 mmol, 1.2 equiv.) and DCM (20 mL) were stirred at room temperature overnight followed by quenched with Sat. Na₂SO₃ aqueous solution (20 mL). The organic layer was separated, dried over Na₂SO₄, concentrated. The resulted residue was dissolved in DMF (50 mL) at 0° C., NaH (1.05 g, 26.2 mmol, 2.0 equiv.) was added to the solution and stirred for 10 min followed by SEMCl (3.5 mL, 19.7 mmol, 1.5 equiv.). After stirred for another 4 h, the mixture was quenched with sat. NaHCO₃ aqueous solution (20 mL), the organic phase was separated, concentrated, and purified by silica gel chromatography (PE-PE/EA=5/1) to yield ZX162-107 (colorless oil, 3.9 g, 82% yield). ¹H NMR (400 MHz, Chloroform-d) δ 7.55 (d, J=8.1 Hz, 1H), 7.47 (d, J=7.5 Hz, 1H), 7.18 (t, J=8.2 Hz, 1H), 5.99 (s, 2H), 3.60 (t, J=7.5 Hz, 2H), 0.93-0.83 (m, 2H), −0.07 (s, 9H).

To a solution of ZX162-107 (250 mg, 0.71 mmol, 1.0 equiv.) in dioxane/water (8 mL, 5:1) was added Cy₃P Pd G2 (42 mg, 0.071 mmol, 0.1 equiv.), Cs₂CO₃ (463 mg, 1.42 mmol, 2.0 equiv.) and tert-butyl 1-(4,4,5,5-tetramethyl-1,3,2-dioxaborolan-2-yl)-3-azabicyclo[3.1.0]hexane-3-carboxylate (219 mg, 0.71 mmol, 1.0 equiv.). The mixture was heated at 125° C. under microwave irradiation condition at nitrogen atmosphere for 1 h, followed by diluted with DCM (20 mL) and washed with brine (20 mL). After dried over Na₂SO₄ and concentrated, the residue was dissolved into DCM/TFA (5 mL, 2:1) and stirred at room temperature for 1 h followed by purified by prep-HPLC to yield title compound (white solid, 99 mg, 40% yield). ¹H NMR (400 MHz, Methanol-d₄) δ 7.67 (d, J=8.3 Hz, 1H), 7.42 (d, J=7.5 Hz, 1H), 7.14 (t, J=7.8 Hz, 1H), 3.90 (s, 2H), 3.75 (dd, J=11.6, 4.0 Hz, 1H), 3.60 (d, J=11.6 Hz, 1H), 2.52 (dt, J=8.7, 4.3 Hz, 1H), 1.64 (t, J=7.6 Hz, 1H), 1.26 (t, J=5.7 Hz, 1H). MS (ESI) m/z: [M+H]⁺ 234.3.

### Example 181

### Synthesis of Compound ZX162-031

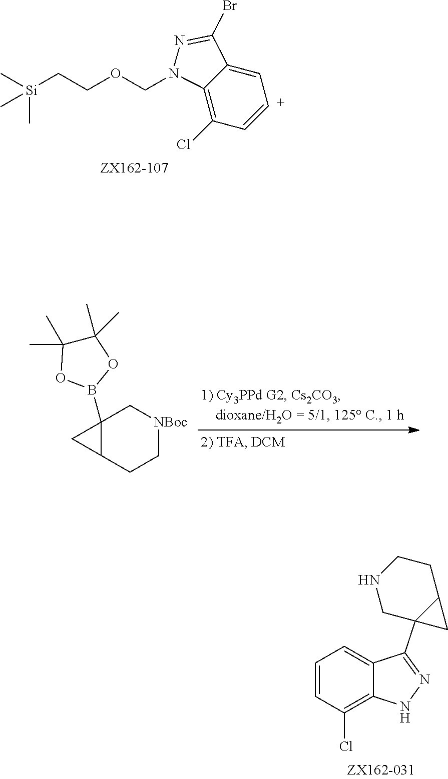 [SMILES: C[Si](C)(C)CCOCn1nc(Br)c2c1=C(Cl)C=[C+]C=2 ; CC(C)(C)OC(=O)N1CCC2CC2(B2OC(C)(C)C(C)(C)O2)C1 ; Clc1cccc2c(C34CNCCC3C4)n[nH]c12]

3-(3-azabicyclo[4.1.0]heptan-1-yl)-7-chloro-1H-indazole (ZX162-031). ZX162-031 was prepared according to the procedure similar to ZX162-100. Yield: 30%. ¹H NMR (400 MHz, Methanol-d₄) δ 7.71 (d, J=8.1 Hz, 1H), 7.40 (d, J=7.5 Hz, 1H), 7.18-7.08 (m, 1H), 4.12 (d, J=13.5 Hz, 1H), 3.60 (d, J=13.5 Hz, 1H), 3.23 (dt, J=12.5, 5.9 Hz, 1H), 3.07-2.95 (m, 1H), 2.48 (ddt, J=15.1, 9.4, 6.2 Hz, 1H), 2.21-2.08 (m, 1H), 2.01 (dtd, J=8.6, 6.5, 2.0 Hz, 1H), 1.63 (dd, J=9.4, 5.8 Hz, 1H), 1.25 (t, J=6.1 Hz, 1H). MS (ESI) m/z: [M+H]⁺ 248.3.

### Example 182

### Synthesis of Compound ZX162-104

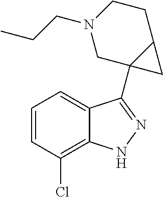 [SMILES: CCCN1CCC2CC2(c2n[nH]c3c(Cl)cccc23)C1]

7-chloro-3-(3-propyl-3-azabicyclo[4.1.0]heptan-1-yl)-1H-indazole (ZX162-104). ZX162-104 was prepared according to the same procedure as ZX147-028 from ZX162-031 and propionaldehyde. ¹H NMR (400 MHz, Methanol-d₄) δ 7.65 (d, J=8.2 Hz, 1H), 7.35 (d, J=7.5 Hz, 1H), 7.07 (t, J=7.9 Hz, 1H), 4.19 (s, 1H), 3.45 (d, J=12.3 Hz, 1H), 2.98 (t, J=7.0 Hz, 2H), 2.89 (s, 1H), 2.54-2.42 (m, 1H), 2.15 (d, J=14.9 Hz, 1H), 1.99 (q, J=7.6, 7.0 Hz, 1H), 1.71 (h, J=7.4 Hz, 2H), 1.57 (dd, J=9.7, 5.6 Hz, 1H), 0.96 (t, J=7.4 Hz, 3H). MS (ESI) m/z: [M+H]⁺290.0.

### Example 183

### Synthesis of Compound ZX162-105

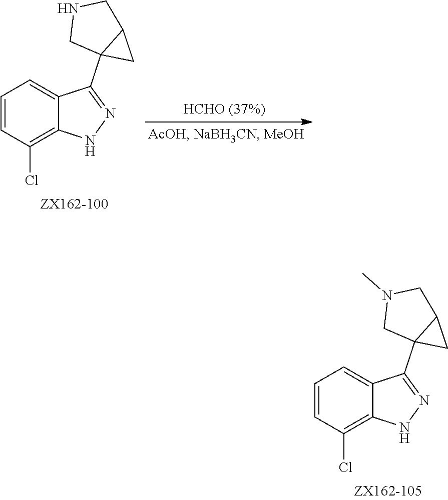 [SMILES: Clc1cccc2c(C34CNCC3C4)n[nH]c12 ; CN1CC2CC2(c2n[nH]c3c(Cl)cccc23)C1]

7-chloro-3-(3-methyl-3-azabicyclo[3.1.0]hexan-1-yl)-1H-indazole (ZX162-105). ZX162-100 was dissolved into methanol, and AcOH (5 equiv.), HCHO (aqueous solution) (10 equiv.), NaBH₃CN (3.0 equiv.) were added and stirred at room temperature for 1 h. Purified by preparative HPLC. ¹H NMR (400 MHz, Methanol-d₄) δ 7.68 (d, J=8.2 Hz, 1H), 7.41 (d, J=7.4 Hz, 1H), 7.13 (t, J=7.9 Hz, 1H), 4.02 (d, J=11.0 Hz, 1H), 3.74 (t, J=11.3 Hz, 2H), 3.56 (dd, J=11.3, 4.0 Hz, 1H), 2.95 (s, 3H), 2.50 (dt, J=8.9, 4.4 Hz, 1H), 1.59 (t, J=7.5 Hz, 1H), 1.41 (t, J=5.7 Hz, 1H). MS (ESI) m/z: [M+H]⁺ 248.3.

### Example 184

### Step 1: Synthesis of Compound ZX162-102

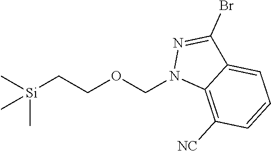 [SMILES: C[Si](C)(C)CCOCn1nc(Br)c2cccc(C#N)c21]

ZX162-102 was prepared according to the procedure similar to ZX162-107. White solid. ¹H NMR (400 MHz, Chloroform-d) δ 7.90 (d, J=8.2 Hz, 1H), 7.85 (d, J=7.4 Hz, 1H), 7.34 (t, J=8.4 Hz, 1H), 5.97 (s, 2H), 3.64 (t, J=7.7 Hz, 2H), 0.92 (t, J=7.7 Hz, 2H), −0.05 (s, 9H).

### Step 2: Synthesis of Compound ZX162-110

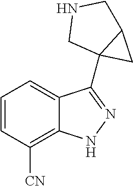 [SMILES: N#Cc1cccc2c(C34CNCC3C4)n[nH]c12]

3-(3-azabicyclo[3.1.0]hexan-1-yl)-1H-indazole-7-carbonitrile (ZX162-110). ZX162-110 was prepared according to the procedure similar to ZX162-100 from ZX162-102 and tert-butyl 1-(4,4,5,5-tetramethyl-1,3,2-dioxaborolan-2-yl)-3-azabicyclo[3.1.0]hexane-3-carboxylate. ¹H NMR (400 MHz, Methanol-d₄) δ 8.06 (d, J=8.3 Hz, 1H), 7.82 (d, J=7.3 Hz, 1H), 7.29 (t, J=7.8 Hz, 1H), 3.92 (s, 2H), 3.75 (dd, J=11.8, 4.0 Hz, 1H), 3.61 (d, J=11.7 Hz, 1H), 2.55 (dt, J=9.0, 4.6 Hz, 1H), 1.66 (t, J=7.7 Hz, 1H), 1.30 (t, J=5.8 Hz, 1H). MS (ESI) m/z: [M+H]⁺ 225.4.

### Example 185

### Step 1: Synthesis of Compound ZX162-101

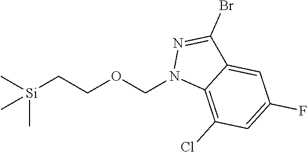 [SMILES: C[Si](C)(C)CCOCn1nc(Br)c2cc(F)cc(Cl)c21]

ZX162-101 was prepared according to the procedure similar to ZX162-107. White solid, yield: 77%. H NMR (400 MHz, Chloroform-d) δ 7.68 (d, J=6.5 Hz, 1H), 7.34 (d, J=8.6 Hz, 1H), 5.63 (s, 2H), 3.55 (t, J=9.2 Hz, 2H), 0.88 (t, J=9.2 Hz, 2H), −0.05 (s, 9H).

### Step 2: Synthesis of Compound ZX162-111

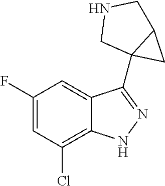 [SMILES: Fc1cc(Cl)c2[nH]nc(C34CNCC3C4)c2c1]

3-(3-azabicyclo[3.1.0]hexan-1-yl)-7-chloro-5-fluoro-1H-indazole (ZX162-111). ZX162-111 was prepared according to the procedure similar to ZX162-100 from ZX162-101 and tert-butyl 1-(4,4,5,5-tetramethyl-1,3,2-dioxaborolan-2-yl)-3-azabicyclo[3.1.0]hexane-3-carboxylate. ¹H NMR (400 MHz, Methanol-d₄) δ 7.87 (d, J=7.0 Hz, 1H), 7.37 (d, J=9.1 Hz, 1H), 3.86 (s, 2H), 3.75 (dd, J=11.6, 4.0 Hz, 1H), 3.60 (d, J=11.6 Hz, 1H), 2.47 (dt, J=8.7, 4.5 Hz, 1H), 1.59 (t, J=7.6 Hz, 1H), 1.26 (t, J=5.7 Hz, 1H). MS (ESI) m/z: [M+H]⁺ 252.3.

### Example 186

### Step 1: Synthesis of Compound ZX162-108

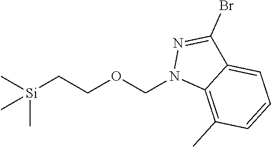 [SMILES: Cc1cccc2c(Br)nn(COCC[Si](C)(C)C)c12]

ZX162-108 was prepared according to the procedure similar to ZX162-107. ¹H NMR (400 MHz, Chloroform-d) δ 7.46 (d, J=8.1 Hz, 1H), 7.22 (d, J=7.1 Hz, 1H), 7.14 (t, J=7.7 Hz, 1H), 5.79 (s, 2H), 3.55 (t, J=7.4 Hz, 2H), 2.75 (s, 3H), 0.86 (t, J=6.9 Hz, 2H), −0.07 (s, 9H).

### Step 2: Synthesis of Compound ZX162-112

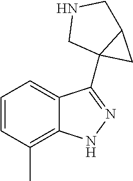 [SMILES: Cc1cccc2c(C34CNCC3C4)n[nH]c12]

3-(3-azabicyclo[3.1.0]hexan-1-yl)-7-methyl-1H-indazole (ZX162-112). ZX162-112 was prepared according to the procedure similar to ZX162-100 from ZX162-108 and tert-butyl 1-(4,4,5,5-tetramethyl-1,3,2-dioxaborolan-2-yl)-3-azabicyclo[3.1.0]hexane-3-carboxylate. ¹H NMR (400 MHz, Methanol-d₄) δ 7.53 (d, J=8.2 Hz, 1H), 7.16 (d, J=6.9 Hz, 1H), 7.06 (t, J=7.6 Hz, 1H), 3.87 (s, 2H), 3.75 (dd, J=11.6, 3.9 Hz, 1H), 3.61 (d, J=11.5 Hz, 1H), 2.53 (s, 3H), 2.46 (dt, J=8.7, 4.5 Hz, 1H), 1.61 (t, J=7.6 Hz, 1H), 1.25 (t, J=5.7 Hz, 1H). MS (ESI) m/z: [M+H]⁺ 214.4.

### Example 187

### Step 1: Synthesis of Compound ZX162-109

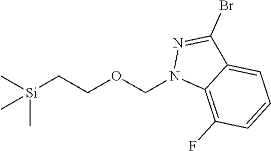 [SMILES: C[Si](C)(C)CCOCn1nc(Br)c2cccc(F)c21]

ZX162-109 was prepared according to the procedure similar to ZX162-107. ¹H NMR (400 MHz, Chloroform-d) δ 7.41 (dt, J=6.8, 2.4 Hz, 1H), 7.20-7.13 (m, 2H), 5.78 (s, 2H), 3.60 (t, J=8.1 Hz, 2H), 0.89 (t, J=8.2 Hz, 2H), −0.07 (s, 9H).

### Step 2: Synthesis of Compound ZX162-113

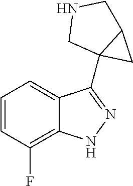 [SMILES: Fc1cccc2c(C34CNCC3C4)n[nH]c12]

3-(3-azabicyclo[3.1.0]hexan-1-yl)-7-fluoro-1H-indazole (ZX162-113). ZX162-113 was prepared according to the procedure similar to ZX162-100 from ZX162-109 and tert-butyl 1-(4,4,5,5-tetramethyl-1,3,2-dioxaborolan-2-yl)-3-azabicyclo[3.1.0]hexane-3-carboxylate. ¹H NMR (400 MHz, Methanol-d₄) δ 7.54-7.48 (m, 1H), 7.15-7.07 (m, 2H), 3.89 (s, 2H), 3.75 (dd, J=11.8, 4.0 Hz, 1H), 3.61 (d, J=11.6 Hz, 1H), 2.55-2.45 (m, 1H), 1.63 (t, J=7.5 Hz, 1H), 1.27 (t, J=5.7 Hz, 1H). MS (ESI) m/z: [M+H]⁺ 218.3.

### Example 188

### Synthesis of Compound ZX162-124

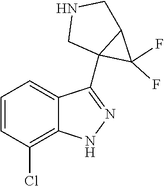 [SMILES: FC1(F)C2CNCC21c1n[nH]c2c(Cl)cccc12]

7-chloro-3-(7,7-difluoro-3-azabicyclo[4.1.0]heptan-1-yl)-1H-indazole (ZX162-124). ZX162-124 was prepared according to the procedure similar to ZX162-100 from ZX162-107 and potassium 3-(tert-butoxycarbonyl)-7,7-difluoro-3-azabicyclo[4.1.0]heptan-1-yl)trifluoroborate. ¹H NMR (400 MHz, Methanol-d₄) δ 7.71 (d, J=8.1 Hz, 1H), 7.45 (d, J=7.4 Hz, 1H), 7.19 (t, J=7.9 Hz, 1H), 3.97-3.83 (m, 2H), 3.35-3.24 (m, 1H), 3.08-2.99 (m, 1H), 2.99-2.90 (m, 1H), 2.50 (h, J=8.2 Hz, 1H), 2.33-2.23 (m, 1H). MS (ESI) m/z: [M+H]⁺ 284.3.

### Example 189

### Synthesis of Compound ZX162-126

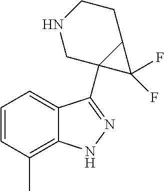 [SMILES: Cc1cccc2c(C34CNCCC3C4(F)F)n[nH]c12]

3-(7,7-difluoro-3-azabicyclo[4.1.0]heptan-1-yl)-7-methyl-1H-indazole (ZX162-126). ZX162-126 was prepared according to the procedure similar to ZX162-100 from ZX162-108 and potassium 3-(tert-butoxycarbonyl)-7,7-difluoro-3-azabicyclo[4.1.0]heptan-1-yl)trifluoroborate. ¹H NMR (400 MHz, Methanol-d₄) δ 7.56 (d, J=8.2 Hz, 1H), 7.20 (d, J=6.9 Hz, 1H), 7.12 (t, J=7.6 Hz, 1H), 3.87 (s, 2H), 3.27 (t, J=6.1 Hz, 1H), 3.09-3.00 (m, 1H), 2.95-2.85 (m, 1H), 2.55 (s, 3H), 2.47 (d, J=8.3 Hz, 1H), 2.33-2.22 (m, 1H). MS (ESI) m/z: [M+H]⁺ 264.4.

### Example 190

### Synthesis of Compound ZX162-127

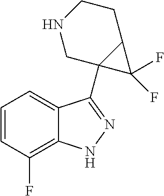 [SMILES: Fc1cccc2c(C34CNCCC3C4(F)F)n[nH]c12]

3-(7,7-difluoro-3-azabicyclo[4.1.0]heptan-1-yl)-7-fluoro-1H-indazole (ZX162-127). ZX162-127 was prepared according to the procedure similar to ZX162-100 from ZX162-109 and potassium 3-(tert-butoxycarbonyl)-7,7-difluoro-3-azabicyclo[4.1.0]heptan-1-yl)trifluoroborate. ¹H NMR (400 MHz, Methanol-d₄) δ 7.58-7.53 (m, 1H), 7.21-7.13 (m, 2H), 3.90 (q, J=14.6, 14.0 Hz, 2H), 3.28 (t, J=6.0 Hz, 1H), 3.09-3.00 (m, 1H), 2.99-2.91 (m, 1H), 2.51 (dt, J=15.9, 7.9 Hz, 1H), 2.29 (dd, J=14.8, 7.0 Hz, 1H). MS (ESI) m/z: [M+H]⁺ 268.3.

### Example 191

### Synthesis of Compound ZX162-128

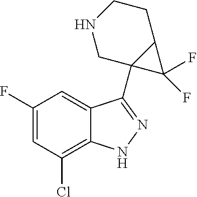 [SMILES: Fc1cc(Cl)c2[nH]nc(C34CNCCC3C4(F)F)c2c1]

7-chloro-3-(7,7-difluoro-3-azabicyclo[4.1.0]heptan-1-yl)-5-fluoro-1H-indazole (ZX162-128). ZX162-128 was prepared according to the procedure similar to ZX162-100 from ZX162-101 and potassium 3-(tert-butoxycarbonyl)-7,7-difluoro-3-azabicyclo[4.1.0]heptan-1-yl)trifluoroborate. ¹H NMR (400 MHz, Methanol-d₄) δ 7.83 (d, J=6.9 Hz, 1H), 7.33 (d, J=9.2 Hz, 1H), 3.88-3.70 (m, 2H), 3.17 (t, J=6.0 Hz, 1H), 2.98-2.87 (m, 1H), 2.80 (tt, J=8.3, 4.3 Hz, 1H), 2.39 (h, J=8.5 Hz, 1H), 2.20-2.13 (m, 1H). MS (ESI) m/z: [M+H]⁺ 302.5.

### Example 192

### Synthesis of Compound ZX162-129

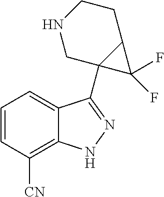 [SMILES: N#Cc1cccc2c(C34CNCCC3C4(F)F)n[nH]c12]

3-(7,7-difluoro-3-azabicyclo[4.1.0]heptan-1-yl)-1H-indazole-7-carbonitrile (ZX162-129). ZX162-129 was prepared according to the procedure similar to ZX162-100 from ZX162-102 and potassium 3-(tert-butoxycarbonyl)-7,7-difluoro-3-azabicyclo[4.1.0]heptan-1-yl)trifluoroborate. ¹H NMR (400 MHz, Methanol-d₄) δ 8.12 (d, J=8.2 Hz, 1H), 7.88 (d, J=7.2 Hz, 1H), 7.39-7.33 (m, 1H), 3.98 (d, J=16.0 Hz, 1H), 3.87 (d, J=14.0 Hz, 1H), 3.35-3.27 (m, 1H), 3.09-2.94 (m, 2H), 2.51 (dq, J=15.9, 7.8 Hz, 1H), 2.33-2.23 (m, 1H). MS (ESI) m/z: [M+H]⁺ 275.5.

### Example 193

### Synthesis of Compound ZX162-138

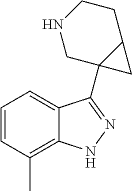 [SMILES: Cc1cccc2c(C34CNCCC3C4)n[nH]c12]

3-(3-azabicyclo[4.1.0]heptan-1-yl)-7-methyl-1H-indazole (ZX162-129). ZX162-129 was prepared according to the procedure similar to ZX162-100 from ZX162-108 and tert-butyl 1-(4,4,5,5-tetramethyl-1,3,2-dioxaborolan-2-yl)-3-azabicyclo[4.1.0]heptane-3-carboxylate. ¹H NMR (400 MHz, Methanol-d₄) δ 7.58 (d, J=8.1 Hz, 1H), 7.14 (d, J=6.9 Hz, 1H), 7.05 (t, J=7.6 Hz, 1H), 4.02 (d, J=13.4 Hz, 1H), 3.62 (d, J=13.6 Hz, 1H), 3.22 (dt, J=12.3, 5.9 Hz, 1H), 3.06-2.97 (m, 1H), 2.51 (s, 3H), 2.47 (dd, J=14.7, 6.9 Hz, 1H), 2.12 (dt, J=15.0, 5.6 Hz, 1H), 1.94 (q, J=7.9, 6.8 Hz, 1H), 1.57 (dd, J=9.5, 5.6 Hz, 1H), 1.24-1.18 (m, 1H). MS (ESI) m/z: [M+H]⁺ 228.5.

### Example 194

### Synthesis of Compound ZX162-139

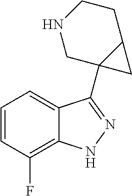 [SMILES: Fc1cccc2c(C34CNCCC3C4)n[nH]c12]

3-(3-azabicyclo[4.1.0]heptan-1-yl)-7-fluoro-1H-indazole (ZX162-139). ZX162-139 was prepared according to the procedure similar to ZX162-100 from ZX162-109 and tert-butyl 1-(4,4,5,5-tetramethyl-1,3,2-dioxaborolan-2-yl)-3-azabicyclo[4.1.0]heptane-3-carboxylate. ¹H NMR (400 MHz, Methanol-d₄) δ 7.58-7.53 (m, 1H), 7.14-7.05 (m, 2H), 4.10 (d, J=13.5 Hz, 1H), 3.61 (d, J=13.5 Hz, 1H), 3.22 (dt, J=12.4, 5.9 Hz, 1H), 3.02 (ddd, J=13.6, 9.1, 4.9 Hz, 1H), 2.48 (td, J=15.5, 5.9 Hz, 1H), 2.13 (dt, J=15.1, 5.6 Hz, 1H), 2.05-1.96 (m, 1H), 1.61 (dd, J=9.7, 5.4 Hz, 1H), 1.27-1.21 (m, 1H). MS (ESI) m/z: [M+H]⁺ 232.4.

### Example 195

### Synthesis of Compound ZX162-140

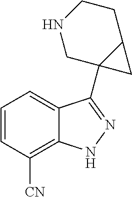 [SMILES: N#Cc1cccc2c(C34CNCCC3C4)n[nH]c12]

3-(3-azabicyclo[4.1.0]heptan-1-yl)-1H-indazole-7-carbonitrile (ZX162-140). ZX162-140 was prepared according to the procedure similar to ZX162-100 from ZX162-102 and tert-butyl 1-(4,4,5,5-tetramethyl-1,3,2-dioxaborolan-2-yl)-3-azabicyclo[4.1.0]heptane-3-carboxylate. ¹H NMR (400 MHz, Methanol-d₄) δ 8.11 (d, J=8.3 Hz, 1H), 7.81 (d, J=7.3 Hz, 1H), 7.28 (t, J=7.8 Hz, 1H), 4.16 (d, J=13.6 Hz, 1H), 3.61 (d, J=13.6 Hz, 1H), 3.24 (dt, J=12.2, 5.8 Hz, 1H), 3.02 (ddd, J=13.8, 9.2, 4.9 Hz, 1H), 2.49 (td, J=15.6, 5.9 Hz, 1H), 2.14 (dt, J=15.1, 5.4 Hz, 1H), 2.08-1.99 (m, 1H), 1.64 (dd, J=9.5, 5.8 Hz, 1H), 1.29 (t, J=6.2 Hz, 1H). MS (ESI) m/z: [M+H]⁺ 239.5.

### Example 196

### Synthesis of Compound ZX162-141

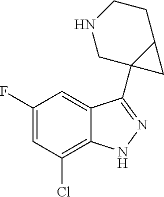 [SMILES: Fc1cc(Cl)c2[nH]nc(C34CNCCC3C4)c2c1]

3-(3-azabicyclo[4.1.0]heptan-1-yl)-7-chloro-5-fluoro-1H-indazole (ZX162-141). ZX162-141 was prepared according to the procedure similar to ZX162-100 from ZX162-101 and tert-butyl 1-(4,4,5,5-tetramethyl-1,3,2-dioxaborolan-2-yl)-3-azabicyclo[4.1.0]heptane-3-carboxylate. ¹H NMR (400 MHz, Methanol-d₄) δ 7.92 (d, J=6.9 Hz, 1H), 7.36 (d, J=9.2 Hz, 1H), 4.04 (d, J=13.5 Hz, 1H), 3.60 (d, J=13.6 Hz, 1H), 3.23 (dt, J=12.5, 5.9 Hz, 1H), 2.99 (ddd, J=14.1, 9.1, 4.9 Hz, 1H), 2.49 (dt, J=15.5, 7.7 Hz, 1H), 2.13 (dt, J=15.0, 5.3 Hz, 1H), 1.99-1.91 (m, 1H), 1.58 (dd, J=9.5, 5.7 Hz, 1H), 1.24 (t, J=5.4 Hz, 1H). MS (ESI) m/z: [M+H]⁺ 266.5.

### Example 197

### Synthesis of Compound ZX162-147

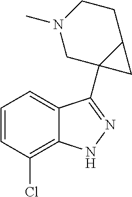 [SMILES: CN1CCC2CC2(c2n[nH]c3c(Cl)cccc23)C1]

7-chloro-3-(3-methyl-3-azabicyclo[4.1.0]heptan-1-yl)-1H-indazole (ZX162-147). ZX162-147 was prepared according to the procedure similar to ZX162-105 from ZX162-031 and formaldehyde. ¹H NMR (400 MHz, Methanol-d₄) δ 7.71 (d, J=8.1 Hz, 1H), 7.40 (d, J=7.5 Hz, 1H), 7.13 (t, J=7.8 Hz, 1H), 4.46 (d, J=13.2 Hz, 1H), 4.06-3.76 (m, 1H), 3.45 (d, J=10.9 Hz, 1H), 2.95 (s, 1H), 2.87 (s, 3H), 2.64-2.50 (m, 1H), 2.29 (d, J=14.9 Hz, 1H), 2.16-1.95 (m, 1H), 1.62 (dd, J=9.7, 5.4 Hz, 1H), 1.34 (t, J=6.6 Hz, 1H). MS (ESI) m/z: [M+H]⁺ 262.5.

### Example 198

### Synthesis of Compound ZX162-148

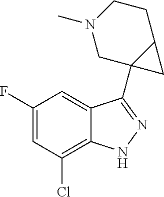 [SMILES: CN1CCC2CC2(c2n[nH]c3c(Cl)cc(F)cc23)C1]

7-chloro-5-fluoro-3-(3-methyl-3-azabicyclo[4.1.0]heptan-1-yl)-1H-indazole (ZX162-148). ZX162-148 was prepared according to the procedure similar to ZX162-105 from ZX162-141 and formaldehyde. ¹HNMR (400 MHz, Methanol-d₄) δ 7.81 (d, J=6.9 Hz, 1H), 7.26 (d, J=9.1 Hz, 1H), 4.28 (d, J=13.6 Hz, 1H), 3.77 (dd, J=80.2, 14.1 Hz, 1H), 3.35 (d, J=14.2 Hz, 1H), 2.85 (s, 1H), 2.76 (s, 3H), 2.59-2.37 (m, 1H), 2.19 (d, J=14.3 Hz, 1H), 1.94-1.87 (m, 1H), 1.47 (dd, J=9.6, 5.8 Hz, 1H), 1.22 (t, J=7.1 Hz, 1H). MS (ESI) m/z: [M+H]⁺ 280.6.

### Example 199

### Synthesis of Compound ZX162-151

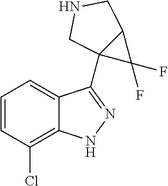 [SMILES: FC1(F)C2CNCC21c1n[nH]c2c(Cl)cccc12]

7-chloro-3-(6,6-difluoro-3-azabicyclo[3.1.0]hexan-1-yl)-1H-indazole (ZX162-151). ZX162-151 was prepared according to the procedure similar to ZX162-100 from ZX162-107 and potassium (3-(tert-butoxycarbonyl)-6,6-difluoro-3-azabicyclo[3.1.0]hexan-1-yl)trifluoroborate. ¹H NMR (400 MHz, Methanol-d₄) δ 7.72 (d, J=8.2 Hz, 1H), 7.46 (d, J=9.2 Hz, 1H), 7.20 (t, J=7.9 Hz, 1H), 4.28 (d, J=12.3 Hz, 1H), 4.11-4.01 (m, 2H), 3.90 (d, J=12.5 Hz, 1H), 3.47 (dd, J=11.4, 5.5 Hz, 1H). MS (ESI) m/z: [M+H]⁺ 270.4.

### Example 200

### Synthesis of Compound ZX162-173

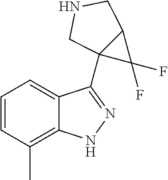 [SMILES: Cc1cccc2c(C34CNCC3C4(F)F)n[nH]c12]

3-(6,6-difluoro-3-azabicyclo[3.1.0]hexan-1-yl)-7-fluoro-1H-indazole (ZX162-173). ZX162-173 was prepared according to the procedure similar to ZX162-100 from ZX162-108 and potassium (3-(tert-butoxycarbonyl)-6,6-difluoro-3-azabicyclo[3.1.0]hexan-1-yl)trifluoroborate. ¹H NMR (400 MHz, Methanol-d₄) δ 7.57 (d, J=8.1 Hz, 1H), 7.21 (d, J=6.9 Hz, 1H), 7.12 (t, J=7.6 Hz, 1H), 4.27 (d, J=12.4 Hz, 1H), 4.09 (dd, J=11.8, 6.0 Hz, 1H), 3.99 (dd, J=12.4, 3.8 Hz, 1H), 3.90 (d, J=12.4 Hz, 1H), 3.41 (dd, J=11.2, 5.5 Hz, 1H). MS (ESI) m/z: [M+H]⁺ 250.4.

### Example 201

### Synthesis of Compound ZX162-174

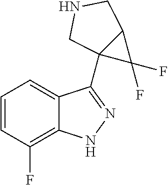 [SMILES: Fc1cccc2c(C34CNCC3C4(F)F)n[nH]c12]

3-(6,6-difluoro-3-azabicyclo[3.1.0]hexan-1-yl)-7-fluoro-1H-indazole (ZX162-174). ZX162-174 was prepared according to the procedure similar to ZX162-100 from ZX162-109 and potassium (3-(tert-butoxycarbonyl)-6,6-difluoro-3-azabicyclo[3.1.0]hexan-1-yl)trifluoroborate. ¹H NMR (400 MHz, Methanol-d₄) δ 7.56 (d, J=7.6 Hz, 1H), 7.20-7.14 (m, 2H), 4.28 (d, J=12.3 Hz, 1H), 4.12-4.01 (m, 2H), 3.90 (d, J=12.5 Hz, 1H), 3.46 (dd, J=11.4, 5.6 Hz, 1H). MS (ESI) m/z: [M+H]⁺: 254.4.

### Example 202

### Synthesis of Compound ZX162-175

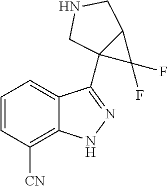 [SMILES: N#Cc1cccc2c(C34CNCC3C4(F)F)n[nH]c12]

3-(6,6-difluoro-3-azabicyclo[3.1.0]hexan-1-yl)-1H-indazole-7-carbonitrile (ZX162-175). ZX162-175 was prepared according to the procedure similar to ZX162-100 from ZX162-102 and potassium (3-(tert-butoxycarbonyl)-6,6-difluoro-3-azabicyclo[3.1.0]hexan-1-yl)trifluoroborate. ¹H NMR (400 MHz, Methanol-d₄) δ 8.12 (d, J=8.3 Hz, 1H), 7.89 (d, J=7.3 Hz, 1H), 7.36 (t, J=7.8 Hz, 1H), 4.31 (d, J=12.4 Hz, 1H), 4.13-4.05 (m, 2H), 3.91 (d, J=12.5 Hz, 1H), 3.52 (dd, J=11.5, 5.5 Hz, 1H). MS (ESI) m/z: [M+H]⁺ 261.4.

### Example 203

### Synthesis of Compound ZX162-176

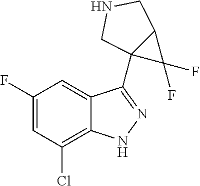 [SMILES: Fc1cc(Cl)c2[nH]nc(C34CNCC3C4(F)F)c2c1]

7-chloro-3-(6,6-difluoro-3-azabicyclo[3.1.0]hexan-1-yl)-5-fluoro-1H-indazole (ZX162-176). ZX162-176 was prepared according to the procedure similar to ZX162-100 from ZX162-101 and potassium (3-(tert-butoxycarbonyl)-6,6-difluoro-3-azabicyclo[3.1.0]hexan-1-yl)trifluoroborate. ¹H NMR (400 MHz, Methanol-d₄) δ 7.93 (d, J=6.9 Hz, 1H), 7.44 (d, J=9.3 Hz, 1H), 4.28 (d, J=12.4 Hz, 1H), 4.12-4.00 (m, 2H), 3.89 (d, J=12.6 Hz, 1H), 3.46 (dd, J=11.4, 5.6 Hz, 1H). MS (ESI) m/z: [M+H]⁺ 288.3.

### Example 204

### Synthesis of YX143-103B

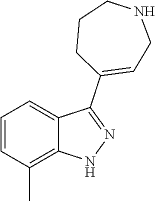 [SMILES: Cc1cccc2c(C3=CCNCCC3)n[nH]c12]

7-methyl-3-(2,5,6,7-tetrahydro-1H-azepin-4-yl)-1H-indazole (YX143-103B) YX143-103B was synthesized following the standard procedure for preparing NS131-179 from tert-butyl 3-bromo-7-methyl-1H-indazole-1-carboxylate and commercial available tert-butyl 5-(4,4,5,5-tetramethyl-1,3,2-dioxaborolan-2-yl)-2,3,4,7-tetrahydro-1H-azepine-1-carboxylate. (white solid, 5 mg, 23% yield). MS (ESI) m/z: [M+H]⁺ calcd for C₁₄H₁₈N₃ 228.1; found 228.4.

### Example 205

### Synthesis of YX143-103C

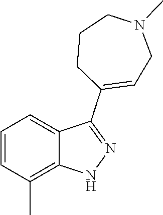 [SMILES: Cc1cccc2c(C3=CCN(C)CCC3)n[nH]c12]

7-methyl-3-(1-methyl-2,5,6,7-tetrahydro-1H-azepin-4-yl)-1H-indazole (YX143-103C) YX143-103C was synthesized following the Method M from YX143-103B, (white solid, 6 mg, 80% yield). MS (ESI) m/z: [M+H]⁺ calcd for C₁₅H₂₀N₃ 242.2; found 242.4.

### Example 206

### Synthesis of YX143-105C

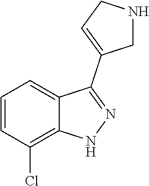 [SMILES: Clc1cccc2c(C3=CCNC3)n[nH]c12]

7-chloro-3-(2,5-dihydro-1H-pyrrol-3-yl)-1H-indazole (YX143-105C) YX143-105C was synthesized following the standard procedure for preparing NS131-179 from tert-butyl 3-bromo-7-methyl-1H-indazole-1-carboxylate and tert-butyl 3-(4,4,5,5-tetramethyl-1,3,2-dioxaborolan-2-yl)-2,5-dihydro-1H-pyrrole-1-carboxylate (brown solid, 17 mg, 25% yield). ¹H NMR (400 MHz, Methanol-d₄) δ 7.85 (d, J=8.3 Hz, 1H), 7.36 (d, J=7.6 Hz, 1H), 7.13 (t, J=7.8 Hz, 1H), 6.54 (p, J=2.2 Hz, 1H), 4.53 (q, J=2.3 Hz, 2H), 4.28 (q, J=2.4 Hz, 2H). MS (ESI) m/z: [M+H]⁺ calcd for C₁₁H₁₁ClN₃ 220.1; found 220.2.

### Example 207

### Synthesis of YX143-108

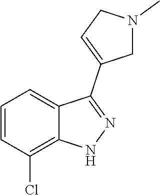 [SMILES: CN1CC=C(c2n[nH]c3c(Cl)cccc23)C1]

7-chloro-3-(1-methyl-2,5-dihydro-1H-pyrrol-3-yl)-1H-indazole (YX143-108). YX143-105C was synthesized following the Method M from YX143-105C (white solid, 8 mg, 80% yield)¹H NMR (400 MHz, Methanol-d₄) δ 7.98 (d, J=8.2 Hz, 1H), 7.50 (d, J=7.5 Hz, 1H), 7.27 (t, J=7.8 Hz, 1H), 6.65 (s, 1H), 4.98 (d, J=14.6 Hz, 1H), 4.70 (d, J=15.7 Hz, 1H), 4.53 (d, J=14.5 Hz, 1H), 4.27 (d, J=15.7 Hz, 1H), 3.18 (s, 3H). MS (ESI) m/z: [M+H]⁺ calcd for C₁₂H₁₃ClN₃ 234.1; found 234.3.

### Example 208

### Synthesis of YX143-110B

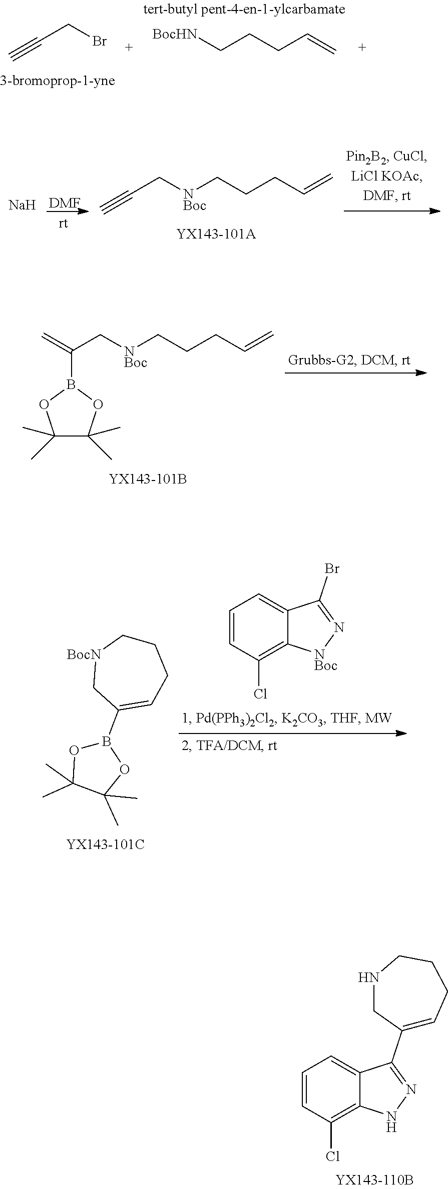

7-chloro-3-(2,5,6,7-tetrahydro-1H-azepin-3-yl)-1H-indazole (YX143-110B) To a solution of tert-butyl pent-4-en-1-ylcarbamate (1 g, 5.4 mmmol) in DMF (10 mL), was added NaH (0.5 g, 12.5 mmol) and continued stirred at room temperature. After 30 min, Propargyl bromide (1 mL, 89% in Tol, 6.7 mmol) was added, the mixture was continued stirred at room temperature. After 24 hours, sat. NH₄C₁ solution (10 mL) was added to quenched the reaction followed by extracted with Et₂O (3×10 mL). The organic layer was collected and concentrated, resulted residue was purified by ISCO to yield intermediate YX143-101A as brown oil (370 mg, 31% yield). ¹H NMR (400 MHz, Chloroform-d) δ 5.86-5.65 (m, 1H), 5.06-4.83 (m, 2H), 4.11-3.83 (m, 2H), 3.25 (t, J=7.5 Hz, 2H), 2.12 (t, J=2.5 Hz, 1H), 1.99 (q, J=7.5 Hz, 2H), 1.60 (p, J=7.5 Hz, 2H), 1.40 (s, 9H).

Intermediate YX143-101A (370 mg, 1.7 mmol), Pin2B2 (518 mg, 5.0 mmol), CuCl (207 mg, 2.1 mmol) LiCl (46 mg, 1.1 mmol) and KOAc (206 mg, 2.1 mmol was mixed in DMF (10 mL). The mixture was stirred at room temperature for 12 hours followed by quenched with water (5 mL) and extracted with Et₂O (3×5 mL). The organic layer was collected and concentrated, resulted residue was purified by ISCO to yield intermediate YX143-101B as brown oil (380 mg, 65% yield). ¹H NMR (400 MHz, Chloroform-d) δ 6.51-6.35 (m, 0.5H), 5.86-5.65 (m, 1.5H), 5.59-5.36 (m, 1H), 5.03-4.80 (m, 2H), 3.98-3.71 (m, 2H), 3.19-2.94 (m, 2H), 2.07-1.89 (m, 2H), 1.62-1.50 (m, 2H), 1.44-1.31 (m, 9H), 1.25-1.13 (m, 12H).

Intermediate YX143-101B (380 mg, 1.1 mmol) was dissolved in DCM (50 mL) followed by Grubbs 2^{nd} generation catalyst (45 mg, 0.055 mmol). The mixture was stirred at room temperature for 2 hour, then concentrated and purified by ISCO to yield the intermediate YX143-101C as brown oil (24 mg, 7% yield). ¹H NMR (400 MHz, Chloroform-d) δ 6.65-6.47 (m, 1H), 4.07-3.84 (m, 2H), 3.56-3.33 (m, 2H), 2.35-2.12 (m, 2H), 1.84-1.61 (m, 2H), 1.37 (s, 9H), 1.19 (s, 12H).

YX143-110B was synthesized following the standard procedure for preparing NS131-179 from tert-butyl 3-bromo-7-chloro-1H-indazole-1-carboxylate and intermediate YX143-101C (brown solid, 3 mg, 15% yield). ¹H NMR (400 MHz, Methanol-d₄) δ 7.82 (d, J=8.3 Hz, 1H), 7.33 (d, J=7.7 Hz, 1H), 7.09 (p, J=6.3 Hz, 1H), 7.01 (t, J=6.5 Hz, 1H), 4.46 (s, 2H), 3.52-3.37 (m, 2H), 2.69-2.56 (m, 2H), 1.99-1.78 (m, 2H). MS (ESI) m/z: [M+H]⁺ calcd for C₁₃H₁₅ClN₃ 248.1; found 248.3.

### Example 209

### Synthesis of YX143-112B

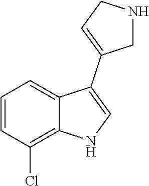 [SMILES: Clc1cccc2c(C3=CCNC3)c[nH]c12]

7-chloro-3-(2,5-dihydro-1H-pyrrol-3-yl)-1H-indole (YX143-112B) YX143-112B was synthesized following the standard procedure for preparing NS131-179 from tert-butyl 3-bromo-7-chloro-1H-indole-1-carboxylate and tert-butyl 3-(4,4,5,5-tetramethyl-1,3,2-dioxaborolan-2-yl)-2,5-dihydro-1H-pyrrole-1-carboxylate (brown solid, 10 mg, 15% yield). ¹H NMR (400 MHz, Methanol-d₄) δ 7.67 (d, J=8.0 Hz, 1H), 7.13 (d, J=7.8 Hz, 1H), 7.03 (t, J=7.8 Hz, 1H), 6.10 (p, J=2.1 Hz, 1H), 4.37 (q, J=2.3 Hz, 2H), 4.20 (q, J=2.3 Hz, 2H). MS (ESI) m/z: [M+H]⁺ calcd for C₁₂H₁₂ClN₂ 219.1; found 219.2.

### Example 210

### Synthesis of YX143-129

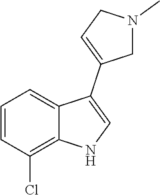 [SMILES: CN1CC=C(c2c[nH]c3c(Cl)cccc23)C1]

7-chloro-3-(1-methyl-2,5-dihydro-1H-pyrrol-3-yl)-1H-indole (YX143-129) YX143-129 was synthesized following the Method M from YX143-112B brown solid (7 mg, 90% yield). ¹H NMR (400 MHz, Methanol-d₄) δ 7.80 (d, J=8.0 Hz, 1H), 7.51 (d, J=2.2 Hz, 1H), 7.26 (d, J=7.6 Hz, 1H), 7.16 (t, J=7.8 Hz, 1H), 6.23-6.16 (m, 1H), 4.79 (d, J=14.1 Hz, 1H), 4.61 (d, J=14.9 Hz, 1H), 4.38 (d, J=14.0 Hz, 1H), 4.18 (d, J=14.8 Hz, 1H), 3.15 (s, 3H). MS (ESI) m/z: [M+H]⁺ calcd for C₁₃H₁₄ClN₂ 233.1; found 233.3.

### Example 211

### Synthesis of YX143-134C

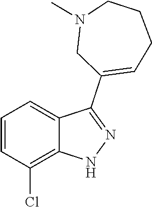 [SMILES: CN1CCCC=C(c2n[nH]c3c(Cl)cccc23)C1]

7-chloro-3-(1-methyl-2,5,6,7-tetrahydro-1H-azepin-3-yl)-1H-indazole (YX143-134C) YX143-134C was synthesized following Method M from YX143-110B brown solid (4 mg, 85% yield). ¹H NMR (400 MHz, Methanol-d₄) δ 7.83 (d, J=8.3 Hz, 1H), 7.34 (d, J=7.5 Hz, 1H), 7.14-7.04 (m, 2H), 4.65 (d, J=14.5 Hz, 1H), 4.52 (d, J=14.4 Hz, 1H), 3.73-3.54 (m, 1H), 3.54-3.34 (m, 1H), 2.92 (s, 3H), 2.72-2.53 (m, 2H), 2.14-1.79 (m, 2H). MS (ESI) m/z: [M+H]⁺ calcd for C₁₄H₁₇ClN₃ 262.1; found 262.2.

### Example 212

### Synthesis of YX143-138C

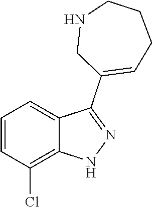 [SMILES: Clc1cccc2c(C3=CCCCNC3)n[nH]c12]

7-chloro-3-(2,5,6,7-tetrahydro-1H-azepin-3-yl)-1H-indole (YX143-138C) YX143-138C was synthesized following the standard procedure for preparing NS131-179 from tert-butyl 3-bromo-7-chloro-1H-indole-1-carboxylate and intermediate YX143-101C brown solid (2 mg, 10% yield). ¹H NMR (400 MHz, Methanol-d₄) δ 7.71 (d, J=8.0 Hz, 1H), 7.45 (s, 1H), 7.21 (d, J=7.6 Hz, 1H), 7.09 (t, J=7.9 Hz, 1H), 6.63 (t, J=6.5 Hz, 1H), 4.26 (s, 2H), 3.61-3.47 (m, 2H), 2.66 (t, J=6.1 Hz, 2H), 2.04 (s, 2H). MS (ESI) m/z: [M+H]⁺ calcd for C₁₄H₁₆ClN₂ 247.1; found 247.2.

### Example 213

### Synthesis of YX143-182C-1

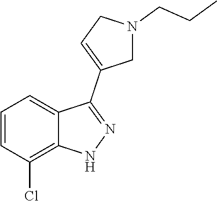 [SMILES: CCCN1CC=C(c2n[nH]c3c(Cl)cccc23)C1]

7-chloro-3-(1-propyl-2,5-dihydro-1H-pyrrol-3-yl)-1H-indazole (YX143-182C-1) YX143-182C-1 was synthesized following the Method M from YX143-105C brown solid (8 mg, 80% yield). ¹H NMR (400 MHz, Methanol-d₄) δ 7.97 (d, J=8.3 Hz, 1H), 7.49 (d, J=7.6 Hz, 1H), 7.26 (t, J=7.8 Hz, 1H), 6.76-6.61 (m, 1H), 4.77-4.14 (m, 4H), 3.43 (d, J=9.2 Hz, 2H), 2.00-1.77 (m, 2H), 1.11 (t, J=7.8 Hz, 3H). MS (ESI) m/z: [M+H]⁺ calcd for Cl₄H₁₇ClN₃ 262.1; found 262.3.

### Example 214

### Synthesis of YX143-183A

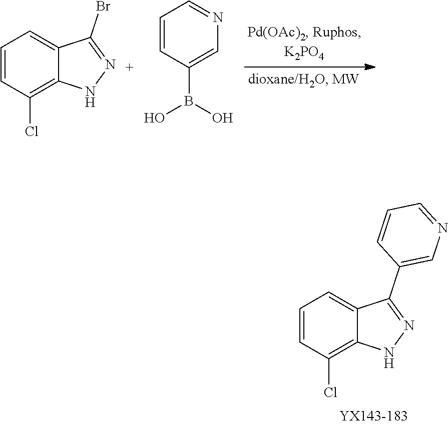 [SMILES: Clc1cccc2c(Br)n[nH]c12 ; OB(O)c1cccnc1 ; Clc1cccc2c(-c3cccnc3)n[nH]c12]

7-chloro-3-(pyridin-3-yl)-1H-indazole (YX143-183A) 3-bromo-7-chloro-1H-indazole (32 mg, 0.14 mmol), pyridin-3-ylboronic acid (24 mg, 0.20 mmol), Pd(OAc)₂ (25 mg, 0.11 mmol), Ruphos (24 mg, 0.051 mmol) and K₃PO₄ (127 mg, 0.60 mmol) were mixed in dioxane/H₂O (4:1, 1.5 mL). The mixture was irradiated under microwave condition at 140° C. for 30 min. after cooling down to room temperature, the mixture was filtered and purified by prep-HPLC to yield title compound as brown oil (2 mg, 6% yield). ¹H NMR (400 MHz, Methanol-d₄) δ 9.36 (s, 1H), 8.92 (d, J=8.2 Hz, 1H), 8.77 (s, 1H), 8.09 (d, J=8.2 Hz, 1H), 8.05-7.92 (m, 1H), 7.55 (d, J=7.7 Hz, 1H), 7.33 (t, J=7.9 Hz, 1H). MS (ESI) m/z: [M+H]⁺ calcd for C₁₂H₉ClN₃ 230.0; found 230.2.

### Examples 215 and 216

### Synthesis of YX143-184B-1 and YX143-184B-2

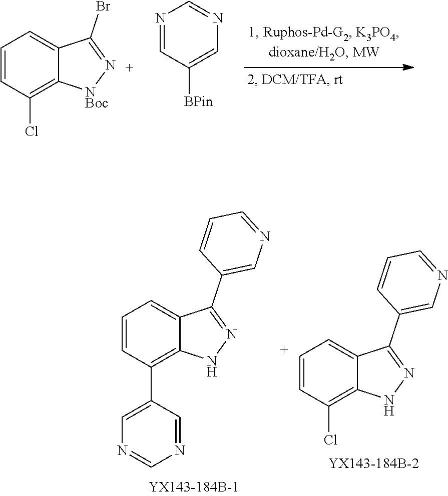

3,7-di(pyrimidin-5-yl)-1H-indazole (YX143-184B-1) tert-butyl 3-bromo-7-chloro-1H-indazole-1-carboxylate (33 mg, 0.10 mmol), 5-(4,4,5,5-tetramethyl-1,3,2-dioxaborolan-2-yl)pyrimidine (26 mg, 0.13 mmol), Ruphos-Pd-G2 (15 mg, 0.019 mmol) and K₃PO₄ (35 mg, 0.17 mmol) were mixed in dioxane/H₂O (9:1, 2 mL). The mixture was irradiated under microwave condition at 150° C. for 30 min. After cooling down to room temperature, the mixture was concentrated and re-dissolved in DCM/TFA (2:1, 1 mL). The solution was stirred at room temperature for 30 min followed by purification by prep-HPLC to yield the title compounds. YX143-184B-1, brown oil (5 mg, 13% yield), ¹H NMR (400 MHz, Methanol-d₄) δ 9.46 (t, J=2.3 Hz, 2H), 9.29 (s, 1H), 9.24 (s, 1H), 9.21-9.14 (m, 2H), 8.24 (d, J=8.3 Hz, 1H), 7.64 (d, J=7.0 Hz, 1H), 7.51 (t, J=7.6 Hz, 1H). MS (ESI) m/z: [M+H]⁺ calcd for C₁₅H₁N₆ 275.1; found 275.3.

7-chloro-3-(pyrimidin-5-yl)-1H-indazole (YX143-184B-2), brown oil (4 mg, 12% yield). ¹H NMR (400 MHz, Methanol-d₄) δ 9.42 (s, 2H), 9.22 (s, 1H), 8.07 (d, J=8.0 Hz, 1H), 7.53 (d, J=7.5 Hz, 1H), 7.31 (d, J=7.8 Hz, 1H). MS (ESI) m/z: [M+H]⁺ calcd for C₁₁H₈ClN₄ 230.0; found 230.2.

### Example 217

### Synthesis of YX143-185B

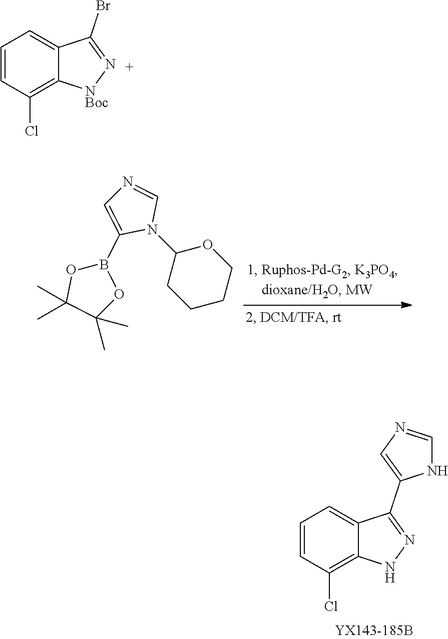 [SMILES: CC(C)(C)OC(=O)n1nc(Br)c2cccc(Cl)c21 ; CC1(C)OB(c2cncn2C2CCCCO2)OC1(C)C ; Clc1cccc2c(-c3cnc[nH]3)n[nH]c12]

7-chloro-3-(1H-imidazol-5-yl)-1H-indazole (YX143-185B) YX143-185B was synthesized following the same procedure for preparing YX143-184B-1 brown solid (7 mg, 22% yield). 1H NMR (400 MHz, Methanol-d₄) δ 9.04 (s, 1H), 8.22 (s, 1H), 8.00 (d, J=8.2 Hz, 1H), 7.54 (d, J=7.4 Hz, 1H), 7.31 (t, J=8.3 Hz, 1H). MS (ESI) m/z: [M+H]⁺ calcd for C₁₀H₈ClN₄ 219.0; found 219.2.

### Example 218

### Synthesis of YX143-186B

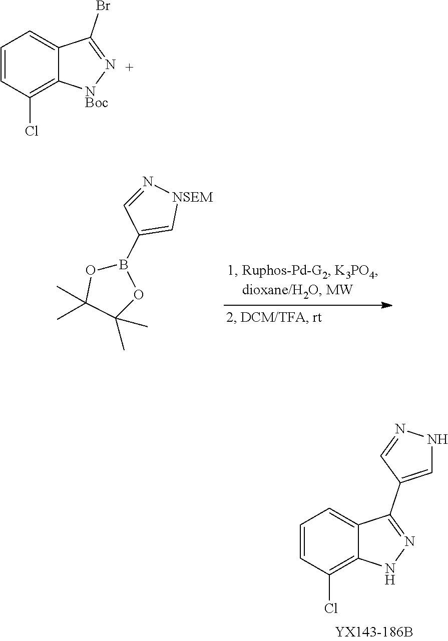 [SMILES: CC(C)(C)OC(=O)n1nc(Br)c2cccc(Cl)c21 ; CC1(C)OB(c2cnn(COCC[Si](C)(C)C)c2)OC1(C)C ; Clc1cccc2c(-c3cn[nH]c3)n[nH]c12]

7-chloro-3-(1H-pyrazol-4-yl)-1H-indazole (YX143-186B) YX143-186B was synthesized following the same procedure for preparing YX143-184B-1 brown solid (4 mg, 18% yield). ¹H NMR (400 MHz, Methanol-d₄) δ 8.25 (s, 2H), 7.96 (d, J=8.3 Hz, 1H), 7.46 (d, J=7.4 Hz, 1H), 7.21 (t, J=7.3 Hz, 1H). MS (ESI) m/z: [M+H]⁺ calcd for C₁₀H₈ClN₄ 219.0; found 219.2.

### Example 219

### Synthesis of YX157-19A

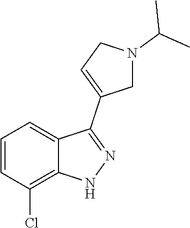 [SMILES: CC(C)N1CC=C(c2n[nH]c3c(Cl)cccc23)C1]

7-chloro-3-(1-isopropyl-2,5-dihydro-1H-pyrrol-3-yl)-1H-indazole (YX157-19A) YX157-19A was synthesized following Method M from YX143-105C, brown solid (5 mg, 79% yield). ¹H NMR (400 MHz, Methanol-d₄) δ 7.96 (d, J=8.4 Hz, 1H), 7.48 (d, J=7.6 Hz, 1H), 7.26 (t, J=8.0 Hz, 1H), 6.74-6.59 (m, 1H), 4.86-4.77 (m, 1H), 4.71-4.51 (m, 2H), 4.47-4.30 (m, 1H), 3.87-3.67 (m, 1H), 1.49 (s, 6H). MS (ESI) m/z: [M+H]⁺ calcd for Cl₄H₁₇ClN₃ 262.1; found 262.1.

### Example 220

### Synthesis of YX157-20A

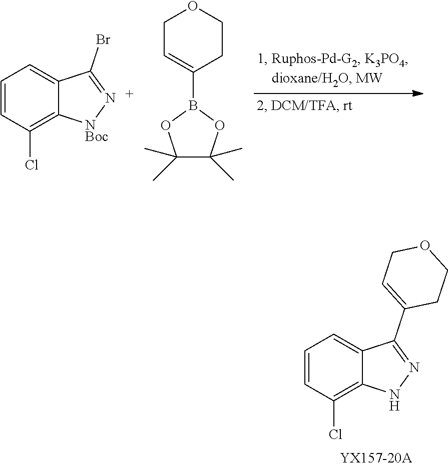

7-chloro-3-(3,6-dihydro-2H-pyran-4-yl)-1H-indazole (YX157-20A) YX157-20A was synthesized following the same procedure for preparing YX143-184B-1 brown solid (2 mg, 180% yield). ¹H NMR (400 MHz, Methanol-d₄) δ 7.93 (d, J=8.6 Hz, 1H), 7.42 (d, J=7.8 Hz, 1H), 7.16 (t, J=8.2 Hz, 1H), 6.59 (s, 1H), 4.48-4.33 (m, 2H), 4.10-3.90 (m, 2H), 2.86-2.69 (m, 2H). MS (ESI) m/z: [M+H]⁺ calcd for Cl₂H₁₂ClN₂O 235.1; found 235.2.

### Example 221

### Synthesis of YX157-29B

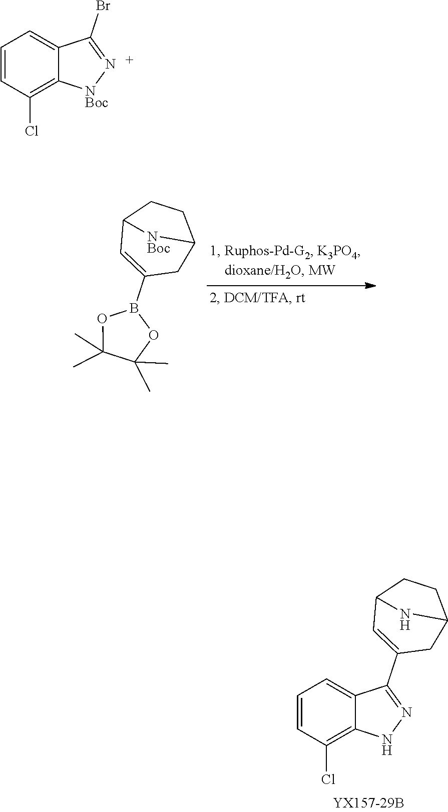 [SMILES: CC(C)(C)OC(=O)n1nc(Br)c2cccc(Cl)c21 ; CC(C)(C)OC(=O)N1C2C=C(B3OC(C)(C)C(C)(C)O3)CC1CC2 ; Clc1cccc2c(C3=CC4CCC(C3)N4)n[nH]c12]

3-(8-azabicyclo[3.2.1]oct-2-en-3-yl)-7-chloro-1H-indazole (YX157-29B) YX157-29B was synthesized following the same procedure for YX143-184B-1, brown oil (10 mg, 40% yield). ¹H NMR (400 MHz, Methanol-d₄) δ 7.85 (t, J=6.4 Hz, 1H), 7.46-7.28 (m, 1H), 7.20-7.00 (m, 1H), 6.82-6.62 (m, 1H), 4.50-4.34 (m, 1H), 4.34-4.21 (m, 1H), 3.69-3.47 (m, 1H), 3.02-2.82 (m, 1H), 2.46-2.11 (m, 3H), 2.04-1.83 (m, 1H). MS (ESI) m/z: [M+H]⁺ calcd for C₁₄H₁₅ClN₃ 260.1; found 260.3.

### Example 222

### Synthesis of YX157-42B

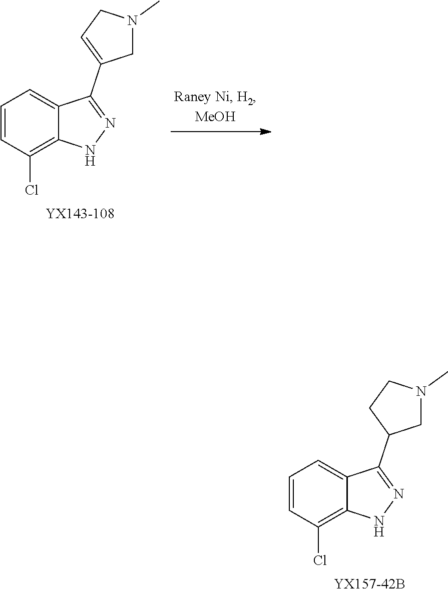 [SMILES: CN1CC=C(c2n[nH]c3c(Cl)cccc23)C1 ; CN1CCC(c2n[nH]c3c(Cl)cccc23)C1]

7-chloro-3-(1-methylpyrrolidin-3-yl)-1H-indazole (YX157-42B) To a solution of YX143-108 (10 mg, 0.029 mmol) in MeOH (2 mL) was added Raney Ni. The resulted mixture was stirred at room temperature under H₂ atmosphere for 2 hour followed by filtered. The filtrate was collected, concentrated and purified by prep-HPLC to yield YX157-42B, brown solid (8 mg, 80% yield). ¹H NMR (400 MHz, Methanol-d₄) δ 7.76 (d, J=8.1 Hz, 1H), 7.44 (d, J=7.4 Hz, 1H), 7.17 (t, J=8.1 Hz, 1H), 4.36-4.06 (m, 2H), 3.98-3.77 (m, 1H), 3.70-3.54 (m, 1H), 3.21-3.01 (m, 3H), 2.86-2.58 (m, 1H), 2.54-2.29 (m, 1H). MS (ESI) m/z: [M+H]⁺ calcd for C₁₂H₁₅ClN₃ 236.1; found 236.4.

### Example 223

### Synthesis of YX157-51B

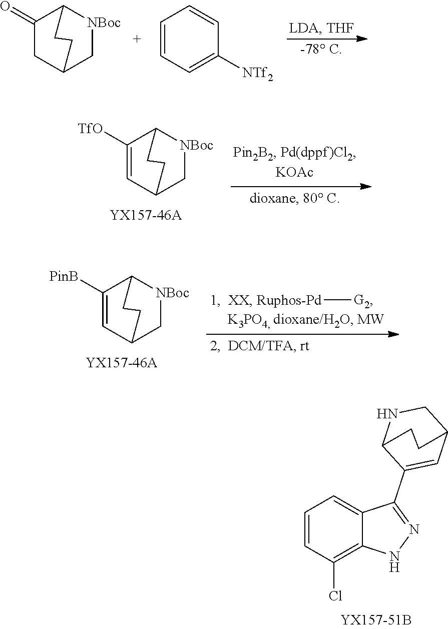

6-(7-chloro-1H-indazol-3-yl)-2-azabicyclo[2.2.2]oct-5-ene (YX157-51B) To tert-butyl 6-oxo-2-azabicyclo[2.2.2]octane-2-carboxylate (250 mg, 1.1 mmmol) in THF (3 mL) in −78° C., 1,1,1-trifluoro-N-phenyl-N-((trifluoromethyl)sulfonyl)methanesulfonamide (548 mg, 1.5 mmol) was added. The solution was warmed up overnight, and quenched with sat. NH₄Cl solution (2 mL), and extracted with EA (3×5 mL). the organic layer was collected and concentration followed by purified by ISCO to yield intermediate YX157-46A, brown oil (237 mg, 0.66 mmol). ¹H NMR (400 MHz, Chloroform-d) δ 6.08 (dd, J=7.5, 2.5 Hz, 1H), 4.91-4.52 (m, 1H), 3.16 (d, J=10.6 Hz, 1H), 3.02-2.81 (m, 2H), 2.04-1.87 (m, 2H), 1.72-1.50 (m, 2H), 1.38 (s, 9H).

Under Nitrogen atmosphere, intermediate YX157-46A (237 mg, 0.66 mmol), Pin₂B₂ (287 mg, 1.16 mmol), Pd(dppf)Cl₂ (154 mg, 0.19 mmol) and KOAc (215 mg, 2.19 mmol) were mixed with dioxane/H₂O (10 mL, 9:1), followed by heated at 80° C. overnight. After cooled down, the mixture was filtered, the filtrated was concentrated and purified by prep-HPLC to yield intermediate YX157-46B as brown oil (85 mg, 38% yield). ¹H NMR (400 MHz, Chloroform-d) δ 7.12 (s, 1H), 4.95-4.70 (m, 1H), 3.37-3.05 (m, 1H), 3.07-2.85 (m, 1H), 2.85-2.61 (m, 1H), 2.07-1.81 (m, 1H), 1.69-1.54 (m, 1H), 1.44 (s, 9H), 1.38-1.11 (m, 14H).

YX157-51B was synthesized following the same procedure for preparing YX143-184B-1 from tert-butyl 3-bromo-7-chloro-1H-indazole-1-carboxylate and YX157-46B, white solid (5 mg, 23% yield). ¹H NMR (400 MHz, Methanol-d₄) δ 7.99 (d, J=8.2 Hz, 1H), 7.47 (d, J=7.4 Hz, 1H), 7.40 (dd, J=7.1, 1.6 Hz, 1H), 7.23 (t, J=7.8 Hz, 1H), 5.27 (s, 1H), 3.25-3.16 (m, 1H), 2.95 (dt, J=11.2, 2.9 Hz, 1H), 2.68 (s, 1H), 2.34-2.20 (m, 1H), 2.02-1.92 (m, 1H), 1.83-1.62 (m, 2H). MS (ESI) m/z: [M+H]⁺ calcd for Cl₄H₁₅ClN₃ 260.1; found 260.3.

### Example 224

### Synthesis of YX157-51C

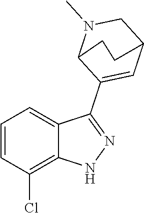 [SMILES: CN1CC2C=C(c3n[nH]c4c(Cl)cccc34)C1CC2]

6-(7-chloro-1H-indazol-3-yl)-2-methyl-2-azabicyclo[2.2.2]oct-5-ene (YX157-51C) YX157-51C was synthesized following Method M from YX157-51B, white solid (4 mg, 73% yield). ¹H NMR (400 MHz, Methanol-d₄) δ 8.05-7.98 (m, 1H), 7.53-7.46 (m, 1H), 7.46-7.35 (m, 1H), 7.30-7.21 (m, 1H), 5.34-5.07 (m, 1H), 3.79-3.60 (m, 1H), 3.26-3.16 (m, 1H), 3.12-2.99 (m, 1H), 2.77 (s, 3H), 2.37-2.22 (m, 1H), 1.95-1.75 (m, 2H), 1.75-1.60 (m, 1H). MS (ESI) m/z: [M+H]⁺ calcd for C₁₅H₁₇ClN₃ 274.1; found 274.3.

### Example 225

### Synthesis of YX157-55A

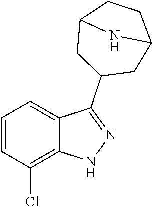 [SMILES: Clc1cccc2c(C3CC4CCC(C3)N4)n[nH]c12]

3-(8-azabicyclo[3.2.1]octan-3-yl)-7-chloro-1H-indazole (YX157-55A) YX157-55A was synthesized following the same procedure for preparing YX157-42B from YX157-29B, white solid (4 mg, 60% yield). ¹H NMR (400 MHz, Methanol-d₄) δ 7.71 (d, J=8.1 Hz, 1H), 7.41 (d, J=7.4 Hz, 1H), 7.14 (t, J=7.8 Hz, 1H), 4.18-4.02 (m, 2H), 3.73 (t, J=8.1 Hz, 1H), 2.88-2.77 (m, 2H), 2.66-2.50 (m, 2H), 2.18-2.03 (m, 2H), 1.98-1.85 (m, 2H). MS (ESI) m/z: [M+H]⁺ calcd for Cl₄H₁₇ClN₃ 262.1; found 262.3.

### Example 226

### Synthesis of XS159-153

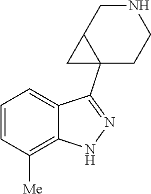 [SMILES: Cc1cccc2c(C34CCNCC3C4)n[nH]c12]

3-(3-azabicyclo[4.1.0]heptan-6-yl)-7-methyl-1H-indazole (XS159-153) XS159-153 was synthesized following the same procedure for preparing ZX162-100. ¹H NMR (400 MHz, Methanol-d₄) δ 7.65 (d, J=8.2 Hz, 1H), 7.16 (d, J=7.0 Hz, 1H), 7.11-7.01 (m, 1H), 3.84 (dd, J=13.5, 7.6 Hz, 1H), 3.35-3.29 (m, 1H), 3.26 (t, J=7.0 Hz, 1H), 3.06-2.99 (m, 1H), 2.65-2.58 (m, 1H), 2.53-2.46 (m, 4H), 1.94-1.87 (m, 1H), 1.51-1.48 (m, 1H), 1.21 (t, J=8.0 Hz, 1H). MS (ESI) m/z: [M+H]⁺ 228.47.

### Example 227

### Synthesis of XS159-155

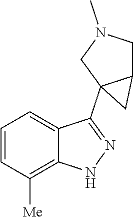 [SMILES: Cc1cccc2c(C34CC3CN(C)C4)n[nH]c12]

7-methyl-3-(3-methyl-3-azabicyclo[3.1.0]hexan-1-yl)-1H-indazole (XS159-155) XS159-155 was synthesized following the same procedure for preparing ZX162-105. ¹H NMR (400 MHz, Methanol-d₄) δ 7.55 (d, J=8.2 Hz, 1H), 7.17 (d, J=7.0 Hz, 1H), 7.11-7.02 (m, 1H), 4.18 (d, J=11.4 Hz, 1H), 3.91-3.82 (m, 2H), 3.71-3.67 (m, 1H), 3.05 (s, 3H), 2.54-2.51 (m, 4H), 1.61 (t, J=7.7 Hz, 1H), 1.37 (t, J=5.9 Hz, 1H). MS (ESI) m/z: [M+H]⁺ 228.4.

### Example 228

### Synthesis of XS159-160

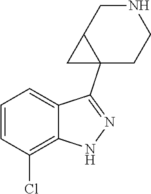 [SMILES: Clc1cccc2c(C34CCNCC3C4)n[nH]c12]

3-(3-azabicyclo[4.1.0]heptan-6-yl)-7-chloro-1H-indazole (XS159-160). XS159-160 was synthesized following the same procedure for preparing ZX162-100. ¹H NMR (400 MHz, Methanol-d₄) δ 7.80 (d, J=8.2 Hz, 1H), 7.42 (d, J=7.4 Hz, 1H), 7.14 (t, J=7.8 Hz, 1H), 3.87-3.82 (m, 1H), 3.35-3.27 (m, 2H), 3.07-3.00 (m, 1H), 2.69-2.62 (m, 1H), 2.55-2.49 (m, 1H), 1.99-1.93 (m, 1H), 1.54-1.51 (m, 1H), 1.25 (t, J=5.7 Hz, 1H). MS (ESI) m/z: [M+H]⁺ 248.2.

### Example 229

### Synthesis of XS159-163

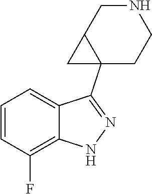 [SMILES: Fc1cccc2c(C34CCNCC3C4)n[nH]c12]

3-(3-azabicyclo[4.1.0]heptan-6-yl)-7-fluoro-1H-indazole (XS165-163). XS165-163 was synthesized following the same procedure for preparing ZX162-100. ¹H NMR (400 MHz, Methanol-d₄) δ 7.65-7.63 (m, 1H), 7.14-7.10 (m, 2H), 3.84 (dd, J=13.5, 7.6 Hz, 1H), 3.35-3.26 (m, 2H), 3.07-3.00 (m, 1H), 2.69-2.62 (m, 1H), 2.54-2.48 (m, 1H), 1.99-1.92 (m, 1H), 1.54-1.50 (m, 1H), 1.26-1.23 (m, 1H). MS (ESI) m/z: [M+H]⁺ 232.3.

### Method N;

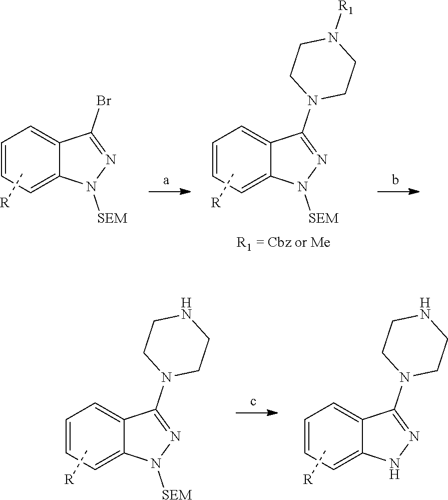

Step a. To a mixture of 3-bromoindazole derivatives (0.1 mmol, 1.0 eq), 1-Cbz-piperazine (0.2 mmol, 2.0 eq), Pd(OAc)₂ (0.01 mmol, 0.1 eq), XantPhos (0.012 mmol, 0.12 eq), and Cs₂CO₃ (0.28 mmol, 2.8 eq) was added 1,4-dioxane (1 mL). The mixture was heated 115° C. under nitrogen atmosphere overnight followed by filtered through a short column of silica gel. The filtrate was collected and concentrated to yield crude product used for next step directly without further purification.

Step b. The obtain crude product from last step was mixed with Pd/C (5%, 1 eq) in MeOH and stirred at room temperature for 6 h under hydrogen atmosphere. The reaction solution was filtered through a short column of silica gel, and the solvent was removed to yield crude product used for next step directly without further purification.

Step c. The obtain crude product from last step was dissolved in DCM followed by TFA (10 eq). After stirred at room temperature for 2 h, the solvent was removed, resulted residue was purified by prep-HPLC to yield desired compound.

### Example 230

### Synthesis of XS159-180

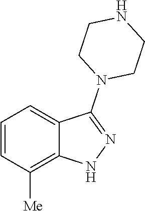 [SMILES: Cc1cccc2c(N3CCNCC3)n[nH]c12]

7-methyl-3-(piperazin-1-yl)-1H-indazole (XS159-180). XS159-180 was synthesized following Method N. H NMR (400 MHz, Methanol-d₄) δ 7.57 (d, J=8.2 Hz, 1H), 7.14 (d, J=6.9 Hz, 1H), 7.03-6.95 (m, 1H), 3.65-3.62 (m, 4H), 3.46-3.43 (m, 4H), 2.50 (s, 3H). MS (ESI) m/z: [M+H]⁺217.4.

### Example 231

### Synthesis of XS159-186

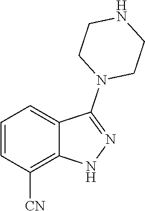 [SMILES: N#Cc1cccc2c(N3CCNCC3)n[nH]c12]

3-(piperazin-1-yl)-1H-indazole-7-carbonitrile (XS159-186). XS159-186 was synthesized following Method N. ¹H NMR (400 MHz, Methanol-d₄) δ 8.13 (d, J=8.2 Hz, 1H), 7.79 (d, J=7.3 Hz, 1H), 7.24-7.16 (m, 1H), 3.69-3.66 (m, 4H), 3.47-3.44 (m, 4H). MS (ESI) m/z: [M+H]⁺ 228.5.

### Example 232

### Synthesis of XS165-3

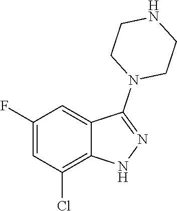 [SMILES: Fc1cc(Cl)c2[nH]nc(N3CCNCC3)c2c1]

7-chloro-5-fluoro-3-(piperazin-1-yl)-1H-indazole (XS165-3) XS165-3 was synthesized following Method N. ¹H NMR (400 MHz, Methanol-d₄) δ 7.95 (d, J=6.9 Hz, 1H), 7.27 (d, J=9.4 Hz, 1H), 3.64-3.57 (m, 4H), 3.45-3.43 (m, 4H). MS (ESI) m/z: [M+H]⁺ 255.4.

### Example 233

### Synthesis of XS165-5

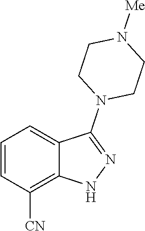 [SMILES: CN1CCN(c2n[nH]c3c(C#N)cccc23)CC1]

3-(4-methylpiperazin-1-yl)-1H-indazole-7-carbonitrile (XS165-5). XS165-5 was synthesized following Method N. ¹H NMR (400 MHz, Methanol-d₄) δ 8.14 (d, J=8.2 Hz, 1H), 7.81 (d, J=7.3 Hz, 1H), 7.26-7.18 (m, 1H), 4.21-4.10 (m, 2H), 3.66-3.62 (m, 3H), 3.49-3.33 (m, 3H), 3.01 (s, 3H). MS (ESI) m/z: [M+H]⁺ 242.3.

### Example 234

### Synthesis of XS165-8

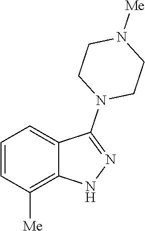 [SMILES: Cc1cccc2c(N3CCN(C)CC3)n[nH]c12]

7-methyl-3-(4-methylpiperazin-1-yl)-1H-indazole (XS165-8). XS165-8 was synthesized following Method N. ¹H NMR (400 MHz, Methanol-d₄) δ 7.56 (d, J=8.2 Hz, 1H), 7.14 (d, J=7.0 Hz, 1H), 6.99 (t, J=7.6 Hz, 1H), 4.06 (d, J=13.6 Hz, 2H), 3.63 (d, J=12.8 Hz, 2H), 3.40 (t, J=12.3 Hz, 2H), 3.26 (d, J=12.8 Hz, 2H), 3.00 (s, 3H), 2.49 (s, 3H). MS (ESI) m/z. [M+H]⁺ 231.6.

### Example 235

### Synthesis of XQ148-93

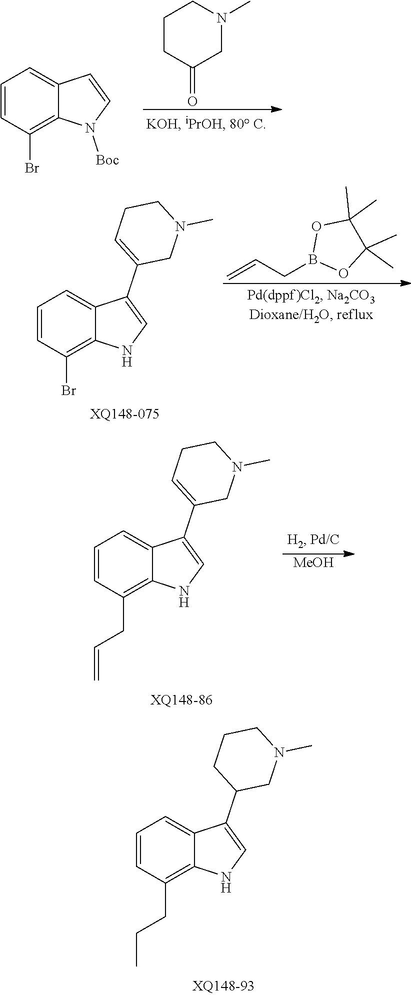

3-(1-methylpiperidin-3-yl)-7-propyl-1H-indole (XQ148-93)

Step 1: To a solution of tert-butyl 7-bromo-1H-indole-1-carboxylate (180 mg, 0.6 mmol, 1 equiv) in ⁱPrOH (4 mL) were added KOH (337 mg, 10 equiv) and 1-methylpiperidin-3-one HCl salt (359 mg, 1.8 mmol, 3 equiv) at rt, then the mixture was stirred for 8 h at 80° C., evaporated and the resulting mixture was purified by C18 column (10%-100% acetonitrile/0.1% TFA in H₂O) to give the product as a yellow solid (18 mg, 60%).

Step 2: to a solution of XQ148-075 (87.4 mg, 0.3 mmol, 1 equiv) in Dioxane (2 mL) were added 2-allyl-4,4,5,5-tetramethyl-1,3,2-dioxaborolane (100.8 mg, 0.6 mmol, 2 equiv), 2M Na₂CO₃ solution (0.3 mL, 0.6 mmol, 2 equiv), Pd(dppf)Cl₂ (22.0 mg, 0.03 mmol, 0.1 equiv), and the atmosphere evacuated and backfilled with nitrogen three times. After being stirred at 110° C. overnight, the resulting mixture was purified by C18 column (10%-100% acetonitrile/0.1% TFA in H₂O) to get compound XQ148-86 as a light yellow solid (23 mg, 30%).

Step 3: to a solution of XQ148-86 (10 mg) in Methanol (2 mL) were added palladium on carbon (catalytic amount), evacuated and recharged with hydrogen for 3 times, then stirred at rt for 3 hours. After filter and concentration, the resulting mixture was purified by prep-HPLC to get compound XQ148-93 as a light yellow solid (10 mg, 100%). ¹H NMR (400 MHz, Methanol-d₄) δ 7.40 (dd, J=7.7, 1.3 Hz, 1H), 7.08 (s, 1H), 6.98-6.86 (m, 2H), 3.61 (d, J=12.2 Hz, 1H), 3.49 (d, J=12.4 Hz, 1H), 3.04-2.92 (m, 2H), 2.84 (s, 2H), 2.82-2.74 (m, 2H), 2.19-2.03 (m, 2H), 1.97-1.86 (m, 1H), 1.85-1.77 (m, 1H), 1.68 (q, J=7.5 Hz, 2H), 1.56 (d, J=7.5 Hz, 1H), 1.29 (s, 1H), 0.93 (t, J=7.3 Hz, 3H). MS (ESI) m/z: calcd for C₁₇H₂₅N₂⁺ [M+H]⁺, 257.2 found, 257.4.

### Example 236

### Synthesis of XQ158-012

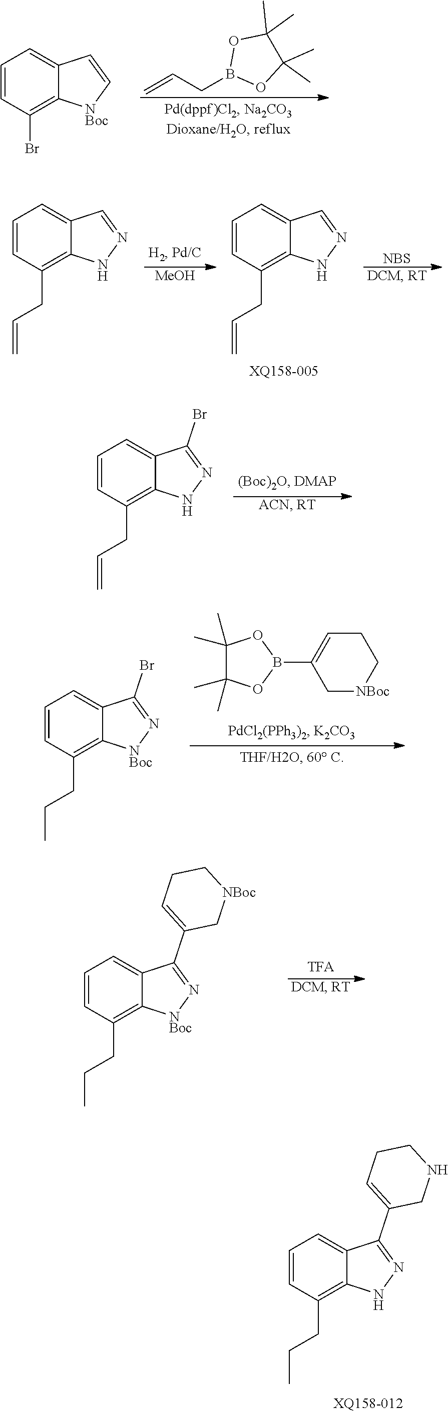

### 3-(piperidin-3-yl)-7-propyl-1H-indazole (XQ158-012)

Step 1 and 2: to a solution of tert-butyl 7-bromo-1H-indazole-1-carboxylate (178.2 mg, 0.6 mmol) in Dioxane (4 mL) were added 2-allyl-4,4,5,5-tetramethyl-1,3,2-dioxaborolane (201.6 mg, 1.2 mmol), 2M Na₂CO₃ solution (0.6 mL, 1.2 mmol), Pd(dppf)Cl₂ (44.0 mg, 0.06 mmol), and the atmosphere evacuated and backfilled with nitrogen three times. After being stirred at 110° C. overnight, the mixture was purified by silica gel (10% to 20% ethyl acetate in hexane), the resulting intermediate was dissolved in Methanol (2 mL), added palladium on carbon (catalytic amount), evacuated and recharged with hydrogen for 3 times, then stirred at rt for 3 hours. After filter and concentration, the resulting mixture was purified by silica gel (10% to 20% ethyl acetate in hexane) to get compound XQ158-005 as a yellow oil (43 mg, 45%).

Step 3: XQ158-012 was synthesized following the standard procedure for preparing NS144-102 from XQ158-005 and tert-butyl 5-(4,4,5,5-tetramethyl-1,3,2-dioxaborolan-2-yl)-3,6-dihydropyridine-1(2H)-carboxylate (yellow solid, 2 mg, 3%). ¹H NMR (400 MHz, Methanol-d₄) δ 7.82 (d, J=8.1 Hz, 1H), 7.21 (d, J=6.9 Hz, 1H), 7.16 (t, J=7.6 Hz, 1H), 6.84 (s, 1H), 4.30 (s, 2H), 3.47 (t, J=6.2 Hz, 2H), 2.92 (t, J=7.6 Hz, 2H), 2.73 (s, 2H), 1.78 (h, J=7.1 Hz, 2H), 1.02 (t, J=7.3 Hz, 3H). MS (ESI) m/z: calcd for C₁₅H₂₀N₃⁺ [M+H]⁺, 242.2 found, 242.3.

### Example 237

### Synthesis of XQ158-055

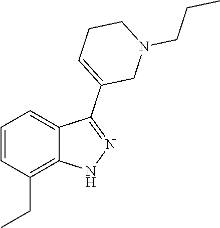 [SMILES: CCCN1CCC=C(c2n[nH]c3c(CC)cccc23)C1]

7-ethyl-3-(1-propyl-1,2,5,6-tetrahydropyridin-3-yl)-1H-indazole (XQ158-055). XQ158-055 was synthesized following the standard procedure for preparing XQ158-115 from compound XQ148-012 (light yellow solid, 6 mg, 22%). ¹H NMR (400 MHz, Methanol-d₄) δ 7.83 (d, J=8.1 Hz, 1H), 7.24 (d, J=7.1 Hz, 1H), 7.18 (t, J=7.8 Hz, 1H), 6.85 (s, 1H), 4.67 (d, J=16.2 Hz, 2H), 4.15 (d, J=16.3 Hz, 2H), 3.76 (s, 2H), 2.97 (q, J=7.2 Hz, 2H), 2.86-2.70 (m, 2H), 2.00-1.86 (m, 2H), 1.37 (t, J=7.5 Hz, 3H), 1.12 (t, J=7.4 Hz, 3H). MS (ESI) m/z: calcd for C₁₇H₂₄N₃⁺ [M+H]⁺, 270.2 found, 270.3.

### Example 238

### Synthesis of XQ158-056

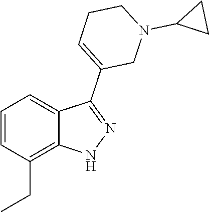 [SMILES: CCc1cccc2c(C3=CCCN(C4CC4)C3)n[nH]c12]

3-(1-cyclopropyl-1,2,5,6-tetrahydropyridin-3-yl)-7-ethyl-1H-indazole (XQ158-056). XQ158-056 was synthesized following the standard procedure for preparing XQ158-115 from compound 148-012 using bromocyclopropane (yellow solid, 14 mg, 52%). ¹H NMR (400 MHz, Methanol-d₄) δ 7.82 (d, J=8.1 Hz, 1H), 7.24 (d, J=7.0 Hz, 1H), 7.18 (t, J=8.0 Hz, 1H), 6.85 (s, 1H), 6.18-6.04 (m, 1H), 5.79-5.66 (m, 2H), 4.65 (d, J=15.9 Hz, 1H), 4.15 (d, J=16.7 Hz, 1H), 4.01 (d, J=7.2 Hz, 2H), 3.76 (s, 1H), 2.97 (q, J=7.3 Hz, 2H), 2.81 (s, 2H), 1.37 (t, J=7.5 Hz, 4H). MS (ESI) m/z: calcd for C₁₇H₂₂N₃⁻ [M+H]⁺, 268.2 found, 268.4.

### Example 239

### Synthesis of XQ158-078

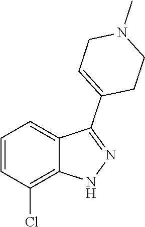 [SMILES: CN1CC=C(c2n[nH]c3c(Cl)cccc23)CC1]

7-chloro-3-(1-methyl-1,2,3,6-tetrahydropyridin-4-yl)-1H-indazole (XQ158-078). XQ158-078 was synthesized following the standard procedure for preparing NS144-102 from 3-bromo-7-chloro-1H-indazole and 1-methyl-4-(4,4,5,5-tetramethyl-1,3,2-dioxaborolan-2-yl)-1,2,3,6-tetrahydropyridine (yellow solid, 55 mg, 74%). ¹H NMR (400 MHz, Methanol-d₄) δ 7.96 (dd, J=8.3, 1.9 Hz, 1H), 7.46 (dd, J=7.5, 1.9 Hz, 1H), 7.26-7.18 (m, 1H), 6.60 (dd, J=4.2, 2.1 Hz, 1H), 4.19 (d, J=16.9 Hz, 1H), 3.94 (d, J=17.1 Hz, 1H), 3.84-3.74 (m, 1H), 3.47 (d, J=14.4 Hz, 1H), 3.24 (d, J=19.8 Hz, 1H), 3.17-3.03 (m, 4H). MS (ESI) m/z: calcd for C₁₃H₁₅ClN₃⁺ [M+H]⁺, 248.1 found, 248.2.

### Example 240

### Synthesis of XQ158-093A

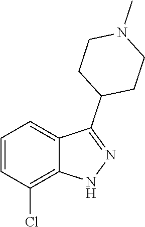 [SMILES: CN1CCC(c2n[nH]c3c(Cl)cccc23)CC1]

7-chloro-3-(1-methylpiperidin-4-yl)-1H-indazole (XQ158-093A). XQ158-093A was synthesized following the standard procedure for preparing YX157-27A-2 from XQ158-078 (yellow solid, 8 mg, 32%). ¹H NMR (400 MHz, Methanol-d₄) δ 7.78 (d, J=8.3 Hz, 1H), 7.43 (d, J=7.3 Hz, 1H), 7.18-7.12 (M, 1H), 3.74-3.64 (m, 2H), 3.59-3.45 (m, 1H), 3.26 (t, J=12.7 Hz, 2H), 2.98 (d, J=2.0 Hz, 3H), 2.37 (d, J=15.2 Hz, 2H), 2.29-2.17 (m, 2H). MS (ESI) m/z: calcd for Cl₃H₁₇ClN₃⁺ [M+H]f, 250.1 found, 250.1.

### Example 241

### Synthesis of XQ158-082

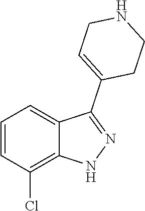 [SMILES: Clc1cccc2c(C3=CCNCC3)n[nH]c12]

7-chloro-3-(1,2,3,6-tetrahydropyridin-4-yl)-1H-indazole (XQ158-082). XQ158-082 was synthesized following the standard procedure for preparing NS144-102 from 3-bromo-7-chloro-1H-indazole and tert-butyl 4-(4,4,5,5-tetramethyl-1,3,2-dioxaborolan-2-yl)-3,6-dihydropyridine-1(2H)-carboxylate (light yellow solid, 48 mg, 69%). ¹H NMR (400 MHz, Methanol-d₄) δ 7.96 (dd, J=8.3, 1.9 Hz, 1H), 7.46 (dd, J=7.4, 1.9 Hz, 1H), 7.25-7.18 (m, 1H), 6.63 (dd, J=3.5, 1.9 Hz, 1H), 3.99 (t, J=2.9 Hz, 2H), 3.58-3.52 (m, 2H), 3.09 (d, J=7.0 Hz, 2H). MS (ESI) m/z: calcd for C₂H₁₃ClN₃ [M+H]⁺, 234.1 found, 234.2.

### Example 242

### Synthesis of XQ158-115

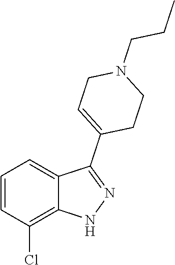 [SMILES: CCCN1CC=C(c2n[nH]c3c(Cl)cccc23)CC1]

7-chloro-3-(1-propyl-1,2,3,6-tetrahydropyridin-4-yl)-1H-indazole (XQ158-115). XQ158-115 was synthesized following similar procedure for preparing NS144-108 (yellow solid, 12 mg, 46%).

¹H NMR (400 MHz, Methanol-d₄) δ 7.96 (dd, J=8.3, 2.0 Hz, 1H), 7.46 (dd, J=7.5, 1.9 Hz, 1H), 7.22 (t, J=8.1 Hz, 1H), 6.65-6.56 (m, 1H), 4.22 (d, J=17.1 Hz, 1H), 4.00-3.76 (m, 2H), 3.42 (s, 1H), 3.31-3.19 (m, 3H), 3.11 (s, 1H), 1.97-1.82 (m, 2H), 1.11 (td, J=7.5, 1.6 Hz, 3H). MS (ESI) m/z: calcd for C₁₅H₁₉ClN₃⁺ [M+H]⁺, 276.1 found, 276.4.

### Example 243

### Synthesis of XQ158-164

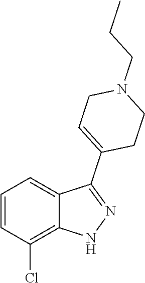 [SMILES: CCCN1CC=C(c2n[nH]c3c(Cl)cccc23)CC1]

7-chloro-3-(1-propyl-1,2,3,6-tetrahydropyridin-4-yl)-1H-indole (XQ158-164). XQ158-164 was synthesized following the standard procedure for preparing XQ158-115 (yellow solid, 6 mg, 38%). H NMR (400 MHz, Methanol-d₄) δ 7.69 (d, J=8.0 Hz, 1H), 7.38 (d, J=1.5 Hz, 1H), 7.10 (d, J=7.6 Hz, 1H), 6.99 (t, J=7.9 Hz, 1H), 6.10 (s, 1H), 4.01 (s, 1H), 3.72 (d, J=38.8 Hz, 2H), 3.29 (s, 1H), 3.17-3.07 (m, 2H), 2.85 (s, 2H), 1.83-1.68 (m, 2H), 0.97 (t, J=6.6 Hz, 3H). MS (ESI) m/z: calcd for C₁₆H₂₀ClN₂⁺ [M+H]⁺, 275.1 found, 275.3.

### Example 244

### Synthesis of XQ158-167

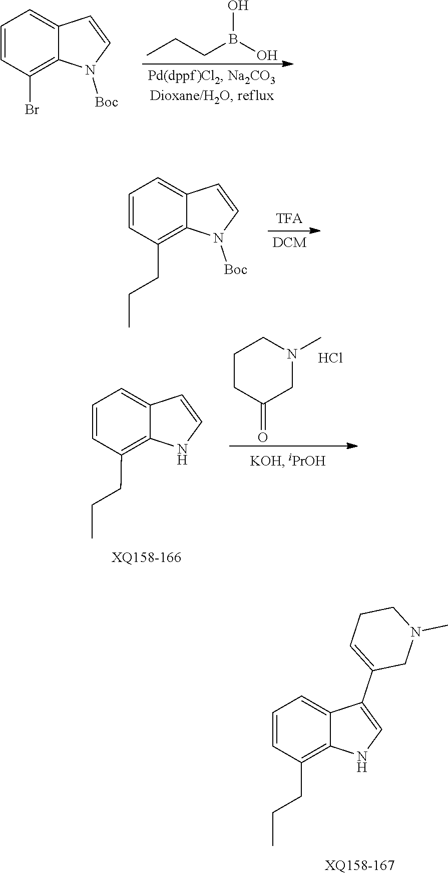

### 3-(1-methyl-1,2,5,6-tetrahydropyridin-3-yl)-7-propyl-1H-indole (XQ158-167)

Step 1 and 2: to a solution of tert-butyl 7-bromo-1H-indole-1-carboxylate (296.2 mg, 1 mmol, 1 equiv) in Dioxane (5 mL) were added propylboronic acid (175.8 mg, 2 mmol, 2 equiv), 2M Na₂CO₃ solution (1 mL, 2 mmol, 2 equiv), Pd(dppf)Cl₂ (36.6 mg, 0.05 mmol, 0.05 equiv), and the atmosphere evacuated and backfilled with nitrogen three times. After refluxed overnight, the resulting mixture was filtered and concentrated. Then treated with TFA (3 mL) in DCM (1 mL) for 1 h. purified by silica gel (10% to 20% ethyl acetate in hexane) to get compound XQ148-166 as a yellow oil (86 mg, 54%).

Step 3: To a solution of XQ158-166 (31.8 mg, 0.2 mmol) in ⁱPrOH (2 mL) were added KOH (112.2 mg, 2 mmol) and 1-methylpiperidin-3-one HCl salt (89.8 mg, 0.6 mmol) at, then the mixture was stirred for 12 h at 80° C., evaporated and the resulting mixture was purified by prep-HPLC to give the final product as a yellow solid (4 mg, 8%). ¹H NMR (400 MHz, Methanol-d₄) δ 7.67 (d, J=7.9 Hz, 1H), 7.37 (d, J=1.8 Hz, 1H), 7.06 (t, J=7.1 Hz, 1H), 7.01 (d, J=6.8 Hz, 1H), 6.40 (s, 1H), 3.51 (s, 1H), 3.16 (s, 1H), 3.08 (s, 1H), 2.90-2.83 (m, 1H), 2.73 (s, 1H), 1.76 (q, J=7.6 Hz, 2H), 1.42-1.29 (m, 4H), 1.04-0.97 (m, 2H), 0.94 (s, 3H). MS (ESI) m/z: calcd for C₁₇H₂₃N₂⁺ [M+H]⁺, 255.2 found, 255.3.

### Example 245

### Synthesis of XQ158-168

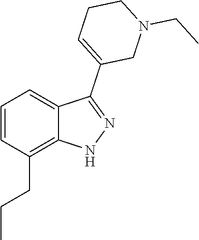 [SMILES: CCCc1cccc2c(C3=CCCN(CC)C3)n[nH]c12]

3-(1-ethyl-1,2,5,6-tetrahydropyridin-3-yl)-7-propyl-1H-indole (XQ158-168). XQ158-168 was synthesized following the standard procedure for preparing XQ158-167 from 1-ethylpiperidin-3-one HCl salt (yellow solid, 3 mg, 6%). ¹H NMR (400 MHz, Methanol-d₄) δ 7.66 (d, J=7.9 Hz, 1H), 7.35 (s, 1H), 7.06 (t, J=7.6 Hz, 1H), 7.00 (d, J=7.2 Hz, 1H), 6.38 (s, 1H), 4.11 (s, 2H), 3.52-3.49 (m, 1H), 3.16 (s, 1H), 3.03 (s, 1H), 2.86 (t, J=7.8 Hz, 1H), 2.72 (s, 1H), 1.76 (q, J=7.5 Hz, 1H), 1.31 (s, 2H), 1.01 (t, J=7.2 Hz, 2H), 0.92 (s, 6H). MS (ESI) m/z: calcd for C₁₈H₂₅N₂+[M+H]⁺, 269.2 found, 269.4.

### Method O;

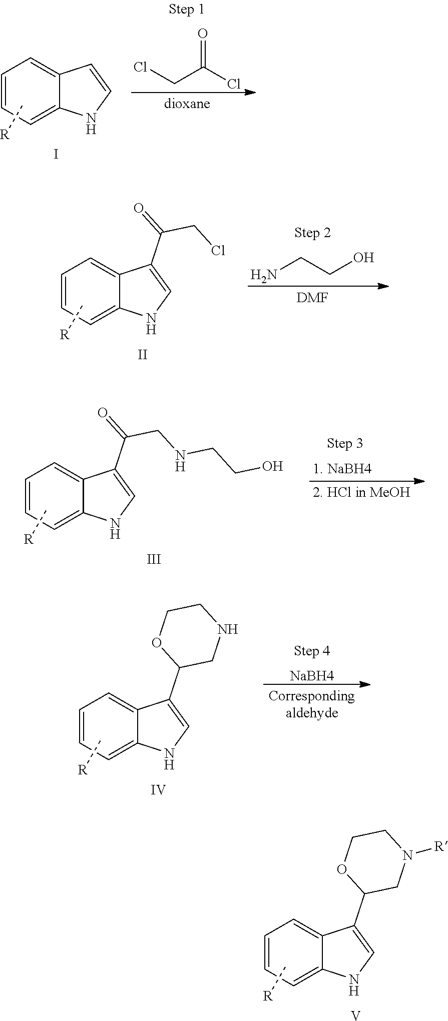

Step 1: A mixture of substrate I (1.0 eq) and 2-chloroacetyl chloride (1.0 eq) in dioxane (0.4 M) was reflux for 1-16 h. After the substrate disappears, the mixture was allowed to cool to rt and then poured to water followed by extracted with ethyl acetate and purified by column chromatography on silica to get compound II.

Step 2: A mixture of compound II (1.0 eq) and ethanolamine (2.5 eq) in DMF (0.2 M) was stirred at rt. for 5 h. After concentration, the residue was purified by column chromatography on silica (eluting with gradient formed from DCM/Methanol/NH₃ aq) to get compound III.

Step 3: A mixture of compound III (1.0 eq) and sodium borohydride (17 eq) in Methanol (0.01 M) was stirred at rt. for 20 h. After concentration, the residue was treated with THF, ethyl acetate and Na₂CO₃ aq. (10%), the aqueous phase extracted with THF/Ethyl acetate. Evaporated to dryness and the residue was taken up in methanol (0.1 M) and treated at 0° C. with 1.25 N HCl in methanol for 45 min. The mixture was evaporated to dryness and purified by column chromatography on silica with a gradient formed from DCM/Methanol/NH₃ aq to get compound IV.

Step 4: To a solution of compound IV (1.0 eq) in methanol was added corresponding aldehyde (10.0 eq) and NaBH₃CN (2.0 eq). The mixture was stirred at rt for 2 h followed by purified by prep-HPLC to get compound V.

### Example 246

### Synthesis of ZD160-34

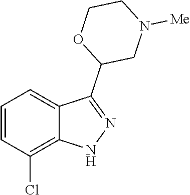 [SMILES: CN1CCOC(c2n[nH]c3c(Cl)cccc23)C1]

2-(7-chloro-1H-indol-3-yl)-4-methylmorpholine (ZD160-34). ZD160-34 was synthesized following Method O from 7-chloro-1H-indole. ¹H NMR (400 MHz, Methanol-d₄) δ 7.67 (dd, J=8.0, 1.8 Hz, 1H), 7.44 (d, J=1.8 Hz, 1H), 7.20 (dd, J=7.6, 1.8 Hz, 1H), 7.08 (td, J=7.8, 1.8 Hz, 1H), 5.08 (d, J=11.1 Hz, 1H), 4.29 (d, J=13.4 Hz, 1H), 4.05 (t, J=12.7 Hz, 1H), 3.72 (d, J=12.4 Hz, 1H), 3.57 (d, J=12.6 Hz, 1H), 3.43 (t, J=11.9 Hz, 1H), 3.30 (d, J=9.6 Hz, OH), 3.01 (s, 3H). MS(ESI) m/z: [M+H]⁺ 251.7.

### Example 247

### Synthesis of ZD160-140

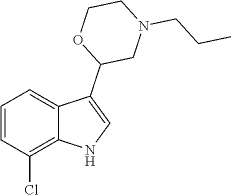 [SMILES: CCCN1CCOC(c2c[nH]c3c(Cl)cccc23)C1]

2-(7-chloro-1H-indol-3-yl)-4-propylmorpholine (ZD160-140). ZD160-140 was synthesized following Method O from 7-chloro-1H-indole. ¹H NMR (400 MHz, Methanol-d₄) δ 7.57-7.53 (m, 1H), 7.32 (d, J=2.0 Hz, 1H), 7.09 (dd, J=7.6, 2.0 Hz, 1H), 6.96 (td, J=7.8, 2.1 Hz, 1H), 4.97 (d, J=11.1 Hz, 1H), 4.18 (d, J=13.3 Hz, 1H), 3.93 (t, J=12.7 Hz, 1H), 3.62 (d, J=12.5 Hz, 1H), 3.51 (d, J=12.8 Hz, 1H), 3.29 (t, J=11.9 Hz, 1H), 3.18-3.03 (m, 3H), 1.79-1.66 (m, 2H), 0.95 (td, J=7.4, 2.1 Hz, 3H). MS(ESI) m/z: [M+H]⁺ 279.5.

### Example 248

### Synthesis of ZD160-141

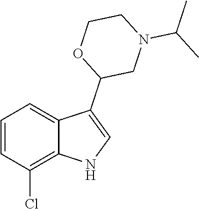 [SMILES: CC(C)N1CCOC(c2c[nH]c3c(Cl)cccc23)C1]

2-(7-chloro-1H-indol-3-yl)-4-isopropylmorpholine (ZD160-141). ZD160-141 was synthesized following Method O from 7-chloro-1H-indole. ¹H NMR (400 MHz, Chloroform-d) δ 8.44 (s, 1H), 7.67 (d, J=7.9 Hz, 1H), 7.31 (d, J=2.3 Hz, 1H), 7.24 (d, J=8.3 Hz, 1H), 7.11 (t, J=7.8 Hz, 1H), 5.35 (d, J=10.7 Hz, 1H), 4.32 (t, J=12.3 Hz, 1H), 4.23 (dd, J=13.5, 3.9 Hz, 1H), 3.63 (d, J=11.8 Hz, 1H), 3.58-3.42 (m, 2H), 3.01 (q, J=14.1, 11.7 Hz, 2H), 1.39 (dd, J=6.5, 2.8 Hz, 6H). MS(ESI) m/z: [M+H]⁺ 279.6.

### Example 249

### Synthesis of ZD160-149

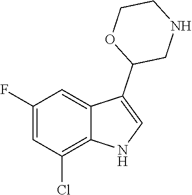 [SMILES: Fc1cc(Cl)c2[nH]cc(C3CNCCO3)c2c1]

2-(7-chloro-5-fluoro-1H-indol-3-yl)morpholine (ZD160-149). ZD160-149 was synthesized following Method O from 7-chloro-5-fluoro-1H-indole. ¹H NMR (400 MHz, Methanol-d₄) δ 7.49 (d, J=1.8 Hz, 1H), 7.40 (dt, J=9.3, 2.4 Hz, 1H), 7.08 (dd, J=9.1, 2.4 Hz, 1H), 5.05 (dt, J=11.1, 2.4 Hz, 1H), 4.33-4.19 (m, 1H), 4.06 (ddt, J=13.1, 9.2, 3.1 Hz, 1H), 3.57-3.51 (m, 1H), 3.44 (ddd, J=12.9, 10.4, 1.8 Hz, 1H), 3.38 (d, J=3.3 Hz, 2H). MS(ESI) m/z: [M+H]⁺ 255.4.

### Method P;

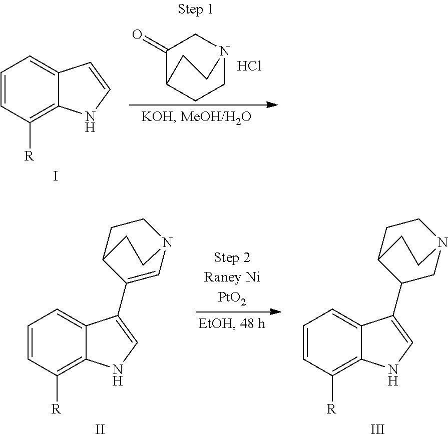

Step 1: A solution of substrate I (1.0 eq) in MeOH/H₂O (1:1, 0.3M) was treated with KOH (5.0 eq), followed by 3-quinoclidine hydrochloride (2.0 eq) at rt. After heated at refluxed for 36 h, the reaction was brought to rt, filtered and washed with MeOH/H₂O (1:1), followed by methanol. The solid was collected and dried under vacuum to obtain the compound II.

Step 2: A solution of compound II (1.0 eq) in EtOH was added Raney Ni (0.1 eq) and PtO₂ (0.1 eq) under H₂, the mixture was stirred at rt for 48 h. Then filted through diatomaceous earth and the residue was purified by prep-HPLC to get compound III.

### Example 250

### Synthesis of ZD160-11

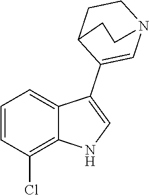 [SMILES: Clc1cccc2c(C3=CN4CCC3CC4)c[nH]c12]

3-(7-chloro-1H-indol-3-yl)-1-azabicyclo[2.2.2]oct-2-ene (ZD160-11). ZD160-11 was synthesized following Method P from 7-chloro-1H-indole. ¹H NMR (400 MHz, Methanol-d₄) δ 7.69 (dd, J=8.0, 0.9 Hz, 1H), 7.48 (s, 1H), 7.19 (dd, J=7.6, 0.9 Hz, 1H), 7.08 (t, J=7.8 Hz, 1H), 6.81 (d, J=1.5 Hz, 1H), 3.18 (dt, J=4.5, 2.3 Hz, 1H), 3.15-3.06 (m, 2H), 2.73 (tdd, J=13.3, 4.9, 2.4 Hz, 2H), 1.89 (dddd, J=11.7, 9.0, 4.7, 2.6 Hz, 2H), 1.71 (tdd, J=13.0, 6.5, 3.9 Hz, 2H). MS(ESI) m/z: [M+H]⁺ 259.3.

### Example 251

### Synthesis of ZD160-133

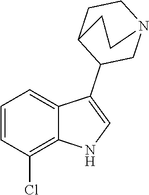 [SMILES: Clc1cccc2c(C3CN4CCC3CC4)c[nH]c12]

3-(7-chloro-1H-indol-3-yl)quinuclidine (ZD160-133). ZD160-133 was synthesized following Method P from 7-chloro-1H-indole. ¹H NMR (400 MHz, Methanol-d₄) δ 7.40 (dd, J=7.9, 2.0 Hz, 1H), 7.32 (s, 1H), 7.07 (dd, J=7.6, 2.0 Hz, 1H), 6.93 (td, J=8.0, 1.8 Hz, 1H), 3.71 (m, J=23.6, 15.1, 10.3 Hz, 2H), 3.47-3.32 (m, 5H), 2.23 (s, 1H), 2.20-2.13 (m, 1H), 2.04 (dt, J=13.2, 9.5 Hz, 1H), 1.97-1.86 (m, 1H), 1.69 (t, J=12.4 Hz, 1H). MS(ESI) m/z: [M+H]⁺ 261.4.

### Example 252

### Synthesis of ZD160-130

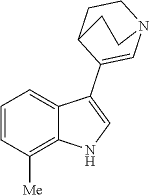 [SMILES: Cc1cccc2c(C3=CN4CCC3CC4)c[nH]c12]

3-(7-methyl-1H-indol-3-yl)-1-azabicyclo[2.2.2]oct-2-ene (ZD160-130). ZD160-130 was synthesized following Method P from 7-methyl-1H-indole. ¹H NMR (400 MHz, Methanol-d₄) δ 7.45 (d, J=7.8 Hz, 1H), 7.28 (d, J=2.0 Hz, 1H), 6.89 (td, J=7.4, 2.0 Hz, 1H), 6.84 (d, J=7.2 Hz, 1H), 6.67 (s, 1H), 3.06 (s, 1H), 3.01-2.93 (m, 2H), 2.67-2.56 (m, 2H), 1.81-1.71 (m, 2H), 1.63-1.53 (m, 2H). MS(ESI) m/z: [M+H]⁺ 239.4.

### Example 253

### Synthesis of ZD160-131

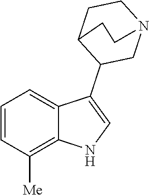 [SMILES: Cc1cccc2c(C3CN4CCC3CC4)c[nH]c12]

3-(7-methyl-1H-indol-3-yl)-1-azabicyclo[2.2.2]oct-2-ene (ZD160-131). ZD160-131 was synthesized following Method P from 7-methyl-1H-indole. ¹H NMR (400 MHz, Methanol-d₄) δ 7.39 (dd, J=6.7, 2.5 Hz, 1H), 7.34 (d, J=2.0 Hz, 1H), 7.01-6.94 (m, 2H), 3.90-3.75 (m, 2H), 3.60-3.44 (m, 4H), 3.29 (s, 1H), 2.38 (d, J=3.7 Hz, 1H), 2.29 (d, J=8.1 Hz, 1H), 2.21-2.13 (m, 1H), 2.07 (m, J=13.2, 10.3, 7.4, 2.2 Hz, 1H), 1.80 (t, J=12.4 Hz, 1H). MS(ESI) m/z: [M+H]⁺ 241.5.

### Example 254

### Synthesis of QC166-005

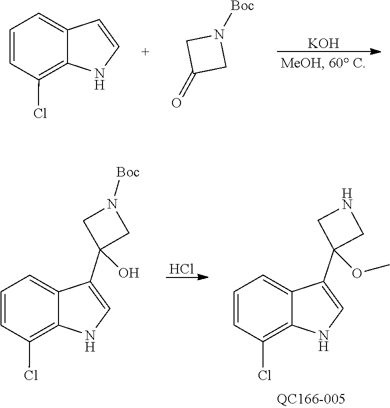 [SMILES: Clc1cccc2cc[nH]c12 ; CC(C)(C)OC(=O)N1CC(=O)C1 ; "KOH" ; CC(C)(C)OC(=O)N1CC(O)(c2c[nH]c3c(Cl)cccc23)C1 ; Cl ; COC1(c2c[nH]c3c(Cl)cccc23)CNC1]

### 7-chloro-3-(3-methoxyazetidin-3-yl)-1H-indole (QC166-005)

Step 1:7-chloro-1H-indole (300 mg, 2.0 mmol, 1.0 eq) and tert-butyl 3-oxoazetidine-1-carboxylate (380 mg, 2.2 mmol, 1.1 eq) were dissolved in MeOH (6 mL). Then KOH (123 mg, 2.2 mmol, 1.1 eq) was added. The mixture was stirred at 60° C. for 12 h. After removal of the solvents, the residue was purified by silica gel (Hexane:Ethyl Acetate=1:1) and obtained tert-butyl 3-(7-chloro-1H-indol-3-yl)-3-hydroxyazetidine-1-carboxylate as a white solid (112 mg, 16% yield). ¹H NMR (400 MHz, MeOD) δ 7.44 (d, J=7.8 Hz, 1H), 7.26 (s, 1H), 7.06 (d, J=7.8 Hz, 1H), 6.91 (t, J=7.8 Hz, 1H), 4.26 (d, J=9.0 Hz, 2H), 4.08 (d, J=9.0 Hz, 2H), 1.37 (s, 9H).

Step 2:tert-butyl 3-(7-chloro-1H-indol-3-yl)-3-hydroxyazetidine-1-carboxylate (15 mg) was dissolved in MeOH (0.5 mL), then HCl in dixoane (4M, 1.0 mL, 4 mmol) was added. The mixture was stirred at room temperature for 2 h. After removal of the solvents, the residue was purified by prep-HPLC to yield title compound (white solid, 6 mg, 58% yield). ¹H NMR (400 MHz, MeOD) δ 7.60 (s, 1H), 7.50 (d, J=8.0, 1H), 7.24 (d, J=8.0 Hz, 1H), 7.07 (t, J=8.0 Hz, 1H), 4.50-4.36 (m, 4H), 3.06 (s, 3H). MS (ESI) m/z: [M+H]⁺ 237.3.

### Examples 255 and 256

### Synthesis of QC166-008 and QC166-032

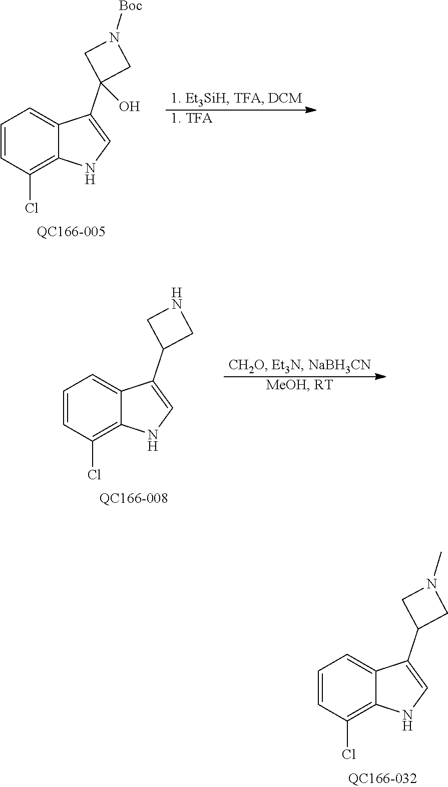

Step 1:3-(azetidin-3-yl)-7-chloro-1H-indole (QC166-008). tert-butyl 3-(7-chloro-1H-indol-3-yl)-3-hydroxyazetidine-1-carboxylate (25 mg, 0.08 mmol, 1.0 eq) and Et₃SiH (90 mg, 0.78 mmol, 10 eq) were dissolved in DCM (2 mL). After stirred at 0° C. for 10 min, TFA (45 mg, 0.4 mmol, 5.0 eq) was added dropwise. The mixture was stirred at 0° C. for 20 min. Then TFA (1 mL) was added. The mixture was stirred at room temperature for another 1 h and removed all the solvents. The residue was purified by prepared HPLC to yield light yellow oil (3.5 mg, 22% yield). ¹H NMR (400 MHz, MeOD) δ 7.52 (d, J=8.0 Hz, 1H), 7.42 (s, 1H), 7.16 (d, J=8.0 Hz, 1H), 7.03 (t, J=8.0 Hz, 1H), 4.48-4.40 (m, 3H), 4.36-4.27 (m, 2H). MS (ESI) m/z: [M+H]⁺ 207.3.

Step 2:7-chloro-3-(1-methylazetidin-3-yl)-1H-indole (QC166-032). 3-(azetidin-3-yl)-7-chloro-1H-indole (20 mg, 0.1 mmol, 1.0 eq), Et₃N (25 mg, 0.3 mmol, 2.5 eq), CH₂O (37% wt in H₂O, 0.1 ml), NaBH₃CN (10 mg, 0.15 mmol, 1.5 eq), were dissolved in 2 mL MeOH. The mixture was stirred at room temperature overnight. After removal all the volatiles, the residue was purified with prepared HPLC to yield colorless oil (4 mg, 17%). ¹H NMR (400 MHz, MeOD) δ 7.41-7.35 (m, 2H), 7.12-7.05 (m, 1H), 6.95 (t, J=7.8 Hz, 1H), 4.63-4.55 (m, 1H), 4.46-4.22 (m, 3H), 4.13 (t, J=9.6 Hz, 1H), 2.94 (d, J=29.4, 3H). MS (ESI) m/z: [M+H]⁺ 221.0.

### Example 257

### Synthesis of XQ148-86

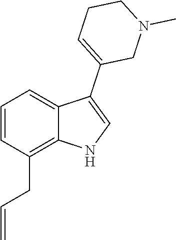 [SMILES: C=CCc1cccc2c(C3=CCCN(C)C3)c[nH]c12]

7-allyl-3-(1-methyl-1,2,5,6-tetrahydropyridin-3-yl)-1H-indole (XQ148-86). XQ148-086 was synthesized following the standard procedure for preparing XQ148-093, light yellow solid (23 mg, 30%). ¹H NMR (400 MHz, Methanol-d₄) δ 7.59 (d, J=8.0 Hz, 1H), 7.18 (s, 1H), 6.95 (t, J=7.6 Hz, 1H), 6.88 (d, J=7.1 Hz, 1H), 6.23-6.14 (m, 1H), 6.06-5.92 (m, 1H), 5.06-4.94 (m, 2H), 3.57-3.47 (m, 4H), 2.84 (t, J=6.0 Hz, 2H), 2.56 (s, 3H), 2.50-2.42 (m, 2H). MS (ESI) m/z: calcd for C₁₇H₂₁N₂⁺ [M+H]⁺, 253.4 found, 253.2.

### Example 258

### Synthesis of Compound QC166-096

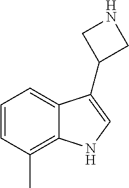

3-(azetidin-3-yl)-7-methyl-1H-indole (QC166-096), was synthesized following the standard procedure for preparing QC166-008. ¹H NMR (400 MHz, Methanol-d₄) δ 7.41 (d, J=6.6 Hz, 1H), 7.32 (s, 1H), 7.02-6.91 (m, 2H), 4.49-4.38 (m, 3H), 4.37-4.27 (m, 2H), 2.48 (s, 3H). MS (ESI) m/z: [M+H]⁺ 187.0.

### Example 259

### Synthesis of Compound QC166-097

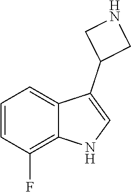 [SMILES: Fc1cccc2c(C3CNC3)c[nH]c12]

3-(azetidin-3-yl)-7-fluoro-1H-indole (QC166-097), was synthesized following the standard procedure for preparing QC166-008. ¹H NMR (400 MHz, Methanol-d₄) δ 7.42-7.37 (m, 1.6 Hz, 2H), 7.06-6.98 (m, 1H), 6.93-6.86 (m, 1H), 4.50-4.42 (m, 3H), 4.39-4.28 (m, 2H). MS (ESI) m/z: [M+H]⁺ 191.2.

### Example 260

### Synthesis of Compound QC179-001

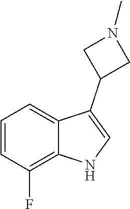

7-fluoro-3-(1-methylazetidin-3-yl)-1H-indole (QC179-001), was synthesized following the standard procedure for preparing QC166-032. ¹H NMR (400 MHz, Methanol-d₄) δ 7.46 (s, 1H), 7.39-7.31 (m, 1H), 7.08-7.00 (m, 1H), 6.96-6.88 (m, 1H), 4.71 (t, J=8.8 Hz, 1H), 4.59-4.34 (m, 3H), 4.24 (d, J=9.4 Hz, 1H), 3.07 (d, J=28.8 Hz, 3H). MS (ESI) m/z: [M+H]⁺ 205.3.

### Example 261

### Synthesis of Compound QC179-002

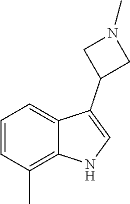

7-methyl-3-(1-methylazetidin-3-yl)-1H-indole (QC179-002), was synthesized following the standard procedure for preparing QC166-032. ¹H NMR (400 MHz, Methanol-d₄) δ 7.43-7.32 (m, 1H), 7.07-6.90 (m, 1H), 4.70 (t, J=8.7 Hz, 1H), 4.59-4.33 (m, 1H), 4.24 (t, J=9.4 Hz, 1H), 3.06 (d, J=28.5 Hz, 1H), 2.51 (s, 1H). MS (ESI) m/z: [M+H]⁺ 201.0.

### Example 262

### Synthesis of Compound QC179-025

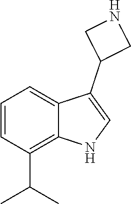 [SMILES: CC(C)c1cccc2c(C3CNC3)c[nH]c12]

3-(azetidin-3-yl)-7-isopropyl-1H-indole (QC179-025), was synthesized following the standard procedure for preparing QC166-008. ¹H NMR (400 MHz, MeOD) δ 7.45-7.41 (m, 1H), 7.34 (s, 1H), 7.09-7.03 (m, 2H), 4.52-4.41 (m, 3H), 4.40-4.32 (m, 2H), 3.42-3.34 (m, 1H), 1.37 (d, J=6.9 Hz, 6H). MS (ESI) m/z: [M+H]⁺ 215.4.

### Example 263

### Synthesis of Compound QC179-032

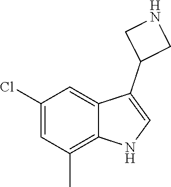

3-(azetidin-3-yl)-5-chloro-7-methyl-1H-indole (QC179-032), was synthesized following the standard procedure for preparing QC166-008. ¹H NMR (400 MHz, Methanol-d₄) δ 7.46 (s, 1H), 7.42 (s, 1H), 6.97 (s, 1H), 4.39-4.50 (m, 3H), 4.38-4.28 (m, 2H), 2.50 (s, 3H). MS (ESI) m/z: [M+H]⁺ 221.3.

### Example 264

### Synthesis of Compound QC179-033

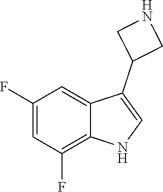 [SMILES: Fc1cc(F)c2[nH]cc(C3CNC3)c2c1]

3-(azetidin-3-yl)-5,7-difluoro-1H-indole (QC179-033), was synthesized following the standard procedure for preparing QC166-008. ¹H NMR (400 MHz, Methanol-d₄) δ 7.50 (s, 1H), 7.18 (d, J=9.1 Hz, 1H), 6.82 (t, J=10.3 Hz, 1H), 4.51-4.39 (m, 3H), 4.38-4.27 (m, 2H). MS (ESI) m/z: [M+H]⁺ 209.3.

### Example 265

### Synthesis of Compound QC179-038

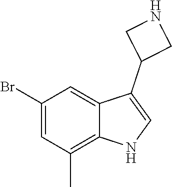

3-(azetidin-3-yl)-5-bromo-7-methyl-1H-indole (QC179-038), was synthesized following the standard procedure for preparing QC166-008. ¹H NMR (400 MHz, Methanol-d₄) δ 7.61 (s, 1H), 7.41 (s, 1H), 7.09 (s, 1H), 4.51-4.39 (m, 3H), 4.36-4.23 (m, 2H), 2.49 (s, 3H). MS (ESI) m/z: [M+H]⁺ 265.3.

### Example 266

### Synthesis of Compound QC179-039

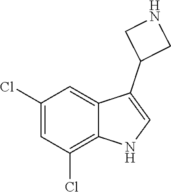 [SMILES: Clc1cc(Cl)c2[nH]cc(C3CNC3)c2c1]

3-(azetidin-3-yl)-5,7-dichloro-1H-indole (QC179-039), was synthesized following the standard procedure for preparing QC166-008. ¹H NMR (400 MHz, Methanol-d₄) δ 7.60 (s, 1H), 7.53 (s, 1H), 7.21 (s, 1H), 4.52-4.40 (m, 3H), 4.38-4.26 (m, 2H). MS (ESI) m/z: [M+H]⁺ 241.3.

### Example 267

### Synthesis of Compound QC179-040

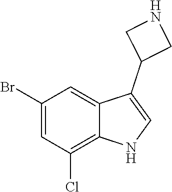 [SMILES: Clc1cc(Br)cc2c(C3CNC3)c[nH]c12]

3-(azetidin-3-yl)-5-bromo-7-chloro-1H-indole (QC179-040), was synthesized following the standard procedure for preparing QC166-008. ¹H NMR (400 MHz, Methanol-d₄) δ 7.74 (s, 1H), 7.52 (s, 1H), 7.32 (s, 1H), 4.51-4.39 (m, 3H), 4.38-4.24 (m, 2H). MS (ESI) m/z: [M+H]⁺ 284.7.

### Example 268

### Synthesis of ZX167-072

### Synthesis of Intermediate ZX167-064

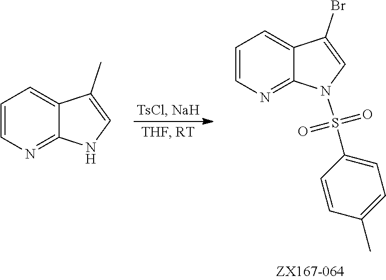

3-bromo-1-tosyl-1H-pyrrolo[2,3-b]pyridine (ZX167-064). 3-Bromo-1H-pyrrolo[2,3-b]pyridine (200 mg, 1.01 mmol, 1.0 equiv) was dissolved into 10 mL THF and cooled to 0° C., NaH (60% in mineral oil) (61 mg, 1.52 mmol, 1.5 equiv) was added to the solution, stirred for 15 min and 4-toluolsulfonyl chloride (212 mg, 1.11 mmol, 1.1 equiv) was added and the reaction mixture was warmed to room temperature and stirred for another 2 h. The reaction mixture was quenched with 20 mL NaHCO₃ aqueous solution and extracted with 20 mL EA twice. The organic phase was combined and washed with brine. Dried over anhydrous Na₂SO₄ and concentrated. The residue was purified by silica gel chromatography (PE-PE/EA=5/1) to yield ZX167-064 (white solid, 0.26 g, 73% yield). ¹H NMR (400 MHz, Chloroform-d) δ 8.47 (d, J=4.6 Hz, 1H), 8.08 (d, J=7.3 Hz, 2H), 7.81 (d, J=7.9 Hz, 1H), 7.79 (s, 1H), 7.29 (d, J=7.6 Hz, 2H), 7.25 (d, J=4.0 Hz, 1H), 2.38 (s, 3H). MS (ESI) m/z: [M+H]⁺ 351.1.

### Synthesis of Compound ZX167-072

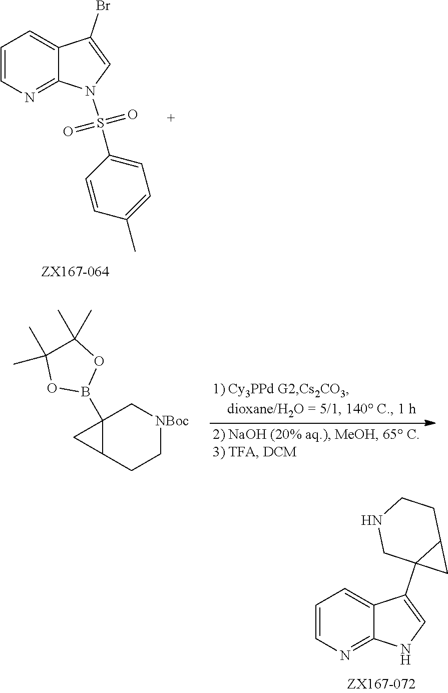 [SMILES: Cc1ccc(S(=O)(=O)n2cc(Br)c3cccnc32)cc1 ; CC(C)(C)OC(=O)N1CCC2CC2(B2OC(C)(C)C(C)(C)O2)C1 ; c1cnc2[nH]cc(C34CNCCC3C4)c2c1]

### 3-(3-azabicyclo[4.1.0]heptan-1-yl)-1H-pyrrolo[2,3-b]pyridine (ZX167-072)

Step 1: To a solution of ZX162-064 (35 mg, 0.1 mmol, 1.0 equiv.) in dioxane/water (2 mL, 5:1) was added Cy₃P Pd G2 (11.8 mg, 0.02 mmol, 0.2 equiv.), Cs₂CO₃ (65 mg, 0.2 mmol, 2.0 equiv.) and tert-butyl 1-(4,4,5,5-tetramethyl-1,3,2-dioxaborolan-2-yl)-3-azabicyclo[4.1.0]hexane-3-carboxylate (39 mg, 0.12 mmol, 1.2 equiv.). The mixture was heated at 140° C. under microwave irradiation condition at nitrogen atmosphere for 1 h, followed by diluted with EA (20 mL) and washed with brine (20 mL). After dried over Na₂SO₄ and concentrated, the residue was purified by pre-HPLC. MS (ESI) m/z: [M+H]468.4.

Step 2: The above product was dissolved into 1 mL MeOH and 0.2 mL 20% NaOH aqueous solution was added. The reaction mixture was stirred at 65° C. for 1 h and cooled to room temperature. The solution was extracted with 10 mL DCM twice, the organic phase was combined and washed with brine. Dried over anhydrous Na₂SO₄ and concentrated.

Step 3: The residue was dissolved into DCM/TFA (5 mL, 2:1) and stirred at room temperature for 1 h followed by purified by prep-HPLC to yield title compound. (17 mg, white solid, 38% yield). ¹H NMR (400 MHz, Methanol-d₄) δ 8.60 (d, J=7.9 Hz, 1H), 8.38 (s, 1H), 7.56 (s, 1H), 7.47-7.42 (m, 1H), 3.71-3.58 (m, 2H), 3.23 (dt, J=11.3, 5.5 Hz, 1H), 3.07-2.96 (m, 1H), 2.53 (dt, J=14.9, 7.5 Hz, 1H), 2.12 (dt, J=15.0, 6.3 Hz, 1H), 1.68 (q, J=6.8 Hz, 1H), 1.28 (t, J=6.6 Hz, 1H), 1.13 (t, J=5.1 Hz, 1H). MS (ESI) m/z: [M+H]⁺ 214.1.

### Example 269

### Synthesis of Compound ZX167-077

### Synthesis of Intermediate ZX167-067

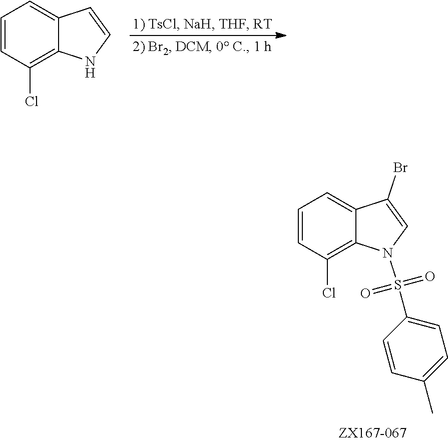 [SMILES: Clc1cccc2cc[nH]c12 ; Cc1ccc(S(=O)(=O)n2cc(Br)c3cccc(Cl)c32)cc1]

### 3-bromo-7-chloro-1-tosyl-1H-indole (ZX167-067)

Step 1: 7-chloro-1H-indole (0.5 g, 3.3 mmol, 1.0 equiv.) was dissolved into 20 mL THF and cooled to 0° C., NaH (60% in mineral oil) (198 mg, 4.9 mmol, 1.5 equiv.) was added to the solution, stirred for 15 min and 4-Toluolsulfonyl chloride (692 mg, 3.6 mmol, 1.1 equiv) was added and the reaction mixture was warmed to room temperature and stirred for another 2 h. The reaction mixture was quenched with 20 mL NaHCO₃ aqueous solution and extracted with 20 mL EA twice. The organic phase was combined and washed with brine. Dried over anhydrous Na₂SO₄ and concentrated. The residue was purified by silica gel chromatography (PE-PE/EA=5/1) (colorless oil, 0.76 g, 75% yield).

Step 2: The above product (250 mg, 0.82 mmol, 1.0 equiv.) was dissolved into 5 mL DCM and cooled to 0° C. To the reaction mixture was added Br₂ (50 μL, 0.98 mmol, 1.2 equiv.). The reaction mixture was stirred at 0° C. for 2 h and quenched with 2 mL Na₂SO₃ aqueous solution. 5 mL NaHCO₃ aqueous solution was added, and the solution was extracted with 5 mL DCM twice. The organic phase was combined and washed with brine. Dried over anhydrous Na₂SO₄ and concentrated. The residue was purified by silica gel chromatography (PE-PE/EA=5/1) to yield ZX167-067 (white solid, 0.30 g, 94% yield). ¹H NMR (400 MHz, Chloroform-d) δ 7.90 (s, 1H), 7.65 (d, J=6.2 Hz, 2H), 7.39 (d, J=7.8 Hz, 1H), 7.24-7.18 (m, 3H), 7.14 (td, J=7.8, 2.2 Hz, 1H), 2.34 (s, 3H).

### Synthesis of Compound ZX167-077

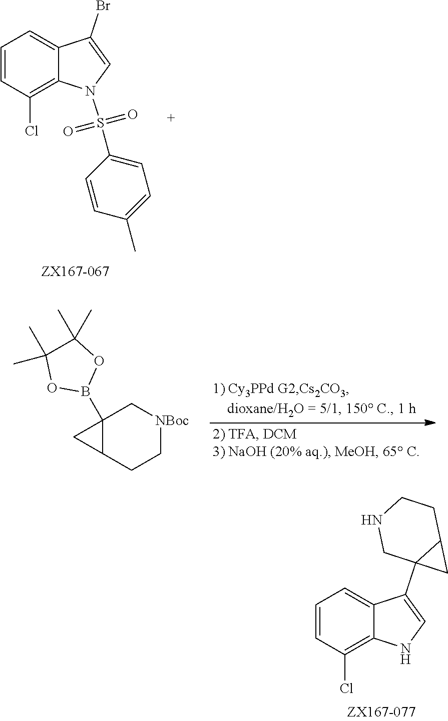 [SMILES: Cc1ccc(S(=O)(=O)n2cc(Br)c3cccc(Cl)c32)cc1 ; CC(C)(C)OC(=O)N1CCC2CC2(B2OC(C)(C)C(C)(C)O2)C1 ; Clc1cccc2c(C34CNCCC3C4)c[nH]c12]

3-(3-azabicyclo[4.1.0]heptan-1-yl)-7-chloro-1H-indole (ZX167-077). ZX167-077 was synthesized following the similar procedure for ZX167-072 except for the temperature was 150° C. in the step 1 and the sequence of step 2 and step 3 was reversed. ¹H NMR (400 MHz, Methanol-d₄) δ 7.62 (d, J=6.9 Hz, 1H), 7.25 (s, 1H), 7.16 (d, J=6.5 Hz, 1H), 7.05 (t, J=7.7 Hz, 1H), 3.63 (d, J=13.4 Hz, 1H), 3.57 (d, J=11.8 Hz, 1H), 3.19 (q, J=6.1, 5.4 Hz, 1H), 3.07-2.97 (m, 1H), 2.50 (h, J=7.9 Hz, 1H), 2.11 (dt, J=13.8, 5.5 Hz, 1H), 1.62 (q, J=7.4 Hz, 1H), 1.27-1.20 (m, 1H), 1.04 (t, J=6.7 Hz, 1H). MS (ESI) m/z: [M+H]⁺ 247.2.

### Example 270

### Synthesis of ZX167-074

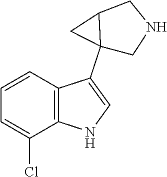 [SMILES: Clc1cccc2c(C34CNCC3C4)c[nH]c12]

3-(3-azabicyclo[3.1.0]hexan-1-yl)-7-chloro-1H-indole (ZX167-074). ZX167-074 was synthesized following the same procedure for ZX167-077 from ZX167-067 and tert-butyl 1-(4,4,5,5-tetramethyl-1,3,2-dioxaborolan-2-yl)-3-azabicyclo[3.1.0]hexane-3-carboxylate at 140° C. White solid (16 mg, 46% yield). ¹H NMR (400 MHz, Methanol-d₄) δ 7.56 (d, J=8.3 Hz, 1H), 7.31 (s, 1H), 7.16 (d, J=8.1 Hz, 1H), 7.04 (t, J=7.8 Hz, 1H), 3.80 (d, J=11.5 Hz, 1H), 3.71 (d, J=11.4 Hz, 1H), 3.59 (s, 1H), 3.56 (s, 1H), 2.07 (h, J=4.0 Hz, 1H), 1.30 (t, J=7.4 Hz, 1H), 1.10 (t, J=5.8 Hz, 1H). MS (ESI) m/z: [M+H]⁺ 233.3.

### Example 271

### Synthesis of ZX167-090

### Synthesis of Intermediate ZX167-087

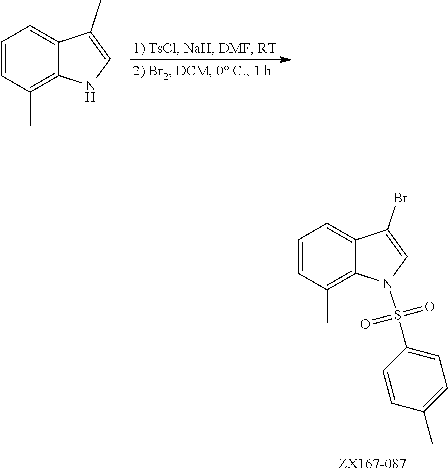 [SMILES: Cc1c[nH]c2c(C)cccc12 ; Cc1ccc(S(=O)(=O)n2cc(Br)c3cccc(C)c32)cc1]

3-bromo-7-methyl-1-tosyl-1H-indole (ZX167-087). ZX167-087 was prepared following the similar procedure for ZX167-067 using DMF instead of THE in the first step. White solid, yield 31% (2 steps). ¹H NMR (400 MHz, Chloroform-d) δ 7.88 (s, 1H), 7.60 (d, J=6.7 Hz, 2H), 7.41 (d, J=7.8 Hz, 1H), 7.29 (d, J=4.0 Hz, 2H), 7.22 (d, J=7.6 Hz, 1H), 7.12 (d, J=7.5 Hz, 1H), 2.58 (s, 3H), 2.41 (s, 3H).

### Synthesis of Compound ZX167-090

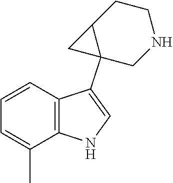 [SMILES: Cc1cccc2c(C34CNCCC3C4)c[nH]c12]

3-(3-azabicyclo[4.1.0]heptan-1-yl)-7-methyl-1H-indole (ZX167-090). ZX167-090 was prepared following the similar procedure for ZX167-077, except for the last step, the reaction mixture was stirred at 80° C. for 12 h. white solid, yield 24%. ¹H NMR (400 MHz, Methanol-d₄) δ 7.49 (d, J=7.8 Hz, 1H), 7.15 (s, 1H), 7.01-6.95 (m, 1H), 6.93 (d, J=7.2 Hz, 1H), 3.60 (s, 2H), 3.17 (q, J=7.4 Hz, 1H), 3.02 (dt, J=12.0, 5.5 Hz, 1H), 2.55-2.48 (m, 1H), 2.46 (s, 3H), 2.16-2.06 (m, 1H), 1.59 (q, J=7.0 Hz, 1H), 1.25-1.20 (m, 1H), 1.01 (t, J=6.1 Hz, 1H). MS (ESI) m/z. [M+H]⁺ 227.1.

### Example 272

### Synthesis of Compound ZX167-091

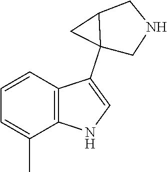 [SMILES: Cc1cccc2c(C34CNCC3C4)c[nH]c12]

3-(3-azabicyclo[3.1.0]hexan-1-yl)-7-methyl-1H-indole (ZX167-091). ZX167-091 was prepared following the similar procedure for ZX167-077, except for the last step, the reaction mixture was stirred at 80° C. for 12 h. white solid, yield 67%. ¹H NMR (400 MHz, Methanol-d₄) δ 7.42 (d, J=7.7 Hz, 1H), 7.21 (s, 1H), 6.97 (t, J=7.2 Hz, 1H), 6.92 (d, J=7.1 Hz, 1H), 3.78 (d, J=11.5 Hz, 1H), 3.68 (d, J=11.4 Hz, 1H), 3.57 (t, J=10.3 Hz, 2H), 2.47 (s, 3H), 2.02 (h, J=4.1 Hz, 1H), 1.28 (t, J=7.5 Hz, 1H), 1.09-1.04 (m, 1H). MS (ESI) m/z: [M+H]⁺ 213.1.

### Examples 273, 274, 275, 276, 277 & 278

General procedure for chiral separation of ZX162-031, ZX162-100 and ZX167-074 enantiomers to generate Example 274: ZX162-100-1 (former peak) and Example 275: ZX162-100-2 (latter peak), Example 276: ZX162-031-1 (former peak) and Example 277: ZX162-031-2 (latter peak), Example 278: ZX167-074-1 (former peak) and Example 279: ZX167-074-2 (latter peak).

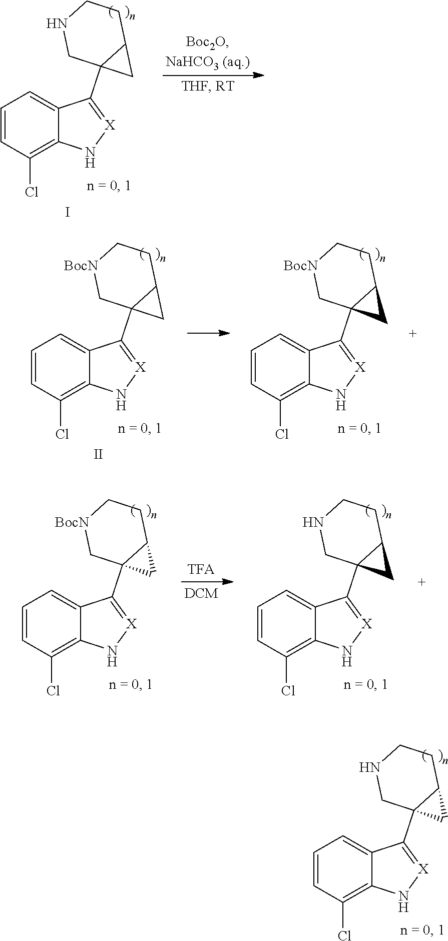

Step 1: compound I (0.13 mmol, 1.0 equiv) was dissolved into 2 mL THF, and Boc₂O (1.5 equiv) and 1 mL NaHCO₃ saturated aqueous solution was added to the solution. The reaction mixture was stirred at room temperature for 2 h until the compound I was consumed. Separated and the aqueous phase was extracted with 5 mL DCM twice. The organic phase was combined and washed with brine, dried over anhydrous Na₂SO₄, filtered and concentrated to yield compound II.

Step 2: Compound II was chiral separated by Lux^{R} 5 μM i-Amylose-3 column (solvent: CH₃CN/H₂O (0.1% TFA): 10%-100%).

Step 3: Chiral Compound II was dissolved into DCM/TFA (2 mL, 2:1), and stirred at room temperature for 1 h, concentrated and purified by pre-HPLC.

### Example 279

### Synthesis of ZX177-057

### Synthesis of Compound ZX177-039

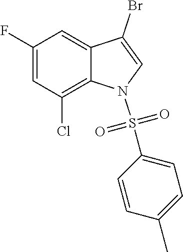 [SMILES: Cc1ccc(S(=O)(=O)n2cc(Br)c3cc(F)cc(Cl)c32)cc1]

3-bromo-7-chloro-5-fluoro-1-tosyl-1H-indole (ZX177-039). ZX177-039 was prepared following the similar procedure for ZX167-067 using DMF instead of THF in the first step. White solid, yield 80% (2 steps). ¹H NMR (400 MHz, Chloroform-d) δ 7.93 (s, 1H), 7.63 (d, J=7.4 Hz, 2H), 7.22 (d, J=7.7 Hz, 2H), 7.07 (d, J=7.8 Hz, 1H), 6.99 (d, J=9.4 Hz, 1H), 2.33 (s, 3H).

### Synthesis of Compound ZX177-057

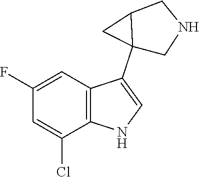 [SMILES: Fc1cc(Cl)c2[nH]cc(C34CNCC3C4)c2c1]

3-(3-azabicyclo[3.1.0]hexan-1-yl)-7-chloro-5-fluoro-1H-indole (ZX177-057) was synthesized following the same procedure for ZX167-077 from ZX177-039 and tert-butyl 1-(4,4,5,5-tetramethyl-1,3,2-dioxaborolan-2-yl)-3-azabicyclo[3.1.0]hexane-3-carboxylate at 125° C. White solid, yield 73%. ¹H NMR (400 MHz, Methanol-d₄) δ 7.39 (s, 1H), 7.29 (d, J=9.2 Hz, 1H), 7.01 (d, J=9.2 Hz, 1H), 3.80 (d, J=11.4 Hz, 1H), 3.70 (d, J=11.6 Hz, 1H), 3.54 (t, J=11.8 Hz, 2H), 2.10-1.99 (m, 1H), 1.26 (t, J=8.9 Hz, 1H), 1.13-1.06 (m, 1H). MS (ESI) m/z: [M+H]⁺ 251.2.

### Examples 280 and 281

### Synthesis of ZX177-058 and ZX177-058BY

### Synthesis of Compound ZX177-040

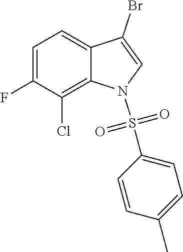 [SMILES: Cc1ccc(S(=O)(=O)n2cc(Br)c3ccc(F)c(Cl)c32)cc1]

3-bromo-7-chloro-6-fluoro-1-tosyl-1H-indole (ZX177-040). ZX177-040 was prepared following the similar procedure for ZX167-067 using DMF instead of THF in the first step. White solid, yield 68% (2 steps). ¹H NMR (400 MHz, Chloroform-d) δ 7.88 (s, 1H), 7.66 (d, J=7.1 Hz, 2H), 7.38-7.30 (m, 1H), 7.23 (d, J=5.7 Hz, 2H), 7.08 (t, J=9.4 Hz, 1H), 2.35 (s, 3H).

### Synthesis of Compound ZX177-058 and ZX177-058BY

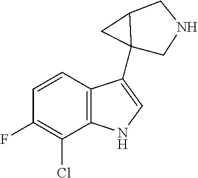 [SMILES: Fc1ccc2c(C34CNCC3C4)c[nH]c2c1Cl]

3-(3-azabicyclo[3.1.0]hexan-1-yl)-7-chloro-6-fluoro-1H-indole (ZX177-058) was synthesized following the same procedure for ZX167-077 from ZX177-040 and tert-butyl 1-(4,4,5,5-tetramethyl-1,3,2-dioxaborolan-2-yl)-3-azabicyclo[3.1.0]hexane-3-carboxylate at 125° C. White solid, yield 53%. ¹H NMR (400 MHz, Methanol-d₄) δ 7.52 (dt, J=8.8, 4.6 Hz, 1H), 7.32 (s, 1H), 7.01-6.93 (m, 1H), 3.79 (d, J=11.7 Hz, 1H), 3.71 (d, J=11.3 Hz, 1H), 3.61-3.52 (m, 2H), 2.07 (dd, J=8.5, 4.3 Hz, 1H), 1.28 (t, J=7.8 Hz, 1H), 1.14-1.07 (m, 1H). MS (ESI) m/z: [M+H]⁺ 251.5.

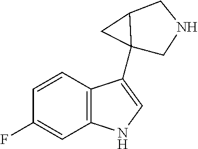 [SMILES: Fc1ccc2c(C34CNCC3C4)c[nH]c2c1]

3-(3-azabicyclo[3.1.0]hexan-1-yl)-6-fluoro-1H-indole (ZX177-058BY) was separated as a byproduct. White solid, yield 16%. ¹H NMR (400 MHz, Methanol-d₄) δ 7.60-7.53 (m, 1H), 7.22 (s, 1H), 7.08 (d, J=9.7 Hz, 1H), 6.89-6.80 (m, 1H), 3.79 (d, J=11.3 Hz, 1H), 3.70 (d, J=11.4 Hz, 1H), 3.56 (d, J=11.5 Hz, 2H), 2.06 (h, J=4.4 Hz, 1H), 1.29 (t, J=8.8 Hz, 1H), 1.09-1.04 (m, 1H). MS (ESI) m/z: [M+H]⁺ 217.1.

### Example 282

### Synthesis of ZX177-059

### Synthesis of Intermediate ZX177-041

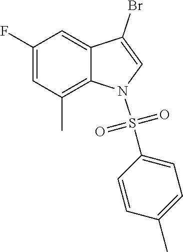 [SMILES: Cc1ccc(S(=O)(=O)n2cc(Br)c3cc(F)cc(C)c32)cc1]

3-bromo-7-chloro-6-fluoro-1-tosyl-1H-indole (ZX177-041). ZX177-041 was prepared following the similar procedure for ZX167-067 using DMF instead of THF in the first step. White solid, yield 48% (2 steps). ¹H NMR (400 MHz, Chloroform-d) δ 7.79 (s, 1H), 7.47 (d, J=7.8 Hz, 2H), 7.18 (d, J=7.9 Hz, 2H), 6.95 (d, J=9.6 Hz, 1H), 6.75 (d, J=9.5 Hz, 1H), 2.47 (s, 3H), 2.31 (s, 3H).

### Synthesis of Compound ZX177-059

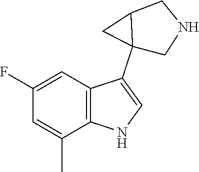 [SMILES: Cc1cc(F)cc2c(C34CNCC3C4)c[nH]c12]

3-(3-azabicyclo[3.1.0]hexan-1-yl)-7-chloro-6-fluoro-1H-indole (ZX177-059) was synthesized following the same procedure for ZX167-077 from ZX177-040 and tert-butyl 1-(4,4,5,5-tetramethyl-1,3,2-dioxaborolan-2-yl)-3-azabicyclo[3.1.0]hexane-3-carboxylate at 125° C. White solid, yield 43%. ¹H NMR (400 MHz, Methanol-d₄) δ 7.28 (s, 1H), 7.10 (d, J=9.4 Hz, 1H), 6.73 (d, J=10.0 Hz, 1H), 3.79 (d, J=11.3 Hz, 1H), 3.67 (d, J=11.4 Hz, 1H), 3.58-3.51 (m, 2H), 2.46 (s, 3H), 2.02 (h, J=4.4 Hz, 1H), 1.26 (t, J=8.8 Hz, 1H), 1.09-1.03 (m, 1H). MS (ESI) m/z: [M+H]⁺ 231.2.

### Example 283

### Synthesis of ZX177-060

### Synthesis of Intermediate ZX177-042

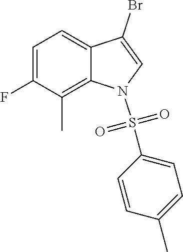 [SMILES: Cc1ccc(S(=O)(=O)n2cc(Br)c3ccc(F)c(C)c32)cc1]

3-bromo-7-chloro-6-fluoro-1-tosyl-1H-indole (ZX177-042). ZX177-042 was prepared following the similar procedure for ZX167-067 using DMF instead of THF in the first step. White solid, yield 47% (2 steps). ¹H NMR (400 MHz, Chloroform-d) δ 7.74 (s, 1H), 7.49 (d, J=8.8 Hz, 2H), 7.26-7.13 (m, 3H), 7.04-6.93 (m, 1H), 2.38 (s, 3H), 2.32 (s, 3H).

### Synthesis of Compound ZX177-060

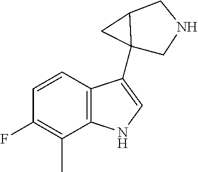 [SMILES: Cc1c(F)ccc2c(C34CNCC3C4)c[nH]c12]

3-(3-azabicyclo[3.1.0]hexan-1-yl)-7-chloro-6-fluoro-1H-indole (ZX177-060) was synthesized following the same procedure for ZX167-077 from ZX177-040 and tert-butyl 1-(4,4,5,5-tetramethyl-1,3,2-dioxaborolan-2-yl)-3-azabicyclo[3.1.0]hexane-3-carboxylate at 125° C. White solid, yield 45%. ¹H NMR (400 MHz, Methanol-d₄) δ 7.38 (p, J=4.8 Hz, 1H), 7.22 (s, 1H), 6.82 (t, J=9.1 Hz, 1H), 3.78 (d, J=115 Hz, 1H), 3.69 (d, J=11.3 Hz, 1H), 3.56 (d, J=11.4 Hz, 2H), 2.38 (s, 3H), 2.03 (h, J=4.4 Hz, 1H), 1.27 (t, J=8.9 Hz, 1H), 1.10-1.04 (m, 1H). MS (ESI) m/z: [M+H]⁺ 231.2.

Example compounds are set forth in Table 1 & 2 below

TABLE 1

| | Compound | | |
|---|---|---|---|
| Examples | code | Structure | Chemical Name |
| 1 | NS131-179 | 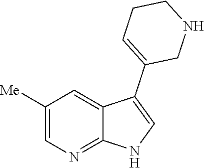 [SMILES: Cc1cnc2[nH]cc(C3=CCCNC3)c2c1] | 5-methyl-3-(1,2,5,6-tetrahydropyridin- 3-yl)-1H-pyrrolo[2,3-b]pyridine |
| 2 | NS131-178 | 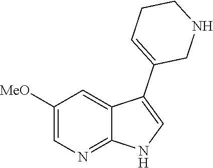 [SMILES: COc1cnc2[nH]cc(C3=CCCNC3)c2c1] | 5-methoxy-3-(1,2,5,6- tetrahydropyridin-3-yl)-1H- pyrrolo[2,3-b]pyridine |
| 3 | NS131-177 | 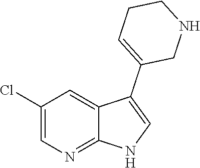 [SMILES: Clc1cnc2[nH]cc(C3=CCCNC3)c2c1] | 5-chloro-3-(1,2,5,6-tetrahydropyridin- 3-yl)-1H-pyrrolo[2,3-b]pyridine |
| 4 | NS136-006 | 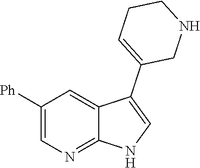 [SMILES: C1=C(c2c[nH]c3ncc(-c4ccccc4)cc23)CNCC1] | 5-phenyl-3-(1,2,5,6-tetrahydropyridin- 3-yl)-1H-pyrrolo[2,3-b]pyridine |
| 5 | NS131-169 | 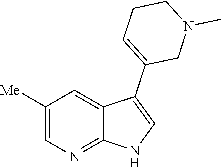 [SMILES: Cc1cnc2[nH]cc(C3=CCCN(C)C3)c2c1] | 5-methyl-3-(1-methyl-1,2,5,6- tetrahydropyridin-3-yl)-1H- pyrrolo[2,3-b]pyridine |
| 6 | NS131-168 | 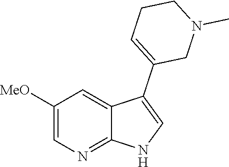 [SMILES: COc1cnc2[nH]cc(C3=CCCN(C)C3)c2c1] | 5-methoxy-3-(1-methyl-1,2,5,6- tetrahydropyridin-3-yl)-1H- pyrrolo[2,3-b]pyridine |
| 7 | NS131-167 | 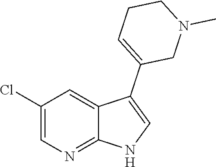 [SMILES: CN1CCC=C(c2c[nH]c3ncc(Cl)cc23)C1] | 5-chloro-3-(1-methyl-1,2,5,6- tetrahydropyridin-3-yl)-1H- pyrrolo[2,3-b]pyridine |
| 8 | NS131-173 | 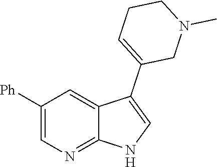 [SMILES: CN1CCC=C(c2c[nH]c3ncc(-c4ccccc4)cc23)C1] | 3-(1-methyl-1,2,5,6-tetrahydropyridin- 3-yl)-5-phenyl-1H-pyrrolo[2,3- b]pyridine |
| 9 | NS131-180 | 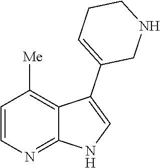 [SMILES: Cc1ccnc2[nH]cc(C3=CCCNC3)c12] | 4-methyl-3-(1,2,5,6-tetrahydropyridin- 3-yl)-1H-pyrrolo[2,3-b]pyridine |
| 10 | RS134-52 | 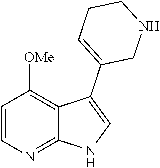 [SMILES: COc1ccnc2[nH]cc(C3=CCCNC3)c12] | 4-methoxy-3-(1,2,5,6- tetrahydropyridin-3-yl)-1H- pyrrolo[2,3-b]pyridine |
| 11 | RS134-48 | 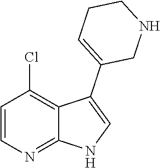 [SMILES: Clc1ccnc2[nH]cc(C3=CCCNC3)c12] | 4-chloro-3-(1,2,5,6-tetrahydropyridin- 3-yl)-1H-pyrrolo[2,3-b]pyridine |
| 12 | NS131-185 | 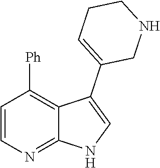 [SMILES: C1=C(c2c[nH]c3nccc(-c4ccccc4)c23)CNCC1] | 4-phenyl-3-(1,2,5,6-tetrahydropyridin- 3-yl)-1H-pyrrolo[2,3-b]pyridine |
| 13 | NS131-170 | 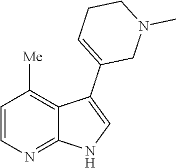 [SMILES: Cc1ccnc2[nH]cc(C3=CCCN(C)C3)c12] | 4-methyl-3-(1-methyl-1,2,5,6- tetrahydropyridin-3-yl)-1H- pyrrolo[2,3-b]pyridine |
| 14 | RS134-45 | 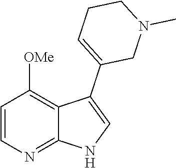 [SMILES: COc1ccnc2[nH]cc(C3=CCCN(C)C3)c12] | 4-methoxy-3-(1-methyl-1,2,5,6- tetrahydropyridin-3-yl)-1H- pyrrolo[2,3-b]pyridine |
| 15 | RS134-40 | 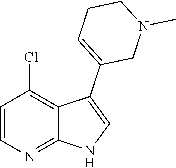 [SMILES: CN1CCC=C(c2c[nH]c3nccc(Cl)c23)C1] | 4-chloro-3-(1-methyl-1,2,5,6- tetrahydropyridin-3-yl)-1H- pyrrolo[2,3-b]pyridine |
| 16 | NS131-184 | 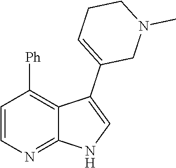 [SMILES: CN1CCC=C(c2c[nH]c3nccc(-c4ccccc4)c23)C1] | 3-(1-methyl-1,2,5,6-tetrahydropyridin- 3-yl)-4-phenyl-1H-pyrrolo[2,3- b]pyridine |
| 17 | RS134-49 | 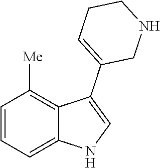 [SMILES: Cc1cccc2[nH]cc(C3=CCCNC3)c12] | 4-methyl-3-(1,2,5,6-tetrahydropyridin- 3-yl)-1H-indole |
| 18 | RS134-53 | 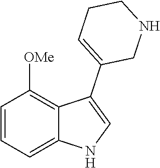 [SMILES: COc1cccc2[nH]cc(C3=CCCNC3)c12] | 4-methoxy-3-(1,2,5,6- tetrahydropyridin-3-yl)-1H-indole |
| 19 | RS134-41 | 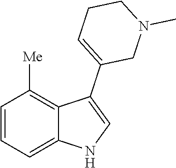 [SMILES: Cc1cccc2[nH]cc(C3=CCCN(C)C3)c12] | 4-methyl-3-(1-methyl-1,2,5,6- tetrahydropyridin-3-yl)-1H-indole |
| 20 | RS134-46 | 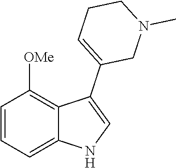 [SMILES: COc1cccc2[nH]cc(C3=CCCN(C)C3)c12] | 4-methoxy-3-(1-methyl-1,2,5,6- tetrahydropyridin-3-yl)-1H-indole |
| 21 | NS131-172 | 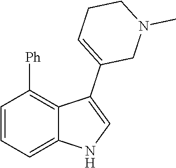 [SMILES: CN1CCC=C(c2c[nH]c3cccc(-c4ccccc4)c23)C1] | 3-(1-methyl-1,2,5,6-tetrahydropyridin- 3-yl)-4-phenyl-1H-indole |
| 22 | RS134-38 | 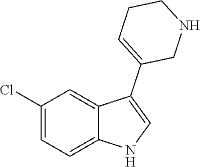 [SMILES: Clc1ccc2[nH]cc(C3=CCCNC3)c2c1] | 5-chloro-3-(1,2,5,6-tetrahydropyridin- 3-yl)-1H-indole |
| 23 | RS134-65 | 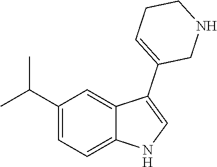 [SMILES: CC(C)c1ccc2[nH]cc(C3=CCCNC3)c2c1] | 5-isopropyl-3-(1,2,5,6- tetrahydropyridin-3-yl)-1H-indole |
| 24 | RS134-62 | 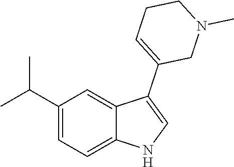 [SMILES: CC(C)c1ccc2[nH]cc(C3=CCCN(C)C3)c2c1] | 5-isopropyl-3-(1-methyl-1,2,5,6- tetrahydropyridin-3-yl)-1H-indole |
| 25 | RS134-70 | 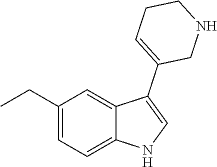 [SMILES: CCc1ccc2[nH]cc(C3=CCCNC3)c2c1] | 5-ethyl-3-(1,2,5,6-tetrahydropyridin-3- yl)-1H-indole |
| 26 | NS136-081 | 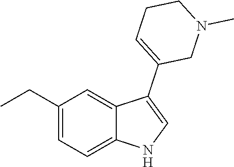 [SMILES: CCc1ccc2[nH]cc(C3=CCCN(C)C3)c2c1] | 5-ethyl-3-(1-methyl-1,2,5,6- tetrahydropyridin-3-yl)-1H-indole |
| 27 | RS134-73 | 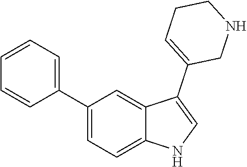 [SMILES: C1=C(c2c[nH]c3ccc(-c4ccccc4)cc23)CNCC1] | 5-phenyl-3-(1,2,5,6-tetrahydropyridin- 3-yl)-1H-indole |
| 28 | RS134-72 | 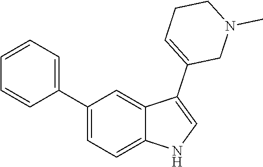 [SMILES: CN1CCC=C(c2c[nH]c3ccc(-c4ccccc4)cc23)C1] | 3-(1-methyl-1,2,5,6-tetrahydropyridin- 3-yl)-5-phenyl-1H-indole |
| 29 | NS136-092 | 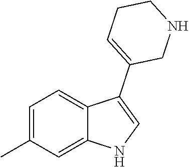 [SMILES: Cc1ccc2c(C3=CCCNC3)c[nH]c2c1] | 6-methyl-3-(1,2,5,6-tetrahydropyridin- 3-yl)-1H-indole |
| 30 | NS136-091 | 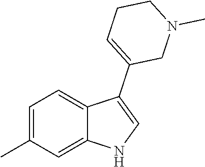 | 6-methyl-3-(1-methyl-1,2,5,6- tetrahydropyridin-3-yl)-1H-indole |
| 31 | NS136-096 | 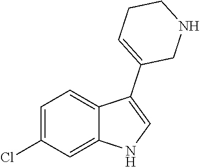 [SMILES: Clc1ccc2c(C3=CCCNC3)c[nH]c2c1] | 6-chloro-3-(1,2,5,6-tetrahydropyridin- 3-yl)-1H-indole |
| 32 | NS136-095 | 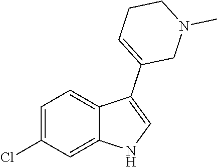 [SMILES: CN1CCC=C(c2c[nH]c3cc(Cl)ccc23)C1] | 6-chloro-3-(1-methyl-1,2,5,6- tetrahydropyridin-3-yl)-1H-indole |
| 33 | NS136-102 | 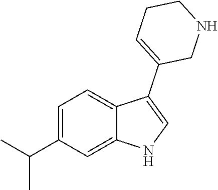 [SMILES: CC(C)c1ccc2c(C3=CCCNC3)c[nH]c2c1] | 6-isopropyl-3-(1,2,5,6- tetrahydropyridin-3-yl)-1H-indole |
| 34 | NS136-101 | 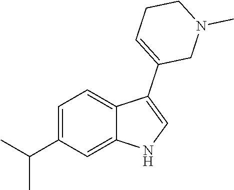 [SMILES: CC(C)c1ccc2c(C3=CCCN(C)C3)c[nH]c2c1] | 6-isopropyl-3-(1-methyl-1,2,5,6- tetrahydropyridin-3-yl)-1H-indole |
| 35 | NS136-115 | 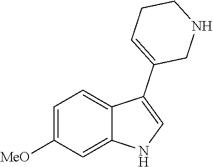 [SMILES: COc1ccc2c(C3=CCCNC3)c[nH]c2c1] | 6-methoxy-3-(1,2,5,6- tetrahydropyridin-3-yl)-1H-indole |
| 36 | NS136-116 | 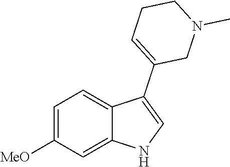 [SMILES: COc1ccc2c(C3=CCCN(C)C3)c[nH]c2c1] | 6-methoxy-3-(1-methyl-1,2,5,6- tetrahydropyridin-3-yl)-1H-indole |
| 37 | NS136-117 | 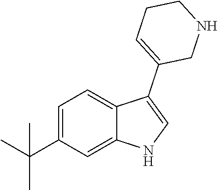 [SMILES: CC(C)(C)c1ccc2c(C3=CCCNC3)c[nH]c2c1] | 6-(tert-butyl)-3-(1,2,5,6- tetrahydropyridin-3-yl)-1H-indole |
| 38 | NS136-118 | 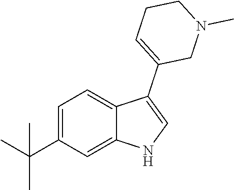 [SMILES: CN1CCC=C(c2c[nH]c3cc(C(C)(C)C)ccc23)C1] | 6-(tert-butyl)-3-(1-methyl-1,2,5,6- tetrahydropyridin-3-yl)-1H-indole |
| 39 | NS136-119 | 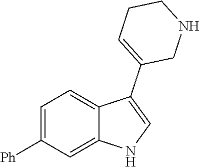 [SMILES: C1=C(c2c[nH]c3cc(-c4ccccc4)ccc23)CNCC1] | 6-phenyl-3-(1,2,5,6-tetrahydropyridin- 3-yl)-1H-indole |
| 40 | NS136-120 | 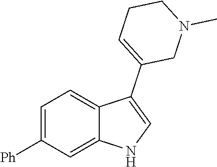 [SMILES: CN1CCC=C(c2c[nH]c3cc(-c4ccccc4)ccc23)C1] | 3-(1-methyl-1,2,5,6-tetrahydropyridin- 3-yl)-6-phenyl-1H-indole |
| 41 | NS136-109 | 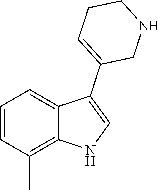 [SMILES: Cc1cccc2c(C3=CCCNC3)c[nH]c12] | 7-methyl-3-(1,2,5,6-tetrahydropyridin- 3-yl)-1H-indole |
| 42 | NS136-110 | 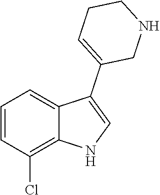 [SMILES: Clc1cccc2c(C3=CCCNC3)c[nH]c12] | 7-chloro-3-(1,2,5,6-tetrahydropyridin- 3-yl)-1H-indole |
| 43 | NS136-111 | 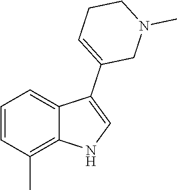 [SMILES: Cc1cccc2c(C3=CCCN(C)C3)c[nH]c12] | 7-methyl-3-(1-methyl-1,2,5,6- tetrahydropyridin-3-yl)-1H-indole |
| 44 | NS136-112 | 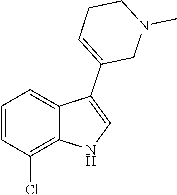 [SMILES: CN1CCC=C(c2c[nH]c3c(Cl)cccc23)C1] | 7-chloro-3-(1-methyl-1,2,5,6- tetrahydropyridin-3-yl)-1H-indole |
| 45 | RS134-37 | 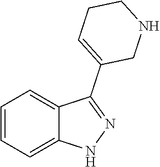 [SMILES: C1=C(c2n[nH]c3ccccc23)CNCC1] | 3-(1,2,5,6-tetrahydropyridin-3-yl)-1H- indazole |
| 46 | RS134-56 | 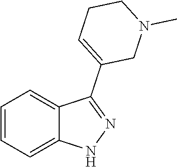 [SMILES: CN1CCC=C(c2n[nH]c3ccccc23)C1] | 3-(1-methyl-1,2,5,6-tetrahydropyridin- 3-yl)-1H-indazole |
| 47 | NS136-002 | 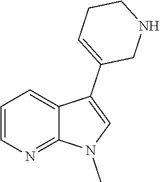 [SMILES: Cn1cc(C2=CCCNC2)c2cccnc21] | 1-methyl-3-(1,2,5,6-tetrahydropyridin- 3-yl)-1H-pyrrolo[2,3-b]pyridine |
| 48 | NS136-004 | 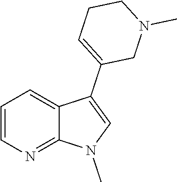 [SMILES: CN1CCC=C(c2cn(C)c3ncccc23)C1] | 1-methyl-3-(1-methyl-1,2,5,6- tetrahydropyridin-3-yl)-1H- pyrrolo[2,3-b]pyridine |
| 49 | RS130-132 | 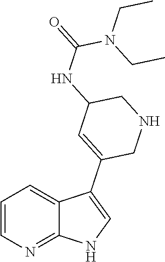 [SMILES: CCN(CC)C(=O)NC1C=C(c2c[nH]c3ncccc23)CNC1] | 3-(5-(1H-pyrrolo[2,3-b]pyridin-3-yl)- 1,2,3,6-tetrahydropyridin-3-yl)-1,1- diethylurea |
| 50 | YX129-177C | 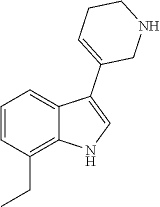 [SMILES: CCc1cccc2c(C3=CCCNC3)c[nH]c12] | 7-ethyl-3-(1,2,5,6-tetrahydropyridin-3- yl)-1H-indole |
| 51 | YX129-180C | 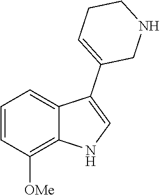 [SMILES: COc1cccc2c(C3=CCCNC3)c[nH]c12] | 7-methoxy-3-(1,2,5,6- tetrahydropyridin-3-yl)-1H-indole |
| 52 | YX143-19 | 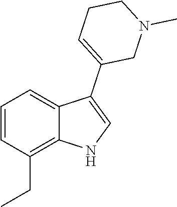 [SMILES: CCc1cccc2c(C3=CCCN(C)C3)c[nH]c12] | 7-ethyl-3-(1-methyl-1,2,5,6- tetrahydropyridin-3-yl)-1H-indole |
| 53 | YX143-20 | 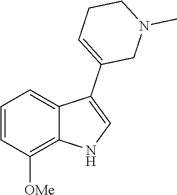 [SMILES: COc1cccc2c(C3=CCCN(C)C3)c[nH]c12] | 7-methoxy-3-(1-methyl-1,2,5,6- tetrahydropyridin-3-yl)-1H-indole |
| 54 | YX143-2 | 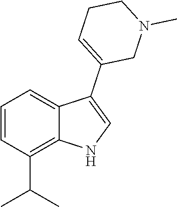 [SMILES: CC(C)c1cccc2c(C3=CCCN(C)C3)c[nH]c12] | 7-isopropyl-3-(1-methyl-1,2,5,6- tetrahydropyridin-3-yl)-1H-indole |
| 55 | YX143-21 | 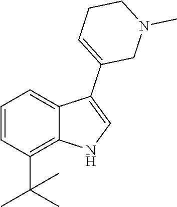 [SMILES: CN1CCC=C(c2c[nH]c3c(C(C)(C)C)cccc23)C1] | 7-(tert-butyl)-3-(1-methyl-1,2,5,6- tetrahydropyridin-3-yl)-1H-indole |
| 56 | NS144-042 | 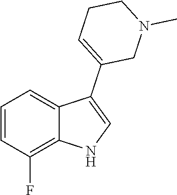 [SMILES: CN1CCC=C(c2c[nH]c3c(F)cccc23)C1] | 7-fluoro-3-(1-methyl-1,2,5,6- tetrahydropyridin-3-yl)-1H-indole |
| 57 | NS144-043 | 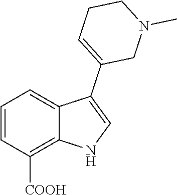 [SMILES: CN1CCC=C(c2c[nH]c3c(C(=O)O)cccc23)C1] | 3-(1-methyl-1,2,5,6-tetrahydropyridin- 3-yl)-1H-indole-7-carboxylic acid |
| 58 | NS144-044 | 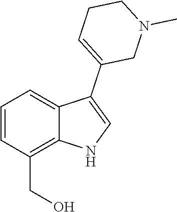 [SMILES: CN1CCC=C(c2c[nH]c3c(CO)cccc23)C1] | (3-(1-methyl-1,2,5,6- tetrahydropyridin-3-yl)-1H-indol-7- yl)methanol |
| 59 | YS135-44 | 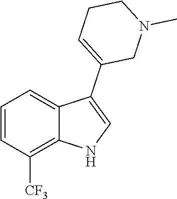 [SMILES: CN1CCC=C(c2c[nH]c3c(C(F)(F)F)cccc23)C1] | 3-(1-methyl-1,2,5,6-tetrahydropyridin- 3-yl)-7-(trifluoromethyl)-1H-indole |
| 60 | YS135-45 | 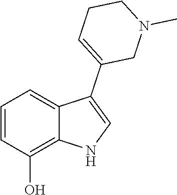 [SMILES: CN1CCC=C(c2c[nH]c3c(O)cccc23)C1] | 3-(1-methyl-1,2,5,6-tetrahydropyridin- 3-yl)-1H-indol-7-o1 |
| 61 | YS135-34 | 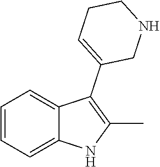 [SMILES: Cc1[nH]c2ccccc2c1C1=CCCNC1] | 2-methyl-3-(1,2,5,6-tetrahydropyridin- 3-yl)-1H-indole |
| 62 | YS135-32 | 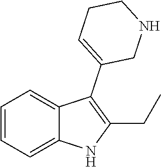 [SMILES: CCc1[nH]c2ccccc2c1C1=CCCNC1] | 2-ethyl-3-(1,2,5,6-tetrahydropyridin-3- yl)-1H-indole |
| 63 | YS135-38 | 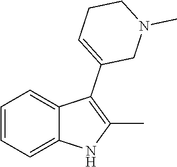 [SMILES: Cc1[nH]c2ccccc2c1C1=CCCN(C)C1] | 2-methyl-3-(1-methyl-1,2,5,6- tetrahydropyridin-3-yl)-1H-indole |
| 64 | YS135-41 | 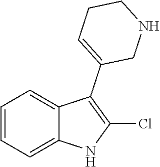 [SMILES: Clc1[nH]c2ccccc2c1C1=CCCNC1] | 2-chloro-3-(1,2,5,6-tetrahydropyridin- 3-yl)-1H-indole |
| 65 | YS135-39 | 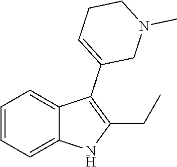 [SMILES: CCc1[nH]c2ccccc2c1C1=CCCN(C)C1] | 2-ethyl-3-(1-methyl-1,2,5,6- tetrahydropyridin-3-yl)-1H-indole |
| 66 | YX143-14A-2 | 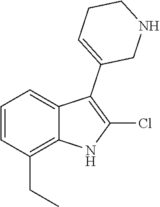 [SMILES: CCc1cccc2c(C3=CCCNC3)c(Cl)[nH]c12] | 2-chloro-7-ethyl-3-(1,2,5,6- tetrahydropyridin-3-yl)-1H-indole |
| 67 | NS144-019 | 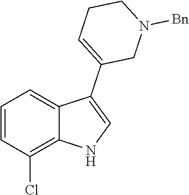 [SMILES: Clc1cccc2c(C3=CCCN(Cc4ccccc4)C3)c[nH]c12] | 3-(1-benzyl-1,2,5,6-tetrahydropyridin- 3-yl)-7-chloro-1H-indole |
| 68 | NS144-021 | 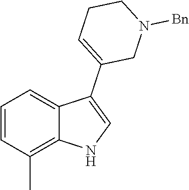 [SMILES: Cc1cccc2c(C3=CCCN(Cc4ccccc4)C3)c[nH]c12] | 3-(1-benzyl-1,2,5,6-tetrahydropyridin- 3-yl)-7-methyl-1H-indole |
| 69 | YX143-15 | 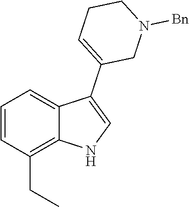 [SMILES: CCc1cccc2c(C3=CCCN(Cc4ccccc4)C3)c[nH]c12] | 3-(1-benzyl-1,2,5,6-tetrahydropyridin- 3-yl)-7-ethyl-1H-indole |
| 70 | YX143-16 | 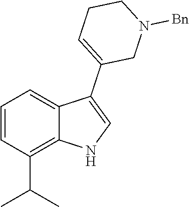 [SMILES: CC(C)c1cccc2c(C3=CCCN(Cc4ccccc4)C3)c[nH]c12] | 3-(1-benzyl-1,2,5,6-tetrahydropyridin- 3-yl)-7-isopropyl-1H-indole |
| 71 | YX143-17C | 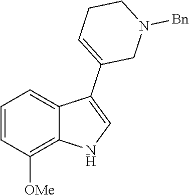 [SMILES: COc1cccc2c(C3=CCCN(Cc4ccccc4)C3)c[nH]c12] | 3-(1-benzyl-1,2,5,6-tetrahydropyridin- 3-yl)-7-methoxy-1H-indole |
| 72 | YX143-18C | 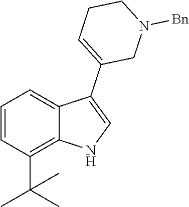 [SMILES: CC(C)(C)c1cccc2c(C3=CCCN(Cc4ccccc4)C3)c[nH]c12] | 3-(1-benzyl-1,2,5,6-tetrahydropyridin- 3-yl)-7-(tert-butyl)-1H-indole |
| 73 | NS144-047 | 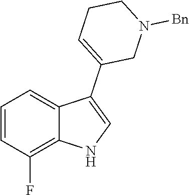 [SMILES: Fc1cccc2c(C3=CCCN(Cc4ccccc4)C3)c[nH]c12] | 3-(1-benzyl-1,2,5,6-tetrahydropyridin- 3-yl)-7-fluoro-1H-indole |
| 74 | NS144-048 | 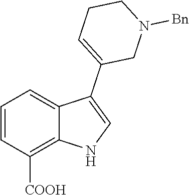 [SMILES: O=C(O)c1cccc2c(C3=CCCN(Cc4ccccc4)C3)c[nH]c12] | 3-(1-benzyl-1,2,5,6-tetrahydropyridin- 3-yl)-1H-indole-7-carboxylic acid |
| 75 | NS144-049 | 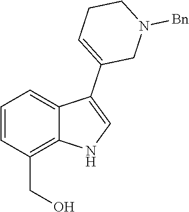 [SMILES: OCc1cccc2c(C3=CCCN(Cc4ccccc4)C3)c[nH]c12] | (3-(1-benzyl-1,2,5,6- tetrahydropyridin-3-yl)-1H-indol-7- yl)methanol |
| 76 | NS136-128 | 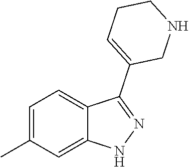 [SMILES: Cc1ccc2c(C3=CCCNC3)n[nH]c2c1] | 6-methyl-3-(1,2,5,6-tetrahydropyridin- 3-yl)-1H-indazole |
| 77 | NS136-129 | 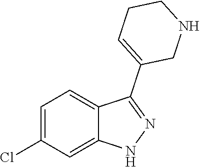 [SMILES: Clc1ccc2c(C3=CCCNC3)n[nH]c2c1] | 6-chloro-3-(1,2,5,6-tetrahydropyridin- 3-yl)-1H-indazole |
| 78 | NS136-130 | 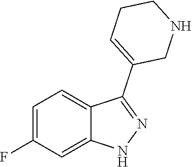 [SMILES: Fc1ccc2c(C3=CCCNC3)n[nH]c2c1] | 6-fluoro-3-(1,2,5,6-tetrahydropyridin- 3-yl)-1H-indazole |
| 79 | NS136-131 | 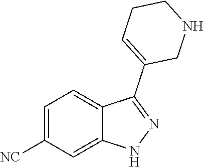 [SMILES: N#Cc1ccc2c(C3=CCCNC3)n[nH]c2c1] | 3-(1,2,5,6-tetrahydropyridin-3-yl)-1H- indazole-6-carbonitrile |
| 80 | NS136-150 | 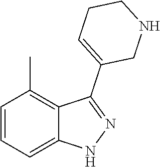 [SMILES: Cc1cccc2[nH]nc(C3=CCCNC3)c12] | 4-methyl-3-(1,2,5,6-tetrahydropyridin- 3-yl)-1H-indazole |
| 81 | NS136-151 | 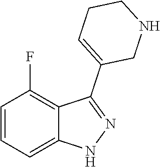 [SMILES: Fc1cccc2[nH]nc(C3=CCCNC3)c12] | 4-fluoro-3-(1,2,5,6-tetrahydropyridin- 3-yl)-1H-indazole |
| 82 | NS136-152 | 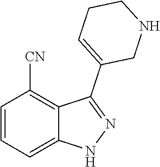 [SMILES: N#Cc1cccc2[nH]nc(C3=CCCNC3)c12] | 3-(1,2,5,6-tetrahydropyridin-3-yl)-1H- indazole-4-carbonitrile |
| 83 | NS136-166 | 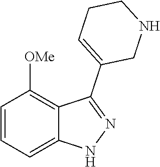 [SMILES: COc1cccc2[nH]nc(C3=CCCNC3)c12] | 4-methoxy-3-(1,2,5,6- tetrahydropyridin-3-yl)-1H-indazole |
| 84 | NS144-011 | 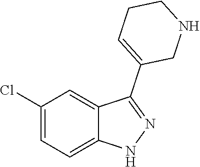 [SMILES: Clc1ccc2[nH]nc(C3=CCCNC3)c2c1] | 5-chloro-3-(1,2,5,6-tetrahydropyridin- 3-yl)-1H-indazole |
| 85 | NS136-158 | 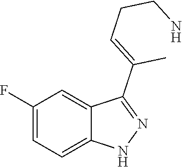 [SMILES: C/C(=C\CCN)c1n[nH]c2ccc(F)cc12] | 5-fluoro-3-(1,2,5,6-tetrahydropyridin- 3-yl)-1H-indazole |
| 86 | NS136-167 | 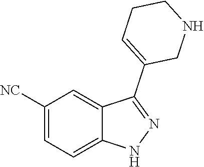 [SMILES: N#Cc1ccc2[nH]nc(C3=CCCNC3)c2c1] | 3-(1,2,5,6-tetrahydropyridin-3-yl)-1H- indazole-5-carbonitrile |
| 87 | NS136-159 | 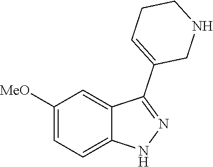 [SMILES: COc1ccc2[nH]nc(C3=CCCNC3)c2c1] | 5-methoxy-3-(1,2,5,6- tetrahydropyridin-3-yl)-1H-indazole |
| 88 | NS136-135 | 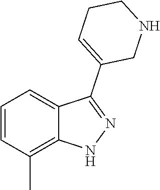 [SMILES: Cc1cccc2c(C3=CCCNC3)n[nH]c12] | 7-methyl-3-(1,2,5,6-tetrahydropyridin- 3-yl)-1H-indazole |
| 89 | NS136-136 | 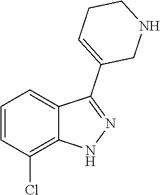 [SMILES: Clc1cccc2c(C3=CCCNC3)n[nH]c12] | 7-chloro-3-(1,2,5,6-tetrahydropyridin- 3-yl)-1H-indazole |
| 90 | NS136-137 | 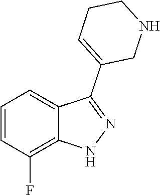 [SMILES: Fc1cccc2c(C3=CCCNC3)n[nH]c12] | 7-fluoro-3-(1,2,5,6-tetrahydropyridin- 3-yl)-1H-indazole |
| 91 | NS144-046 | 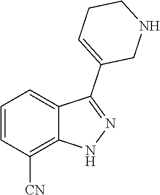 [SMILES: N#Cc1cccc2c(C3=CCCNC3)n[nH]c12] | 3-(1,2,5,6-tetrahydropyridin-3-yl)-1H- indazole-7-carbonitrile |
| 92 | NS144-045 | 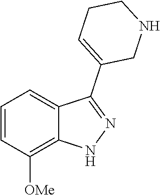 [SMILES: COc1cccc2c(C3=CCCNC3)n[nH]c12] | 7-methoxy-3-(1,2,5,6- tetrahydropyridin-3-yl)-1H-indazole |
| 93 | NS136-140 | 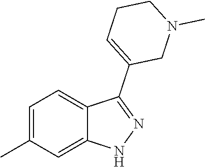 | 6-methyl-3-(1-methyl-1,2,5,6- tetrahydropyridin-3-yl)-1H-indazole |
| 94 | NS136-141 | 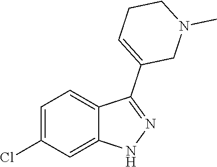 [SMILES: CN1CCC=C(c2n[nH]c3cc(Cl)ccc23)C1] | 6-chloro-3-(1-methyl-1,2,5,6- tetrahydropyridin-3-yl)-1H-indazole |
| 95 | NS136-142 | 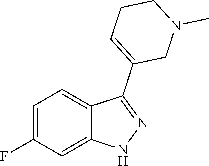 [SMILES: CN1CCC=C(c2n[nH]c3cc(F)ccc23)C1] | 6-fluoro-3-(1-methyl-1,2,5,6- tetrahydropyridin-3-yl)-1H-indazole |
| 96 | NS136-143 | 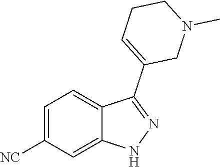 [SMILES: CN1CCC=C(c2n[nH]c3cc(C#N)ccc23)C1] | 3-(1-methyl-1,2,5,6-tetrahydropyridin- 3-yl)-1H-indazole-6-carbonitrile |
| 97 | NS136-153 | 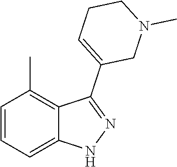 [SMILES: Cc1cccc2[nH]nc(C3=CCCN(C)C3)c12] | 4-methyl-3-(1-methyl-1,2,5,6- tetrahydropyridin-3-yl)-1H-indazole |
| 98 | NS136-154 | 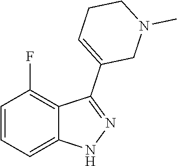 [SMILES: CN1CCC=C(c2n[nH]c3cccc(F)c23)C1] | 4-fluoro-3-(1-methyl-1,2,5,6- tetrahydropyridin-3-yl)-1H-indazole |
| 99 | NS136-155 | 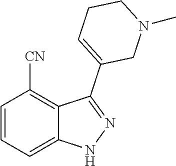 [SMILES: CN1CCC=C(c2n[nH]c3cccc(C#N)c23)C1] | 3-(1-methyl-1,2,5,6-tetrahydropyridin- 3-yl)-1H-indazole-4-carbonitrile |
| 100 | NS136-175 | 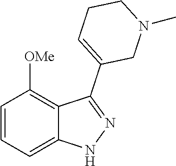 [SMILES: COc1cccc2[nH]nc(C3=CCCN(C)C3)c12] | 4-methoxy-3-(1-methyl-1,2,5,6- tetrahydropyridin-3-yl)-1H-indazole |
| 101 | NS144-016 | 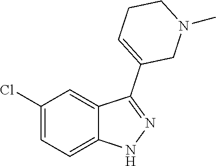 [SMILES: CN1CCC=C(c2n[nH]c3ccc(Cl)cc23)C1] | 5-chloro-3-(1-methyl-1,2,5,6- tetrahydropyridin-3-yl)-1H-indazole |
| 102 | NS136-160 | 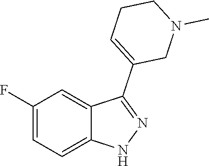 [SMILES: CN1CCC=C(c2n[nH]c3ccc(F)cc23)C1] | 5-fluoro-3-(1-methyl-1,2,5,6- tetrahydropyridin-3-yl)-1H-indazole |
| 103 | NS136-176 | 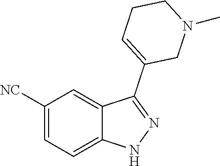 [SMILES: CN1CCC=C(c2n[nH]c3ccc(C#N)cc23)C1] | 3-(1-methyl-1,2,5,6-tetrahydropyridin- 3-yl)-1H-indazole-5-carbonitrile |
| 104 | NS136-161 | 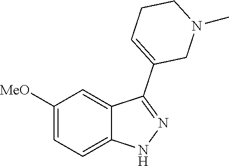 [SMILES: COc1ccc2[nH]nc(C3=CCCN(C)C3)c2c1] | 5-methoxy-3-(1-methyl-1,2,5,6- tetrahydropyridin-3-yl)-1H-indazole |
| 105 | NS136-144 | 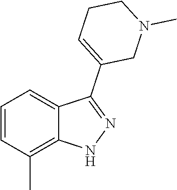 [SMILES: Cc1cccc2c(C3=CCCN(C)C3)n[nH]c12] | 7-methyl-3-(1-methyl-1,2,5,6- tetrahydropyridin-3-yl)-1H-indazole |
| 106 | NS136-145 | 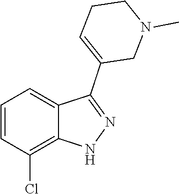 [SMILES: CN1CCC=C(c2n[nH]c3c(Cl)cccc23)C1] | 7-chloro-3-(1-methyl-1,2,5,6- tetrahydropyridin-3-yl)-1H-indazole |
| 107 | NS136-146 | 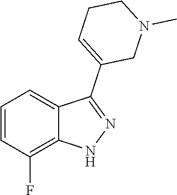 [SMILES: CN1CCC=C(c2n[nH]c3c(F)cccc23)C1] | 7-fluoro-3-(1-methyl-1,2,5,6- tetrahydropyridin-3-yl)-1H-indazole |
| 108 | NS144-051 | 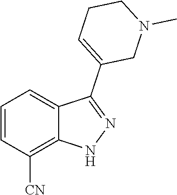 [SMILES: CN1CCC=C(c2n[nH]c3c(C#N)cccc23)C1] | 3-(1-methyl-1,2,5,6-tetrahydropyridin- 3-yl)-1H-indazole-7-carbonitrile |
| 109 | NS144-050 | 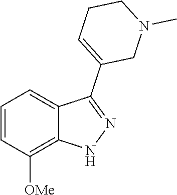 [SMILES: COc1cccc2c(C3=CCCN(C)C3)n[nH]c12] | 7-methoxy-3-(1-methyl-1,2,5,6- tetrahydropyridin-3-yl)-1H-indazole |
| 110 | YX143-41C | 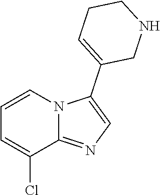 [SMILES: Clc1cccn2c(C3=CCCNC3)cnc12] | 8-chloro-3-(1,2,5,6-tetrahydropyridin- 3-yl)imidazo[1,2-a]pyridine |
| 111 | YX143-42C | 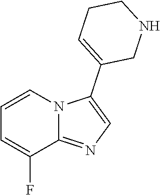 [SMILES: Fc1cccn2c(C3=CCCNC3)cnc12] | 8-fluoro-3-(1,2,5,6-tetrahydropyridin- 3-yl)imidazo[1,2-a]pyridine |
| 112 | YX143-43D | 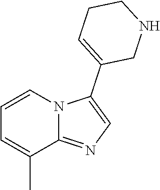 [SMILES: Cc1cccn2c(C3=CCCNC3)cnc12] | 8-methyl-3-(1,2,5,6-tetrahydropyridin- 3-yl)imidazo[1,2-a]pyridine |
| 113 | NS144-059-2 | 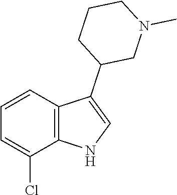 [SMILES: CN1CCCC(c2c[nH]c3c(Cl)cccc23)C1] | 7-chloro-3-(1-methylpiperidin-3-yl)- 1H-indole |
| 114 | NS144-054-2 | 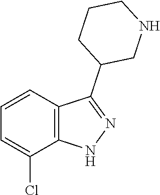 [SMILES: Clc1cccc2c(C3CCCNC3)n[nH]c12] | 7-chloro-3-(piperidin-3-yl)-1H- indazole |
| 115 | NS144-067 | 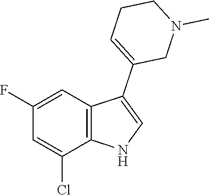 [SMILES: CN1CCC=C(c2c[nH]c3c(Cl)cc(F)cc23)C1] | 7-chloro-5-fluoro-3-(1-methyl-1,2,5,6- tetrahydropyridin-3-yl)-1H-indole |
| 116 | NS144-085 | 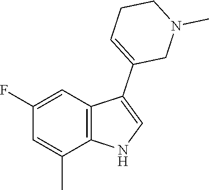 [SMILES: Cc1cc(F)cc2c(C3=CCCN(C)C3)c[nH]c12] | 5-fluoro-7-methyl-3-(1-methyl- 1,2,5,6-tetrahydropyridin-3-yl)-1H- indole |
| 117 | NS144-093 | 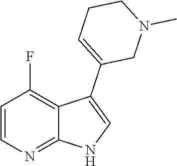 [SMILES: CN1CCC=C(c2c[nH]c3nccc(F)c23)C1] | 4-fluoro-3-(1-methyl-1,2,5,6- tetrahydropyridin-3-yl)-1H- pyrrolo[2,3-b]pyridine |
| 118 | NS144-094 | 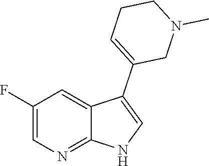 [SMILES: CN1CCC=C(c2c[nH]c3ncc(F)cc23)C1] | 5-fluoro-3-(1-methyl-1,2,5,6- tetrahydropyridin-3-yl)-1H- pyrrolo[2,3-b]pyridine |
| 119 | NS144-095 | 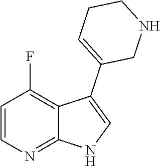 [SMILES: Fc1ccnc2[nH]cc(C3=CCCNC3)c12] | 4-fluoro-3-(1,2,5,6-tetrahydropyridin- 3-yl)-1H-pyrrolo[2,3-b]pyridine |
| 120 | NS144-096 | 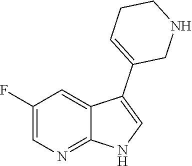 [SMILES: Fc1cnc2[nH]cc(C3=CCCNC3)c2c1] | 5-fluoro-3-(1,2,5,6-tetrahydropyridin- 3-yl)-1H-pyrrolo[2,3-b]pyridine |
| 121 | XQ148-012 | 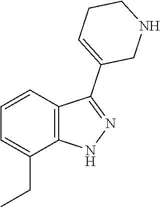 [SMILES: CCc1cccc2c(C3=CCCNC3)n[nH]c12] | 7-ethyl-3-(1,2,5,6-tetrahydropyridin-3- yl)-1H-indazole |
| 122 | XQ148-023 | 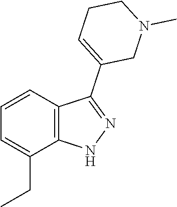 [SMILES: CCc1cccc2c(C3=CCCN(C)C3)n[nH]c12] | 7-ethyl-3-(1-methyl-1,2,5,6- tetrahydropyridin-3-yl)-1H-indazole |
| 123 | ZX147-015 | 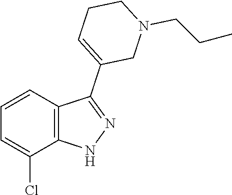 [SMILES: CCCN1CCC=C(c2n[nH]c3c(Cl)cccc23)C1] | 7-chloro-3-(1-propyl-1,2,5,6- tetrahydropyridin-3-yl)-1H-indazole |
| 124 | ZX147-016 | 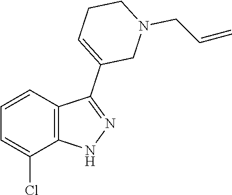 [SMILES: C=CCN1CCC=C(c2n[nH]c3c(Cl)cccc23)C1] | 3-(1-allyl-1,2,5,6-tetrahydropyridin-3- yl)-7-chloro-1H-indazole |
| 125 | ZX147-017 | 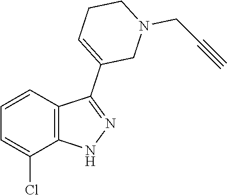 [SMILES: C#CCN1CCC=C(c2n[nH]c3c(Cl)cccc23)C1] | 7-chloro-3-(1-(prop-2-yn-1-yl)- 1,2,5,6-tetrahydropyridin-3-yl)-1H- indazole |
| 126 | ZX147-019 | 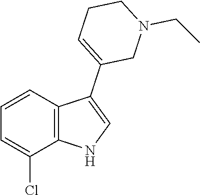 [SMILES: CCN1CCC=C(c2c[nH]c3c(Cl)cccc23)C1] | 7-chloro-3-(1-ethyl-1,2,5,6- tetrahydropyridin-3-yl)-1H-indole |

TABLE 2

| Examples | Compound code | Structure | Chemical Name |
|---|---|---|---|
| 127 | NS144-097 | 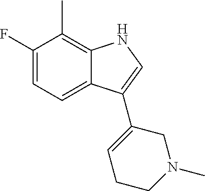 [SMILES: Cc1c(F)ccc2c(C3=CCCN(C)C3)c[nH]c12] | 6-fluoro-7-methyl-3-(1-methyl- 1,2,5,6-tetrahydropyridin-3-yl)- 1H-indole |
| 128 | NS144-098 | 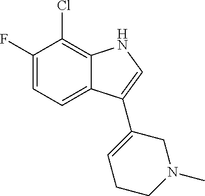 [SMILES: CN1CCC=C(c2c[nH]c3c(Cl)c(F)ccc23)C1] | 7-chloro-6-fluoro-3-(1-methyl- 1,2,5,6-tetrahydropyridin-3-yl)- 1H-indole |
| 129 | NS144-102 | 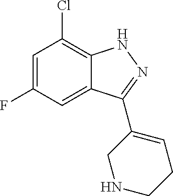 [SMILES: Fc1cc(Cl)c2[nH]nc(C3=CCCNC3)c2c1] | 7-chloro-5-fluoro-3-(1,2,5,6- tetrahydropyridin-3-yl)-1H- indazole |
| 130 | NS144-101 | 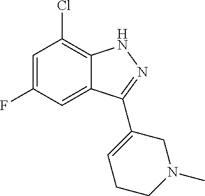 [SMILES: CN1CCC=C(c2n[nH]c3c(Cl)cc(F)cc23)C1] | 7-chloro-5-fluoro-3-(1-methyl- 1,2,5,6-tetrahydropyridin-3-yl)- 1H-indazole |
| 131 | NS144-107 | 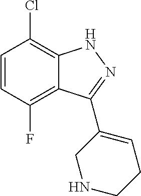 [SMILES: Fc1ccc(Cl)c2[nH]nc(C3=CCCNC3)c12] | 7-chloro-4-fluoro-3-(1,2,5,6- tetrahydropyridin-3-yl)-1H- indazole |
| 132 | NS144-108 | 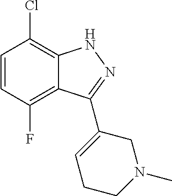 [SMILES: CN1CCC=C(c2n[nH]c3c(Cl)ccc(F)c23)C1] | 7-chloro-4-fluoro-3-(1-methyl- 1,2,5,6-tetrahydropyridin-3-yl)- 1H-indazole |
| 133 | NS144-109 | 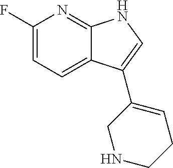 [SMILES: Fc1ccc2c(C3=CCCNC3)c[nH]c2n1] | 6-fluoro-3-(1,2,5,6- tetrahydropyridin-3-yl)-1H- pyrrolo[2,3-b]pyridine |
| 134 | NS144-110 | 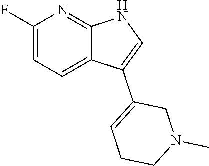 [SMILES: CN1CCC=C(c2c[nH]c3nc(F)ccc23)C1] | 6-fluoro-3-(1-methyl-1,2,5,6- tetrahydropyridin-3-yl)-1H- pyrrolo[2,3-b]pyridine |
| 135 | YS135-52 | 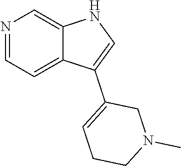 [SMILES: CN1CCC=C(c2c[nH]c3cnccc23)C1] | 3-(1-methyl-1,2,5,6- tetrahydropyridin-3-yl)-1H- pyrrolo[2,3-c]pyridine |
| 136 | YS135-53 | 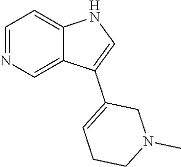 [SMILES: CN1CCC=C(c2c[nH]c3ccncc23)C1] | 3-(1-methyl-1,2,5,6- tetrahydropyridin-3-yl)-1H- pyrrolo[3,2-c]pyridine |
| 137 | YS135-54 | 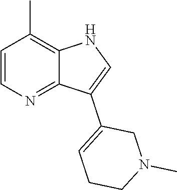 [SMILES: Cc1ccnc2c(C3=CCCN(C)C3)c[nH]c12] | 7-methyl-3-(1-methyl-1,2,5,6- tetrahydropyridin-3-yl)-1H- pyrrolo[3,2-b]pyridine |
| 138 | YS135-80 | 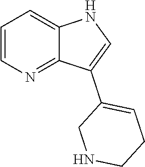 [SMILES: C1=C(c2c[nH]c3cccnc23)CNCC1] | 3-(1,2,5,6-tetrahydropyridin-3- yl)-1H-pyrrolo[3,2-b]pyridine |
| 139 | YS135-81 | 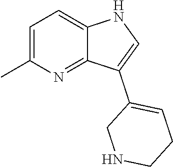 [SMILES: Cc1ccc2[nH]cc(C3=CCCNC3)c2n1] | 5-methyl-3-(1,2,5,6- tetrahydropyridin-3-yl)-1H- pyrrolo[3,2-b]pyridine |
| 140 | YS135-82 | 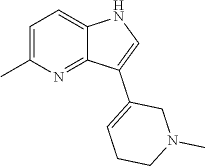 | 5-methyl-3-(1-methyl-1,2,5,6- tetrahydropyridin-3-yl)-1H- pyrrolo[3,2-b]pyridine |
| 141 | YS135-96 | 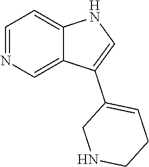 [SMILES: C1=C(c2c[nH]c3ccncc23)CNCC1] | 3-(1,2,5,6-tetrahydropyridin-3- yl)-1H-pyrrolo[3,2-c]pyridine |
| 142 | YS135-98 | 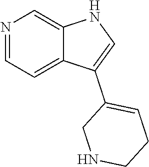 [SMILES: C1=C(c2c[nH]c3cnccc23)CNCC1] | 3-(1,2,5,6-tetrahydropyridin-3- yl)-1H-pyrrolo[2,3-c]pyridine |
| 143 | YS135-99 | 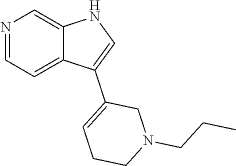 [SMILES: CCCN1CCC=C(c2c[nH]c3cnccc23)C1] | 3-(1-propyl-1,2,5,6- tetrahydropyridin-3-yl)-1H- pyrrolo[2,3-c]pyridine |
| 144 | YS135-100 | 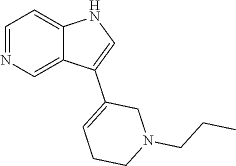 [SMILES: CCCN1CCC=C(c2c[nH]c3ccncc23)C1] | 3-(1-propyl-1,2,5,6- tetrahydropyridin-3-yl)-1H- pyrrolo[3,2-c]pyridine |
| 145 | ZX147-026-01 | 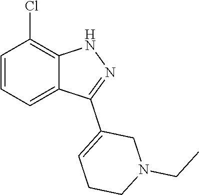 [SMILES: CCN1CCC=C(c2n[nH]c3c(Cl)cccc23)C1] | 7-chloro-3-(1-ethyl-1,2,5,6- tetrahydropyridin-3-yl)-1H- indazole |
| 146 | ZX147-026-02 | 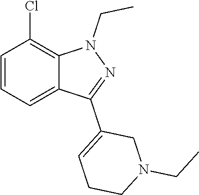 [SMILES: CCN1CCC=C(c2nn(CC)c3c(Cl)cccc23)C1] | 7-chloro-1-ethyl-3-(1-ethyl- 1,2,5,6-tetrahydropyridin-3-yl)- 1H-indazole |
| 147 | ZX147-027 | 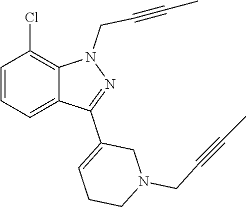 [SMILES: CC#CCN1CCC=C(c2nn(CC#CC)c3c(Cl)cccc23)C1] | 1-(but-2-yn-1-yl)-3-(1-(but-2- yn-1-yl)-1,2,5,6- tetrahydropyridin-3-yl)-7- chloro-1H-indazole |
| 148 | ZX147-028 | 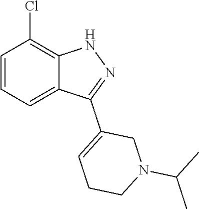 [SMILES: CC(C)N1CCC=C(c2n[nH]c3c(Cl)cccc23)C1] | 7-chloro-3-(1-isopropyl-1,2,5,6- tetrahydropyridin-3-yl)-1H- indazole |
| 149 | ZX147-029 | 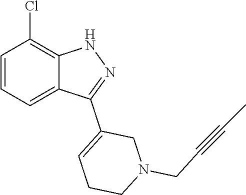 [SMILES: CC#CCN1CCC=C(c2n[nH]c3c(Cl)cccc23)C1] | 3-(1-(but-2-yn-1-yl)-1,2,5,6- tetrahydropyridin-3-yl)-7- chloro-1H-indazole |
| 150 | ZX147-031 | 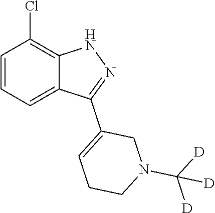 [SMILES: [2H]C([2H])([2H])N1CCC=C(c2n[nH]c3c(Cl)cccc23)C1] | 7-chloro-3-(1-(methyl-d3)- 1,2,5,6-tetrahydropyridin-3-yl)- 1H-indazole |
| 151 | ZX147-054 | 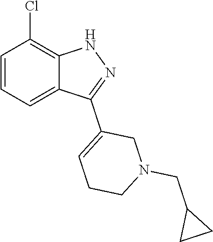 [SMILES: Clc1cccc2c(C3=CCCN(CC4CC4)C3)n[nH]c12] | 7-chloro-3-(1- (cyclopropylmethyl)-1,2,5,6- tetrahydropyridin-3-yl)-1H- indazole |
| 152 | ZX147-055 | 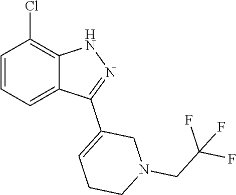 [SMILES: FC(F)(F)CN1CCC=C(c2n[nH]c3c(Cl)cccc23)C1] | 7-chloro-3-(1-(2,2,2- trifluoroethyl)-1,2,5,6- tetrahydropyridin-3-yl)-1H- indazole |
| 153 | ZX147-056 | 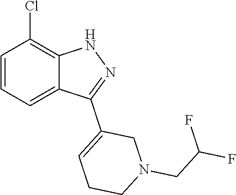 [SMILES: FC(F)CN1CCC=C(c2n[nH]c3c(Cl)cccc23)C1] | 7-chloro-3-(1-(2,2- difluoroethyl)-1,2,5,6- tetrahydropyridin-3-yl)-1H- indazole |
| 154 | ZX147-092 | 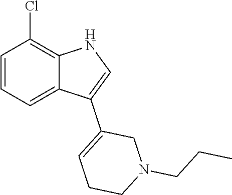 [SMILES: CCCN1CCC=C(c2c[nH]c3c(Cl)cccc23)C1] | 7-chloro-3-(1-propyl-1,2,5,6- tetrahydropyridin-3-yl)-1H- indole |
| 155 | ZX147-093 | 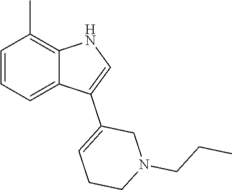 [SMILES: CCCN1CCC=C(c2c[nH]c3c(C)cccc23)C1] | 7-methyl-3-(1-propyl-1,2,5,6- tetrahydropyridin-3-yl)-1H- indole |
| 156 | ZX147-094 | 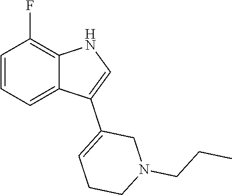 [SMILES: CCCN1CCC=C(c2c[nH]c3c(F)cccc23)C1] | 7-fluoro-3-(1-propyl-1,2,5,6- tetrahydropyridin-3-yl)-1H- indole |
| 157 | ZX147-095 | 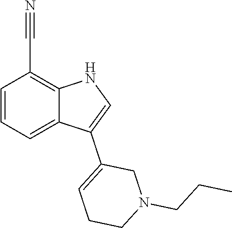 [SMILES: CCCN1CCC=C(c2c[nH]c3c(C#N)cccc23)C1] | 3-(1-propyl-1,2,5,6- tetrahydropyridin-3-yl)-1H- indole-7-carbonitrile |
| 158 | ZX147-096 | 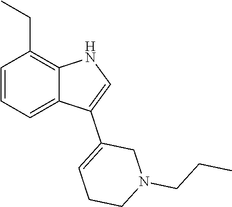 [SMILES: CCCN1CCC=C(c2c[nH]c3c(CC)cccc23)C1] | 7-ethyl-3-(1-propyl-1,2,5,6- tetrahydropyridin-3-yl)-1H- indole |
| 159 | ZX147-097 | 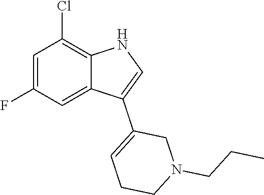 [SMILES: CCCN1CCC=C(c2c[nH]c3c(Cl)cc(F)cc23)C1] | 7-chloro-5-fluoro-3-(1-propyl- 1,2,5,6-tetrahydropyridin-3-yl)- 1H-indole |
| 160 | ZX147-098 | 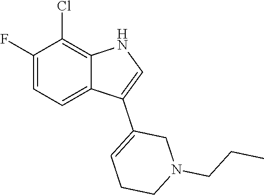 [SMILES: CCCN1CCC=C(c2c[nH]c3c(Cl)c(F)ccc23)C1] | 7-chloro-6-fluoro-3-(1-propyl- 1,2,5,6-tetrahydropyridin-3-yl)- 1H-indole |
| 161 | ZX147-099 | 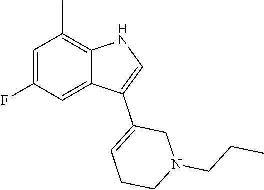 [SMILES: CCCN1CCC=C(c2c[nH]c3c(C)cc(F)cc23)C1] | 5-fluoro-7-methyl-3-(1-propyl- 1,2,5,6-tetrahydropyridin-3-yl)- 1H-indole |
| 162 | ZX147-100 | 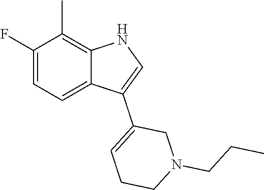 [SMILES: CCCN1CCC=C(c2c[nH]c3c(C)c(F)ccc23)C1] | 6-fluoro-7-methyl-3-(1-propyl- 1,2,5,6-tetrahydropyridin-3-yl)- 1H-indole |
| 163 | ZX147-128 | 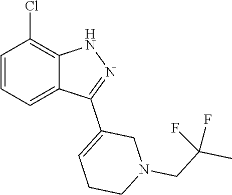 [SMILES: CC(F)(F)CN1CCC=C(c2n[nH]c3c(Cl)cccc23)C1] | 7-chloro-3-(1-(2,2- difluoropropyl)-1,2,5,6- tetrahydropyridin-3-yl)-1H- indazole |
| 164 | ZX147-129 | 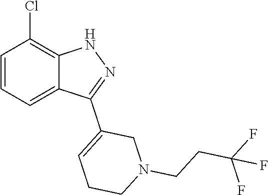 [SMILES: FC(F)(F)CCN1CCC=C(c2n[nH]c3c(Cl)cccc23)C1] | 7-chloro-3-(1-(3,3,3- trifluoropropyl)-1,2,5,6- tetrahydropyridin-3-yl)-1H- indazole |
| 165 | ZX147-130 | 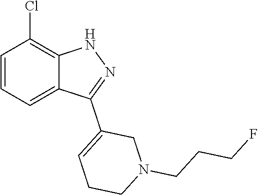 [SMILES: FCCCN1CCC=C(c2n[nH]c3c(Cl)cccc23)C1] | 7-chloro-3-(1-(3-fluoropropyl)- 1,2,5,6-tetrahydropyridin-3-yl)- 1H-indazole |
| 166 | ZX147-131 | 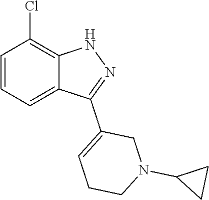 [SMILES: Clc1cccc2c(C3=CCCN(C4CC4)C3)n[nH]c12] | 7-chloro-3-(1-cyclopropyl- 1,2,5,6-tetrahydropyridin-3-yl)- 1H-indazole |
| 167 | ZX147-137 | 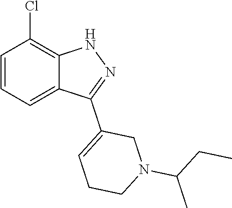 [SMILES: CCC(C)N1CCC=C(c2n[nH]c3c(Cl)cccc23)C1] | 3-(1-(sec-butyl)-1,2,5,6- tetrahydropyridin-3-yl)-7- chloro-1H-indazole |
| 168 | ZX147-183 | 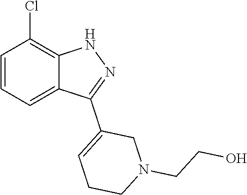 [SMILES: OCCN1CCC=C(c2n[nH]c3c(Cl)cccc23)C1] | 2-(5-(7-chloro-1H-indazol-3- yl)-3,6-dihydropyridin-1(2H)- yl)ethan-1-ol |
| 169 | ZX156-011 | 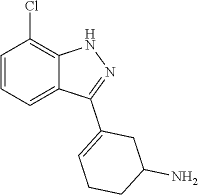 [SMILES: NC1CCC=C(c2n[nH]c3c(Cl)cccc23)C1] | 3-(7-chloro-1H-indazol-3- yl)cyclohex-3-en-1-amine |
| 170 | ZX156-012 | 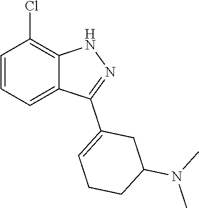 [SMILES: CN(C)C1CCC=C(c2n[nH]c3c(Cl)cccc23)C1] | 3-(7-chloro-1H-indazol-3-yl)- N,N-dimethylcyclohex-3-en-1- amine |
| 171 | ZX156-014-1 | 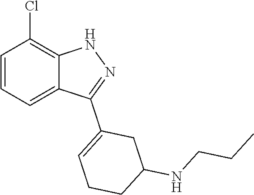 [SMILES: CCCNC1CCC=C(c2n[nH]c3c(Cl)cccc23)C1] | 3-(7-chloro-1H-indazol-3-yl)- N-propylcyclohex-3-en-1-amine |
| 172 | ZX156-014-2 | 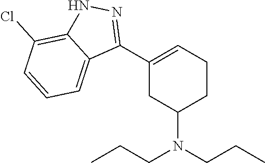 [SMILES: CCCN(CCC)C1CCC=C(c2n[nH]c3c(Cl)cccc23)C1] | 3-(7-chloro-1H-indazol-3-yl)- N,N-dipropylcyclohex-3-en-1- amine |
| 173 | ZX156-019 | 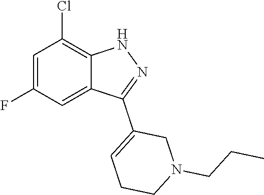 [SMILES: CCCN1CCC=C(c2n[nH]c3c(Cl)cc(F)cc23)C1] | 7-chloro-5-fluoro-3-(1-propyl- 1,2,5,6-tetrahydropyridin-3-yl)- 1H-indazole |
| 174 | ZX156-059 | 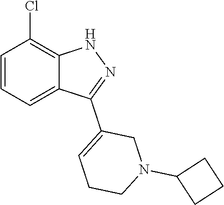 [SMILES: Clc1cccc2c(C3=CCCN(C4CCC4)C3)n[nH]c12] | 7-chloro-3-(1-cyclobutyl- 1,2,5,6-tetrahydropyridin-3-yl)- 1H-indazole |
| 175 | ZX156-069 | 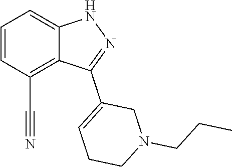 [SMILES: CCCN1CCC=C(c2n[nH]c3cccc(C#N)c23)C1] | 3-(1-propyl-1,2,5,6- tetrahydropyridin-3-yl)-1H- indazole-4-carbonitrile |
| 176 | ZX156-070 | 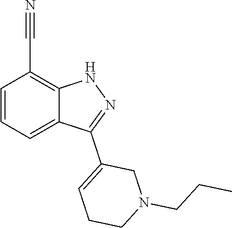 [SMILES: CCCN1CCC=C(c2n[nH]c3c(C#N)cccc23)C1] | 3-(1-propyl-1,2,5,6- tetrahydropyridin-3-yl)-1H- indazole-7-carbonitrile |
| 177 | ZX156-071 | 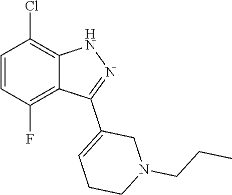 [SMILES: CCCN1CCC=C(c2n[nH]c3c(Cl)ccc(F)c23)C1] | 7-chloro-4-fluoro-3-(1-propyl- 1,2,5,6-tetrahydropyridin-3-yl)- 1H-indazole |
| 178 | ZX156-089 | 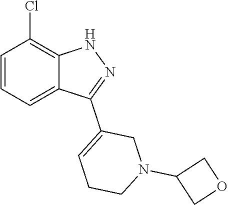 [SMILES: Clc1cccc2c(C3=CCCN(C4COC4)C3)n[nH]c12] | 7-chloro-3-(1-(oxetan-3-yl)- 1,2,5,6-tetrahydropyridin-3-yl)- 1H-indazole |
| 179 | ZX156-090 | 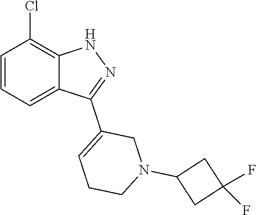 [SMILES: FC1(F)CC(N2CCC=C(c3n[nH]c4c(Cl)cccc34)C2)C1] | 7-chloro-3-(1-(3,3- difluorocyclobutyl)-1,2,5,6- tetrahydropyridin-3-yl)-1H- indazole |
| 180 | ZX162-100 | 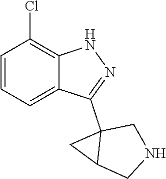 [SMILES: Clc1cccc2c(C34CNCC3C4)n[nH]c12] | 3-(3-azabicyclo[3.1.0]hexan-1- yl)-7-chloro-1H-indazole |
| 181 | ZX162-031 | 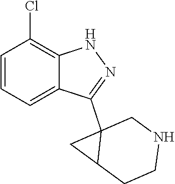 [SMILES: Clc1cccc2c(C34CNCCC3C4)n[nH]c12] | 3-(3-azabicyclo[4.1.0]heptan-1- yl)-7-chloro-1H-indazole |
| 182 | ZX162-104 | 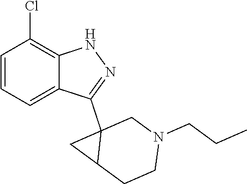 [SMILES: CCCN1CCC2CC2(c2n[nH]c3c(Cl)cccc23)C1] | 7-chloro-3-(3-propyl-3- azabicyclo[4.1.0]heptan-1-yl)- 1H-indazole |
| 183 | ZX162-105 | 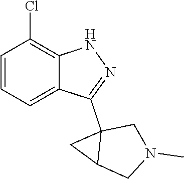 [SMILES: CN1CC2CC2(c2n[nH]c3c(Cl)cccc23)C1] | 7-chloro-3-(3-methyl-3- azabicyclo[3.1.0]hexan-1-yl)- 1H-indazole |
| 184 | ZX162-110 | 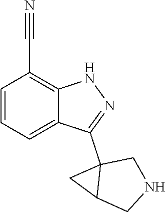 [SMILES: N#Cc1cccc2c(C34CNCC3C4)n[nH]c12] | 3-(3-azabicyclo[3.1.0]hexan-1- yl)-1H-indazole-7-carbonitrile |
| 185 | ZX162-111 | 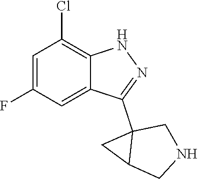 [SMILES: Fc1cc(Cl)c2[nH]nc(C34CNCC3C4)c2c1] | 3-(3-azabicyclo[3.1.0]hexan-1- yl)-7-chloro-5-fluoro-1H- indazole |
| 186 | ZX162-112 | 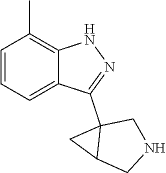 [SMILES: Cc1cccc2c(C34CNCC3C4)n[nH]c12] | 3-(3-azabicyclo[3.1.0]hexan-1- yl)-7-methyl-1H-indazole |
| 187 | ZX162-113 | 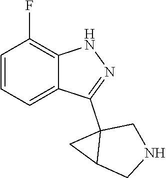 [SMILES: Fc1cccc2c(C34CNCC3C4)n[nH]c12] | 3-(3-azabicyclo[3.1.0]hexan-1- yl)-7-fluoro-1H-indazole |
| 188 | ZX162-124 | 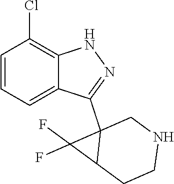 [SMILES: FC1(F)C2CCNCC21c1n[nH]c2c(Cl)cccc12] | 7-chloro-3-(7,7-difluoro-3- azabicyclo[4.1.0]heptan-1-yl)- 1H-indazole |
| 189 | ZX162-126 | 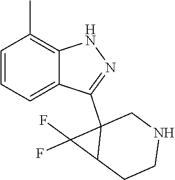 [SMILES: Cc1cccc2c(C34CNCCC3C4(F)F)n[nH]c12] | 3-(7,7-difluoro-3- azabicyclo[4.1.0]heptan-1-yl)- 7-methyl-1H-indazole |
| 190 | ZX162-127 | 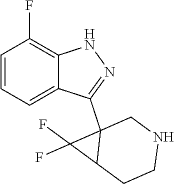 [SMILES: Fc1cccc2c(C34CNCCC3C4(F)F)n[nH]c12] | 3-(7, 7-difluoro-3- azabicyclo[4.1.0]heptan-1-yl)- 7-fluoro-1H-indazole |
| 191 | ZX162-128 | 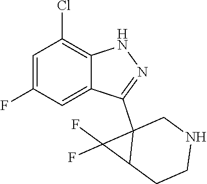 [SMILES: Fc1cc(Cl)c2[nH]nc(C34CNCCC3C4(F)F)c2c1] | 7-chloro-3-(7,7-difluoro-3- azabicyclo[4.1.0]heptan-1-yl)- 5-fluoro-1H-indazole |
| 192 | ZX162-129 | 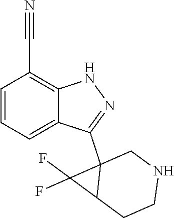 [SMILES: N#Cc1cccc2c(C34CNCCC3C4(F)F)n[nH]c12] | 3-(7,7-difluoro-3- azabicyclo[4.1.0]heptan-1-yl)- 1H-indazole-7-carbonitrile |
| 193 | ZX162-138 | 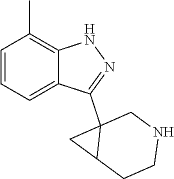 [SMILES: Cc1cccc2c(C34CNCCC3C4)n[nH]c12] | 3-(3-azabicyclo[4.1.0]heptan-1- yl)-7-methyl-1H-indazole |
| 194 | ZX162-139 | 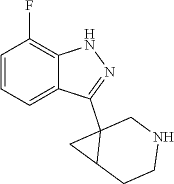 [SMILES: Fc1cccc2c(C34CNCCC3C4)n[nH]c12] | 3-(3-azabicyclo[4.1.0]heptan-1- yl)-7-fluoro-1H-indazole |
| 195 | ZX162-140 | 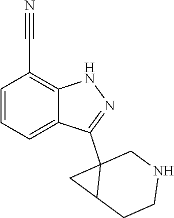 [SMILES: N#Cc1cccc2c(C34CNCCC3C4)n[nH]c12] | 3-(3-azabicyclo[4.1.0]heptan-1- yl)-1H-indazole-7-carbonitrile |
| 196 | ZX162-141 | 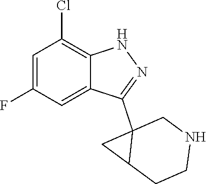 [SMILES: Fc1cc(Cl)c2[nH]nc(C34CNCCC3C4)c2c1] | 3-(3-azabicyclo[4.1.0]heptan-1- yl)-7-chloro-5-fluoro-1H- indazole |
| 197 | ZX162-147 | 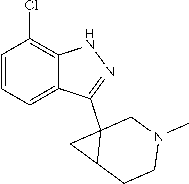 [SMILES: CN1CCC2CC2(c2n[nH]c3c(Cl)cccc23)C1] | 7-chloro-3-(3-methyl-3- azabicyclo[4.1.0]heptan-1-yl)- 1H-indazole |
| 198 | ZX162-148 | 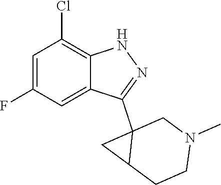 [SMILES: CN1CCC2CC2(c2n[nH]c3c(Cl)cc(F)cc23)C1] | 7-chloro-5-fluoro-3-(3-methyl- 3-azabicyclo[4.1.0]heptan-1- yl)-1H-indazole |
| 199 | ZX162-151 | 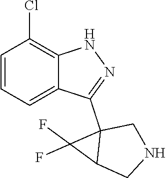 [SMILES: FC1(F)C2CNCC21c1n[nH]c2c(Cl)cccc12] | 7-chloro-3-(6,6-difluoro-3- azabicyclo[3.1.0]hexan-1-yl)- 1H-indazole |
| 200 | ZX162-173 | 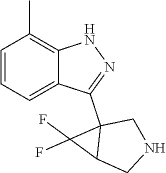 [SMILES: Cc1cccc2c(C34CNCC3C4(F)F)n[nH]c12] | 3-(6,6-difluoro-3- azabicyclo[3.1.0]hexan-1-yl)-7- methyl-1H-indazole |
| 201 | ZX162-174 | 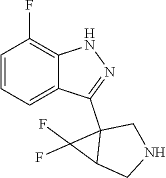 [SMILES: Fc1cccc2c(C34CNCC3C4(F)F)n[nH]c12] | 3-(6,6-difluoro-3- azabicyclo[3.1.0]hexan-1-yl)-7- fluoro-1H-indazole |
| 202 | ZX162-175 | 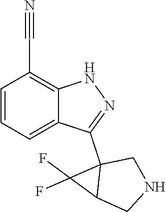 [SMILES: N#Cc1cccc2c(C34CNCC3C4(F)F)n[nH]c12] | 3-(6,6-difluoro-3- azabicyclo[3.1.0]hexan-1-yl)- 1H-indazole-7-carbonitrile |
| 203 | ZX162-176 | 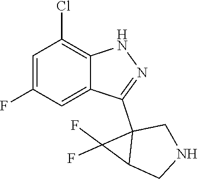 [SMILES: Fc1cc(Cl)c2[nH]nc(C34CNCC3C4(F)F)c2c1] | 7-chloro-3-(6,6-difluoro-3- azabicyclo[3.1.0]hexan-1-yl)-5- fluoro-1H-indazole |
| 204 | YX143-103B | 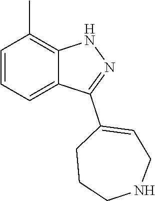 [SMILES: Cc1cccc2c(C3=CCNCCC3)n[nH]c12] | 7-methyl-3-(2,5,6,7-tetrahydro- 1H-azepin-4-yl)-1H-indazole |
| 205 | YX143-103C | 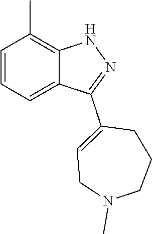 [SMILES: Cc1cccc2c(C3=CCN(C)CCC3)n[nH]c12] | 7-methyl-3-(1-methyl-2,5,6,7- tetrahydro-1H-azepin-4-yl)-1H- indazole |
| 206 | YX143-105C | 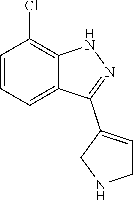 [SMILES: Clc1cccc2c(C3=CCNC3)n[nH]c12] | 7-chloro-3-(2,5-dihydro-1H- pyrrol-3-yl)-1H-indazole |
| 207 | YX143-108 | 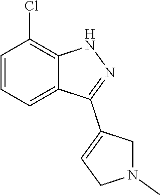 [SMILES: CN1CC=C(c2n[nH]c3c(Cl)cccc23)C1] | 7-chloro-3-(1-methyl-2,5- dihydro-1H-pyrrol-3-yl)-1H- indazole |
| 208 | YX143-110B | 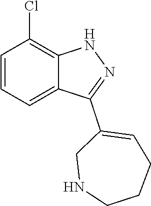 [SMILES: Clc1cccc2c(C3=CCCCNC3)n[nH]c12] | 7-chloro-3-(2,5,6,7-tetrahydro- 1H-azepin-3-yl)-1H-indazole |
| 209 | YX143-112B | 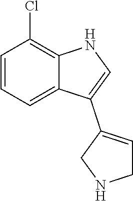 [SMILES: Clc1cccc2c(C3=CCNC3)c[nH]c12] | 7-chloro-3-(2,5-dihydro-1H- pyrrol-3-yl)-1H-indole |
| 210 | YX143-129 | 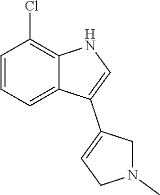 [SMILES: CN1CC=C(c2c[nH]c3c(Cl)cccc23)C1] | 7-chloro-3-(1-methyl-2,5- dihydro-1H-pyrrol-3-yl)-1H- indole |
| 211 | YX143-134C | 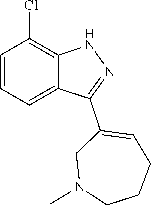 [SMILES: CN1CCCC=C(c2n[nH]c3c(Cl)cccc23)C1] | 7-chloro-3-(1-methyl-2,5,6,7- tetrahydro-1H-azepin-3-yl)-1H- indazole |
| 212 | YX143-138C | 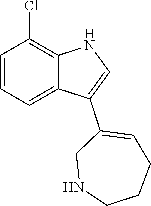 [SMILES: Clc1cccc2c(C3=CCCCNC3)c[nH]c12] | 7-chloro-3-(2,5,6,7-tetrahydro- 1H-azepin-3-yl)-1H-indole |
| 213 | YX143-182C-1 | 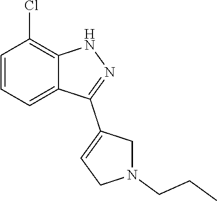 [SMILES: CCCN1CC=C(c2n[nH]c3c(Cl)cccc23)C1] | 7-chloro-3-(1-propyl-2,5- dihydro-1H-pyrrol-3-yl)-1H- indazole |
| 214 | YX143-183A | 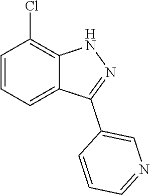 [SMILES: Clc1cccc2c(-c3cccnc3)n[nH]c12] | 7-chloro-3-(pyridin-3-yl)-1H- indazole |
| 215 | YX143-184B-1 | 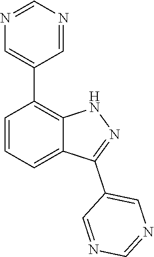 [SMILES: c1cc(-c2cncnc2)c2[nH]nc(-c3cncnc3)c2c1] | 3,7-di(pyrimidin-5-yl)-1H- indazole |
| 216 | YX143-184B-2 | 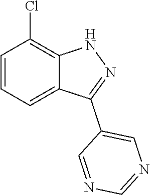 [SMILES: Clc1cccc2c(-c3cncnc3)n[nH]c12] | 7-chloro-3-(pyrimidin-5-yl)- 1H-indazole |
| 217 | YX143-185B | 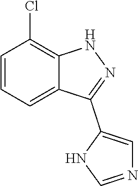 [SMILES: Clc1cccc2c(-c3cnc[nH]3)n[nH]c12] | 7-chloro-3-(1H-imidazol-5-yl)- 1H-indazole |
| 218 | YX143-186B | 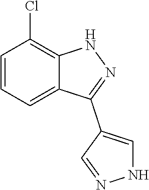 [SMILES: Clc1cccc2c(-c3cn[nH]c3)n[nH]c12] | 7-chloro-3-(1H-pyrazol-4-yl)- 1H-indazole |
| 219 | YX157-19A | 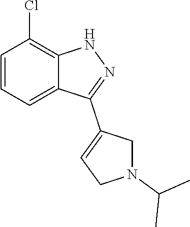 [SMILES: CC(C)N1CC=C(c2n[nH]c3c(Cl)cccc23)C1] | 7-chloro-3-(1-isopropyl-2,5- dihydro-1H-pyrrol-3-yl)-1H- indazole |
| 220 | YX157-20A | 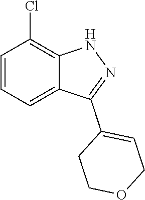 [SMILES: Clc1cccc2c(C3=CCOCC3)n[nH]c12] | 7-chloro-3-(3,6-dihydro-2H- pyran-4-yl)-1H-indazole |
| 221 | YX157-29B | 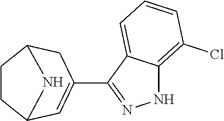 [SMILES: Clc1cccc2c(C3=CC4CCC(C3)N4)n[nH]c12] | 3-(8-azabicyclo[3.2.1]oct-2-en- 3-yl)-7-chloro-1H-indazole |
| 222 | YX157-42B | 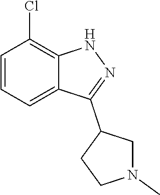 [SMILES: CN1CCC(c2n[nH]c3c(Cl)cccc23)C1] | 7-chloro-3-(1-methylpyrrolidin- 3-yl)-1H-indazole |
| 223 | YX157-51B | 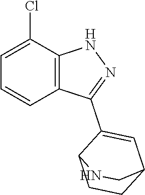 [SMILES: Clc1cccc2c(C3=CC4CCC3NC4)n[nH]c12] | 6-(7-chloro-1H-indazol-3-yl)-2- azabicyclo[2.2.2]oct-5-ene |
| 224 | YX157-51C | 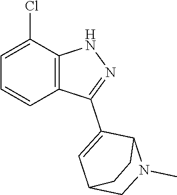 [SMILES: CN1CC2C=C(c3n[nH]c4c(Cl)cccc34)C1CC2] | 6-(7-chloro-1H-indazol-3-yl)-2- methyl-2-azabicyclo[2.2.2]oct- 5-ene |
| 225 | YX157-55A | 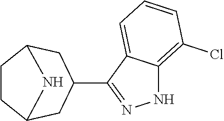 [SMILES: Clc1cccc2c(C3CC4CCC(C3)N4)n[nH]c12] | 3-(8-azabicyclo[3.2.1]octan-3- yl)-7-chloro-1H-indazole |
| 226 | XS159-153 | 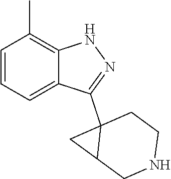 [SMILES: Cc1cccc2c(C34CCNCC3C4)n[nH]c12] | 3-(3-azabicyclo[4.1.0]heptan-6- yl)-7-methyl-1H-indazole |
| 227 | XS159-155 | 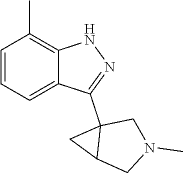 [SMILES: Cc1cccc2c(C34CC3CN(C)C4)n[nH]c12] | 7-methyl-3-(3-methyl-3- azabicyclo[3.1.0]hexan-1-yl)- 1H-indazole |
| 228 | XS159-160 | 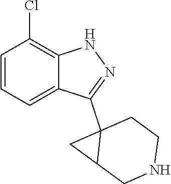 [SMILES: Clc1cccc2c(C34CCNCC3C4)n[nH]c12] | 3-(3-azabicyclo[4.1.0]heptan-6- yl)-7-chloro-1H-indazole |
| 229 | XS159-163 | 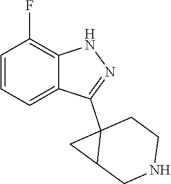 [SMILES: Fc1cccc2c(C34CCNCC3C4)n[nH]c12] | 3-(3-azabicyclo[4.1.0]heptan-6- yl)-7-fluoro-1H-indazole |
| 230 | XS159-180 | 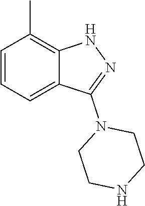 [SMILES: Cc1cccc2c(N3CCNCC3)n[nH]c12] | 7-methyl-3-(piperazin-1-yl)- 1H-indazole |
| 231 | XS159-186 | 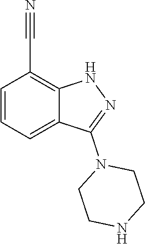 [SMILES: N#Cc1cccc2c(N3CCNCC3)n[nH]c12] | 3-(piperazin-1-yl)-1H-indazole- 7-carbonitrile |
| 232 | XS165-3 | 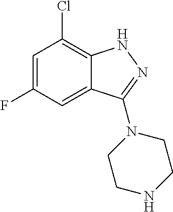 [SMILES: Fc1cc(Cl)c2[nH]nc(N3CCNCC3)c2c1] | 7-chloro-5-fluoro-3-(piperazin- 1-yl)-1H-indazole |
| 233 | XS165-5 | 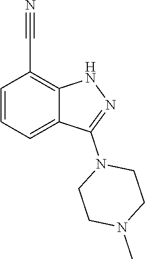 [SMILES: CN1CCN(c2n[nH]c3c(C#N)cccc23)CC1] | 3-(4-methylpiperazin-1-yl)-1H- indazole-7-carbonitrile |
| 234 | XS165-8 | 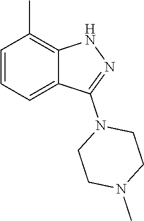 [SMILES: Cc1cccc2c(N3CCN(C)CC3)n[nH]c12] | 7-methyl-3-(4-methylpiperazin- 1-yl)-1H-indazole |
| 235 | XQ148-93 | 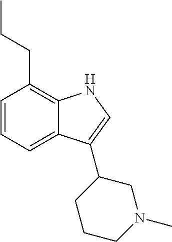 [SMILES: CCCc1cccc2c(C3CCCN(C)C3)c[nH]c12] | 3-(1-methylpiperidin-3-yl)-7- propyl-1H-indole |
| 236 | XQ158-012 | 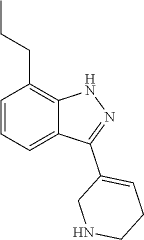 [SMILES: CCCc1cccc2c(C3=CCCNC3)n[nH]c12] | 7-propyl-3-(1,2,5,6- tetrahydropyridin-3-yl)-1H- indazole |
| 237 | XQ158-055 | 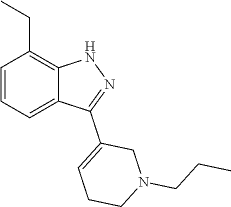 [SMILES: CCCN1CCC=C(c2n[nH]c3c(CC)cccc23)C1] | 7-ethyl-3-(1-propyl-1,2,5,6- tetrahydropyridin-3-yl)-1H- indazole |
| 238 | XQ158-056 | 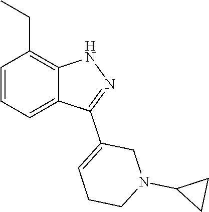 [SMILES: CCc1cccc2c(C3=CCCN(C4CC4)C3)n[nH]c12] | 3-(1-cyclopropyl-1,2,5,6- tetrahydropyridin-3-yl)-7-ethyl- 1H-indazole |
| 239 | XQ158-078 | 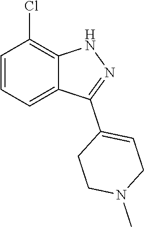 [SMILES: CN1CC=C(c2n[nH]c3c(Cl)cccc23)CC1] | 7-chloro-3-(1-methyl-1,2,3,6- tetrahydropyridin-4-yl)-1H- indazole |
| 240 | XQ158-093A | 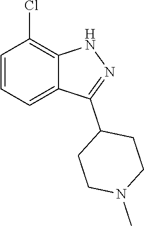 [SMILES: CN1CCC(c2n[nH]c3c(Cl)cccc23)CC1] | 7-chloro-3-(1-methylpiperidin- 4-yl)-1H-indazole |
| 241 | XQ158-082 | 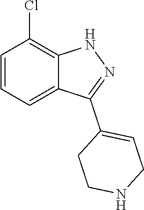 [SMILES: Clc1cccc2c(C3=CCNCC3)n[nH]c12] | 7-chloro-3-(1,2,3,6- tetrahydropyridin-4-yl)-1H- indazole |
| 242 | XQ158-115 | 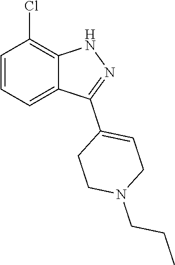 [SMILES: CCCN1CC=C(c2n[nH]c3c(Cl)cccc23)CC1] | 7-chloro-3-(1-propyl-1,2,3,6- tetrahydropyridin-4-yl)-1H- indazole |
| 243 | XQ158-164 | 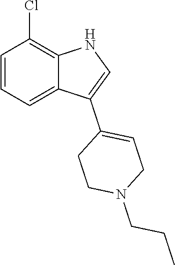 [SMILES: CCCN1CC=C(c2c[nH]c3c(Cl)cccc23)CC1] | 7-chloro-3-(1-propyl-1,2,3,6- tetrahydropyridin-4-yl)-1H- indole |
| 244 | XQ158-167 | 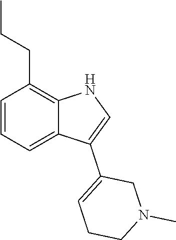 [SMILES: CCCc1cccc2c(C3=CCCN(C)C3)c[nH]c12] | 3-(1-methyl-1,2,5,6- tetrahydropyridin-3-yl)-7- propyl-1H-indole |
| 245 | XQ158-168 | 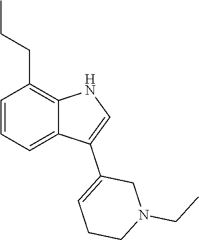 [SMILES: CCCc1cccc2c(C3=CCCN(CC)C3)c[nH]c12] | 3-(1-ethyl-1,2,5,6- tetrahydropyridin-3-yl)-7- propyl-1H-indole |
| 246 | ZD160-34 | 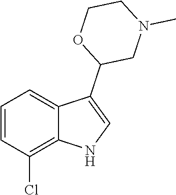 [SMILES: CN1CCOC(c2c[nH]c3c(Cl)cccc23)C1] | 2-(7-chloro-1H-indol-3-yl)-4- methylmorpholine |
| 247 | ZD160-140 | 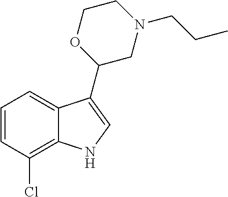 [SMILES: CCCN1CCOC(c2c[nH]c3c(Cl)cccc23)C1] | 2-(7-chloro-1H-indol-3-yl)-4- propylmorpholine |
| 248 | ZD160-141 | 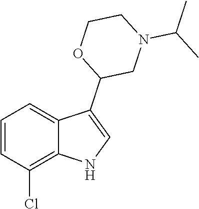 [SMILES: CC(C)N1CCOC(c2c[nH]c3c(Cl)cccc23)C1] | 2-(7-chloro-1H-indol-3-yl)-4- isopropylmorpholine |
| 249 | ZD160-149 | 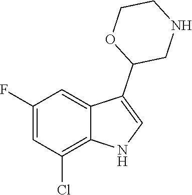 [SMILES: Fc1cc(Cl)c2[nH]cc(C3CNCCO3)c2c1] | 2-(7-chloro-5-fluoro-1H-indol- 3-yl)morpholine |
| 250 | ZD160-11 | 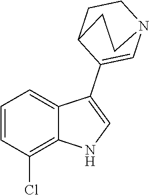 [SMILES: Clc1cccc2c(C3=CN4CCC3CC4)c[nH]c12] | 3-(7-chloro-1H-indol-3-yl)-1- azabicyclo[2.2.2]oct-2-ene |
| 251 | ZD160-133 | 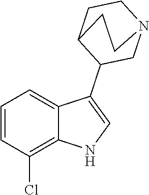 [SMILES: Clc1cccc2c(C3CN4CCC3CC4)c[nH]c12] | 3-(7-chloro-1H-indol-3- yl)quinuclidine |
| 252 | ZD160-130 | 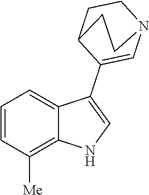 [SMILES: Cc1cccc2c(C3=CN4CCC3CC4)c[nH]c12] | 3-(7-methyl-1H-indol-3-yl)-1- azabicyclo[2.2.2]oct-2-ene |
| 253 | ZD160-131 | 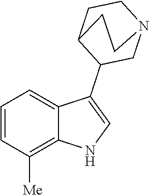 [SMILES: Cc1cccc2c(C3CN4CCC3CC4)c[nH]c12] | 3-(7-methyl-1H-indol-3- yl)quinuclidine |
| 254 | QC166-005 | 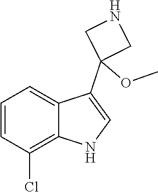 [SMILES: COC1(c2c[nH]c3c(Cl)cccc23)CNC1] | 7-chloro-3-(3-methoxyazetidin- 3-yl)-1H-indole |
| 255 | QC166-008 | 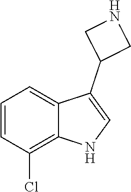 [SMILES: Clc1cccc2c(C3CNC3)c[nH]c12] | 3-(azetidin-3-yl)-7-chloro-1H- indole |
| 256 | QC-166-032 | 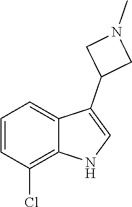 | 7-chloro-3-(1-methylazetidin-3- yl)-1H-indole |
| 257 | XQ148-86 | 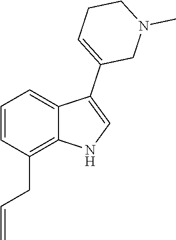 [SMILES: C=CCc1cccc2c(C3=CCCN(C)C3)c[nH]c12] | 7-allyl-3-(1-methyl-1,2,5,6- tetrahydropyridin-3-yl)-1H- indole |
| 258 | QC166-096 | 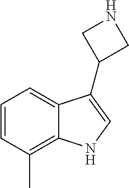 | 3-(azetidin-3-yl)-7-methyl-1H- indole |
| 259 | QC166-097 | 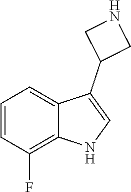 [SMILES: Fc1cccc2c(C3CNC3)c[nH]c12] | 3-(azetidin-3-yl)-7-fluoro-1H- indole |
| 260 | QC179-001 | 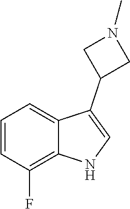 | 7-fluoro-3-(1-methylazetidin-3- yl)-1H-indole |
| 261 | QC179-002 | 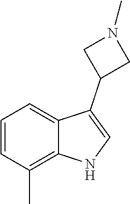 | 7-methyl-3-(1-methylazetidin- 3-yl)-1H-indole |
| 262 | QC179-025 | 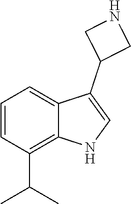 [SMILES: CC(C)c1cccc2c(C3CNC3)c[nH]c12] | 3-(azetidin-3-yl)-7-isopropyl- 1H-indole |
| 263 | QC-179-032 | 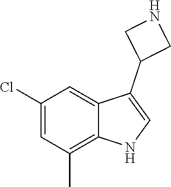 | 3-(azetidin-3-yl)-5-chloro-7- methyl-1H-indole |
| 264 | QC-179-033 | 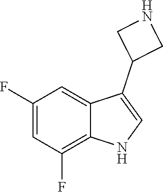 [SMILES: Fc1cc(F)c2[nH]cc(C3CNC3)c2c1] | 3-(azetidin-3-yl)-5,7-difluoro- 1H-indole |
| 265 | QC179-038 | 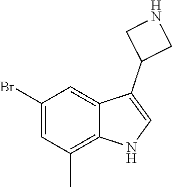 | 3-(azetidin-3-yl)-5-bromo-7- methyl-1H-indole |
| 266 | QC179-039 | 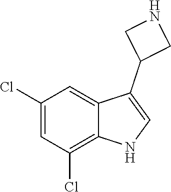 [SMILES: Clc1cc(Cl)c2[nH]cc(C3CNC3)c2c1] | 3-(azetidin-3-yl)-5,7-dichloro- 1H-indole |
| 267 | QC179-040 | 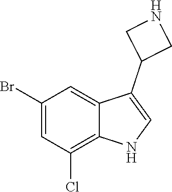 [SMILES: Clc1cc(Br)cc2c(C3CNC3)c[nH]c12] | 3-(azetidin-3-yl)-5-bromo-7- chloro-1H-indole |
| 268 | ZX167-072 | 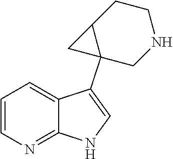 [SMILES: c1cnc2[nH]cc(C34CNCCC3C4)c2c1] | 3-(3-azabicyclo[4.1.0]heptan-1- yl)-1H-pyrrolo[2,3-b]pyridine |
| 269 | ZX167-077 | 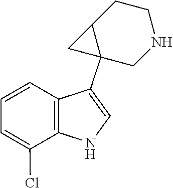 [SMILES: Clc1cccc2c(C34CNCCC3C4)c[nH]c12] | 3-(3-azabicyclo[4.1.0]heptan-1- yl)-7-chloro-1H-indole |
| 270 | ZX167-074 | 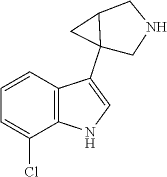 [SMILES: Clc1cccc2c(C34CNCC3C4)c[nH]c12] | 3-(3-azabicyclo[3.1.0]hexan-1- yl)-7-chloro-1H-indole |
| 271 | ZX167-090 | 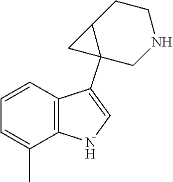 [SMILES: Cc1cccc2c(C34CNCCC3C4)c[nH]c12] | 3-(3-azabicyclo[4.1.0]heptan-1- yl)-7-methyl-1H-indole |
| 272 | ZX167-091 | 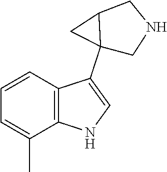 [SMILES: Cc1cccc2c(C34CNCC3C4)c[nH]c12] | 3-(3-azabicyclo[3.1.0]hexan-1- yl)-7-methyl-1H-indole |
| 273 | ZX162-100-1 (Enantiomer 1 of ZX162- 100) | 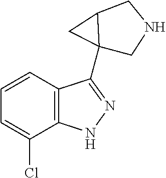 [SMILES: Clc1cccc2c(C34CNCC3C4)n[nH]c12] | 3-(3-azabicyclo[3.1.0]hexan-1- yl)-7-chloro-1H-indazole |
| 274 | ZX162-100-2 (Enantiomer 2 of ZX162- 100) | 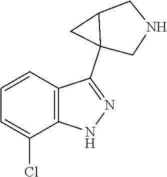 [SMILES: Clc1cccc2c(C34CNCC3C4)n[nH]c12] | 3-(3-azabicyclo[3.1.0]hexan-1- yl)-7-chloro-1H-indazole |
| 275 | ZX162-031-1 (Enantiomer 1 of ZX162- 031) | 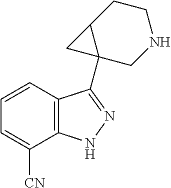 [SMILES: N#Cc1cccc2c(C34CNCCC3C4)n[nH]c12] | 3-(3-azabicyclo[4.1.0]heptan-1- yl)-7-chloro-1H-indazole |
| 276 | ZX162-031-2 (Enantiomer 2 of ZX162- 031) | 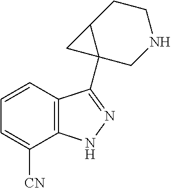 [SMILES: N#Cc1cccc2c(C34CNCCC3C4)n[nH]c12] | 3-(3-azabicyclo[4.1.0]heptan-1- yl)-7-chloro-1H-indazole |
| 277 | ZX167-074-1 (Enantiomer 1 of ZX167- 074) | 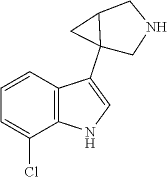 [SMILES: Clc1cccc2c(C34CNCC3C4)c[nH]c12] | 3-(3-azabicyclo[3.1.0]hexan-1- yl)-7-chloro-1H-indole |
| 278 | ZX167-074-2 (Enantiomer 2 of ZX167- 074) | 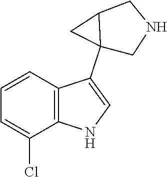 [SMILES: Clc1cccc2c(C34CNCC3C4)c[nH]c12] | 3-(3-azabicyclo[3.1.0]hexan-1- yl)-7-chloro-1H-indole |
| 279 | ZX177-057 | 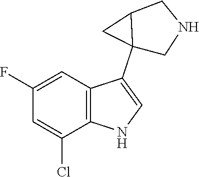 [SMILES: Fc1cc(Cl)c2[nH]cc(C34CNCC3C4)c2c1] | 3-(3-azabicyclo[3.1.0]hexan-1- yl)-7-chloro-5-fluoro-1H-indole |
| 280 | ZX177-058 | 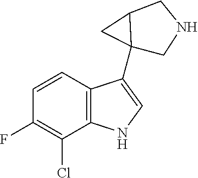 [SMILES: Fc1ccc2c(C34CNCC3C4)c[nH]c2c1Cl] | 3-(3-azabicyclo[3.1.0]hexan-1- yl)-7-chloro-6-fluoro-1H-indole |
| 281 | ZX177- 058BY | 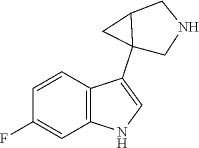 [SMILES: Fc1ccc2c(C34CNCC3C4)c[nH]c2c1] | 3-(3-azabicyclo[3.1.0]hexan-1- yl)-6-fluoro-1H-indole |
| 282 | ZX177-059 | 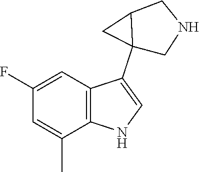 [SMILES: Cc1cc(F)cc2c(C34CNCC3C4)c[nH]c12] | 3-(3-azabicyclo[3.1.0]hexan-1- yl)-5-fluoro-7-methyl-1H- indole |
| 283 | ZX177-060 | 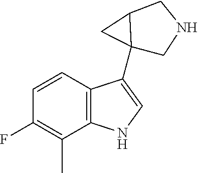 [SMILES: Cc1c(F)ccc2c(C34CNCC3C4)c[nH]c12] | 3-(3-azabicyclo[3.1.0]hexan-1- yl)-6-fluoro-7-methyl-1H- indole |

Compounds corresponding to Examples 1-126 have been synthesized and are provided with a Compound Code in Table 1.

Compounds corresponding to Examples 127-283 have been synthesized and are provided with a Compound Code in Table 2.

As used herein, in case of discrepancy between the structure and chemical name provided for a particular compound, the given structure shall control.

### General Chemistry Methods

For the synthesis of intermediates and examples, HPLC spectra for all compounds were acquired using an Agilent 1200 Series system with DAD detector. Chromatography was performed on a 2.1×150 mm Zorbax 300SB-C18 5 μm column with water containing 0.1% formic acid as solvent A and acetonitrile containing 0.1% formic acid as solvent B at a flow rate of 0.4 ml/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Nuclear Magnetic Resonance (NMR) spectra were acquired on a Bruker DRX-600 spectrometer with 600 MHz for proton (¹H NMR) and 150 MHz for carbon (¹³C NMR); chemical shifts are reported in (6). Preparative HPLC was performed on Agilent Prep 1200 series with UV detector set to 254 nm. Samples were injected onto a Phenomenex Luna 250×30 mm, 5 μm, C18 column at room temperature. The flow rate was 40 ml/min. A linear gradient was used with 10% (or 50%) of MeOH (A) in H₂O (with 0.1% TFA) (B) to 100% of MeOH (A). HPLC was used to establish the purity of target compounds. All final compounds had >95% purity using the HPLC methods described above.

### Example 284

### Biological Methods:

All biological assays are performed in HEK 293T cells (bioluminescence resonance energy transfer (BRET) assays) or Flp-In T-REx 293 cells (binding and calcium flux assays).

Binding assays: Two binding assays, referred to as primary and secondary binding assays, are used to identify compounds that bind and to measure the affinities of their binding, respectively, to 5HT2A, 5HT2B, and 5HT2C receptors. Crude membranes are prepared from Flp-In T Rex 293 cells stably expressing the receptor of interest in a doxycycline/tetracycline-inducible manner.

For primary binding assays, membranes are co-incubated with compound (10 μM) and radioligand (0.5-1.0 K_{d}; for the 5HT2A and 5HT2B receptors, the radioligand is [3H]-LSD, and for the 5-HT2C receptor the radioligand is [3H]-mesulergine) in standard binding buffer (50 mM Tris HCl, 10 mM MgCl₂, 0.1 mM EDTA, pH 7.4). Total binding is determined by replacing compound with buffer only, and non-specific binding is determined by replacing compound with a positive control, a known high-affinity binder (either clozapine or LSD). All conditions are tested in technical quadruplicate. Compounds, radioligands, and membranes are incubated at room temperature prior to harvesting via vacuum filtration onto 0.3% polyethylimine-soaked filter mats and subsequent washing/vacuuming with wash buffer (50 mM Tris HCl, pH 7.4, cold). Scintillation wax is then melted onto the filter mats, and measurements are taken using a microbeta counter. Compounds that show at least 50% displacement of radioligand relative to the total specific binding are then assessed in secondary assays to determine their binding affinities.

Secondary assays are performed similarly to primary binding assays, except in the set up of the conditions. Here, compounds are half-logarithmically diluted to yield final concentrations between 10 μM and 0.1 μM with a final well supplemented with buffer instead of drug (i.e., total binding). Each well is incubated with radioligand, as described for primary assays. Compounds are tested in technical triplicate, with the exception of the positive control, which is tested in technical duplicate, and in biological triplicate. The highest concentration of the positive control yields the non-specific binding. Binding curves are analyzed in GraphPad Prism, with Kᵢs determined from the experimental IC₅₀s using the Cheng-Prusoff equation.

BRET assays: Two complimentary BRET assays are used to quantitatively measure 5-HT2A activation of Gαq heterotrimeric G proteins and recruitment of beta-arrestin2 in response to compounds. The G protein assay is from the BRET2-based TRUPATH platform, in which Renilla luciferase (RLuc) has been fused to Gαq and GFP2 has been fused to Gγ9. These plasmids, along with those encoding 5HT2A receptor and Gβ3, are co-transfected in HEK293T cells. 96-well plates containing transfected cells are then aspirated of media, and incubated for 30 minutes at 37° C. with compound (logarithmically diluted to yield final concentrations between 10 μM and 0.1 μM) in assay buffer (HBSS, 20 mM HEPES, pH 7.4) (each plate contains a 5-HT dilution series as a positive control to which values are normalized during analysis). 10 minutes prior to reading (20 minutes after incubation begins), the BRET2 substrate coelenterazine 400a (5 μM final concentration) is added. Reading takes place in a microplate reader to measure RLuc luminescence and GFP2 fluorescence. The beta-arrestin2 assay is BRET1-based, with RLuc fused to the C-terminus of the receptor and mVenus fused to the N-terminus of beta-arrestin2. Plasmids encoding these constructs, along with a plasmid encoding G protein-coupled receptor kinase 2, are co-transfected. Procedurally, the BRET1 assay is performed the same as the BRET2 assays, but uses a different substrate, coelenterazine h, to account for the different acceptor fluorophore. Data are analyzed in GraphPad Prism, and the responses of all compounds on a given plate are normalized to that produced by 5-HT on the same plate to yield measurements of potency and relative efficacy.

Calcium flux assay (calcium mobilization assay): Calcium flux assay: Flp-In TREx 293 cells stably expressing GPCR (5HT2A, 5HT2B, or 5HT2C) were plated in black 384-well plates in 40 uL/well Pro293 medium (Lonza) supplemented with 20 mM L-glutamine (Gibco) and incubated at 37 C and 5% CO2 overnight. Prior to running the experiment, medium was removed from the plates and replaced with Fluo-4 dye (Fisher) prepared according to vendor protocols and returned to incubation for 1 hour. Drug dilutions were prepared in HBSS with 0.1% bovine serum albumin. Plates were run using a FLIPR TETRA (Molecular Devices) with 384-well liquid handling system. Baseline fluorescence was recorded for ten seconds prior to drug addition, and fluorescence was recorded once per second for two minutes after drug addition. The maximum fluorescence signal during those two minutes was plotted against delivered drug concentration to obtain concentration-response curves, which were then analyzed in Prism (GraphPad).

### Table 3 & 4. Binding Affinities and Functional Activities of Synthesized Compounds for 5HT2A Receptor.

TABLE 3

| | | 5HT2A | 5HT2A Gq | | 5HT2A β-Arr | |
|---|---|---|---|---|---|---|
| Examples | Compound code | Kᵢ (nM) | Eₘₐₓ (%) | EC₅₀ (nM) | Eₘₐₓ (%) | EC₅₀ (nM) |
| 1 | NS131-179 | 1719.0 | N.D. | N.D. | N.D. | N.D. |
| 2 | NS131-178 | 293.8 | 73.7 | 135.1 | 61.6 | 1043.0 |
| 3 | NS131-177 | 142.7 | 82.0 | 29.4 | 43.8 | 229.4 |
| 4 | NS136-006 | 613.40 | 67.0 | 294.2 | N.D. | N.D. |
| 5 | NS131-169 | 716.8 | N.D. | N.D. | N.D. | N.D. |
| 6 | NS131-168 | 694.7 | 65.9 | 44.1 | 33.4 | 792.1 |
| 7 | NS131-167 | 208.6 | 73.4 | 56.9 | 20.7 | 18.7 |
| 8 | NS131-173 | 155.7 | 95.5 | 67.0 | 58.3 | 811.5 |
| 9 | NS131-180 | 8507.0 | 74.2 | 489.1 | 72.8 | 748.8 |
| 10 | RS134-52 | 311.6 | 66.5 | 124.6 | N.D. | N.D. |
| 11 | RS134-48 | 272.5 | 84.7 | 79.9 | 43.7 | 903.6 |
| 12 | NS131-185 | 190.8 | N.D. | N.D. | N.D. | N.D. |
| 13 | NS131-170 | 63.5 | 68.7 | 105.8 | 38.9 | 764.7 |
| 14 | RS134-45 | 43.0 | 67.1 | 72.7 | 32.2 | 74.1 |
| 15 | RS134-40 | 465.0 | 68.8 | 23.5 | 31.5 | 683.3 |
| 16 | NS131-184 | 163.8 | N.D. | N.D. | 49.6 | 2136.0 |
| 17 | RS134-49 | 11.5 | 85.7 | 22.0 | 42.0 | 147.4 |
| 18 | RS134-53 | 50.5 | 96.1 | 3.9 | 51.8 | 71.2 |
| 19 | RS134-41 | 136.0 | 75.9 | 9.0 | 40.4 | 101.5 |
| 20 | RS134-46 | 60.0 | 91.0 | 3.0 | 47.7 | 61.8 |
| 21 | NS131-172 | 14.0 | 59.6 | 874.3 | 15.1 | 358.4 |
| 22 | RS134-38 | 44.3 | 102.5 | 7.2 | 77.3 | 78.4 |
| 23 | RS134-65 | 98.6 | 58.8 | 65.7 | 31.4 | 734.9 |
| 24 | RS134-62 | 104.5 | N.D. | N.D. | N.D. | N.D. |
| 25 | RS134-70 | 940.7 | 73.3 | 592.1 | N.D. | N.D. |
| 26 | NS136-081 | 165.8 | 64.2 | 176.6 | N.D. | N.D. |
| 27 | RS134-73 | 1476.0 | N.D. | N.D. | N.D. | N.D. |
| 28 | RS134-72 | 497.5 | N.D. | N.D. | N.D. | N.D. |
| 29 | NS136-092 | 1201.0 | N.D. | N.D. | N.D. | N.D. |
| 30 | NS136-091 | 646.9 | N.D. | N.D. | N.D. | N.D. |
| 31 | NS136-096 | 160.1 | 93.0 | 106.2 | 111.7 | 1497.0 |
| 32 | NS136-095 | 236.5 | 77.5 | 303.8 | 45.5 | 709.4 |
| 33 | NS136-102 | 1263.0 | N.D. | N.D. | N.D. | N.D. |
| 34 | NS136-101 | 486.3 | N.D. | N.D. | N.D. | N.D. |
| 35 | NS136-115 | >10,000 | N.D. | N.D. | N.D. | N.D. |
| 36 | NS136-116 | 8495.0 | N.D. | N.D. | N.D. | N.D. |
| 37 | NS136-117 | 9909.0 | N.D. | N.D. | N.D. | N.D. |
| 38 | NS136-118 | 7883.0 | N.D. | N.D. | N.D. | N.D. |
| 39 | NS136-119 | 6755.0 | N.D. | N.D. | N.D. | N.D. |
| 40 | NS136-120 | 1332.0 | N.D. | N.D. | N.D. | N.D. |
| 41 | NS136-109 | 1376.0 | 92.6 | 234.1. | 83.2 | 1410.00 |
| 42 | NS136-110 | 144.9 | 101.6 | 15.8 | 76.5 | 180.0 |
| 43 | NS136-111 | 405.2 | 86.8 | 68.1 | 48.9 | 486.7 |
| 44 | NS136-112 | 76.3 | 86.6 | 18.9 | 37.6 | 78.8 |
| 45 | RS134-37 | 112.5 | 98.3 | 9.9 | 68.1 | 138.2 |
| 46 | RS134-56 | 87.3 | 62.6 | 3.3 | 21.9 | 114.8 |
| 47 | NS136-002 | 795.9 | 48.7 | 378.6 | N.D. | N.D. |
| 48 | NS136-004 | 423.4 | N.D. | N.D. | N.D. | N.D. |
| 49 | RS130-132 | 5583.0 | N.D. | N.D. | N.D. | N.D. |
| 50 | YX129-177C | 244.3 | 92.6 | 4.7 | 79.1 | 67.0 |
| 51 | YX129-180C | >10,000 | N.D. | N.D. | N.D. | N.D. |
| 52 | YX143-19 | 42.0 | 89.3 | 25.0 | 73.4 | 880.0 |
| 53 | YX143-20 | 373.7 | 77.8 | 165.7 | N.D. | N.D. |
| 54 | YX143-2 | 510.2 | N.D. | N.D. | N.D. | N.D. |
| 55 | YX143-21 | 373.7 | N.D. | N.D. | N.D. | N.D. |
| 56 | NS144-042 | 59.8 | 96.9 | 16.8 | 49.5 | 57.7 |
| 57 | NS144-043 | >10,000 | N.D. | N.D. | N.D. | N.D. |
| 58 | NS144-044 | >10,000 | N.D. | N.D. | N.D. | N.D. |
| 59 | YS135-44 | 25.8 | 81.7 | 17.8 | 56.9 | 75.5 |
| 60 | YS135-45 | 795.4 | 64.3 | 439.9 | N.D. | N.D. |
| 61 | YS135-34 | 804.6 | 67.4 | 363.0 | N.D. | N.D. |
| 62 | YS135-32 | 809.6 | 49.1 | 75.7 | 45.1 | 633.3 |
| 63 | YS135-38 | 822.6 | 79.0 | 162.1 | N.D. | N.D. |
| 64 | YS135-41 | 185.6 | 91.8 | 61.9 | 60.2 | 218.9 |
| 65 | YS135-39 | 221.3 | 51.2 | 124.5 | N.D. | N.D. |
| 66 | YX143-14A-2 | 115.0 | 79.0 | 212.9 | N.D. | N.D. |
| 67 | NS144-019 | 90.2 | 48.8 | 44.1 | 46.6 | 756.0 |
| 68 | NS144-021 | 115.3 | 40.2 | 127.3 | 42.4 | 489.8 |
| 69 | YX143-15 | 199.2 | 49.7 | 98.2 | 63.5 | 1743.0 |
| 70 | YX143-16 | 2050.0 | N.D. | N.D. | N.D. | N.D. |
| 71 | YX143-17C | 448.4 | 65.2 | 530.2 | N.D. | N.D. |
| 72 | YX143-18C | 733.9 | N.D. | N.D. | N.D. | N.D. |
| 73 | NS144-047 | 150.9 | 81.9 | 116.3 | 56.5 | 331.3 |
| 74 | NS144-048 | >10,000 | N.D. | N.D. | N.D. | N.D. |
| 75 | NS144-049 | >10,000 | N.D. | N.D. | N.D. | N.D. |
| 76 | NS136-128 | 2462.0 | 93.6 | 117.6 | 88.7 | 1503.0 |
| 77 | NS136-129 | 438.9 | 93.9 | 38.9 | 76.7 | 429.5 |
| 78 | NS136-130 | 57.6 | 79.4 | 11.5 | 59.4 | 115.3 |
| 79 | NS136-131 | 441.2 | 48.6 | 874.3 | 82.1 | 1711.0 |
| 80 | NS136-150 | 254.5 | 83.4 | 73.8 | 71.8 | 697.8 |
| 81 | NS136-151 | 348.1 | 92.3 | 26.5 | 75.7 | 210.6 |
| 82 | NS136-152 | 153.4 | 95.9 | 12.0 | 87.9 | 561.7 |
| 83 | NS136-166 | 78.8 | 81.7 | 12.5 | 75.9 | 108.6 |
| 84 | NS144-011 | 25.3 | 94.8 | 3.4 | 97.0 | 52.4 |
| 85 | NS136-158 | 33.4 | 88.3 | 2.8 | 96.0 | 68.6 |
| 86 | NS136-167 | 370.4 | 90.2 | 15.9 | 95.1 | 354.5 |
| 87 | NS136-159 | 119.3 | 84.2 | 17.8 | 80.0 | 241.3 |
| 88 | NS136-135 | 211.4 | 72.6 | 52.8 | 64.8 | 227.3 |
| 89 | NS136-136 | 85.2 | 93.7 | 11.4 | 85.1 | 83.5 |
| 90 | NS136-137 | 1393.0 | 95.6 | 9.2 | 70.0 | 86.0 |
| 91 | NS144-046 | 236.9 | 126.3 | 17.2 | 86.0 | 105.1 |
| 92 | NS144-045 | 1089.0 | N.D. | N.D. | N.D. | N.D. |
| 93 | NS136-140 | 2920.0 | N.D. | N.D. | N.D. | N.D. |
| 94 | NS136-141 | 1301.0 | 72.1 | 308.7 | N.D. | N.D. |
| 95 | NS136-142 | 47.3 | 47.1 | 21.1 | 27.0 | 466.8 |
| 96 | NS136-143 | 5466.0 | N.D. | N.D. | N.D. | N.D. |
| 97 | NS136-153 | 786.0 | 69.0 | 80.4 | 64.6 | 604.8 |
| 98 | NS136-154 | 835.3 | 58.5 | 105.3 | 54.0 | 483.2 |
| 99 | NS136-155 | 491.2 | 80.0 | 96.4 | 43.8 | 1013.0 |
| 100 | NS136-175 | 348.9 | 63.9 | 91.6 | 52.4 | 460.2 |
| 101 | NS144-016 | 141.1 | 74.2 | 16.2 | 80.7 | 292.3 |
| 102 | NS136-160 | 245.8 | 84.5 | 9.0 | 79.8 | 128.7 |
| 103 | NS136-176 | 1390.0 | 64.9 | 611.9 | N.D. | N.D. |
| 104 | NS136-161 | 500.5 | 88.2 | 82.8 | 83.7 | 681.4 |
| 105 | NS136-144 | 1338.0 | 75.4 | 35.4 | 45.7 | 432.4 |
| 106 | NS136-145 | 332.4 | 85.5 | 39.1 | 66.8 | 176.4 |
| 107 | NS136-146 | 882.7 | 68.1 | 34.5 | 36.3 | 212.8 |
| 108 | NS144-051 | 1375 | 101.9 | 108.1 | 72.5 | 494.1 |
| 109 | NS144-050 | >10,000 | N.D. | N.D. | N.D. | N.D. |
| 110 | YX143-41C | >10,000 | N.D. | N.D. | N.D. | N.D. |
| 111 | YX143-42C | >10,000 | N.D. | N.D. | N.D. | N.D. |
| 112 | YX143-43D | 9036 | N.D. | N.D. | N.D. | N.D. |
| 113 | NS144-059-2 | 1426. | 84.9 | 279.2 | 62.6 | 1524.0 |
| 114 | NS144-054-2 | 1237 | 74.5 | 289.8 | 29.3 | 468.4 |
| 115 | NS144-067 | 20.8 | 113.6 | 51.1 | 59.7 | 575.8 |
| 116 | NS144-085 | 99.6 | 125.2 | 74.0 | 56.1 | 129.4 |
| 117 | NS144-093 | 1365.0 | 110.2 | 252.9 | 51.1 | 865.2 |
| 118 | NS144-094 | 80.3 | 109.0 | 31.6 | 56.7 | 49.4 |
| 119 | NS144-095 | 844.4 | 88.2 | 193.1 | 53.83 | 1524 |
| 120 | NS144-096 | 53.6 | 100.4 | 21.5 | 60.4 | 67.4 |
| 121 | XQ148-012 | 422.3 | 102.6 | 20.2 | 85.7 | 75.8 |
| 122 | XQ148-023 | 816.4 | 102.1 | 151.1 | 42.9 | 247.2 |
| 123 | ZX147-015 | 139.8 | 66.9 | 142.1 | N.D. | N.D. |
| 124 | ZX147-016 | 159.1 | 64.9 | 49.6 | 35.3 | 1008 |
| 125 | ZX147-017 | 1970.0 | 93.5 | 75.0 | 63.3 | 290.9 |
| 126 | ZX147-019 | 42.6 | 96.6 | 40.2 | 62.9 | 74.9 |

TABLE 4

| | | 5HT2A | 5HTA Gq | | 5HT2A β-Arr | |
|---|---|---|---|---|---|---|
| Examples | Compound code | Kᵢ (nM) | Eₘₐₓ (%) | EC₅₀ (nM) | Eₘₐₓ (%) | EC₅₀ (nM) |
| 127 | NS144-097 | 120.8 | 90.2 | 26.6 | 48.9 | 62.9 |
| 128 | NS144-098 | 15.3 | 89.2 | 11.9 | 56.5 | 66.1 |
| 129 | NS144-102 | 162.4 | 88.8 | 3.1 | 95.0 | 20.1 |
| 130 | NS144-101 | 19.5 | 91.4 | 12.9 | 88.5 | 165.2 |
| 131 | NS144-107 | 47.5 | 106.3 | 7.2 | 72.7 | 17.4 |
| 132 | NS144-108 | 84.6 | 95.4 | 24.0 | 50.9 | 93.0 |
| 133 | NS144-109 | 291.6 | 77.2 | 166.5 | 38.1 | 896.8 |
| 134 | NS144-110 | 283.6 | 87.4 | 139.9 | 51.1 | 1046.0 |
| 135 | YS135-52 | 3538.0 | 77.8 | 1611.0 | N.D. | N.D. |
| 136 | YS135-53 | 1809.0 | 92.5 | 529.5 | N.D. | N.D. |
| 137 | YS135-54 | 530.9 | 74.4 | 57.6 | 44.6 | 372.4 |
| 138 | YS135-80 | 147.3 | 79.0 | 118.1 | 100.9 | 1421.0 |
| 139 | YS135-81 | 4180.0 | 112.0 | 784.9 | N.D. | N.D. |
| 140 | YS135-82 | 1131.0 | 62.6 | 247.0 | N.D. | N.D. |
| 141 | YS135-96 | 739.0 | 91.3 | 630.9 | N.D. | N.D. |
| 142 | YS135-98 | 152.3 | N.D. | N.D. | N.D. | N.D. |
| 143 | YS135-99 | 9760.0 | N.D. | N.D. | N.D. | N.D. |
| 144 | YS135-100 | 8248.9 | 67.2 | 1181.0 | N.D. | N.D. |
| 145 | ZX147-026-01 | 123.0 | 64.4 | 23.3 | 42.4 | 77.0 |
| 146 | ZX147-026-02 | 170.8 | 54.5 | 51.7 | 56.7 | 1529.0 |
| 147 | ZX147-027 | >10,000 | 62.2 | 1367.0 | N.D. | N.D. |
| 148 | ZX147-028 | 221.6 | 78.0 | 51.7 | 33.60 | 153.7 |
| 149 | ZX147-029 | 363.7 | 49.8 | 10.1 | 24.2 | 113.5 |
| 150 | ZX147-031 | 842.5 | 95.3 | 13.3 | 60.9 | 29.9 |
| 151 | ZX147-054 | 198.4 | 38.8 | 61.1 | N.D. | N.D. |
| 152 | ZX147-055 | 389.0 | 89.8 | 1574.0 | 66.45 | 1050 |
| 153 | ZX147-056 | 634.0 | 83.9 | 1243.0 | 77.42 | 1197 |
| 153 | ZX147-092 | 196.9 | 66.8 | 58.9 | 27.40 | 142.0 |
| 155 | ZX147-093 | 539.4 | 68.5 | 29.5 | 34.38 | 532.4 |
| 156 | ZX147-094 | 163.8 | 103.2 | 20.2 | 58.43 | 61.18 |
| 157 | ZX147-095 | 163.9 | 78.0 | 93.0 | 39.30 | 500.0 |
| 158 | ZX147-096 | 368.3 | 82.2 | 78.4 | 32.60 | 120.3 |
| 159 | ZX147-097 | 125.5 | 90.3 | 17.5 | 39.66 | 66.88 |
| 160 | ZX147-098 | 150.6 | 84.1 | 74.5 | 30.93 | 144.3 |
| 161 | ZX147-099 | 488.2 | 93.2 | 84.1 | 35.22 | 311.3 |
| 162 | ZX147-100 | 1137.0 | 80.6 | 260.1 | 23.38 | 302.6 |
| 163 | ZX147-128 | >10,000 | 82.4 | 604.4 | N.D. | N.D. |
| 164 | ZX147-129 | >10,000 | 52.9 | 517.8 | 23.28 | 836.4 |
| 165 | ZX147-130 | 342.3 | 65.0 | 85.8 | 28.67 | 157.2 |
| 166 | ZX147-131 | 306.6 | 64.0 | 47.1 | 24.85 | 120.2 |
| 167 | ZX147-137 | 524.5 | 25.7 | 173.2 | N.D. | N.D. |
| 168 | ZX147-183 | 8636.0 | 68.2 | 183.2 | 41.60 | 1052 |
| 169 | ZX156-011 | >10,000 | N.D. | N.D. | N.D. | N.D. |
| 170 | ZX156-012 | >10,000 | N.D. | N.D. | N.D. | N.D. |
| 171 | ZX156-014-1 | >10,000 | N.D. | N.D. | N.D. | N.D. |
| 172 | ZX156-014-2 | >10,000 | 66.4 | 4016.0 | N.D. | N.D. |
| 173 | ZX156-019 | 838.8 | 57.3 | 84.6 | N.D. | N.D. |
| 174 | ZX156-059 | 9760.0 | N.D. | N.D. | N.D. | N.D. |
| 175 | ZX156-069 | 8249.0 | 46.3 | 88.8 | 31.6 | 343.4 |
| 176 | ZX156-070 | >10,000 | N.D. | N.D. | N.D. | N.D. |
| 177 | ZX156-071 | 1831.0 | N.D. | N.D. | N.D. | N.D. |
| 178 | ZX156-089 | >10,000 | N.D. | N.D. | N.D. | N.D. |
| 179 | ZX156-090 | 8616.0 | N.D. | N.D. | N.D. | N.D. |
| 180 | ZX162-100 | 227.3 | 109.0 | 174.4 | N.D. | N.D. |
| 181 | ZX162-031 | 842.5 | 61.26 | 120.0 | N.D. | N.D. |
| 182 | ZX162-104 | 2465.0 | 78.9 | 786.4 | N.D. | N.D. |
| 183 | ZX162-105 | 595.4 | 69.7 | 251.0 | N.D. | N.D. |
| 184 | ZX162-110 | 5676.0 | 102.9 | 423.4 | 68.2 | 1972.0 |
| 185 | ZX162-111 | 6743.0 | 78.1 | 289.9 | 46.07 | 542.3 |
| 186 | ZX162-112 | 748.5 | 98.8 | 231.0 | 46.85 | 481.3 |
| 187 | ZX162-113 | 699.1 | 71.6 | 136.4 | 34.96 | 647.8 |
| 188 | ZX162-124 | 2105.0 | 83.7 | 1456.0 | N.D. | N.D. |
| 189 | ZX162-126 | 6925.0 | N.D. | N.D. | N.D. | N.D. |
| 190 | ZX162-127 | 5368.0 | N.D. | N.D. | N.D. | N.D. |
| 191 | ZX162-128 | 3044.0 | 83.0 | 1617.0 | N.D. | N.D. |
| 192 | ZX162-129 | >10,000 | N.D. | N.D. | N.D. | N.D. |
| 193 | ZX162-138 | >10,000 | N.D. | N.D. | N.D. | N.D. |
| 194 | ZX162-139 | 2476.0 | 64.4 | 794.7 | N.D. | N.D. |
| 195 | ZX162-140 | >10,000 | 77.4 | 1086.0 | 21.66 | 1040.0 |
| 196 | ZX162-141 | 910.0 | 67.0 | 220.4 | N.D. | N.D. |
| 197 | ZX162-147 | 1197.0 | N.D. | N.D. | N.D. | N.D. |
| 198 | ZX162-148 | 1453.0 | 45.1 | 1159.0 | N.D. | N.D. |
| 199 | ZX162-151 | 3435.0 | 87.1 | 173.9 | 76.5 | 955.9 |
| 200 | ZX162-173 | >10,000 | N.D. | N.D. | N.D. | N.D. |
| 201 | ZX162-174 | 4308 | N.D. | N.D. | N.D. | N.D. |
| 202 | ZX162-175 | >10,000 | N.D. | N.D. | N.D. | N.D. |
| 203 | ZX162-176 | 1080.0 | 39.5 | 700.5 | N.D. | N.D. |
| 204 | YX143-103B | 158.3 | N.D. | N.D. | N.D. | N.D. |
| 205 | YX143-103C | 216.6 | N.D. | N.D. | N.D. | N.D. |
| 206 | YX143-105C | 272.1 | 116.7 | 14.4 | 107.8 | 44.8 |
| 207 | YX143-108 | 203.0 | 94.7 | 50.9 | 47.8 | 128.9 |
| 208 | YX143-110B | >10,000 | 95.7 | 849.3 | 48.9 | 1898.0 |
| 209 | YX143-112B | 160.4 | 100.5 | 8.1 | 88.1 | 60.7 |
| 210 | YX143-129 | 86.1 | 74.9 | 33.1 | 33.6 | 119.0 |
| 211 | YX143-134C | 1184.0 | 59.9 | 732.0 | N.D. | N.D. |
| 212 | YX143-138C | 6236.0 | 72.5 | 654.5 | N.D. | N.D. |
| 213 | YX143-182C-1 | 1768.0 | 61.3 | 72.7 | 31.5 | 210.7 |
| 214 | YX143-183A | >10,000 | N.D. | N.D. | N.D. | N.D. |
| 215 | YX143-184B-1 | 3278.0 | N.D. | N.D. | N.D. | N.D. |
| 216 | YX143-184B-2 | 4386.0 | 85.1 | 27.5 | 53.8 | 729.3 |
| 217 | YX143-185B | >10,000 | N.D. | N.D. | N.D. | N.D. |
| 218 | YX143-186B | 1723.0 | N.D. | N.D. | N.D. | N.D. |
| 219 | YX157-19A | 312.8 | 55.5 | 25.5 | 71.9 | 536.6 |
| 220 | YX157-20A | >10,000 | N.D. | N.D. | N.D. | N.D. |
| 221 | YX157-29B | 1397.0 | 59.3 | 108.4 | 66.89 | 1316 |
| 222 | YX157-42B | 6976.0 | N.D. | N.D. | N.D. | N.D. |
| 223 | YX157-51B | 1918.0 | 89.7 | 717.3 | N.D. | N.D. |
| 224 | YX157-51C | 8879.0 | N.D. | N.D. | N.D. | N.D. |
| 225 | YX157-55A | 2113.0 | 105.0 | 500.4 | N.D. | N.D. |
| 226 | XS159-153 | 4629.0 | 91.0 | 1350 | 52.13 | 2005 |
| 227 | XS159-155 | 1450.0 | 32.5 | 684.1 | N.D. | N.D. |
| 228 | XS159-160 | 5241.0 | 76.4 | 1073 | N.D. | N.D. |
| 229 | XS159-163 | 1900.0 | 83.7 | 235.9 | 90.2 | 2067.0 |
| 230 | XS159-180 | 714.8 | 66.1 | 248.1 | 45.3 | 917.3 |
| 231 | XS159-186 | 3014.0 | N.D. | N.D. | N.D. | N.D. |
| 232 | XS165-3 | 1540.0 | 48.4 | 170.0 | 29.0 | 1973.0 |
| 233 | XS165-5 | 4832.0 | N.D. | N.D. | N.D. | N.D. |
| 234 | XS165-8 | 259.4 | N.D. | N.D. | N.D. | N.D. |
| 235 | XQ148-93 | 5548.0 | N.D. | N.D. | N.D. | N.D. |
| 236 | XQ158-012 | 293.6 | N.D. | N.D. | N.D. | N.D. |
| 237 | XQ158-055 | 389.0 | 37.7 | 33.6 | 39.8 | 1581.0 |
| 238 | XQ158-056 | 634.0 | 42.2 | 100.7 | 50.0 | 1023.0 |
| 239 | XQ158-078 | 603.1 | N.D. | N.D. | N.D. | N.D. |
| 240 | XQ158-093A | 378.4 | N.D. | N.D. | N.D. | N.D. |
| 241 | XQ158-082 | 375.7 | 63.4 | 65.00 | N.D. | N.D. |
| 242 | XQ158-115 | 329.6 | N.D. | N.D. | N.D. | N.D. |
| 243 | XQ158-164 | 27.18 | 41.9 | 104.4 | N.D. | N.D. |
| 244 | XQ158-167 | 1555.0 | 77.2 | 397.3 | N.D. | N.D. |
| 245 | XQ158-168 | 600.4 | 87.5 | 391.8 | 44.79 | 1302 |
| 246 | ZD160-34 | 2141.0 | 69.6 | 620.0 | N.D. | N.D. |
| 247 | ZD160-140 | 2558.0 | N.D. | N.D. | N.D. | N.D. |
| 248 | ZD160-141 | 5888.0 | N.D. | N.D. | N.D. | N.D. |
| 249 | ZD160-149 | 1015.0 | 72.2 | 118.4 | N.D. | N.D. |
| 250 | ZD160-11 | 1628.0 | 73.1 | 1286.0 | N.D. | N.D. |
| 251 | ZD160-133 | 1103.0 | N.D. | N.D. | N.D. | N.D. |
| 252 | ZD160-130 | 6017.0 | N.D. | N.D. | N.D. | N.D. |
| 253 | ZD160-131 | >10,000 | N.D. | N.D. | N.D. | N.D. |
| 254 | QC166-005 | 1085.0 | 88.3 | 294.3 | 77.6 | 1456.0 |
| 255 | QC166-008 | 158.9 | 81.1 | 68.18 | 42.30 | 302.4 |
| 256 | QC-166-032 | 2014.0 | 97.5 | 366.6 | 31.68 | 1003.0 |
| 257 | XQ148-86 | 97.0 | 90.7 | 44.9 | 52.1 | 104.6 |
| 258 | QC166-096 | 674.4 | 82.1 | 444.2 | N.D. | N.D. |
| 259 | QC166-097 | 168.2 | 81.6 | 943.8 | N.D. | N.D. |
| 260 | QC179-001 | 356.5 | 63.1 | 422.0 | N.D. | N.D. |
| 261 | QC179-002 | 1684.0 | 69.3 | 195.6 | 26.1 | 1647.0 |
| 262 | QC179-025 | 1907.0 | 114.0 | 316.0 | N.D. | N.D. |
| 263 | QC-179-032 | 244.3 | 63.7 | 127.1 | N.D. | N.D. |
| 264 | QC-179-033 | 98.75 | 44.7 | 164.8 | N.D. | N.D. |
| 265 | QC179-038 | 217.1 | N.D. | N.D. | N.D. | N.D. |
| 266 | QC179-039 | 244.3 | N.D. | N.D. | N.D. | N.D. |
| 267 | QC179-040 | 174.9 | 64.0 | 948.6 | 20.8 | 1055.0 |
| 268 | ZX167-072 | 5936.0 | 98.0 | 740.3 | N.D. | N.D. |
| 269 | ZX167-077 | 743.4 | 88.8 | 257.2 | 29.96 | 1378 |
| 270 | ZX167-074 | 133.9 | 93.8 | 29.79 | 21.22 | 821.6 |
| 271 | ZX167-090 | 3151.0 | 80.1 | 326.7 | N.D. | N.D. |
| 272 | ZX167-091 | 890.2 | 89.5 | 100.2 | 37.99 | 1496.0 |
| 273 | ZX162-100-1 | 6301.0 | 81.0 | 226.2 | 20.73 | 1298.0 |
| 274 | ZX162-100-2 | 1583.0 | 117.2 | 61.47 | 50.96 | 733.3 |
| 275 | ZX162-031-1 | 2772.0 | 91.5 | 654.6 | 24.07 | 1817.0 |
| 276 | ZX162-031-2 | 3131.0 | 97.5 | 366.6 | 31.68 | 1003.0 |
| 277 | ZX167-074-1 | 80.5 | 86.5 | 83.75 | 25.45 | 212.1 |
| 278 | ZX167-074-2 | 33.0 | 58.2 | 9.227 | N.D. | N.D. |
| 279 | ZX177-057 | 75.2 | 98.6 | 109.6 | 60.90 | 380.2 |
| 280 | ZX177-058 | 63.8 | 81.6 | 38.1 | 55.6 | 255.9 |
| 281 | ZX177-058BY | 61.9 | 66.3 | 52.06 | 27.90 | 368.7 |
| 282 | ZX177-059 | 257.3 | 96.8 | 165.0 | 44.8 | 1284.0 |
| 283 | ZX177-060 | 140.9 | 104.4 | 88.2 | 41.5 | 797.0 |

N.D.:
Not Determined; is used for compounds that did either not produce a response or did not produce a response from which reliable measurements of the Eₘₐₓ and/or EC₅₀ could be determined over the tested concentration range.

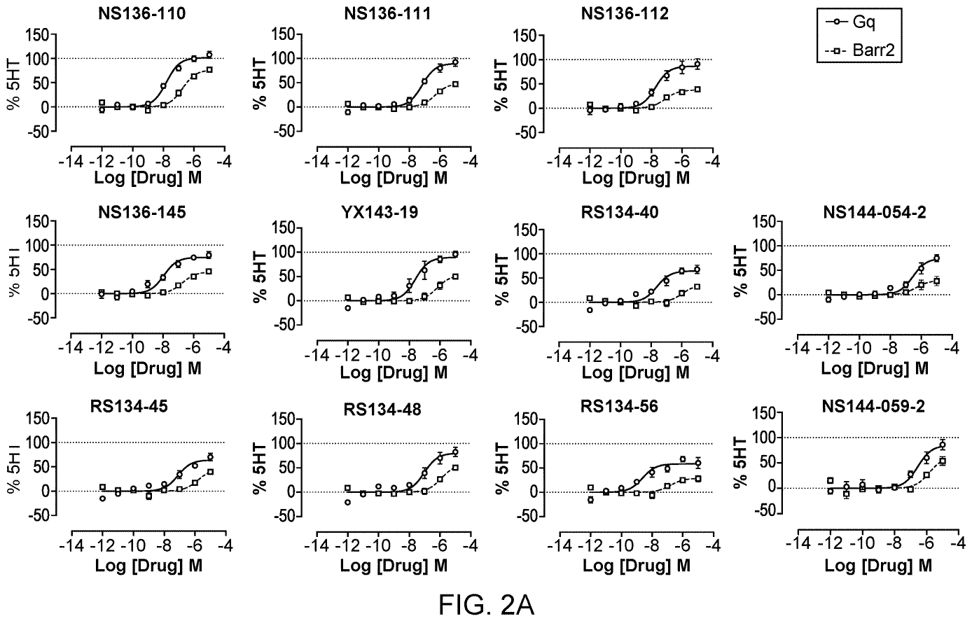

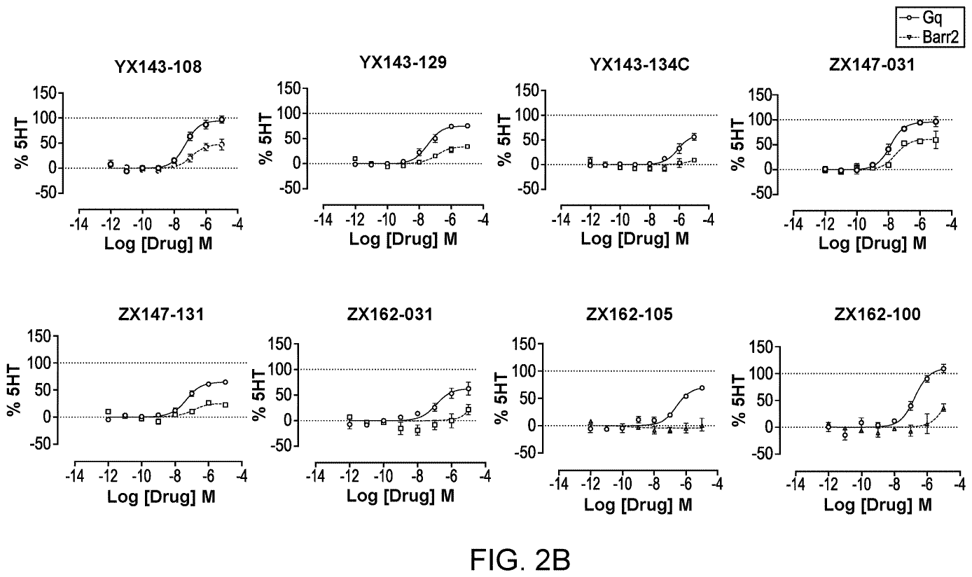

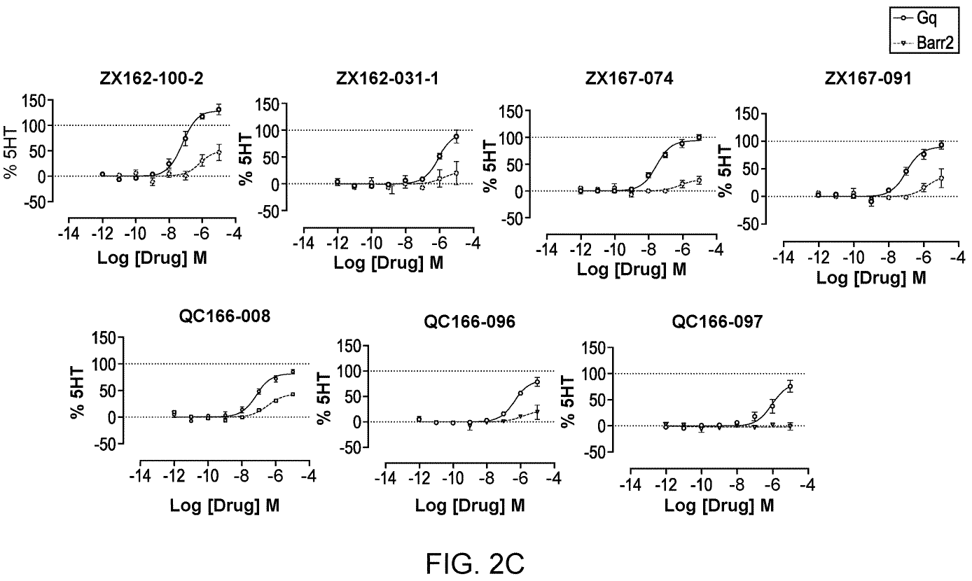

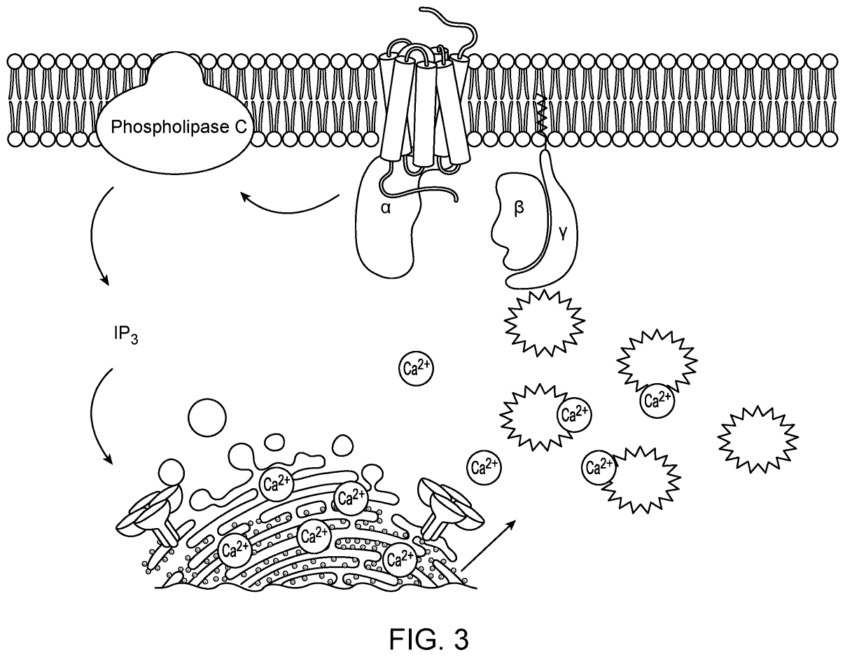

Example 285: Several compounds display 5HT2A biased signaling towards Gαq (blue) versus beta-arrestin2 (red) signaling as measured in the BRET assays (FIG. 2). Biased signaling is represented by either preferential efficacy, potency, or both through the G protein over beta-arrestin2 pathway.

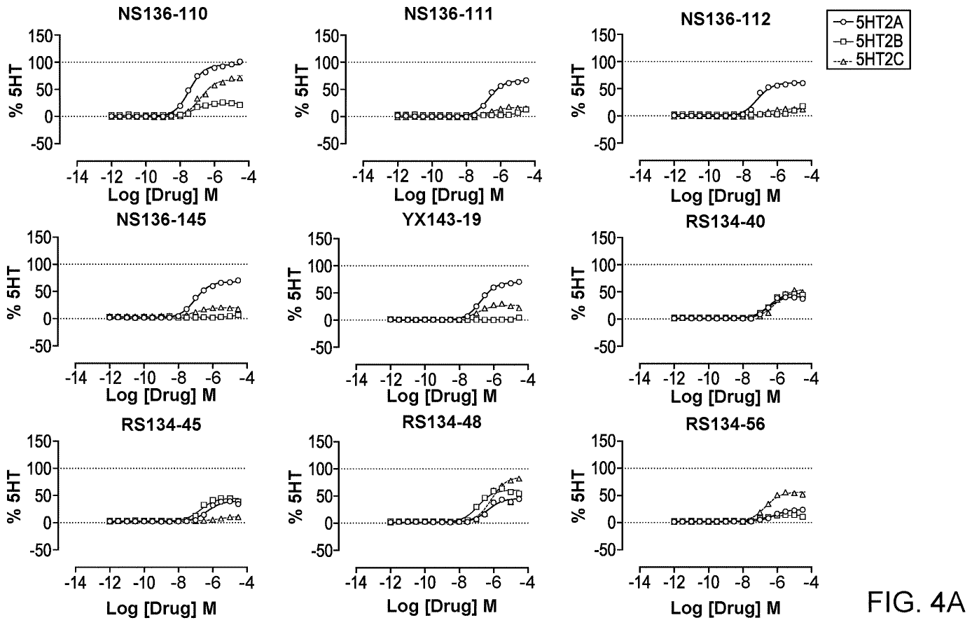

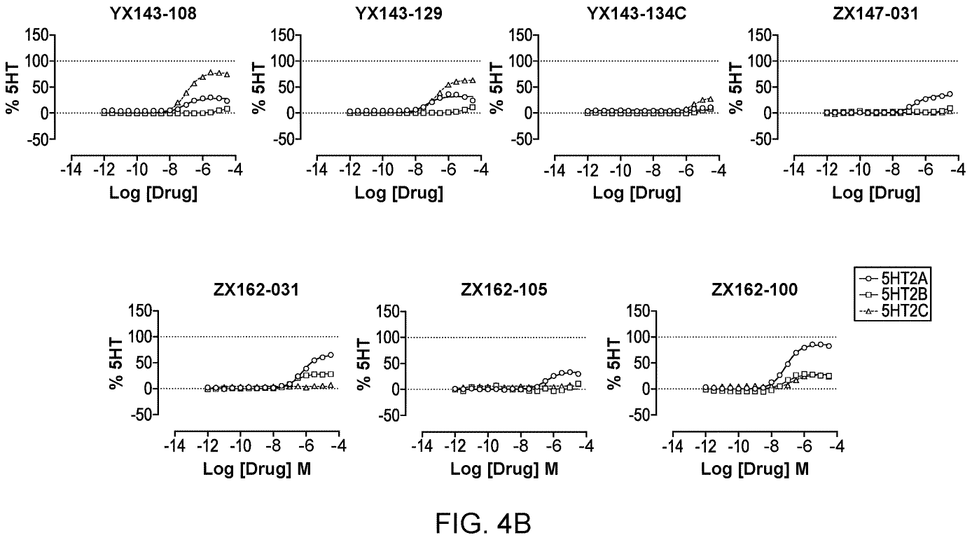

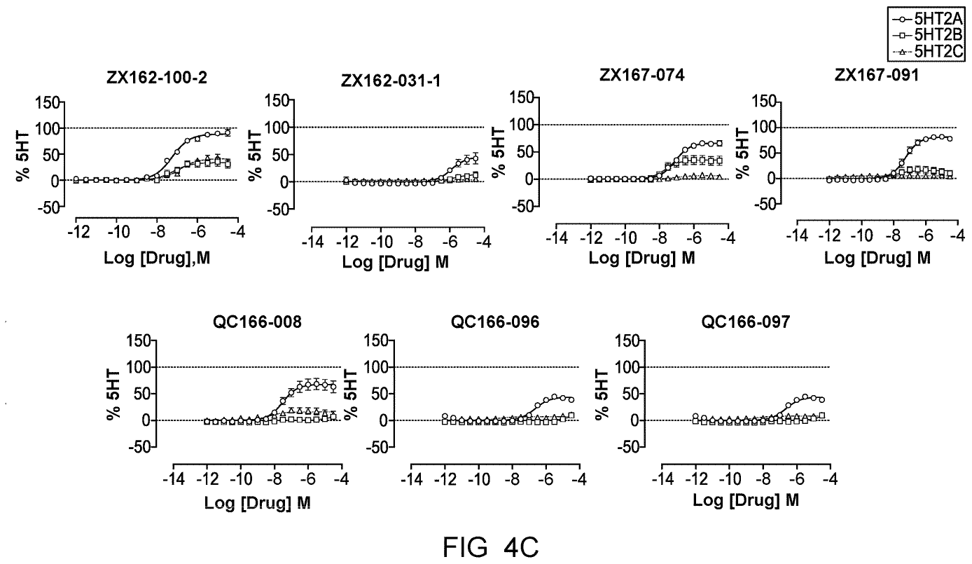

Example 286: Several compounds display selective activation 5HT2A alone as compared by compound-induced calcium flux (calcium mobilization assay) at 5HT2A (blue), 5HT2B (red), 5HT2C (green) (FIG. 4).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Hutcheson, J. D., Setola, V., Roth, B. L., and Merryman, W. D. (2011). Serotonin receptors and heart valve disease—it was meant 2B. Pharmacol Ther 132, 146-157. 10.1016/j.pharmthera.2011.03.008.
Kim, K., Che, T., Panova, O., DiBerto, J. F., Lyu, J., Krumm, BE., Wacker, D., Robertson, M. J., Seven, A. B., Nichols, D. E., et al. (2020). Structure of a Hallucinogen-Activated Gq-Coupled 5-HT2A Serotonin Receptor. Cell 182, 1574-1588 e1519. 10.1016/j.cell.2020.08.024.
McCorvy, J. D., and Roth, B. L. (2015). Structure and function of serotonin G protein-coupled receptors. Pharmacol Ther 150, 129-142. 10.1016/j.pharmthera.2015.01.009.
Roth, B. L. (2007). Drugs and valvular heart disease. N Engl J Med 356, 6-9. 10.1056/NEJMp068265.
Slocum, S. T., DiBerto, J. F., and Roth, B. L. (2021). Molecular Insights into Psychedelic Drug Action. J Neurochem. 10.1111/jnc.15540.

What is claimed is:

1. A 5HT2A agonist, comprising a compound having the structure of FORMULA 1, 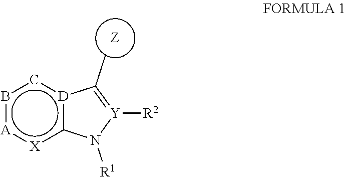
wherein,
A is selected from N, CH or CR⁶;
B is selected from N, CH or CR⁵;
C is selected from N, CH or CR⁴;
D is selected from N or C;
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R¹³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R¹³, NR¹⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring; and
\textcircled{z} is at each occurrence independently selected from an optionally substituted 3-10 membered carbocyclyl, optionally substituted 3-10 membered heterocyclyl, optionally substituted 4-13 membered fused carbocyclyl, optionally substituted 4-13 membered fused heterocyclyl, optionally substituted 4-13 membered bridged carbocyclyl, optionally substituted 4-13 membered bridged heterocyclyl, optionally substituted 4-13 membered spiro carbocyclyl, optionally substituted 4-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl, and
pharmaceutically acceptable salts thereof.

2. A 5HT2A agonist, comprising a compound having the structure of FORMULA 1, 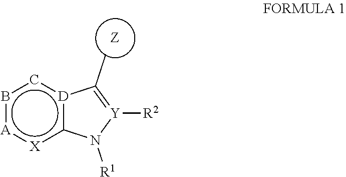
wherein,
A is selected from N, CH or CR⁶;
B is selected from N, CH or CR⁵;
C is selected from N, CH or CR⁴;
D is selected from N or C;
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR², SR²³, NR²³R²⁴, C(O)R²³, C(O)OR²³, C(O)NR²³R²⁴, S(O)R²³, S(O)₂R²³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring; and
\textcircled{z} at each occurrence is selected from the following groups, or their optionally substituted analogs, wherein * denotes the point of attachment: 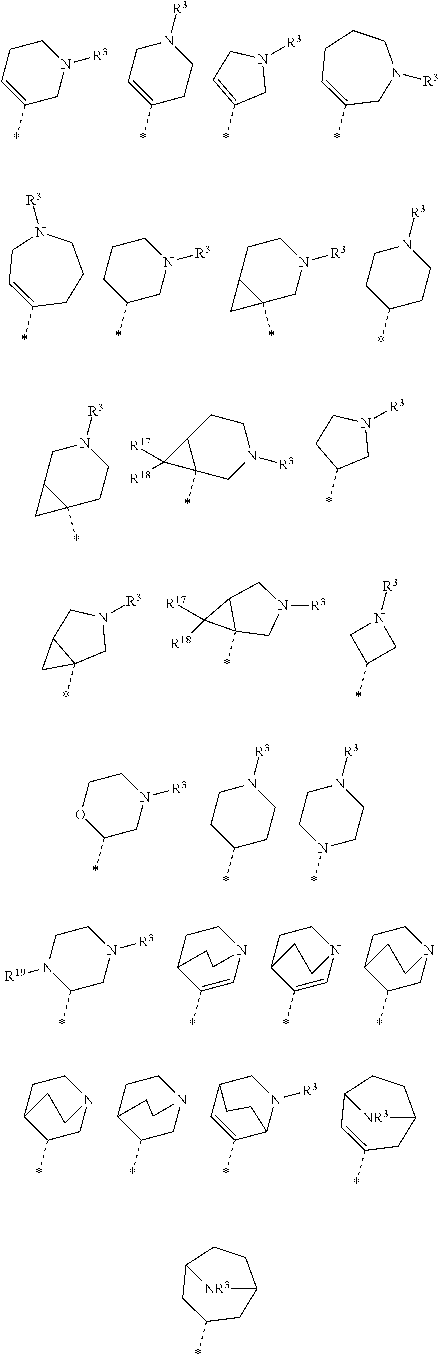
wherein,
R³ at each occurrence, are independently selected from hydrogen, methyl, ethyl, n-propyl, C₁-C₈ alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring;
R¹⁷ and R¹⁸ at each occurrence, are independently selected from hydrogen, halogen, C₁-C₈ alkyl, oxo, Ph, CN, NO₂, OR²⁸, SR²⁸, NR²⁸R²⁹, C(O)R²⁸, C(O)OR²⁹, C(O)NR²⁸R²⁹, S(O)R²⁸, S(O)₂R²⁸, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³¹S(O)₂R²⁸, NR³⁰S(O)₂NR²⁸R²⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein;
R²⁸, R²⁹, and R³⁰ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁸ and R²⁹, R²⁸ and R³⁰, R²⁹ and R³⁰ together with the atom to which they are connected form an optionally substituted 3-20 membered cycloalkyl or heterocyclyl ring; and
pharmaceutically acceptable salts thereof.

3. A 5HT2A agonist, comprising a compound having the structure of FORMULA 2, 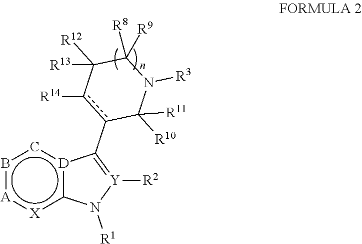
wherein
A is selected from N, CH or CR⁶;
B is selected from N, CH or CR⁵;
C is selected from N, CH or CR⁴;
D is selected from N or C;
X is selected from N, CH or CR⁷;
Y is selected from N or C;
n is selected from 0, 1 or 2;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R¹³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring;
R⁸, R⁹, R¹⁰, R¹¹, R¹², R¹³ and R¹⁴, at each occurrence, are independently selected from hydrogen, halogen, C₁-C₈ alkyl, oxo, Ph, CN, NO₂, OR³¹, SR³¹, NR³¹R³², C(O)R³¹, C(O)OR³¹, C(O)NR³¹R³², S(O)R³¹, S(O)₂R³¹, S(O)₂NR³¹R³², NR³³C(O)OR³, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³¹ and R³², R³¹ and R³³, R³² and R³³ together with the atom to which they are connected form an optionally substituted 3-8 membered cycloalkyl or heterocyclyl ring;
R³ is selected from hydrogen, methyl, ethyl, n-propyl, C₁-C₈alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring; and
pharmaceutically acceptable salts thereof.

4. A 5HT2A agonist, comprising a compound having the structure of FORMULA 2A or FORMULA 2B, 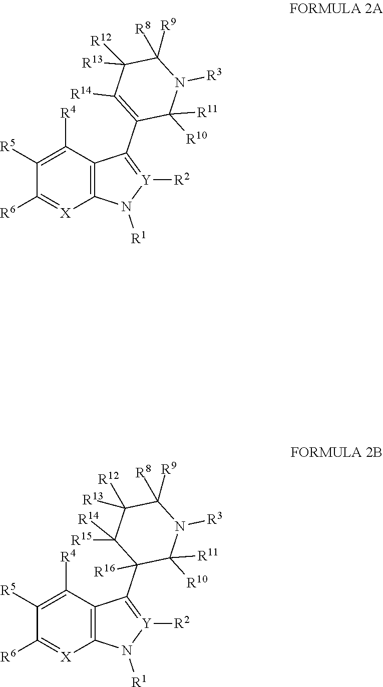
wherein;
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R¹³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R¹³, NR¹⁵S(O)₂NR¹³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring;
R⁸, R⁹, R¹⁰, Ru, R¹, R¹³ and R¹⁴, at each occurrence, are independently selected from hydrogen, halogen, C₁-C₈ alkyl, oxo, Ph, CN, NO₂, OR³¹, SR³, NR³¹R³², C(O)R³¹, C(O)OR³, C(O)NR³¹R³², S(O)R³¹, S(O)₂R³¹, S(O)₂NR³¹R³², NR³³C(O)OR³, NR³³C(O)R³¹, NR³³C(O)NR³¹R³², NR³³S(O)R³, NR³³S(O)₂R³, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³¹ and R³², R³¹ and R³³, R³² and R³³ together with the atom to which they are connected form an optionally substituted 3-8 membered cycloalkyl or heterocyclyl ring;
R³ is selected from hydrogen, methyl, ethyl, n-propyl, C₁-C₈ alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring;
R¹⁵ and R¹⁶, at each occurrence, are independently selected from hydrogen, halogen, C₁-C₈ alkyl, oxo, Ph, CN, N₀₂, OR³⁴, SR³⁴, NR³⁴R³⁵, C(O)R³⁴, C(O)OR³⁴, C(O)NR³⁴R³⁵, S(O)R³⁴, S(O)₂R³⁴, S(O)₂NR³⁴R³⁵, NR³⁶C(O)OR³⁴, NR³⁶C(O)R³⁴, NR³⁶C(O)NR³⁴R³⁵, NR³⁶S(O)R³⁴, NR³⁶S(O)₂R³⁴, NR³⁶S(O)₂NR³⁴R³⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³⁴, R³, and R³⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³⁴ and R³⁵, R³⁴ and R³⁶, R³⁵ and R³⁶ together with the atom to which they are connected form an optionally substituted 3-8 membered cycloalkyl or heterocyclyl ring;
optionally, R¹⁵ and R¹⁶ together with the atom to which they are connected form an optionally substituted 3-8 membered cycloalkyl or heterocyclyl ring; and
pharmaceutically acceptable salts thereof.

5. A 5HT2A agonist, comprising a compound having the structure of FORMULA 2C or FORMULA 2D, 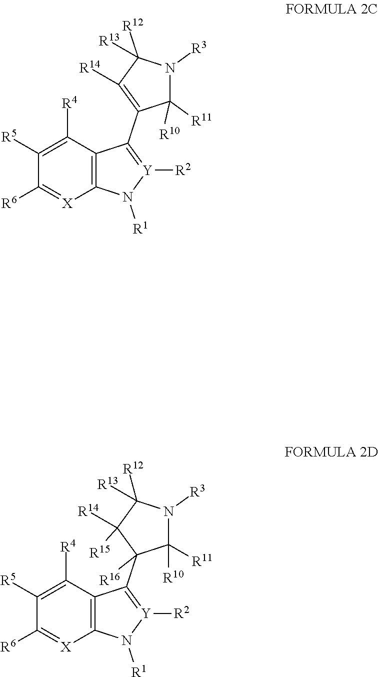
wherein,
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR¹, C(O)NR²³R²⁴, S(O)R²³, S(O)₂R²³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring;
R⁸, R⁹, R¹⁰, R¹¹, R¹², R¹³ and R¹⁴, at each occurrence, are independently selected from hydrogen, halogen, C₁-C₈ alkyl, oxo, Ph, CN, NO₂, OR³¹, SR³¹, NR³¹R³², C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³¹ and R³², R³¹ and R³³, R³² and R³³ together with the atom to which they are connected form an optionally substituted 3-8 membered cycloalkyl or heterocyclyl ring;
R³ is selected from hydrogen, methyl, ethyl, n-propyl, C₁-C₈ alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring;
R¹⁵ and R¹⁶, at each occurrence, are independently selected from hydrogen, halogen, C₁-C₈ alkyl, oxo, Ph, CN, NO₂, OR³⁴, SR³⁴, NR³⁴R³⁵, C(O)R³⁴, C(O)OR³⁴, C(O)NR³⁴R³⁵, S(O)R³⁴, S(O)₂R³⁴, S(O)₂NR³⁴R³⁵, NR³⁶C(O)OR³⁴, NR³⁶C(O)R³⁴, NR³⁶C(O)NR³⁴R³⁵, NR³⁶S(O)R³⁴, NR³⁶S(O)₂R³⁴, NR³⁶S(O)₂NR³⁴R³⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³⁴, and R³⁵, and R³⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³⁴ and R³⁵, R³⁴ and R³⁶, R³⁵ and R³⁶ together with the atom to which they are connected form an optionally substituted 3-8 membered cycloalkyl or heterocyclyl ring;
optionally, R¹⁵ and R¹⁶ together with the atom to which they are connected form an optionally substituted 3-8 membered cycloalkyl or heterocyclyl ring; and
pharmaceutically acceptable salts thereof.

6. A 5HT2A agonist, comprising a compound having the structure of FORMULA 2E or FORMULA 2F 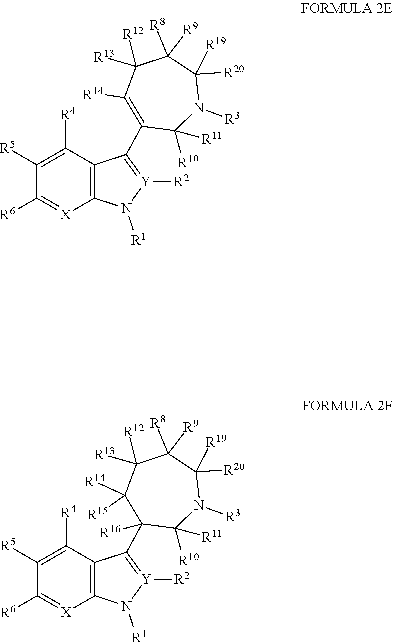
wherein,
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R¹³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring;
R⁸, R⁹, R¹⁰, R¹¹, R¹², R¹³ and R¹⁴, at each occurrence, are independently selected from hydrogen, halogen, C₁-C₈ alkyl, oxo, Ph, CN, NO₂, OR³¹, SR³¹, NR³¹R³², C(O)R³¹, C(O)OR³¹, C(O)NR³¹R³², S(O)R³¹, S(O)₂R³¹, S(O)₂NR³¹R³², NR³³C(O)OR³, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³¹ and R³², R³¹ and R³³, R³² and R³³ together with the atom to which they are connected form an optionally substituted 3-8 membered cycloalkyl or heterocyclyl ring;
R³ is selected from hydrogen, methyl, ethyl, n-propyl, C₁-C₈alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring;
R¹⁵ and R¹⁶, at each occurrence, are independently selected from hydrogen, halogen, C₁-C₈ alkyl, oxo, Ph, CN, NO₂, OR³⁴, SR³⁴, NR³⁴R³⁵, C(O)R³⁴, C(O)OR³⁴, C(O)NR³⁴R³⁵, S(O)R³⁴, S(O)₂R³⁴, S(O)₂NR³⁴R³⁵, NR³⁶C(O)OR³⁴, NR³⁶C(O)R³⁴, NR³⁶C(O)NR³⁴R³⁵, NR³⁶S(O)R³⁴, NR³⁶S(O)₂R³⁴, NR³⁶S(O)₂NR³⁴R³⁵, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³⁴, and R³⁵, and R³⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³⁴ and R³⁵, R³⁴ and R³⁶, R³⁵ and R³⁶ together with the atom to which they are connected form an optionally substituted 3-8 membered cycloalkyl or heterocyclyl ring;
optionally, R¹⁵ and R¹⁶ together with the atom to which they are connected form an optionally substituted 3-8 membered cycloalkyl or heterocyclyl ring;
R¹⁹ and R²⁰, at each occurrence, are independently selected from hydrogen, halogen, C₁-C₈ alkyl, oxo, Ph, CN, NO₂, OR³⁷, SR³⁷, NR³⁷R³⁸, C(O)R³⁷, C(O)OR³⁸, C(O)NR³⁷R³⁸, S(O)R³⁷, S(O)₂R³⁷, S(O)₂NR³⁷R³⁸, NR³⁹C(O)OR³⁷, NR³⁹C(O)R³⁷, NR³⁹C(O)NR³⁷R³⁸, NR³⁹S(O)R³⁷, NR³⁹S(O)₂R³⁷, NR³⁶S(O)₂NR³⁷R³⁸, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³⁷, R³⁸, and R³⁹ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³⁷ and R³⁸, R³⁷ and R³⁹, R³⁸ and R³⁹ together with the atom to which they are connected form an optionally substituted 3-8 membered cycloalkyl or heterocyclyl ring; and
pharmaceutically acceptable salts thereof.

7. A 5HT2A agonist, comprising a compound having the structure of FORMULA 3 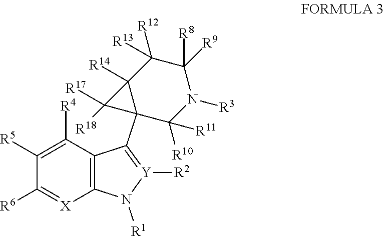
wherein;
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R²³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring;
R³ is selected from hydrogen, methyl, ethyl, n-propyl, C₁-C₈ alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring; and
R¹⁷ and R¹⁸ at each occurrence, are independently selected from hydrogen, halogen, C₁-C₈alkyl, oxo, Ph, CN, NO₂, OR²⁸, SR²⁸, NR²⁸R²⁹, C(O)R²⁸, C(O)OR²⁹, C(O)NR²⁸R²⁹, S(O)R²⁸, S(O)₂R²⁸, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein;
R²⁸, R²⁹, and R³⁰ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁸ and R²⁹, R²⁸ and R³⁰, R²⁹ and R³⁰ together with the atom to which they are connected form an optionally substituted 3-20 membered cycloalkyl or heterocyclyl ring;
R⁸, R⁹, R¹⁰, R¹¹, R¹², R¹³ and R¹⁴, at each occurrence, are independently selected from hydrogen, halogen, C₁-C₈ alkyl, oxo, Ph, CN, NO₂, OR³¹, SR³¹, NR³¹R³², C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³⁹ and R³², R³⁹ and R³³, R³² and R³³ together with the atom to which they are connected form an optionally substituted 3-8 membered cycloalkyl or heterocyclyl ring; and
pharmaceutically acceptable salts thereof.

8. A 5HT2A agonist, comprising a compound having the structure of FORMULA 4 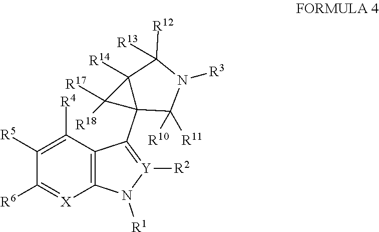
wherein;
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R²³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring;
R³ is selected from hydrogen, methyl, ethyl, n-propyl, C₁-C₈ alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring; and
R¹⁷ and R¹⁸ at each occurrence, are independently selected from hydrogen, halogen, C₁-C₈alkyl, oxo, Ph, CN, NO₂, OR²⁸, SR²⁸, NR²⁸R²⁹, C(O)R²⁸, C(O)OR²⁹, C(O)NR²⁸R²⁹, S(O)R²⁸, S(O)₂R²⁸, S(O)₂NR²⁸R²⁹, NR³⁰C(O)OR²⁸, NR³⁰C(O)R²⁸, NR³⁰C(O)NR²⁸R²⁹, NR³⁰S(O)R²⁸, NR³⁰S(O)₂R²⁸, NR³⁰S(O)₂NR²⁸R²⁹, optionally substituted C₁-C₈alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein;
R²⁸, R²⁹, and R³⁰ are independently selected from hydrogen, optionally substituted C₁-C₈alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁸ and R²⁹, R²⁸ and R³⁰, R²⁹ and R³⁰ together with the atom to which they are connected form an optionally substituted 3-20 membered cycloalkyl or heterocyclyl ring;
R¹⁰, R¹¹, R¹², R¹³ and R¹⁴, at each occurrence, are independently selected from hydrogen, halogen, C₁-C₈ alkyl, oxo, Ph, CN, NO₂, OR³¹, SR³¹, NR³¹R³², C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³¹, NR³³S(O)₂NR³¹R³¹, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³¹ and R³², R³¹ and R³³, R³² and R³³ together with the atom to which they are connected form an optionally substituted 3-8 membered cycloalkyl or heterocyclyl ring; and
pharmaceutically acceptable salts thereof.

9. A 5HT2A agonist, comprising a compound having the structure of FORMULA 5 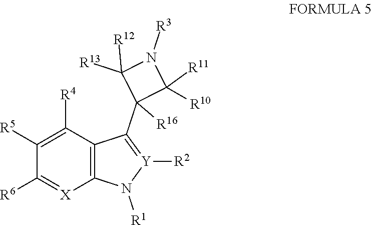
wherein;
X is selected from N, CH or CR⁷;
Y is selected from N or C;
R¹ and R² at each occurrence, are independently selected from null, hydrogen, halogen, C₁-C₈ alkyl, oxo, Ph, C(O)R²¹, C(O)OR²¹, C(O)NR²¹R²², S(O)R²¹, S(O)₂R²¹, S(O)₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²¹ and R²² are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²¹ and R²², together with the atom to which they are connected form an optionally substituted 3-8 membered cycloalkyl or heterocyclyl ring;
R⁴, R⁵, R⁶ and R⁷ at each occurrence, are independently selected from hydrogen, halogen, C₁-C₈ alkyl, oxo, Ph, CN, NO₂, OR²³, SR²³, NR²³R²⁴, C(O)R²³, C(O)OR²³, C(O)NR²³R²⁴, S(O)R²³, S(O)₂R¹³, S(O)₂NR²³R²⁴, NR²⁵C(O)OR²³, NR²⁵C(O)R²³, NR²⁵C(O)NR²³R²⁴, NR²⁵S(O)R²³, NR²⁵S(O)₂R²³, NR²⁵S(O)₂NR²³R²⁴, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²³, R²⁴, and R²⁵ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²³ and R²⁴, R²³ and R²⁵, R²⁴ and R²⁵ together with the atom to which they are connected form an optionally substituted 3-8 membered cycloalkyl or heterocyclyl ring;
R³ is selected from hydrogen, methyl, ethyl, n-propyl, C₁-C₈ alkyl, CD₃, Ph, C(O)R²⁶, C(O)OR²⁶, C(O)NR²⁶R²⁷, S(O)R²⁶, S(O)₂R²⁶, S(O)₂NR²⁶R²⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R²⁶ and R²⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R²⁶ and R²⁷, together with the atom to which they are connected form an optionally substituted 3-8 membered cycloalkyl or heterocyclyl ring; and
R¹⁰, R¹¹, R¹² and R¹³, at each occurrence, are independently selected from hydrogen, halogen, C₁-C₈ alkyl, oxo, Ph, CN, NO₂, OR³¹, SR³¹, NR³¹R³², C(O)R³¹, C(O)OR³¹, C(O)NR³¹R³², S(O)R³¹, S(O)₂R³¹, S(O)₂NR³¹R³², NR³³C(O)OR³¹, NR³³C(O)R³¹, NR³³C(O)NR³¹R³², NR³³S(O)R³¹, NR³³S(O)₂R³, NR³³S(O)₂NR³¹R³², optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈alkyl, optionally substituted C₁-C₈ alkylaminoC₁-C₈alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³¹, R³², and R³³ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³¹ and R³², R³¹ and R³³, R³² and R³³ together with the atom to which they are connected form an optionally substituted 3-8 membered cycloalkyl or heterocyclyl ring;
R¹⁶, at each occurrence, is independently selected from hydrogen, halogen, C₁-C₈ alkyl, oxo, Ph, CN, NO₂, OR³⁴, SR³⁴, NR³⁴R³⁵, C(O)R³⁴, C(O)OR³⁴, C(O)NR³⁴R³⁵, S(O)R³⁴, S(O)₂R³⁴, S(O)₂NR³⁴R³⁵, NR³⁶C(O)OR³⁴, NR³⁶C(O)R³⁴, NR³⁶C(O)NR³⁴R³⁵, NR³⁶S(O)R³⁴, NR³⁶S(O)₂R³⁴, NR³⁶S(O)₂NR³⁴R³⁵, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈ alkyl, optionally substituted C₁-C₈ alkylaminoC₁-C₈ alkyl, optionally substituted 3-8 membered cycloalkyl, optionally substituted C₃-C₈ cycloalkoxy, optionally substituted 3-8 membered heterocyclyl, optionally substituted aryl, and optionally substituted heteroaryl;
wherein
R³⁴, and R³⁵, and R³⁶ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted C₃-C₁₀ cycloalkyl, optionally substituted 3-20 membered heterocyclyl, optionally substituted C₂-C₈ alkenyl, optionally substituted C₂-C₈ alkynyl, optionally substituted aryl, and optionally substituted heteroaryl, or
R³⁴ and R³⁵, R³⁴ and R³⁶, R³⁵ and R³⁶ together with the atom to which they are connected form an optionally substituted 3-8 membered cycloalkyl or heterocyclyl ring;
and pharmaceutically acceptable salts thereof.

10. A 5HT2A agonist comprising a compound selected from the group consisting of:
NS131-179, NS131-178, NS131-177, NS136-006, NS131-169, NS131-168, NS131-167, NS131-173, NS131-180, RS134-52, RS134-48, NS131-185, NS131-170, RS134-45, RS134-40, NS131-184, RS134-49, RS134-53, RS134-41, RS134-46, NS131-172, RS134-38, RS134-65, RS134-62, RS134-70, NS136-081, RS134-73, RS134-72, NS136-092, NS136-091, NS136-096, NS136-095, NS136-102, NS136-101, NS136-115, NS136-116, NS136-117, NS136-118, NS136-119, NS136-120, NS136-109, NS136-110, NS136-111, NS136-112, RS134-37, RS134-56, NS136-002, NS136-004, RS130-132, YX129-177C, YX129-180C, YX143-19, YX143-20, YX143-2, YX143-21, NS144-042, NS144-043, NS144-044, YS135-44, YS135-45, YS135-34, YS135-32, YS135-38, YS135-41, YS135-39, YX143-14A-2, NS144-019, NS144-021, YX143-15, YX143-16, YX143-17C, YX143-18C, NS144-047, NS144-048, NS144-049, NS136-128, NS136-129, NS136-130, NS136-131, NS136-150, NS136-151, NS136-152, NS136-166, NS144-011, NS136-158, NS136-167, NS136-159, NS136-135, NS136-136, NS136-137, NS144-046, NS144-045, NS136-140, NS136-141, NS136-142, NS136-143, NS136-153, NS136-154, NS136-155, NS136-175, NS144-016, NS136-160, NS136-176, NS136-161, NS136-144, NS136-145, NS136-146, NS144-051, NS144-050, YX143-41C, YX143-42C, YX143-43D, NS144-059-2, NS144-054-2, NS144-067, NS144-085, NS144-093, NS144-094, NS144-095, NS144-096, XQ148-012, XQ148-023, ZX147-015, ZX147-016, ZX147-017, ZX147-019, NS144-097, NS144-098, NS144-102, NS144-101, NS144-107, NS144-108, NS144-109, NS144-110, YS135-52, YS135-53, YS135-54, YS135-80, YS135-81, YS135-82, YS135-96, YS135-98, YS135-99, YS135-100, ZX147-026-01, ZX147-026-02, ZX147-027, ZX147-028, ZX147-029, ZX147-031, ZX147-054, ZX147-055, ZX147-056, ZX147-092, ZX147-093, ZX147-094, ZX147-095, ZX147-096, ZX147-097, ZX147-098, ZX147-099, ZX147-100, ZX147-128, ZX147-129, ZX147-130, ZX147-131, ZX147-137, ZX147-183, ZX156-011, ZX156-012, ZX156-014-1, ZX156-014-2, ZX156-019, ZX156-059, ZX156-069, ZX156-070, ZX156-071, ZX156-089, ZX156-090, ZX162-100, ZX162-031, ZX162-104, ZX162-105, ZX162-110, ZX162-111, ZX162-112, ZX162-113, ZX162-124, ZX162-126, ZX162-127, ZX162-128, ZX162-129, ZX162-138, ZX162-139, ZX162-140, ZX162-141, ZX162-147, ZX162-148, ZX162-151, ZX162-173, ZX162-174, ZX162-175, ZX162-176, YX143-103B, YX143-103C, YX143-105C, YX143-108, YX143-110B, YX143-112B, YX143-129, YX143-134C, YX143-138C, YX143-182C-1, YX143-183A, YX143-184B-1, YX143-184B-2, YX143-185B, YX143-186B, YX157-19A, YX157-20A, YX157-29B, YX157-42B, YX157-51B, YX157-51C, YX157-55A, XS159-153, XS159-155, XS159-160, XS159-163, XS159-180, XS159-186, XS165-3, XS165-5, XS165-8, XQ148-93, XQ158-012, XQ158-055, XQ158-056, XQ158-078, XQ158-093A, XQ158-082, XQ158-115, XQ158-164, XQ158-167, XQ158-168, ZD160-34, ZD160-140, ZD160-141, ZD160-149, ZD160-11, ZD160-133, ZD160-130, ZD160-131, QC166-005, QC166-008, QC-166-032, XQ148-86, QC166-096, QC166-097, QC179-001, QC179-002, QC179-025, QC-179-032, QC-179-033, QC179-038, QC179-039, QC179-040, ZX167-072, ZX167-077, ZX167-074, ZX167-090, ZX167-091, ZX162-100-1 (Enantiomer 1 of ZX162-100), ZX162-100-2 (Enantiomer 2 of ZX162-100), ZX162-031-1 (Enantiomer 1 of ZX162-031), ZX162-031-2 (Enantiomer 2 of ZX162-031), ZX167-074-1 (Enantiomer 1 of ZX167-074), ZX167-074-2 (Enantiomer 2 of ZX167-074), ZX177-057, ZX177-058, ZX177-058BY, ZX177-059 and ZX177-060 and pharmaceutically acceptable salts thereof.

11. A 5HT2A agonist comprising a compound selected from the group consisting of:
NS131-179, NS131-178, NS131-177, NS136-006, NS131-169, NS131-168, NS131-167, NS131-173, NS131-180, RS134-52, RS134-48, NS131-185, NS131-170, RS134-45, RS134-40, NS131-184, RS134-49, RS134-53, RS134-41, RS134-46, NS131-172, RS134-38, RS134-65, RS134-62, RS134-70, NS136-081, RS134-73, RS134-72, NS136-092, NS136-091, NS136-096, NS136-095, NS136-102, NS136-101, NS136-115, NS136-116, NS136-117, NS136-118, NS136-119, NS136-120, RS134-37, RS134-56, NS136-002, NS136-004, YS135-34, YS135-32, YS135-38, YS135-41, YS135-39, YX143-14A-2, NS144-019, NS144-021, YX143-15, YX143-16, YX143-17C, YX143-18C, NS144-047, NS144-048, NS144-049, NS136-128, NS136-129, NS136-130, NS136-131, NS136-150, NS136-151, NS136-152, NS136-166, NS144-011, NS136-158, NS136-167, NS136-159, NS136-140, NS136-141, NS136-142, NS136-143, NS136-153, NS136-154, NS136-155, NS136-175, NS144-016, NS136-160, NS136-176, NS136-161, NS144-093, NS144-094, NS144-095, NS144-096, YS135-80, YS135-81, YS135-82, YS135-96, YS135-98 and ZX156-069, and pharmaceutically acceptable salts thereof.

12. A 5HT2A agonist comprising a compound selected from the group consisting of:
RS130-132, YX129-177C, YX129-180C, YX143-19, YX143-20, YX143-2, YX143-21, NS144-042, NS144-043, NS144-044, YS135-44, YS135-45, NS136-135, NS136-136, NS136-137, NS144-046, NS144-045, NS136-144, NS136-145, NS136-146, NS144-051, NS144-050, YX143-41C, YX143-42C, YX143-43D, NS144-059-2, NS144-054-2, NS144-067, NS144-085, XQ148-012, XQ148-023, ZX147-015, ZX147-016, ZX147-017, ZX147-019, NS144-097, NS144-098, NS144-102, NS144-101, NS144-107, NS144-108, NS144-109, NS144-110, YS135-52, YS135-53, YS135-54, YS135-99, YS135-100, ZX147-026-01, ZX147-026-02, ZX147-027, ZX147-028, ZX147-029, ZX147-054, ZX147-055, ZX147-056, ZX147-092, ZX147-093, ZX147-094, ZX147-095, ZX147-096, ZX147-097, ZX147-098, ZX147-099, ZX147-100, ZX147-128, ZX147-129, ZX147-130, ZX147-137, ZX147-183, ZX156-019, ZX156-059, ZX156-070, ZX156-071, ZX156-089, ZX156-090, XQ148-93, XQ158-012, XQ158-055, XQ158-056, XQ148-86, and pharmaceutically acceptable salts thereof.

13. A 5HT2A agonist comprising a compound selected from the group consisting of:
YX143-103B, YX143-103C, YX143-105C, YX143-108, YX143-110B, YX143-112B, YX143-129, YX143-134C, YX143-138C, YX143-182C-1, YX143-183A, YX143-184B-1, YX143-184B-2, YX143-185B, YX143-186B, YX157-19A, YX157-20A, YX157-29B, YX157-42B, YX157-51B, YX157-51C, YX157-55A, and pharmaceutically acceptable salts thereof.

14. A 5HT2A agonist comprising a compound selected from the group consisting of:
XS159-180, XS159-186, XS165-3, XS165-5, XS165-8, XQ158-078, XQ158-093A, XQ158-082, XQ158-115, XQ158-164, XQ158-167, XQ158-168, ZD160-34, ZD160-140, ZD160-141, ZD160-149, ZD160-11, ZD160-133, ZD160-130, ZD160-131, and pharmaceutically acceptable salts thereof.

15. A 5HT2A agonist comprising a compound selected from the group consisting of:
QC166-005, QC166-008, QC-166-032, QC166-096, QC166-097, QC179-001, QC179-002, QC179-025, QC-179-032, QC-179-033, QC179-038, QC179-039, QC179-040, and pharmaceutically acceptable salts thereof.

16. A 5HT2A agonist comprising a compound selected from the group consisting of:
ZX162-100, ZX162-031, ZX162-104, ZX162-105, ZX162-110, ZX162-111, ZX162-112, ZX162-113, ZX162-124, ZX162-126, ZX162-127, ZX162-128, ZX162-129, ZX162-138, ZX162-139, ZX162-140, ZX162-141, ZX162-147, ZX162-148, ZX162-151, ZX162-173, ZX162-174, ZX162-175, ZX162-176, XS159-153, XS159-155, XS159-160, XS159-163, ZX167-072, ZX167-077, ZX167-074, ZX167-090, ZX167-091, ZX162-100-1 (Enantiomer 1 of ZX162-100), ZX162-100-2 (Enantiomer 2 of ZX162-100), ZX162-031-1 (Enantiomer 1 of ZX162-031), ZX162-031-2 (Enantiomer 2 of ZX162-031), ZX167-074-1 (Enantiomer 1 of ZX167-074), ZX167-074-2 (Enantiomer 2 of ZX167-074), ZX177-057, ZX177-058, ZX177-058BY, ZX177-059, ZX177-060, and pharmaceutically acceptable salts thereof.

17. A 5HT2A agonist comprising a compound selected from the group consisting of:
NS136-109, NS136-110, NS136-111, NS136-112, NS136-145, RS134-40, RS134-45, RS134-48, RS134-46, YX143-19, and pharmaceutically acceptable salts thereof.

18. A 5HT2A agonist comprising a compound selected from the group consisting of:
YX143-108, YX143-129, YX143-134C, ZX147-031, ZX147-131, ZX162-031, ZX162-031-1, ZX162-100, ZX162-100-2, ZX162-105, ZX167-074, ZX167-091, QC166-008, QC166-096, QC166-097, and pharmaceutically acceptable salts thereof.

19. A 5HT2A agonist comprising a compound selected from the group consisting of:
ZX162-031, ZX162-031-1, ZX162-100, ZX162-100-2, ZX162-105, ZX167-074, ZX167-091, QC166-008, QC166-096, QC166-097, and pharmaceutically acceptable salts thereof.

20. A 5HT2A agonist comprising a compound selected from the group consisting of:
3-(3-azabicyclo[4.1.0]heptan-1-yl)-7-chloro-1H-indazole (ZX162-031);
3-(3-azabicyclo[3.1.0]hexan-1-yl)-7-chloro-1H-indazole (ZX162-100);
3-(3-azabicyclo[3.1.0]hexan-1-yl)-7-chloro-1H-indole (ZX167-074); and
3-(3-azabicyclo[3.1.0]hexan-1-yl)-7-methyl-1H-indole (ZX167-091) and enantiomers, pharmaceutically acceptable salts, solvent complexes, morphological forms, and deuterated and fluorinated derivatives thereof.

21. A 5HT2A agonist comprising a compound selected from the group consisting of:
3-(azetidin-3-yl)-7-chloro-1H-indole (QC166-008),
3-(azetidin-3-yl)-7-methyl-1H-indole (QC166-096),
3-(azetidin-3-yl)-7-fluoro-1H-indole (QC166-097), and enantiomers, pharmaceutically acceptable salts, solvent complexes, morphological forms, and deuterated and fluorinated derivatives thereof.

22. A pharmaceutical composition, comprising:
a. a 5HT2A agonist according to any one of claims 1-21; and
b. a pharmaceutically acceptable carrier.

23. The pharmaceutical composition of claim 22, formulated to be administered orally, parenterally, intradermally, subcutaneously, topically, and/or rectally.

24. A method of treating a psychiatric disorder, comprising administering to a subject in need thereof, a 5HT2A agonist according to any one of claims 1-23.

25. The method of claim 24, wherein the psychiatric disorder is depression, anxiety, psychosis, dyskinesias, hallucination or substance abuse.

26. A Gq-biased 5HT2A agonist selective for 5HT2A over 5HT2B and SERT, according to any one of claims 1-23.

27. Use of a 5HT2A agonist according to any one of claims 1-20 for the treatment of a psychiatric disorder.

28. A compound according to any one of claims 1-9, wherein Y is N and R² is null.

29. A compound according to any one of claims 1-9, wherein Y is C and R² is hydrogen.

30. A compound according to any one of claims 1-9, 28 and 29, wherein R⁴, R⁵, R⁶ and R⁷ at each occurrence, are independently selected from hydrogen, halogen, C₁-C₈ alkyl, Ph, CN, optionally substituted C₁-C₈ alkyl, optionally substituted C₂-C₈ alkenyl, optionally substituted C₁-C₈ alkoxy, optionally substituted heteroaryl and hydroxy.
